(19) United States
(12) Patent Application Publication
(10) Pub. No.: US 2022/0233568 A1
(43) Pub. Date: Jul. 28, 2022

(54) NOVEL ARTIFICIAL NUCLEIC ACID MOLECULES

(71) Applicant: CureVac AG, Tübingen (DE)

(72) Inventors: Thomas SCHLAKE, Tübingen (DE); Andreas THESS, Tübingen (DE); Moritz THRAN, Tübingen (DE); Frédéric CHEVESSIER-TÜNNESEN, Tübingen (DE); Marion PÖNISCH, Tübingen (DE)

(21) Appl. No.: 16/757,289

(22) PCT Filed: Oct. 17, 2018
(86) PCT No.: PCT/EP2018/078453

§ 371 Date: Apr. 17, 2020

(30) Foreign Application Priority Data
Oct. 19, 2017 (EP) PCT/EP2017/076741
Oct. 19, 2017 (EP) PCT/EP2017/076775
Mar. 23, 2018 (EP) PCT/EP2018/057552
Sep. 26, 2018 (EP) PCT/EP2018/076185

(51) Int. Cl. A61K 31/7088; C12N 15/67; C07K 14/47

(52) U.S. Cl. CPC A61K 31/7088; C07K 14/47; C12N 15/67

(57) ABSTRACT

The present invention provides artificial nucleic acid molecules comprising novel combinations of 5′ and 3′ untranslated region (UTR) elements. The inventive nucleic acid molecules are preferably characterized by increased expression efficacies of coding regions operably linked to said UTR elements. The artificial nucleic acids can be used for treatment or prophylaxis of various diseases. The invention further provides (pharmaceutical) compositions, vaccines and kits comprising said artificial nucleic acid molecules. Further, in vitro methods for preparing artificial nucleic acid molecules according to the invention are provided.

This application is a national phase application under 35 U.S.C. § 371 of International Application No. PCT/EP2018/078453, filed Oct. 17, 2018, which claims benefit of International Application No. PCT/EP2018/076185, filed Sep. 26, 2018, International Application No. PCT/EP2018/057552, filed Mar. 23, 2018, International Application No. PCT/EP2017/076775, filed Oct. 19, 2017, and International Application No. PCT/EP2017/076741, filed Oct. 19, 2017, the entire contents of each of which are hereby incorporated by reference.

To date, therapeutic nucleic acids in the form of naked DNA, viral or bacterial DNA vectors are exploited for a variety of purposes. Gene therapy seeks to treat diseases by transferring one or more therapeutic nucleic acids to a patient's cells (gene addition therapy) or by correcting a defective gene (gene replacement therapy), for example by gene editing. This technology transfer holds the promise of providing lasting therapies for diseases that are not—or only temporarily—curable with conventional treatment options, and even to provide treatments for diseases previously classified as untreatable. Currently available gene therapy strategies are typically based on either in vivo gene delivery to postmitotic target cells or tissues or ex vivo gene delivery into autologous cells followed by adoptive transfer back into the patient (Kumar et al. Mol Ther Methods Clin Dev. 2016; 3: 16034). For some time, clinical gene therapy was characterized by some encouraging results, but also several setbacks. The preferred method of gene delivery, in terms of defined composition and manufacturing reproducibility, would involve naked DNA provided in a suitable carrier such as synthetic particles, for example, using lipids or polymers. However, these methods have not yet achieved efficient uptake and sustained gene expression in vivo. Thus, gene replacement therapy trials that have demonstrated some clinical benefit, relied on viral vectors for gene delivery. Among the various viral based vector systems, adeno-associated virus (AAV) DNA vectors are most commonly used for in vivo gene delivery. The use of retroviral vectors (γ-retroviral or lentivirus derived), which are capable of integrating into the target cells' genome, is somewhat hampered by safety and ethical issues. Concerns regarding retroviral gene therapy are based on the possible generation of replication competent retroviruses during vector production, mobilisation of the vector by endogenous retroviruses in genome, insertional mutagenesis leading to cancer, germline alteration and dissemination of new viruses from gene therapy patients. Although AAV-based vectors generally do not integrate into the patient's genome and thus avoid many of these potential risks, remaining concerns emanate from occasionally observed site-specific integration events, the shedding of vectors from treated patients and potential adverse effects caused by immune responses to viral structural proteins.

Immunotherapy is the second, important field of application for therapeutic nucleic acids. In particular, DNA vaccines encoding tumor antigens have been evaluated for cancer immunotherapy. In principle, harnessing the patient's own adaptive immunity to fight cancer cells seems appealing. DNA-based vaccines based on non-viral DNA vectors can generally be easily engineered and produced rapidly in large quantities. These DNA vectors are stable and can be easily stored and transported. Unlike live attenuated bacterial or viral vaccines, there is no risk of pathogenic infection or the induction of an anti-viral immune response. Naked DNA does not easily spread from cell to cell in vivo. APCs do not readily take up expressed antigens and activate satisfactory immune responses (Yang et al. Hum Vaccin Immunother. 2014 November; 10(11): 3153-3164). On the other hand, the limited uptake and consequent limited antigen-transcription by transfected cells is the major drawback of non-viral DNA-based vaccines. Indeed, anti-tumor vaccination with tumor-antigen encoding DNAs achieved some success in immunization-protection experiments, and several types of anti-cancer vaccines have been designed, manufactured, and pre-clinically tested. However, effectiveness in inducing a measurable immune response and in extending patients' overall survival has been modest in clinical trials.

Administration through electroporation or viral-mediated delivery solves the issue but opens new problems. In the case of electroporation, the availability of clinically approved devices and patients' compliance have limited their use in clinic. In the case of viral-mediated delivery, the problems are mainly related to potential dangers associated with the administration of live virus together with the presence of anti-viral neutralizing antibodies in patients (Lollini et al. Vaccines. 2015 June; 3(2): 467-489).

Since their initial development, nucleic acid-based vaccine and gene therapy technologies have come a long way. Unfortunately, when applied to human subjects inadequate uptake and transcription only achieved limited clinical success due to insufficient gene or antigen expression. Inadequate delivery of therapeutic proteins (in case of gene therapy) or immunogenicity (in case of immunotherapy) are still the biggest challenge for practical use of therapeutic DNAs. Li and Petrovsky Expert Rev Vaccines. 2016; 15(3): 313-329. Although RNA-based therapeutics overcome many of the shortcomings of therapeutic DNAs, there is still room for improvement with regard to the expression efficacies currently observed for available therapeutic RNAs. Thus, effective strategies that help enhance therapeutic nucleic acid potency are urgently needed. It is an object of the present invention to comply with the needs set out above.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±1%, 2%, 3%, 4%, 5%, 6%, 7%, 8%, 9% or 10%.

In the present invention, if not otherwise indicated, different features of alternatives and embodiments may be combined with each other.

For the sake of clarity and readability the following definitions are provided. Any technical feature mentioned for these definitions may be read on each and every embodiment of the invention. Additional definitions and explanations may be specifically provided in the context of these embodiments.

### Definitions

Artificial nucleic acid molecule: An artificial nucleic acid molecule may typically be understood to be a nucleic acid molecule, e.g. a DNA or an RNA, which does not occur naturally. In other words, an artificial nucleic acid molecule may be understood as a non-natural nucleic acid molecule. Such nucleic acid molecule may be non-natural due to its individual sequence (which does not occur naturally) and/or due to other modifications, e.g. structural modifications of nucleotides, which do not occur naturally. An artificial nucleic acid molecule may be a DNA molecule, an RNA molecule or a hybrid-molecule comprising DNA and RNA portions. Typically, artificial nucleic acid molecules may be designed and/or generated by genetic engineering methods to correspond to a desired artificial sequence of nucleotides (heterologous sequence). In this context an artificial sequence is usually a sequence that may not occur naturally, i.e. it differs from the wild type sequence by at least one nucleotide. The term “wild type” may be understood as a sequence occurring in nature. Further, the term “artificial nucleic acid molecule” is not restricted to mean “one single molecule” but is, typically, understood to comprise an ensemble of identical molecules. Accordingly, it may relate to a plurality of identical molecules contained in an aliquot.

DNA: DNA is the usual abbreviation for deoxy-ribonucleic acid. It is a nucleic acid molecule, i.e. a polymer consisting of nucleotides. These nucleotides are usually deoxy-adenosine-monophosphate, deoxy-thymidine-monophosphate, deoxy-guanosine-monophosphate and deoxy-cytidine-monophosphate monomers which are—by themselves—composed of a sugar moiety (deoxyribose), a base moiety and a phosphate moiety, and polymerize by a characteristic backbone structure. The backbone structure is, typically, formed by phosphodiester bonds between the sugar moiety of the nucleotide, i.e. deoxyribose, of a first and a phosphate moiety of a second, adjacent monomer. The specific order of the monomers, i.e. the order of the bases linked to the sugar/phosphate-backbone, is called the DNA sequence. DNA may be single stranded or double stranded. In the double stranded form, the nucleotides of the first strand typically hybridize with the nucleotides of the second strand, e.g. by A/T-base-pairing and G/C-base-pairing.

Heterologous sequence: Two sequences are typically understood to be ‘heterologous’ if they are not derivable from the same gene. I.e., although heterologous sequences may be derivable from the same organism, they naturally (in nature) do not occur in the same nucleic acid molecule, such as in the same mRNA.

Cloning site: A cloning site is typically understood to be a segment of a nucleic acid molecule, which is suitable for insertion of a nucleic acid sequence, e.g., a nucleic acid sequence comprising an open reading frame. Insertion may be performed by any molecular biological method known to the one skilled in the art, e.g. by restriction and ligation. A cloning site typically comprises one or more restriction enzyme recognition sites (restriction sites). These one or more restrictions sites may be recognized by restriction enzymes which cleave the DNA at these sites. A cloning site which comprises more than one restriction site may also be termed a multiple cloning site (MCS) or a poly-linker.

Nucleic acid molecule: A nucleic acid molecule is a molecule comprising, preferably consisting of nucleic acid components. The term nucleic acid molecule preferably refers to DNA or RNA molecules. It is preferably used synonymous with the term “polynucleotide”. Preferably, a nucleic acid molecule is a polymer comprising or consisting of nucleotide monomers, which are covalently linked to each other by phosphodiester-bonds of a sugar/phosphate-backbone. The term “nucleic acid molecule” also encompasses modified nucleic acid molecules, such as base-modified, sugar-modified or backbone-modified etc. DNA or RNA molecules.

Open reading frame: An open reading frame (ORF) in the context of the invention may typically be a sequence of several nucleotide triplets, which may be translated into a peptide or protein. An open reading frame preferably contains a start codon, i.e. a combination of three subsequent nucleotides coding usually for the amino acid methionine (ATG), at its 5′-end and a subsequent region, which usually exhibits a length which is a multiple of 3 nucleotides. An ORF is preferably terminated by a stop-codon (e.g., TAA, TAG, TGA). Typically, this is the only stop-codon of the open reading frame. Thus, an open reading frame in the context of the present invention is preferably a nucleotide sequence, consisting of a number of nucleotides that may be divided by three, which starts with a start codon (e.g. ATG) and which preferably terminates with a stop codon (e.g., TAA, TGA, or TAG). The open reading frame may be isolated or it may be incorporated in a longer nucleic acid sequence, for example in a vector or an mRNA. An open reading frame may also be termed “(protein) coding sequence” or, preferably, “coding sequence”.

Peptide: A peptide or polypeptide is typically a polymer of amino acid monomers, linked by peptide bonds. It typically contains less than 50 monomer units. Nevertheless, the term peptide is not a disclaimer for molecules having more than 50 monomer units. Long peptides are also called polypeptides, typically having between 50 and 600 monomeric units.

Protein A protein typically comprises one or more peptides or polypeptides. A protein is typically folded into 3-dimensional form, which may be required for the protein to exert its biological function.

Restriction site: A restriction site, also termed restriction enzyme recognition site, is a nucleotide sequence recognized by a restriction enzyme. A restriction site is typically a short, preferably palindromic nucleotide sequence, e.g. a sequence comprising 4 to 8 nucleotides. A restriction site is preferably specifically recognized by a restriction enzyme. The restriction enzyme typically cleaves a nucleotide sequence comprising a restriction site at this site. In a double-stranded nucleotide sequence, such as a double-stranded DNA sequence, the restriction enzyme typically cuts both strands of the nucleotide sequence.

RNA, mRNA: RNA is the usual abbreviation for ribonucleic-acid. It is a nucleic acid molecule, i.e. a polymer consisting of nucleotides. These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e. ribose, of a first and a phosphate moiety of a second, adjacent monomer. The specific succession of the monomers is called the RNA-sequence. Usually RNA may be obtainable by transcription of a DNA-sequence, e.g., inside a cell. In eukaryotic cells, transcription is typically performed inside the nucleus or the mitochondria. In vivo, transcription of DNA usually results in the so-called premature RNA which has to be processed into so-called messenger-RNA, usually abbreviated as mRNA. Processing of the premature RNA, e.g. in eukaryotic organisms, comprises a variety of different posttranscriptional-modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino-acid sequence of a particular peptide or protein. Typically, a mature mRNA comprises a 5′-cap, a 5′-UTR, an open reading frame, a 3′-UTR and a poly(A) sequence. Aside from messenger RNA, several non-coding types of RNA exist which may be involved in regulation of transcription and/or translation.

Sequence of a nucleic acid molecule: The sequence of a nucleic acid molecule is typically understood to be the particular and individual order, i.e. the succession of its nucleotides. The sequence of a protein or peptide is typically understood to be the order, i.e. the succession of its amino acids.

Sequence identity: Two or more sequences are identical if they exhibit the same length and order of nucleotides or amino acids. The percentage of identity typically describes the extent to which two sequences are identical, i.e. it typically describes the percentage of nucleotides that correspond in their sequence position with identical nucleotides of a reference-sequence. For determination of the degree of identity (“% identity), the sequences to be compared are typically considered to exhibit the same length, i.e. the length of the longest sequence of the sequences to be compared. This means that a first sequence consisting of 8 nucleotides is 80% identical to a second sequence consisting of 10 nucleotides comprising the first sequence. In other words, in the context of the present invention, identity of sequences preferably relates to the percentage of nucleotides or amino acids of a sequence which have the same position in two or more sequences having the same length. Specifically, the “% identity” of two amino acid sequences or two nucleic acid sequences may be determined by aligning the sequences for optimal comparison purposes (e.g., gaps can be introduced in either sequences for best alignment with the other sequence) and comparing the amino acids or nucleotides at corresponding positions. Gaps are usually regarded as non-identical positions, irrespective of their actual position in an alignment. The “best alignment” is typically an alignment of two sequences that results in the highest percent identity. The percent identity is determined by the number of identical nucleotides in the sequences being compared (i.e., % identity=# of identical positions/total # of positions×100). The determination of percent identity between two sequences can be accomplished using a mathematical algorithm known to those of skill in the art.

Stabilized nucleic acid molecule: A stabilized nucleic acid molecule is a nucleic acid molecule, preferably a DNA or RNA molecule that is modified such, that it is more stable to disintegration or degradation, e.g., by environmental factors or enzymatic digest, such as by an exo- or endonuclease degradation, than the nucleic acid molecule without the modification. Preferably, a stabilized nucleic acid molecule in the context of the present invention is stabilized in a cell, such as a prokaryotic or eukaryotic cell, preferably in a mammalian cell, such as a human cell. The stabilization effect may also be exerted outside of cells, e.g. in a buffer solution etc., for example, in a manufacturing process for a pharmaceutical composition comprising the stabilized nucleic acid molecule.

Transfection: The term “transfection” refers to the introduction of nucleic acid molecules, such as DNA or RNA (e.g. mRNA) molecules, into cells, preferably into eukaryotic cells. In the context of the present invention, the term “transfection” encompasses any method known to the skilled person for introducing nucleic acid molecules into cells, preferably into eukaryotic cells, such as into mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. Preferably, the introduction is non-viral.

Vector: The term “vector” refers to a nucleic acid molecule, preferably to an artificial nucleic acid molecule. A vector in the context of the present invention is suitable for incorporating or harboring a desired nucleic acid sequence, such as a nucleic acid sequence comprising an open reading frame. Such vectors may be storage vectors, expression vectors, cloning vectors, transfer vectors etc. A storage vector is a vector, which allows the convenient storage of a nucleic acid molecule, for example, of an mRNA molecule. Thus, the vector may comprise a sequence corresponding, e.g., to a desired mRNA sequence or a part thereof, such as a sequence corresponding to the coding sequence and the 3′-UTR of an mRNA. An expression vector may be used for production of expression products such as RNA, e.g. mRNA, or peptides, polypeptides or proteins. For example, an expression vector may comprise sequences needed for transcription of a sequence stretch of the vector, such as a promoter sequence, e.g. an RNA polymerase promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector, which is suitable for transferring nucleic acid molecules into cells or organisms, for example, viral vectors. A vector in the context of the present invention may be, e.g., an RNA vector or a DNA vector. Preferably, a vector is a DNA molecule. Preferably, a vector in the sense of the present application comprises a cloning site, a selection marker, such as an antibiotic resistance factor, and a sequence suitable for multiplication of the vector, such as an origin of replication.

Vehicle: A vehicle is typically understood to be a material that is suitable for storing, transporting, and/or administering a compound, such as a pharmaceutically active compound. For example, it may be a physiologically acceptable liquid, which is suitable for storing, transporting, and/or administering a pharmaceutically active compound.

In nature, precise control of gene expression is vital to rapidly adjust to environmental stimuli that alter the physiological status of the cell, like cellular stress or infection. Gene expression programs undergo constant regulation and are tightly regulated by multi-layered regulatory elements acting in both cis and trans. For such precise control the cellular machinery has evolved regulators at several stages from transcription to translation fine-tuning gene expression. These include structural and chemical modifications of chromosomal DNA, transcriptional regulation, post-transcriptional control of messenger RNA (mRNA), varying translational efficiency and protein turnover. These mechanisms in concert determine the spatio-temporal control of genes. Messenger RNA is composed of a protein-coding region, and 5′ and 3 untranslated regions (UTRs). The 3′ UTR is variable in sequence and size; it spans between the stop codon and the poly(A) tail. Importantly, the 3′ UTR sequence harbours several regulatory motifs that determine mRNA turnover, stability and localization, and thus governs many aspects of post-transcriptional gene regulation (Schwerk and Savan. J Immunol. 2015 Oct. 1; 195(7): 2963-2971). In gene therapy and immunotherapy applications, the tight regulation of transgene expression is of paramount importance to therapeutic safety and efficacy. Transgenes need to be expressed in optimal thresholds at the right places. However, the ability to control the level of transgene expression in order to provide a balance between therapeutic efficacy and nonspecific toxicity still remains a major challenge of present gene therapy and immunotherapy applications. The present inventors surprisingly discovered that certain combinations of 5′ and 3′-untranslated regions (UTRs) act in concert to synergistically enhance the expression of operably linked nucleic acid sequences. Artificial nucleic acid molecules harbouring the inventive UTR combinations advantageously enable the rapid and transient expression of high amounts of (poly-)peptides or proteins delivered for gene therapy or immunotherapy purposes. Furthermore, the novel nucleic acid-based therapeutics disclosed herein preferably offer additional advantages over currently available treatment options, including the reduced risk of insertional mutagenesis, and a greater efficacy of non-viral delivery and uptake. Accordingly, the artificial nucleic acids provided herein are particularly useful for various therapeutic applications in vivo, including, for instance gene therapy, cancer immunotherapy or the vaccination against infective agents.

Accordingly, in a first aspect, the present invention thus relates to an artificial nucleic acid molecule comprising at least one 5′ untranslated region (5′ UTR) element derived from a 5′ UTR of a gene selected from the group consisting of HSD17B4, ASAH1, ATP5A1, MP68, NDUFA4, NOSIP, RPL31, SLC7A3, TUBB4B and UBQLN2; at least one 3′ untranslated region (3′ UTR) element derived from a 3′ UTR of a gene selected from the group consisting of PSMB3, CASP1, COX6B1, GNAS, NDUFA1 and RPS9; and optionally at least one coding region operably linked to said 3′ UTR and said 5′ UTR.

The term “UTR” refers to an “untranslated region” located upstream (5′) and/or downstream (3′) a coding region of a nucleic acid molecule as described herein, thereby typically flanking said coding region. Accordingly, the term “UTR” generally encompasses 3′untranslated regions (“3′-UTRs”) and 5′-untranslated regions (“5′-UTRs”). UTRs may typically comprise or consist of nucleic acid sequences that are not translated into protein. Typically, UTRs comprise “regulatory elements”. The term “regulatory element” refers to a nucleic acid sequences having gene regulatory activity, the ability to affect the expression, in particular transcription or translation, of an operably (in cisor trans) linked transcribable nucleic acid sequence. The term includes promoters, enhancers, internal ribosomal entry sites (IRES), introns, leaders, transcription termination signals, such as polyadenylation signals and poly-U sequences and other expression control elements. Regulatory elements may act constitutively or in a time- and/or cell specific manner. Optionally, regulatory elements may exert their function via interacting with (e.g. recruiting and binding) of regulatory proteins capable of modulating (inducing, enhancing, reducing, abrogating, or preventing) the expression, in particular transcription of a gene. UTRs are preferably “operably linked”, i.e. placed in a functional relationship, to a coding region, preferably in a manner that allows them to control (i.e. modulate or regulate, preferably enhance) the expression of said coding sequence. A “UTR” preferably comprises or consists of a nucleic acid sequence, which is derived from the (naturally occurring, wild-type) UTR of a gene, preferably a gene as exemplified herein. The term “UTR element” as used herein typically refers to nucleic acid sequence corresponding to the shorter sub-sequence of the UTR of the parent gene (“parent” UTR). In this context, the term “corresponding to” means that the UTR element may comprise or consist of the RNA sequence transcribed from gene from which the “parent” UTR is derived (i.e. equal to the RNA sequence used for defining said “parent” UTR), or the respective DNA sequence (including sense and antisense strand, mature and immature) equivalent to said RNA sequence, or a mixture thereof.

When referring to an UTR element “derived from” the UTR of a certain gene, the UTR element may be derived from any naturally occurring homolog, variant or fragment of said gene. I.e., when referring to a UTR element “derived from” a HSD17B4 gene, the respective UTR element may consist of a nucleic acid sequence corresponding to a shorter sub-sequence of the UTR of the “parent” HSD17B4 gene, or any HSD17B4 homolog, variant or fragment (in particular including HSD17B4 homologs, variants or fragments including variations in the UTR region as compared to the “parent” HSD17B4 gene).

The term “derived from” as used throughout the present specification in the context of an artificial nucleic acid, i.e. for an artificial nucleic acid “derived from” (another) artificial nucleic acid, also means that the (artificial) nucleic acid, which is derived from (another) artificial nucleic acid, shares e.g. at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid from which it is derived. The skilled person is aware that sequence identity is typically calculated for the same types of nucleic acids, i.e. for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing the uracils (U) by thymidines (T) throughout the sequence) or, vice versa, the DNA sequence is converted into the corresponding RNA sequence (in particular by replacing the T by U throughout the sequence). Thereafter, the sequence identity of the DNA sequences or the sequence identity of the RNA sequences is determined. Preferably, a nucleic acid “derived from” a nucleic acid also refers to nucleic acid, which is modified in comparison to the nucleic acid from which it is derived, e.g. in order to increase RNA stability even further and/or to prolong and/or increase protein production. In the context of amino acid sequences (e.g. antigenic peptides or proteins) the term “derived from” means that the amino acid sequence, which is derived from (another) amino acid sequence, shares e.g. at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence from which it is derived.

The term “homolog” in the context of genes (or nucleic acid sequences derived therefrom or comprised by said gene, like a UTR) refers to a gene (or a nucleic acid sequences derived therefrom or comprised by said gene) related to a second gene (or such nucleic acid sequence) by descent from a common ancestral DNA sequence. The term, “homolog” includes genes separated by the event of speciation (“ortholog”) and genes separated by the event of genetic duplication (“paralog”).

The term “variant” in the context of nucleic acid sequences of genes refers to nucleic acid sequence variants, i.e. nucleic acid sequences or genes comprising a nucleic acid sequence that differs in at least one nucleic acid from a reference (or “parent”) nucleic acid sequence of a reference (or “parent”) nucleic acid or gene. Variant nucleic acids or genes may thus preferably comprise, in their nucleic acid sequence, at least one mutation, substitution, insertion or deletion as compared to their respective reference sequence. Preferably, the term “variant” as used herein includes naturally occurring variants, and engineered variants of nucleic acid sequences or genes. Therefore, a “variant” as defined herein can be derived from, isolated from, related to, based on or homologous to the reference nucleic acid sequence. “Variants” may preferably have a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, to a nucleic acid sequence of the respective naturally occurring (wild-type) nucleic acid sequence or gene, or a homolog, fragment or derivative thereof.

Also, the term “variant” as used throughout the present specification in the context of proteins or peptides will be recognized and understood by the person of ordinary skill in the art, and is e.g. intended to refer to a proteins or peptide variant having an amino acid sequence which differs from the original sequence in one or more mutation(s), such as one or more substituted, inserted and/or deleted amino acid(s). Preferably, these fragments and/or variants have the same biological function or specific activity compared to the full-length native protein, e.g. its specific antigenic property. “Variants” of proteins or peptides as defined herein may comprise conservative amino acid substitution(s) compared to their native, i.e. non-mutated physiological, sequence. Those amino acid sequences as well as their encoding nucleotide sequences in particular fall under the term variants as defined herein. Substitutions in which amino acids, which originate from the same class, are exchanged for one another are called conservative substitutions. In particular, these are amino acids having aliphatic side chains, positively or negatively charged side chains, aromatic groups in the side chains or amino acids, the side chains of which can enter into hydrogen bridges, e.g. side chains which have a hydroxyl function. This means that e.g. an amino acid having a polar side chain is replaced by another amino acid having a likewise polar side chain, or, e.g., an amino acid characterized by a hydrophobic side chain is substituted by another amino acid having a likewise hydrophobic side chain (e.g. serine (threonine) by threonine (serine) or leucine (isoleucine) by isoleucine (leucine)). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra). A “variant” of a protein or peptide may have at least 70%, 75%, 80%, 85%, 90%, 95%, 98% or 99% amino acid identity over a stretch of at least 10, 20, 30, 50, 75 or 100 amino acids of such protein or peptide. Preferably, a variant of a protein comprises a functional variant of the protein, which means that the variant exerts the same effect or functionality or at least 40%, 50%, 60%, 70%, 80%, 90%, or 95% of the effect or functionality as the protein it is derived from.

The term “fragment” in the context of nucleic acid sequences or genes refers to a continuous subsequence of the full-length reference (or “parent”) nucleic acid sequence or gene. In other words, a “fragment” may typically be a shorter portion of a full-length nucleic acid sequence or gene. Accordingly, a fragment, typically, consists of a sequence that is identical to the corresponding stretch within the full-length nucleic acid sequence or gene. The term includes naturally occurring fragments as well as engineered fragments. A preferred fragment of a sequence in the context of the present invention, consists of a continuous stretch of nucleic acids corresponding to a continuous stretch of entities in the nucleic acid or gene the fragment is derived from, which represents at least 20%, preferably at least 30%, more preferably at least 40%, more preferably at least 50%, even more preferably at least 60%, even more preferably at least 70%, and most preferably at least 80% of the total (i.e. full-length) nucleic acid sequence or gene from which the fragment is derived. A sequence identity indicated with respect to such a fragment preferably refers to the entire nucleic acid sequence or gene. Preferably, a “fragment” may comprise a nucleic acid sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, to a reference nucleic acid sequence or gene that it is derived from.

UTR elements are preferably “functional”, i.e. capable of eliciting the same desired biological effect as the parent UTRs that they are derived from, i.e. in particular of modulating, controlling or regulating (inducing, enhancing, reducing, abrogating, or preventing, preferably inducing or enhancing) the expression of an operably linked coding sequence. The term “expression” as used herein generally includes all step of protein biosynthesis, inter alia transcription, mRNA processing and translation. UTR elements, in particular 3′-UTR elements and 5′UTR elements in the combinations specified herein, may for instance (typically via the action of regulatory regions comprised by said UTR elements) regulate polyadenylation, translation initiation, translation efficiency, localization, and/or stability of the nucleic acid comprising said UTR elements.

Artificial nucleic acid molecules of the invention advantageously comprise at least one 5′ UTR element and at least one 3′ UTR element, each derived from a gene selected from the groups disclosed herein. Suitable 5′ UTR elements are preferably selected from 5′-UTR elements derived from a 5′ UTR of a gene selected from the group consisting of HSD17B4, ASAH1, ATP5A1, MP68, NDUFA4, NOSIP, RPL31, SLC7A3, TUBB4B and UBQLN2, preferably as defined herein. Suitable 3′ UTR elements are preferably selected from 3′ UTR elements derived from a 3′ UTR of a gene selected from the group consisting of PSMB3, CASP1, COX6B1, GNAS, NDUFA1 and RPS9, preferably as defined herein. Further, the artificial nucleic acid molecules of the invention may optionally comprise at least one coding region operably linked to said 3′UTR element and said 5′ UTR element. Preferably, the inventive artificial nucleic acid molecules may therefore comprise, in a 5′→3′ direction, a 5′-UTR element as defined herein, operably linked to a coding region (cds) encoding a (poly-)peptide or protein of interest, and a 3′ UTR element, operably linked to said coding region:
5′-UTR-cds-3′ UTR.

Typically, the 5′- and/or 3′-UTR elements of the inventive artificial nucleic acid molecules may be “heterologous” to the at least one coding sequence. The term “heterologous” is used herein to refer to a nucleic acid sequence that is typically derived from a different species than a reference nucleic acid sequence. A “heterologous sequence” may thus be derived from a gene that is of a different origin as compared to a reference sequence, and may typically differ, in its sequence of nucleic acids, from the reference sequence and/or may encode a different gene product.

### UTRs

### 5′ UTR

The artificial nucleic acid described herein comprises at least one 5′-UTR element derived from a 5′ UTR of a gene as indicated herein, or a homolog, variant, fragment or derivative thereof.

The term “5′-UTR” refers to a part of a nucleic acid molecule, which is located 5′ (i.e. “upstream”) of an open reading frame and which is not translated into protein. In the context of the present invention, a 5′-UTR starts with the transcriptional start site and ends one nucleotide before the start codon of the open reading frame. The 5′-UTR may comprise elements for regulating gene expression, also called “regulatory elements”. Such regulatory elements may be, for example, ribosomal binding sites. The 5′-UTR may be post-transcriptionally modified, for example by addition of a 5′-Cap. Thus, 5′-UTRs may preferably correspond to the sequence of a nucleic acid, in particular a mature mRNA, which is located between the 5′-Cap and the start codon, and more specifically to a sequence, which extends from a nucleotide located 3′ to the 5′-Cap, preferably from the nucleotide located immediately 3′ to the 5′-Cap, to a nucleotide located 5′ to the start codon of the protein coding sequence (transcriptional start site), preferably to the nucleotide located immediately 5′ to the start codon of the protein coding sequence (transcriptional start site). The nucleotide located immediately 3′ to the 5′-Cap of a mature mRNA typically corresponds to the transcriptional start site. 5′ UTRs typically have a length of less than 500, 400, 300, 250 or less than 200 nucleotides. In some embodiments its length may be in the range of at least 10, 20, 30 or 40, preferably up to 100 or 150, nucleotides.

Preferably, the at least one 5′UTR element comprises or consists of a nucleic acid sequence derived from the 5′ UTR of a chordate gene, preferably a vertebrate gene, more preferably a mammalian gene, most preferably a human gene, or from a variant of the 3′UTR of a chordate gene, preferably a vertebrate gene, more preferably a mammalian gene, most preferably a human gene.

Some of the 5′UTR elements specified herein may be derived from the 5′UTR of a TOP gene or from a homolog, variant or fragment thereof. “TOP genes” are typically characterized by the presence of a 5′terminal oligo pyrimidine tract (TOP), and further, typically by a growth-associated translational regulation. However, TOP genes with a tissue specific translational regulation are also known. mRNA that contains a 5′TOP is often referred to as TOP mRNA. Accordingly, genes that provide such messenger RNAs are referred to as TOP genes. TOP sequences have, for example, been found in genes and mRNAs encoding peptide elongation factors and ribosomal proteins. The 5′terminal oligo pyrimidine tract (“STOP” or “TOP”) is typically a stretch of pyrimidine nucleotides located in the 5′ terminal region of a nucleic acid molecule, such as the 5′ terminal region of certain mRNA molecules or the 5′ terminal region of a functional entity, e.g. the transcribed region, of certain genes. The 5′UTR of a TOP gene corresponds to the sequence of a 5′UTR of a mature mRNA derived from a TOP gene, which preferably extends from the nucleotide located 3′ to the 5′-CAP to the nucleotide located 5′ to the start codon. The TOP sequence typically starts with a cytidine, which usually corresponds to the transcriptional start site, and is followed by a stretch of usually about 3 to 30 pyrimidine nucleotides. The pyrimidine stretch and thus the 5′ TOP ends one nucleotide 5′ to the first purine nucleotide located downstream of the TOP.

A 5′UTR of a TOP gene typically does not comprise any start codons, preferably no upstream AUGs (uAUGs) or upstream open reading frames (uORFs). Therein, upstream AUGs and upstream open reading frames are typically understood to be AUGs and open reading frames that occur 5′ of the start codon (AUG) of the open reading frame that should be translated. The 5′UTRs of TOP genes are generally rather short. The lengths of 5′UTRs of TOP genes may vary between 20 nucleotides up to 500 nucleotides, and are typically less than about 200 nucleotides, preferably less than about 150 nucleotides, more preferably less than about 100 nucleotides. For example, a TOP may comprise 3, 4, 5, 6, 7, 8, 9, 10, 11, 12, 13, 14, 15, 16, 17, 18, 19, 20, 21, 22, 23, 24, 25, 26, 27, 28, 29, 30 or even more nucleotides. As used herein, the term “TOP motif” refers to a nucleic acid sequence which corresponds to a STOP as defined above. Thus, a “TOP motif” is preferably a stretch of pyrimidine nucleotides having a length of 3-30 nucleotides. Preferably, the TOP-motif consists of at least 3, preferably at least 4, more preferably at least 6, more preferably at least 7, and most preferably at least 8 pyrimidine nucleotides, wherein the stretch of pyrimidine nucleotides preferably starts at its 5′end with a cytosine nucleotide. In TOP genes and TOP mRNAs, the “TOP-motif” preferably starts at its 5′end with the transcriptional start site and ends one nucleotide 5′ to the first purine residue in said gene or mRNA. A “TOP motif” is preferably located at the 5′end of a sequence, which represents a 5′UTR, or at the 5′end of a sequence, which codes for a 5′UTR. Thus, preferably, a stretch of 3 or more pyrimidine nucleotides is called “TOP motif” if this stretch is located at the 5′end of a respective sequence, such as the artificial nucleic acid molecule, the 5′UTR element of the artificial nucleic acid molecule, or the nucleic acid sequence which is derived from the 5′UTR of a TOP gene as described herein. In other words, a stretch of 3 or more pyrimidine nucleotides, which is not located at the 5′-end of a 5′UTR or a 5′UTR element but anywhere within a 5′UTR or a 5′UTR element, is preferably not referred to as “TOP motif”.

In one embodiment, the 5′-end of an mRNA is “gggaga”.

The 5′UTR elements derived from 5′UTRs of TOP genes exemplified herein may preferably lack a TOP-motif or a 5′TOP, as defined above. Thus, the nucleic acid sequence of the 5′UTR element, which is derived from a 5′UTR of a TOP gene, may terminate at its 3′-end with a nucleotide located at position 1, 2, 3, 4, 5, 6, 7, 8, 9 or 10 upstream of the start codon (e.g. A(U/T)G) of the gene or mRNA it is derived from. Thus, the 5′UTR element does not comprise any part of the protein coding sequence. Thus, preferably, the only amino acid coding part of the artificial nucleic acid is provided by the coding sequence.

Particular 5′-UTR elements envisaged in accordance with the present invention are described in detail below.

### HSD17B4-Derived 5′ UTR Elements

Artificial nucleic acids according to the invention may comprise a 5′UTR element derived from a 5′UTR of a gene encoding a 17-beta-hydroxysteroid dehydrogenase 4, or a homolog, variant, fragment or derivative thereof, preferably lacking the 5′TOP motif.

Such 5′UTR elements preferably comprise or consist of a nucleic acid sequence which is derived from the 5′UTR of a 17-beta-hydroxysteroid dehydrogenase 4 (also referred to as peroxisomal multifunctional enzyme type 2) gene, preferably from a vertebrate, more preferably mammalian, most preferably human 17-beta-hydroxysteroid dehydrogenase 4 (HSD17B4) gene, or a homolog, variant, fragment or derivative thereof, wherein preferably the 5′UTR element does not comprise the 5′TOP of said gene. Said gene may preferably encode a 17-beta-hydroxysteroid dehydrogenase 4 protein corresponding to human 17-beta-hydroxysteroid dehydrogenase 4 (UniProt Ref. No. Q9BPX1, entry version #139 of Aug. 30, 2017), or a homolog, variant, fragment or derivative thereof.

Accordingly, artificial nucleic acids according to the invention may comprise a 5′UTR element derived from a HSD17B4 gene, in particular derived from the 5′ UTR of said HSD17B4 gene, preferably wherein said 5′UTR element comprises or consists of a DNA sequence according to SEQ ID NO: 1 or a homolog, variant, fragment or derivative thereof, in particular a DNA sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to a nucleic acid sequence according to SEQ ID NO: 1, or wherein said 5′UTR element comprises or consists of an RNA sequence according to SEQ ID NO: 2, or a or a homolog, variant, fragment or derivative thereof, in particular an RNA sequence having, in increasing order of preference, at least at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to a nucleic acid sequence according to SEQ ID NO: 2.

### ASAH1-Derived 5′ UTR Elements

Artificial nucleic acids according to the invention may comprise a 5′UTR element derived from a 5′UTR of a gene encoding acid ceramidase (ASAH1), or a homolog, variant, fragment or derivative thereof.

Such 5′UTR elements preferably comprise or consist of a nucleic acid sequence which is derived from the 5′UTR of an acid ceramidase (ASAH1) gene, preferably a vertebrate, more preferably mammalian, most preferably human acid ceramidase (ASAH1) gene, or a homolog, variant, fragment or derivative thereof. Said gene preferably encodes an acid ceramidase protein corresponding to human acid ceramidase (UniProt Ref. No. Q13510, entry version #177 of Jun. 7, 2017), or a homolog, variant, fragment or derivative thereof.

Accordingly, artificial nucleic acids according to the invention may comprise a 5′UTR element derived from an ASAH1 gene, in particular derived from the 5′ UTR of said ASAH1 gene, preferably wherein said 5′UTR element comprises or consists of a DNA sequence according to SEQ ID NO: 3 or a homolog, variant, fragment or derivative thereof, in particular a DNA sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to a nucleic acid sequence according to SEQ ID NO: 3, or wherein said 5′UTR element comprises or consists of an RNA sequence according to SEQ ID NO: 4, or a or a homolog, variant, fragment or derivative thereof, in particular an RNA sequence having, in increasing order of preference, at least at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to a nucleic acid sequence according to SEQ ID NO: 4.

### A TP5A1-Derived 5′-UTR Elements

Artificial nucleic acids according to the invention may comprise a 5′UTR element which is derived from a 5′UTR of a gene encoding mitochondrial ATP synthase subunit alpha (ATP5A1), or a homolog, variant, fragment or derivative thereof, wherein said 5′ UTR element preferably lacks the 5′TOP motif.

Such 5′UTR elements preferably comprise or consist of a nucleic acid sequence which is derived from the 5′UTR of a mitochondrial ATP synthase subunit alpha (ATP5A1) gene, preferably from a vertebrate, more preferably a mammalian and most preferably a human mitochondrial ATP synthase subunit alpha (ATP5A1) gene, or a homolog, variant, fragment or derivative thereof, wherein the 5′UTR element preferably does not comprise the 5′TOP of said gene. Said gene may preferably encode a mitochondrial ATP synthase subunit alpha protein corresponding to human acid mitochondrial ATP synthase subunit alpha (UniProt Ref. No. P25705, entry version #208 of Aug. 30, 2017), or a homolog, variant, fragment or derivative thereof.

Accordingly, artificial nucleic acids according to the invention may comprise a 5′UTR element derived from a ATP5A1 gene, in particular derived from the 5′ UTR of said ATP5A1 gene, preferably wherein said 5′UTR element comprises or consists of a DNA sequence according to SEQ ID NO: 5 or a homolog, variant, fragment or derivative thereof, in particular a DNA sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 5, or wherein said 5′UTR element comprises or consists of an RNA sequence according to SEQ ID NO: 6, or a homolog, variant, fragment or derivative thereof, in particular an RNA sequence having, in increasing order of preference, at least at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 6.

### MP68-Derived 5′ UTR Elements

Artificial nucleic acids according to the invention may comprise a 5′UTR element which is derived from a 5′UTR of a gene encoding MP68, or a homolog, variant, fragment or derivative thereof.

Such 5′UTR elements preferably comprise or consist of a nucleic acid sequence which is derived from the 5′UTR of a 6.8 kDa mitochondrial proteolipid (MP68) gene, preferably from a vertebrate, more preferably a mammalian and most preferably a human 6.8 kDa mitochondrial proteolipid (MP68) gene, or a homolog, variant, fragment or derivative thereof. Said gene may preferably encode a 6.8 kDa mitochondrial proteolipid (MP68) protein corresponding to human 6.8 kDa mitochondrial proteolipid (MP68) (UniProt Ref. No. P56378, entry version #127 of 15 Feb. 2017), or a homolog, variant, fragment or derivative thereof.

Accordingly, artificial nucleic acids according to the invention may comprise a 5′UTR element derived from a MP68 gene, in particular derived from the 5′ UTR of said MP68 gene, preferably wherein said 5′UTR element comprises or consists of a DNA sequence according to SEQ ID NO: 7 or a homolog, variant, fragment or derivative thereof, in particular a DNA sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 7, or wherein said 5′UTR element comprises or consists of an RNA sequence according to SEQ ID NO: 8, or a homolog, variant, fragment or derivative thereof, in particular an RNA sequence having, in increasing order of preference, at least at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 8.

### NDUFA4-Derived 5′-UTR Elements

Artificial nucleic acids according to the invention may comprise a 5′UTR element which is derived from a 5′UTR of a gene encoding a Cytochrome c oxidase subunit (NDUFA4), or a homolog, fragment or variant thereof.

Such 5′UTR elements preferably comprise or consist of a nucleic acid sequence which is derived from the 5′UTR of a Cytochrome c oxidase subunit (NDUFA4) gene, preferably from a vertebrate, more preferably a mammalian, most preferably a human Cytochrome c oxidase subunit (NDUFA4) gene, or a homolog, variant, fragment or derivative thereof. Said gene may preferably encode a Cytochrome c oxidase subunit (NDUFA4) protein corresponding to a human Cytochrome c oxidase subunit (NDUFA4) protein (UniProt Ref. No. 000483, entry version #149 of 30 Aug. 2017).

Accordingly, artificial nucleic acids according to the invention may comprise a 5′UTR element derived from a NDUFA4 gene, wherein said 5′UTR element comprises or consists of a DNA sequence according to SEQ ID NO: 9 or a homolog, variant, fragment or derivative thereof, in particular a DNA sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 9, or wherein said 5′UTR element comprises or consists of an RNA sequence according to SEQ ID NO: 10, or a homolog, variant, fragment or derivative thereof, in particular an RNA sequence having, in increasing order of preference, at least at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 10.

### NOSIP-Derived 5′ UTR Elements

Artificial nucleic acids according to the invention may comprise a 5′UTR element which is derived from a 5′UTR of a gene encoding a Nitric oxide synthase-interacting (NOSIP) protein, or a homolog, variant, fragment or derivative thereof.

Such 5′UTR elements preferably comprise or consist of a nucleic acid sequence which is derived from the 5′UTR of a Nitric oxide synthase-interacting protein (NOSIP) gene, preferably from a vertebrate, more preferably a mammalian, most preferably a human Nitric oxide synthase-interacting protein (NOSIP) gene, or a homolog, variant, fragment or derivative thereof. Said gene may preferably encode a Nitric oxide synthase-interacting protein (NOSIP) protein corresponding to a human Nitric oxide synthase-interacting protein (NOSIP) protein (UniProt Ref. No. Q9Y314, entry version #130 of 7 Jun. 2017).

Accordingly, artificial nucleic acids according to the invention may comprise a 5′UTR element derived from a NOSIP gene, wherein said 5′UTR element comprises or consists of a DNA sequence according to SEQ ID NO: 11 or a homolog, variant, fragment or derivative thereof, in particular a DNA sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 11, or wherein said 5′UTR element comprises or consists of an RNA sequence according to SEQ ID NO: 12, or a homolog, variant, fragment or derivative thereof, in particular an RNA sequence having, in increasing order of preference, at least at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 12.

### RPL31-Derived 5′-UTR Elements

Artificial nucleic acids according to the invention may comprise a 5′UTR element which is derived from a 5′UTR of a gene encoding a 60S ribosomal protein L31, or a homolog, variant, fragment or derivative thereof, wherein said 5′ UTR element preferably lacks the 5′TOP motif.

Such 5′UTR elements preferably comprise or consist of a nucleic acid sequence which is derived from the 5′UTR of a 60S ribosomal protein L31 (RPL31) gene, preferably from a vertebrate, more preferably a mammalian, most preferably a human 60S ribosomal protein L31 (RPL31) gene, or a homolog, variant, fragment or derivative thereof, wherein the 5′UTR element preferably does not comprise the 5′TOP of said gene. Said gene may preferably encode a 60S ribosomal protein L31 (RPL31) corresponding to a human 60S ribosomal protein L31 (RPL31) (UniProt Ref. No. P62899, entry version #138 of 30 Aug. 2017).

Accordingly, artificial nucleic acids according to the invention may comprise a 5′UTR element derived from a RPL31 gene, wherein said 5′UTR element comprises or consists of a DNA sequence according to SEQ ID NO: 13 or a homolog, variant, fragment or derivative thereof, in particular a DNA sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 13, or wherein said 5′UTR element comprises or consists of an RNA sequence according to SEQ ID NO: 14, or a homolog, variant, fragment or derivative thereof, in particular an RNA sequence having, in increasing order of preference, at least at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 14.

### SLC7A3-Derived 5′-UTR Elements

Artificial nucleic acids according to the invention may comprise a 5′UTR element which is derived from a 5′UTR of a gene encoding a cationic amino acid transporter 3 (solute carrier family 7 member 3, SLC7A3) protein, or a homolog, variant, fragment or derivative thereof.

Such 5′UTR elements preferably comprise or consist of a nucleic acid sequence which is derived from the 5′UTR of a cationic amino acid transporter 3 (SLC7A3) gene, preferably from a vertebrate, more preferably a mammalian, most preferably a human cationic amino acid transporter 3 (SLC7A3) gene, or a homolog, variant, fragment or derivative thereof. Said gene may preferably encode a cationic amino acid transporter 3 (SLC7A3) protein corresponding to a human cationic amino acid transporter 3 (SLC7A3) protein (UniProt Ref. No. Q8WY07, entry version #139 of 30 Aug. 2017).

Accordingly, artificial nucleic acids according to the invention may comprise a 5′UTR element derived from a SLC7A3 gene, wherein said 5′UTR element comprises or consists of a DNA sequence according to SEQ ID NO: 15 or a homolog, variant, fragment or derivative thereof, in particular a DNA sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 15, or wherein said 5′UTR element comprises or consists of an RNA sequence according to SEQ ID NO: 16, or a homolog, variant, fragment or derivative thereof, in particular an RNA sequence having, in increasing order of preference, at least at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 16.

### TUBB4B-Derived 5′ UTR Elements

Artificial nucleic acids according to the invention may comprise a 5′UTR element which is derived from a 5′UTR of a gene encoding a tubulin beta-4B chain (TUBB4B) protein, or a homolog, variant, fragment or derivative thereof.

Such 5′UTR elements preferably comprise or consist of a nucleic acid sequence which is derived from the 5′UTR of a tubulin beta-4B chain (TUBB4B) gene, preferably from a vertebrate, more preferably a mammalian, most preferably a human tubulin beta-4B chain (TUBB4B) gene, or a homolog, variant, fragment or derivative thereof. Said gene may preferably encode a tubulin beta-4B chain (TUBB4B) protein corresponding to a human tubulin beta-4B chain (TUBB4B) protein (UniProt Ref. No. Q8WY07, entry version #142 of 30 Aug. 2017).

Accordingly, artificial nucleic acids according to the invention may comprise a 5′UTR element derived from a tubulin beta-4B chain (TUBB4B) gene, wherein said 5′UTR element comprises or consists of a DNA sequence according to SEQ ID NO: 17 or a homolog, variant, fragment or derivative thereof, in particular a DNA sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 17, or wherein said 5′UTR element comprises or consists of an RNA sequence according to SEQ ID NO: 18, or a homolog, variant, fragment or derivative thereof, in particular an RNA sequence having, in increasing order of preference, at least at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 18.

### UBQLN2-Derived 5′-UTR Elements

Artificial nucleic acids according to the invention may comprise a 5′UTR element which is derived from a 5′UTR of a gene encoding an ubiquilin-2 (UBQLN2) protein, or a homolog, variant, fragment or derivative thereof.

Such 5′UTR elements preferably comprise or consist of a nucleic acid sequence which is derived from the 5′UTR of a ubiquilin-2 (UBQLN2) gene, preferably from a vertebrate, more preferably a mammalian, most preferably a human ubiquilin-2 (UBQLN2) gene, or a homolog, variant, fragment or derivative thereof. Said gene may preferably encode an ubiquilin-2 (UBQLN2) protein corresponding to a human ubiquilin-2 (UBQLN2) protein (UniProt Ref. No. Q9UHD9, entry version #151 of 30 Aug. 2017).

Accordingly, artificial nucleic acids according to the invention may comprise a 5′UTR element derived from a ubiquilin-2 (UBQLN2) gene, wherein said 5′UTR element comprises or consists of a DNA sequence according to SEQ ID NO: 19 or a homolog, variant, fragment or derivative thereof, in particular a DNA sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 19, or wherein said 5′UTR element comprises or consists of an RNA sequence according to SEQ ID NO: 20, or a homolog, variant, fragment or derivative thereof, in particular an RNA sequence having, in increasing order of preference, at least at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 20.

### 3′ UTR

The artificial nucleic acid described herein further comprises at least one 3′-UTR element derived from a 3′ UTR of a gene as defined herein, or a homolog, variant or fragment of said gene. The term “3′-UTR” refers to a part of a nucleic acid molecule, which is located 3′ (i.e. “downstream”) of an open reading frame and which is not translated into protein. In the context of the present invention, a 3′-UTR corresponds to a sequence which is located between the stop codon of the protein coding sequence, preferably immediately 3′ to the stop codon of the protein coding sequence, and the poly(A) sequence of the artificial nucleic acid (RNA) molecule.

Preferably, the at least one 3′UTR element comprises or consists of a nucleic acid sequence derived from the 3′UTR of a chordate gene, preferably a vertebrate gene, more preferably a murine gene, even more preferably a mammalian gene, most preferably a human gene, or from a variant of the 3′UTR of a chordate gene, preferably a vertebrate gene, more preferably a murine gene, even more preferably a mammalian gene, most preferably a human gene.

### PSMB3-Derived 3′-UTR Elements

Artificial nucleic acids according to the invention may comprise a 3′UTR element which is derived from a 3′UTR of a gene encoding a proteasome subunit beta type-3 (PSMB3) protein, or a homolog, variant, fragment or derivative thereof.

Such 3′UTR elements preferably comprises or consists of a nucleic acid sequence which is derived from the 3′UTR of a proteasome subunit beta type-3 (PSMB3) gene, preferably from a vertebrate, more preferably a mammalian, most preferably a human proteasome subunit beta type-3 (PSMB3) gene, or a homolog, variant, fragment or derivative thereof.

Said gene may preferably encode a proteasome subunit beta type-3 (PSMB3) protein corresponding to a human proteasome subunit beta type-3 (PSMB3) protein (UniProt Ref. No. P49720, entry version #183 of 30 Aug. 2017).

Accordingly, artificial nucleic acids according to the invention may comprise a 3′UTR element derived from a PSMB3 gene, wherein said 3′UTR element comprises or consists of a DNA sequence according to SEQ ID NO: 23 or a homolog, variant, fragment or derivative thereof, in particular a DNA sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 23, or wherein said 3′UTR element comprises or consists of an RNA sequence according to SEQ ID NO: 24, or a homolog, variant, fragment or derivative thereof, in particular an RNA sequence having, in increasing order of preference, at least at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 24.

### CASP1-Derived 3′-UTR Elements

Artificial nucleic acids according to the invention may comprise a 3′UTR element which is derived from a 3′UTR of a gene encoding a Caspase-1 (CASP1) protein, or a homolog, variant, fragment or derivative thereof.

Such 3′UTR elements preferably comprises or consists of a nucleic acid sequence which is derived from the 3′UTR of a Caspase-1 (CASP1) gene, preferably from a vertebrate, more preferably a mammalian, most preferably a human Caspase-1 (CASP1) gene, or a homolog, variant, fragment or derivative thereof.

Accordingly, artificial nucleic acids according to the invention may comprise a 3′UTR element derived from a CASP1 gene, wherein said 3′UTR element comprises or consists of a DNA sequence according to SEQ ID NO: 25 or a homolog, variant, fragment or derivative thereof, in particular a DNA sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 25, or wherein said 3′UTR element comprises or consists of an RNA sequence according to SEQ ID NO: 26, or a homolog, variant, fragment or derivative thereof, in particular an RNA sequence having, in increasing order of preference, at least at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 26.

### COX6B1-Derived 3′-UTR Elements

Artificial nucleic acids according to the invention may comprise a 3′UTR element which is derived from a 3′UTR of a COX6B1 gene encoding a cytochrome c oxidase subunit 6B1 (COX6B1) protein, or a homolog, variant, fragment or derivative thereof.

Such 3′UTR elements preferably comprises or consists of a nucleic acid sequence which is derived from the 3′UTR of a cytochrome c oxidase subunit 6B1 (COX6B1) gene, preferably from a vertebrate, more preferably a mammalian, most preferably a human cytochrome c oxidase subunit 6B1 (COX6B1) gene, or a homolog, variant, fragment or derivative thereof. Said gene may preferably encode a cytochrome c oxidase subunit 6B1 (COX6B1) protein corresponding to a human cytochrome c oxidase subunit 6B1 (COX6B1) protein (UniProt Ref. No. P14854, entry version #166 of 30 Aug. 2017).

Accordingly, artificial nucleic acids according to the invention may comprise a 3′UTR element derived from a COX6B1 gene, wherein said 3′UTR element comprises or consists of a DNA sequence according to SEQ ID NO: 27 or a homolog, variant, fragment or derivative thereof, in particular a DNA sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 27, or wherein said 3′UTR element comprises or consists of an RNA sequence according to SEQ ID NO: 28, or a homolog, variant, fragment or derivative thereof, in particular an RNA sequence having, in increasing order of preference, at least at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 28.

### GNAS-Derived 3′-UTR Elements

Artificial nucleic acids according to the invention may comprise a 3′UTR element derived from a 3′UTR of a gene encoding a Guanine nucleotide-binding protein G(s) subunit alpha isoforms short (GNAS) protein, or a homolog, variant, fragment or derivative thereof.

Such 3′UTR elements preferably comprises or consists of a nucleic acid sequence which is derived from the 3′UTR of a Guanine nucleotide-binding protein G(s) subunit alpha isoforms short (GNAS) gene, preferably from a vertebrate, more preferably a mammalian, most preferably a human Guanine nucleotide-binding protein G(s) subunit alpha isoforms short (GNAS) gene, or a homolog, variant, fragment or derivative thereof. Said gene may preferably encode a Guanine nucleotide-binding protein G(s) subunit alpha isoforms short (GNAS) protein corresponding to a human Guanine nucleotide-binding protein G(s) subunit alpha isoforms short (GNAS) protein (UniProt Ref. No. P63092, entry version #153 of 30 Aug. 2017).

Accordingly, artificial nucleic acids according to the invention may comprise a 3′ UTR element derived from a GNAS gene, wherein said 3′UTR element comprises or consists of a DNA sequence according to SEQ ID NO: 29 or a homolog, variant, fragment or derivative thereof, in particular a DNA sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 29, or wherein said 3′UTR element comprises or consists of an RNA sequence according to SEQ ID NO: 30, or a homolog, variant, fragment or derivative thereof, in particular an RNA sequence having, in increasing order of preference, at least at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 30.

### NDUFA1-Derived 3′ UTR Elements

Artificial nucleic acids according to the invention may comprise a 3′UTR element which is derived from a 3′UTR of a gene encoding a NADH dehydrogenase [ubiquinone] 1 alpha subcomplex subunit 1 (NDUFA1) protein, or a homolog, variant, fragment or derivative thereof.

Such 3′UTR elements preferably comprises or consists of a nucleic acid sequence which is derived from the 3′UTR of a NADH dehydrogenase [ubiquinone] 1 alpha subcomplex subunit 1 (NDUFA1) gene, preferably from a vertebrate, more preferably a mammalian, most preferably a human NADH dehydrogenase [ubiquinone] 1 alpha subcomplex subunit 1 (NDUFA1) gene, or a homolog, variant, fragment or derivative thereof. Said gene may preferably encode a NADH dehydrogenase [ubiquinone] 1 alpha subcomplex subunit 1 (NDUFA1) protein corresponding to a human NADH dehydrogenase [ubiquinone] 1 alpha subcomplex subunit 1 (NDUFA1) protein (UniProt Ref. No. 015239, entry version #152 of 30 Aug. 2017).

Accordingly, artificial nucleic acids according to the invention may comprise a 3′UTR element derived from a NDUFA1 gene, wherein said 3′UTR element comprises or consists of a DNA sequence according to SEQ ID NO: 31 or a homolog, variant, fragment or derivative thereof, in particular a DNA sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 31, or wherein said 3′UTR element comprises or consists of an RNA sequence according to SEQ ID NO: 32, or a homolog, variant, fragment or derivative thereof, in particular an RNA sequence having, in increasing order of preference, at least at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 32.

### RPS9-Derived 3′-UTRs

Artificial nucleic acids according to the invention may comprise a 3′UTR element which comprises or consists of a nucleic acid sequence, which is derived from a 3′UTR of a gene encoding a 40S ribosomal protein S9 (RPS9) protein, or a homolog, variant, fragment or derivative thereof.

Such 3′UTR elements preferably comprises or consists of a nucleic acid sequence which is derived from the 3′UTR of a 40S ribosomal protein S9 (RPS9) gene, preferably from a vertebrate, more preferably a mammalian, most preferably a human 40S ribosomal protein S9 (RPS9) gene, or a homolog, variant, fragment or derivative thereof. Said gene may preferably encode a 40S ribosomal protein S9 (RPS9) protein corresponding to a 40S ribosomal protein S9 (RPS9) protein (UniProt Ref. No. P46781, entry version #179 of 30 Aug. 2017).

Accordingly, artificial nucleic acids according to the invention may comprise a 3′UTR element derived from a RPS9 gene, wherein said 3′UTR element comprises or consists of a DNA sequence according to SEQ ID NO: 33 or a homolog, variant, fragment or derivative thereof, in particular a DNA sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 33, or wherein said 5′UTR element comprises or consists of an RNA sequence according to SEQ ID NO: 34, or a homolog, variant, fragment or derivative thereof, in particular an RNA sequence having, in increasing order of preference, at least at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the nucleic acid sequence according to SEQ ID NO: 34.

### UTR Combinations

Preferably, the at least one 5′UTR element and the at least one 3′UTR element act synergistically to modulate, more preferably induce or enhance, the expression of the at least one coding sequence operably linked to said UTR elements. It is envisaged herein to utilize each 5′- and 3′-UTR element exemplified herein in any conceivable combination.

Preferred combinations of 5′- and 3′-UTR elements are listed in table 1 below.

TABLE 1
UTR combinations

| | 5′ UTR element | SEQ | 3′ UTR element | SEQ |
|---|---|---|---|---|
| # | derived from | ID NO: | derived from | ID NO: |
| 1 | ASAH1 | 4 | CASP1 | 26 |
| 2 | ASAH1 | 4 | COX6B1 | 28 |
| 3 | ASAH1 | 4 | GNAS | 30 |
| 4 | ASAH1 | 4 | NDUFA1 | 32 |
| 5 | ASAH1 | 4 | PSMB3 | 24 |
| 6 | ASAH1 | 4 | RPS9 | 34 |
| 7 | ATP5A1 | 6 | CASP1 | 26 |
| 8 | ATP5A1 | 6 | COX6B1 | 28 |
| 9 | ATP5A1 | 6 | GNAS | 30 |
| 10 | ATP5A1 | 6 | NDUFA1 | 32 |
| 11 | ATP5A1 | 6 | PSMB3 | 24 |
| 12 | ATP5A1 | 6 | RPS9 | 34 |
| 13 | HSD17B4 | 2 | CASP1 | 26 |
| 14 | HSD17B4 | 2 | COX6B1 | 28 |
| 15 | HSD17B4 | 2 | GNAS | 30 |
| 16 | HSD17B4 | 2 | NDUFA1 | 32 |
| 17 | HSD17B4 | 2 | PSMB3 | 24 |
| 18 | HSD17B4 | 2 | RPS9 | 34 |
| 19 | MP68 | 8 | CASP1 | 26 |
| 20 | MP68 | 8 | COX6B1 | 28 |
| 21 | MP68 | 8 | GNAS | 30 |
| 22 | MP68 | 8 | NDUFA1 | 32 |
| 23 | MP68 | 8 | PSMB3 | 24 |
| 24 | MP68 | 8 | RPS9 | 34 |
| 25 | NDUFA4 | 10 | CASP1 | 26 |
| 26 | NDUFA4 | 10 | COX6B1 | 28 |
| 27 | NDUFA4 | 10 | GNAS | 30 |
| 28 | NDUFA4 | 10 | NDUFA1 | 32 |
| 29 | NDUFA4 | 10 | PSMB3 | 24 |
| 30 | NDUFA4 | 10 | RPS9 | 34 |
| 31 | NOSIP | 12 | CASP1 | 26 |
| 32 | NOSIP | 12 | COX6B1 | 28 |
| 33 | NOSIP | 12 | GNAS | 30 |
| 34 | NOSIP | 12 | NDUFA1 | 32 |
| 35 | NOSIP | 12 | PSMB3 | 24 |
| 36 | NOSIP | 12 | RPS9 | 34 |
| 37 | RPL31 | 14 | CASP1 | 26 |
| 38 | RPL31 | 14 | COX6B1 | 28 |
| 39 | RPL31 | 14 | GNAS | 30 |
| 40 | RPL31 | 14 | NDUFA1 | 32 |
| 41 | RPL31 | 14 | PSMB3 | 24 |
| 42 | RPL31 | 14 | RPS9 | 34 |
| 43 | SLC7A3 | 16 | CASP1 | 26 |
| 44 | SLC7A3 | 16 | COX6B1 | 28 |
| 45 | SLC7A3 | 16 | GNAS | 30 |
| 46 | SLC7A3 | 16 | NDUFA1 | 32 |
| 47 | SLC7A3 | 16 | PSMB3 | 24 |
| 48 | SLC7A3 | 16 | RPS9 | 34 |
| 49 | TUBB4B | 18 | CASP1 | 26 |
| 50 | TUBB4B | 18 | COX6B1 | 28 |
| 51 | TUBB4B | 18 | GNAS | 30 |
| 52 | TUBB4B | 18 | NDUFA1 | 32 |
| 53 | TUBB4B | 18 | PSMB3 | 24 |
| 54 | TUBB4B | 18 | RPS9 | 34 |
| 55 | UBQLN2 | 20 | CASP1 | 26 |
| 56 | UBQLN2 | 20 | COX6B1 | 28 |
| 57 | UBQLN2 | 20 | GNAS | 30 |
| 58 | UBQLN2 | 20 | NDUFA1 | 32 |
| 59 | UBQLN2 | 20 | PSMB3 | 24 |
| 60 | UBQLN2 | 20 | RPS9 | 34 |

Especially the following UTR-combinations are preferred: 5′UTR: ASAH1+3′UTR: CASP1; 5′UTR: ASAH1+3′UTR: COX6B1; 5′UTR: ASAH1+3′UTR: Gnas; 5′UTR: ASAH1+3′UTR: Ndufa1.1; 5′UTR: ASAH1+3′UTR: PSMB3; 5′UTR: ASAH1+3′UTR: RPS9; 5′UTR: ATP5A1+3′UTR: CASP1; 5′UTR: ATP5A1+3′UTR: COX6B1; 5′UTR: ATP5A1+3′UTR: Gnas; 5′UTR: ATP5A1+3′UTR: Ndufa1.1; 5′UTR: ATP5A1+3′UTR: PSMB3; 5′UTR: ATP5A1+3′UTR: RPS9; 5′UTR: HSD17B4+3′UTR: CASP1; 5′UTR: HSD17B4+3′UTR: COX6B1; 5′UTR: HSD17B4+3′UTR: Ndufa1.1; 5′UTR: HSD17B4+3′UTR: PSMB3; 5′UTR: HSD17B4+3′UTR: RPS9; 5′UTR: Mp68+3′UTR: CASP1; 5′UTR: Mp68+3′UTR: COX6B1; 5′UTR: Mp68+3′UTR: Gnas; 5′UTR: Mp68+3′UTR: Ndufa1.1; 5′UTR: Mp68+3′UTR: PSMB3; 5′UTR: Mp68+3′UTR: RPS9; 5′UTR: Ndufa4+3′UTR: CASP1; 5′UTR: Ndufa4+3′UTR: COX6B1; 5′UTR: Ndufa4+3′UTR: Gnas; 5′UTR: Ndufa4+3′UTR: Ndufa1.1; 5′UTR: Ndufa4+3′UTR: PSMB3; 5′UTR: Ndufa4+3′UTR: RPS9; 5′UTR: Nosip+3′UTR: CASP1; 5′UTR: Nosip+3′UTR: COX6B1; 5′UTR: Nosip+3′UTR: Gnas; 5′UTR: Nosip+3′UTR: Ndufa1.1; 5′UTR: Nosip+3′UTR: PSMB3; 5′UTR: Nosip+3′UTR: RPS9; 5′UTR: Rpl31+3′UTR: CASP1; 5′UTR: Rpl31+3′UTR: COX6B1; 5′UTR: Rpl31+3′UTR: Gnas; 5′UTR: Rpl31+3′UTR: Ndufa1.1; 5′UTR: Rpl31+3′UTR: PSMB3; 5′UTR: Rpl31+3′UTR: RPS9; 5′UTR: Slc7a3+3′UTR: CASP1; 5′UTR: Slc7a3+3′UTR: COX6B1; 5′UTR: Slc7a3+3′UTR: Ndufa1.1; 5′UTR: Slc7a3+3′UTR: PSMB3; 5′UTR: Slc7a3+3′UTR: RPS9; 5′UTR: TUBB4B+3′UTR: CASP1; 5′UTR: TUBB4B+3′UTR: COX6B1; 5′UTR: TUBB4B+3′UTR: Gnas; 5′UTR: TUBB4B+3′UTR: Ndufa1.1; 5′UTR: TUBB4B+3′UTR: PSMB3; 5′UTR: TUBB4B+3′UTR: RPS9; 5′UTR: Ubqln2+3′UTR: CASP1; 5′UTR: Ubqln2+3′UTR: COX6B1; 5′UTR: Ubqln2+3′UTR: Gnas; 5′UTR: Ubqln2+3′UTR: Ndufa1.1; 5′UTR: Ubqln2+3′UTR: PSMB3; and 5′UTR: Ubqln2+3′UTR: RPS9, preferably the UTR-combination 5′UTR: HSD17B4+3′UTR: Gnas, more preferably the UTR-combination 5′UTR: Slc7a3+3′UTR: Gnas.

Each of the UTR elements defined in table 1 by reference to a specific SEQ ID NO may include variants or fragments of the nucleic acid sequence defined by said specific SEQ ID NO, exhibiting at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to the respective nucleic acid sequence defined by reference to its specific SEQ ID NO. Each of the sequences identified in table 1 by reference to their specific SEQ ID NO may also be defined by its corresponding DNA sequence, as indicated herein. Each of the sequences identified in table 1 by reference to their specific SEQ ID NO may be modified (optionally independently from each other) as described herein below.

Preferred artificial nucleic acids according to the invention may comprise:
a-1. at least one 5′ UTR element derived from a 5′UTR of a HSD17B4 gene, or from a corresponding RNA sequence, homolog, fragment or variant thereof and at least one 3′ UTR element derived from a 3′UTR of a PSMB3 gene, or from a corresponding RNA sequence, homolog, fragment or variant thereof; or
a-2. at least one 5′ UTR element derived from a 5′UTR of a NDUFA4 gene, or from a corresponding RNA sequence, homolog, fragment or variant thereof and at least one 3′ UTR element derived from a 3′UTR of a PSMB3 gene, or from a corresponding RNA sequence, homolog, fragment or variant thereof; or
a-3. at least one 5′ UTR element derived from a 5′UTR of a SLC7A3 gene, or from a corresponding RNA sequence, homolog, fragment or variant thereof and at least one 3′ UTR element derived from a 3′UTR of a PSMB3 gene, or from a corresponding RNA sequence, homolog, fragment or variant thereof; or
a-4. at least one 5′ UTR element derived from a 5′UTR of a NOSIP gene, or from a corresponding RNA sequence, homolog, fragment or variant thereof and at least one 3′ UTR element derived from a 3′UTR of a PSMB3 gene, or from a corresponding RNA sequence, homolog, fragment or variant thereof; or
a-5. at least one 5′ UTR element derived from a 5′UTR of a MP68 gene, or from a corresponding RNA sequence, homolog, fragment or variant thereof and at least one 3′ UTR element derived from a 3′UTR of a PSMB3 gene, or from a corresponding RNA sequence, homolog, fragment or variant thereof; or
b-1. at least one 5′ UTR element derived from a 5′UTR of a UBQLN2 gene, or from a corresponding RNA sequence, homolog, fragment or variant thereof and at least one 3′ UTR element derived from a 3′UTR of a RPS9 gene, or from a corresponding RNA sequence, homolog, fragment or variant thereof; or
b-2. at least one 5′ UTR element derived from a 5′UTR of a ASAH1 gene, or from a corresponding RNA sequence, homolog, fragment or variant thereof and at least one 3′ UTR element derived from a 3′UTR of a RPS9 gene, or from a corresponding RNA sequence, homolog, fragment or variant thereof; or
b-3. at least one 5′ UTR element derived from a 5′UTR of a HSD17B4 gene, or from a corresponding RNA sequence, homolog, fragment or variant thereof and at least one 3′ UTR element derived from a 3′UTR of a RPS9 gene, or from a corresponding RNA sequence, homolog, fragment or variant thereof; or
b-4. at least one 5′ UTR element derived from a 5′UTR of a HSD17B4 gene, or from a corresponding RNA sequence, homolog, fragment or variant thereof and at least one 3′ UTR element derived from a 3′UTR of a CASP1 gene, or from a corresponding RNA sequence, homolog, fragment or variant thereof; or
b-5. at least one 5′ UTR element derived from a 5′UTR of a NOSIP gene, or from a corresponding RNA sequence, homolog, fragment or variant thereof and at least one 3′ UTR element derived from a 3′UTR of a COX6B1 gene, or from a corresponding RNA sequence, homolog, fragment or variant thereof; or
c-1. at least one 5′ UTR element derived from a 5′UTR of a NDUFA4 gene, or from a corresponding RNA sequence, homolog, fragment or variant thereof and at least one 3′ UTR element derived from a 3′UTR of a RPS9 gene, or from a corresponding RNA sequence, homolog, fragment or variant thereof; or
c-2. at least one 5′ UTR element derived from a 5′UTR of a NOSIP gene, or from a corresponding RNA sequence, homolog, fragment or variant thereof and at least one 3′ UTR element derived from a 3′UTR of a NDUFA1 gene, or from a corresponding RNA sequence, homolog, fragment or variant thereof; or
c-3. at least one 5′ UTR element derived from a 5′UTR of a NDUFA4 gene, or from a corresponding RNA sequence, homolog, fragment or variant thereof and at least one 3′ UTR element derived from a 3′UTR of a COX6B1 gene, or from a corresponding RNA sequence, homolog, fragment or variant thereof; or
c-4. at least one 5′ UTR element derived from a 5′UTR of a NDUFA4 gene, or from a corresponding RNA sequence, homolog, fragment or variant thereof and at least one 3′ UTR element derived from a 3′UTR of a NDUFA1 gene, or from a corresponding RNA sequence, homolog, fragment or variant thereof; or
c-5. at least one 5′ UTR element derived from a 5′UTR of a ATP5A1 gene, or from a corresponding RNA sequence, homolog, fragment or variant thereof and at least one 3′ UTR element derived from a 3′UTR of a PSMB3 gene, or from a corresponding RNA sequence, homolog, fragment or variant thereof; or
d-1. at least one 5′ UTR element derived from a 5′UTR of a RPL31 gene, or from a corresponding RNA sequence, homolog, fragment or variant thereof and at least one 3′ UTR element derived from a 3′UTR of a PSMB3 gene, or from a corresponding RNA sequence, homolog, fragment or variant thereof; or
d-2. at least one 5′ UTR element derived from a 5′UTR of a ATP5A1 gene, or from a corresponding RNA sequence, homolog, fragment or variant thereof and at least one 3′ UTR element derived from a 3′UTR of a CASP1 gene, or from a corresponding RNA sequence, homolog, fragment or variant thereof; or
d-3. at least one 5′ UTR element derived from a 5′UTR of a SLC7A3 gene, or from a corresponding RNA sequence, homolog, fragment or variant thereof and at least one 3′ UTR element derived from a 3′UTR of a GNAS1 gene, or from a corresponding RNA sequence, homolog, fragment or variant thereof; or
d-4. at least one 5′ UTR element derived from a 5′UTR of a HSD17B4 gene, or from a corresponding RNA sequence, homolog, fragment or variant thereof and at least one 3′ UTR element derived from a 3′UTR of a NDUFA1 gene, or from a corresponding RNA sequence, homolog, fragment or variant thereof; or
d-5. at least one 5′ UTR element derived from a 5′UTR of a SLC7A3 gene, or from a corresponding RNA sequence, homolog, fragment or variant thereof and at least one 3′ UTR element derived from a 3′UTR of a NDUFA1 gene, or from a corresponding RNA sequence, homolog, fragment or variant thereof; or
e-1. at least one 5′ UTR element derived from a 5′UTR of a TUBB4B gene, or from a corresponding RNA sequence, homolog, fragment or variant thereof and at least one 3′ UTR element derived from a 3′UTR of a RPS9 gene, or from a corresponding RNA sequence, homolog, fragment or variant thereof; or
e-2. at least one 5′ UTR element derived from a 5′UTR of a RPL31 gene, or from a corresponding RNA sequence, homolog, fragment or variant thereof and at least one 3′ UTR element derived from a 3′UTR of a RPS9 gene, or from a corresponding RNA sequence, homolog, fragment or variant thereof; or
e-3. at least one 5′ UTR element derived from a 5′UTR of a MP68 gene, or from a corresponding RNA sequence, homolog, fragment or variant thereof and at least one 3′ UTR element derived from a 3′UTR of a RPS9 gene, or from a corresponding RNA sequence, homolog, fragment or variant thereof; or
e-4. at least one 5′ UTR element derived from a 5′UTR of a NOSIP gene, or from a corresponding RNA sequence, homolog, fragment or variant thereof and at least one 3′ UTR element derived from a 3′UTR of a RPS9 gene, or from a corresponding RNA sequence, homolog, fragment or variant thereof; or
e-5. at least one 5′ UTR element derived from a 5′UTR of a ATP5A1 gene, or from a corresponding RNA sequence, homolog, fragment or variant thereof and at least one 3′ UTR element derived from a 3′UTR of a RPS9 gene, or from a corresponding RNA sequence, homolog, fragment or variant thereof; or
e-6. at least one 5′ UTR element derived from a 5′UTR of a ATP5A1 gene, or from a corresponding RNA sequence, homolog, fragment or variant thereof and at least one 3′ UTR element derived from a 3′UTR of a COX6B1 gene, or from a corresponding RNA sequence, homolog, fragment or variant thereof; or
f-1. at least one 5′ UTR element derived from a 5′UTR of a ATP5A1 gene, or from a corresponding RNA sequence, homolog, fragment or variant thereof and at least one 3′ UTR element derived from a 3′UTR of a GNAS gene, or from a corresponding RNA sequence, homolog, fragment or variant thereof; or
f-2. at least one 5′ UTR element derived from a 5′UTR of a ATP5A1 gene, or from a corresponding RNA sequence, homolog, fragment or variant thereof and at least one 3′ UTR element derived from a 3′UTR of a NDUFA1 gene, or from a corresponding RNA sequence, homolog, fragment or variant thereof; or
f.3 at least one 5′ UTR element derived from a 5′UTR of a HSD17B4 gene, or from a corresponding RNA sequence, homolog, fragment or variant thereof and at least one 3′ UTR element derived from a 3′UTR of a COX6B1 gene, or from a corresponding RNA sequence, homolog, fragment or variant thereof; or
f-4 at least one 5′ UTR element derived from a 5′UTR of a HSD17B4 gene, or from a corresponding RNA sequence, homolog, fragment or variant thereof and at least one 3′ UTR element derived from a 3′UTR of a GNAS1 gene, or from a corresponding RNA sequence, homolog, fragment or variant thereof; or
f-5. at least one 5′ UTR element derived from a 5′UTR of a MP68 gene, or from a corresponding RNA sequence, homolog, fragment or variant thereof and at least one 3′ UTR element derived from a 3′UTR of a COX6B1 gene, or from a corresponding RNA sequence, homolog, fragment or variant thereof; or
g-1. at least one 5′ UTR element derived from a 5′UTR of a MP68 gene, or from a corresponding RNA sequence, homolog, fragment or variant thereof and at least one 3′ UTR element derived from a 3′UTR of a NDUFA1 gene, or from a corresponding RNA sequence, homolog, fragment or variant thereof; or
g-2. at least one 5′ UTR element derived from a 5′UTR of a NDUFA4 gene, or from a corresponding RNA sequence, homolog, fragment or variant thereof and at least one 3′ UTR element derived from a 3′UTR of a CASP1 gene, or from a corresponding RNA sequence, homolog, fragment or variant thereof; or
g-3. at least one 5′ UTR element derived from a 5′UTR of a NDUFA4 gene, or from a corresponding RNA sequence, homolog, fragment or variant thereof and at least one 3′ UTR element derived from a 3′UTR of a GNAS gene, or from a corresponding RNA sequence, homolog, fragment or variant thereof; or
g-4 at least one 5′ UTR element derived from a 5′UTR of a NOSIP gene, or from a corresponding RNA sequence, homolog, fragment or variant thereof and at least one 3′ UTR element derived from a 3′UTR of a CASP1 gene, or from a corresponding RNA sequence, homolog, fragment or variant thereof; or
g-5 at least one 5′ UTR element derived from a 5′UTR of a RPL31 gene, or from a corresponding RNA sequence, homolog, fragment or variant thereof and at least one 3′ UTR element derived from a 3′UTR of a CASP1 gene, or from a corresponding RNA sequence, homolog, fragment or variant thereof; or
h-1 at least one 5′ UTR element derived from a 5′UTR of a RPL31 gene, or from a corresponding RNA sequence, homolog, fragment or variant thereof and at least one 3′ UTR element derived from a 3′UTR of a COX6B1 gene, or from a corresponding RNA sequence, homolog, fragment or variant thereof; or
h-2 at least one 5′ UTR element derived from a 5′UTR of a RPL31 gene, or from a corresponding RNA sequence, homolog, fragment or variant thereof and at least one 3′ UTR element derived from a 3′UTR of a GNAS gene, or from a corresponding RNA sequence, homolog, fragment or variant thereof; or
h-3 at least one 5′ UTR element derived from a 5′UTR of a RPL31 gene, or from a corresponding RNA sequence, homolog, fragment or variant thereof and at least one 3′ UTR element derived from a 3′UTR of a NDUFA1 gene, or from a corresponding RNA sequence, homolog, fragment or variant thereof; or
h-4 at least one 5′ UTR element derived from a 5′UTR of a SLC7A3 gene, or from a corresponding RNA sequence, homolog, fragment or variant thereof and at least one 3′ UTR element derived from a 3′UTR of a CASP1 gene, or from a corresponding RNA sequence, homolog, fragment or variant thereof; or
h-5 at least one 5′ UTR element derived from a 5′UTR of a SLC7A3 gene, or from a corresponding RNA sequence, homolog, fragment or variant thereof and at least one 3′ UTR element derived from a 3′UTR of a COX6B1 gene, or from a corresponding RNA sequence, homolog, fragment or variant thereof; or
i-1 at least one 5′ UTR element derived from a 5′UTR of a SLC7A3 gene, or from a corresponding RNA sequence, homolog, fragment or variant thereof and at least one 3′ UTR element derived from a 3′UTR of a RPS9 gene, or from a corresponding RNA sequence, homolog, fragment or variant thereof; or
i-2 at least one 5′ UTR element derived from a 5′UTR of a Ndufa4.1 gene, or from a corresponding RNA sequence, homolog, fragment or variant thereof and at least one 3′ UTR element derived from a 3′UTR of a CASP1 gene, or from a corresponding RNA sequence, homolog, fragment or variant thereof.

Particularly preferred artificial nucleic acids may comprise a combination of UTRs according to a-1, a-2, a-3, a-4 or a-5, preferably according to a-1.

Surprisingly it was discovered that certain combinations of 5′ and 3′-untranslated regions (UTRs) as disclosed herein act in concert to synergistically enhance the expression of operably linked nucleic acid sequences. Testing for synergy of UTR combinations is routine for a skilled person in the art, f.e. a test for synergy can be performed by Luciferase expression after mRNA transfection to prove that effects of synergy are present, i.e. more than an additive effect.

### Expression in the Liver

Any of the UTR combinations disclosed herein is envisaged to modulate, preferably induce and more preferably enhance, the expression of an operably linked coding sequence (cds). Without wishing to be bound by specific theory, some of the UTR combinations disclosed herein may be particularly useful when used in connection with specific coding sequences and/or when used in connection with a specific target cells or tissues.

In some embodiments, the artificial nucleic acid molecule according to the invention may comprise UTR elements according to a-2 (NDUFA4/PSMB3); a-5 (MP68/PSMB3); c-1 (NDUFA4/RPS9); a-1 (HSD17B4/PSMB3); e-3 (MP68/RPS9); e-4 (NOSIP/RPS9); a-4 (NOSIP/PSMB3); e-2 (RPL31/RPS9); e-5 (ATP5A1/RPS9); d-4 (HSD17B4/NUDFA1); b-5 (NOSIP/COX6B1); a-3 (SLC7A3/PSMB3); b-1 (UBQLN2/RPS9); b-2 (ASAH1/RPS9); b-4 (HSD17B4/CASP1); e-6 (ATP5A1/COX6B1); b-3 (HSD17B4/RPS9); g-5 (RPL31/CASP1); h-1 (RPL31/COX6B1); and/or c-5 (ATP5A1/PSMB3) as defined above. Such artificial nucleic acid molecules may be particularly useful for expression of an encoded (poly-)peptide or protein of interest in the liver. Accordingly, such artificial nucleic acid molecules are particularly envisaged for systemical administration, in particular intravenous, intraperitoneal, intramuscular or intratracheal administration or injection and optionally in combination with liver-targeting elements herein (as discussed below). Furthermore, without wishing to imply any particular limitation, the aforementioned UTR combinations may be particularly useful for artificial nucleic acids encoding, in their at least one coding region, a therapeutic (poly-)peptide or protein, an antigenic or allergic (poly-)peptide or protein as disclosed herein, for instance a protein useful in treating a disease selected from the group consisting of genetic diseases, allergies, autoimmune diseases, infectious diseases, neoplasms, cancer, and tumor-related diseases, inflammatory diseases, diseases of the blood and blood-forming organs, endocrine, nutritional and metabolic diseases, diseases of the nervous system, diseases of the circulatory system, diseases of the respiratory system, diseases of the digestive system, diseases of the skin and subcutaneous tissue, diseases of the musculoskeletal system and connective tissue, and diseases of the genitourinary system, independently if they are inherited or acquired, and combinations thereof.

### Dermis, Epidermis and Subcutaneous Expression

In some embodiments, the artificial nucleic acid molecule according to the invention may comprise UTR elements according to a-1 (HSD17B4/PSMB3); a-3 (SLC7A3/PSMB3); e-2 (RPL31/RPS9); a-5 (MP68/PSMB3); d-1 (RPL31/PSMB3); a-2 (NDUFA4/PSMB3); h-1 (RPL31/COX6B1); b-1 (UBQLN2/RPS9); a-4 (NOSIP/PSMB3); c-5 (ATP5A1/PSMB3); b-5 (NOSIP/COX6B1); d-4 (HSD17B4/NDUFA1); i-1 (SLC7A3/RPS9); f-3 (HSD17B4/COX6B1); b-4 (HSD17B4/CASP1); g-5 (RPL31/CASP1); c-2 (NOSIP/NDUFA1); e-4 (NOSIP/RPS9); c-4 (NDUFA4/NDUFA1); and/or d-5 (SLC7A3/NDUFA1) as defined above. Such artificial nucleic acid molecules may be particularly useful for expression of an encoded (poly-)peptide or protein of interest in the skin. Accordingly, such artificial nucleic acid molecules are particularly envisaged for intra-dermal administration, in particular topical, transdermal, intra-dermal injection, subcutaneous, or epicutaneous administration or injection herein. Furthermore, without wishing to imply any particular limitation, the aforementioned UTR combinations may be particularly useful for artificial nucleic acids encoding, in their at least one coding region, a therapeutic (poly-)peptide or protein, an antigenic or allergic (poly-)peptide or protein as disclosed herein, for instance a protein useful in treating a disease selected from the group consisting of genetic diseases, allergies, autoimmune diseases, infectious diseases, neoplasms, cancer, and tumor-related diseases, inflammatory diseases, diseases of the blood and blood-forming organs, endocrine, nutritional and metabolic diseases, diseases of the nervous system, diseases of the circulatory system, diseases of the respiratory system, diseases of the digestive system, diseases of the skin and subcutaneous tissue, diseases of the musculoskeletal system and connective tissue, and diseases of the genitourinary system, independently if they are inherited or acquired, and combinations thereof.

### Expression in the Muscle

In some embodiments, the artificial nucleic acid molecule according to the invention may comprise UTR elements according to a-4 (NOSIP/PSMB3); a-1 (HSD17B4/PSMB3); a-5 (MP68/PSMB3); d-3 (SLC7A3/GNAS); a-2 (NDUFA4/PSMB3); a-3 (SLC7A3/PSMB3); d-5 (SLC7A3/NDUFA1); i-1 (SLC7A3/RPS9); d-1 (RPL31/PSMB3); d-4 (HSD17B4/NDUFA1); b-3 (HSD17B4/RPS9); f-3 (HSD17B4/COX6B1); f-4 (HSD17B4/GNAS); h-5 (SLC7A3/COX6B1); g-4 (NOSIP/CASP1); c-3 (NDUFA4/COX6B1); b-1 (UBQLN2/RPS9); c-5 (ATP5A1/PSMB3); h-4 (SLC7A3/CASP1); h-2 (RPL31/GNAS); e-1 (TUBB4B/RPS9); f-2 (ATP5A1/NDUFA1); c-2 (NOSIP/NDUFA1); b-5 (NOSIP/COX6B1); and/or e-4 (NOSIP/RPS9) as defined above. Such artificial nucleic acid molecules may be particularly useful for expression of an encoded (poly-)peptide or protein of interest in the skeletal muscle, smooth muscle or cardiac muscle. Accordingly, such artificial nucleic acid molecules are particularly envisaged for intra-muscular administration, more preferably intra-muscular injection or intracardiac injection, herein. Furthermore, without wishing to imply any particular limitation, the aforementioned UTR combinations may be particularly useful for artificial nucleic acids encoding, in their at least one coding region, a therapeutic (poly-)peptide or protein, an antigenic or allergic (poly-)peptide or protein as disclosed herein, for instance a protein useful in treating a disease selected from the group consisting of genetic diseases, allergies, autoimmune diseases, infectious diseases, neoplasms, cancer, and tumor-related diseases, inflammatory diseases, diseases of the blood and blood-forming organs, endocrine, nutritional and metabolic diseases, diseases of the nervous system, diseases of the circulatory system, diseases of the respiratory system, diseases of the digestive system, diseases of the skin and subcutaneous tissue, diseases of the musculoskeletal system and connective tissue, and diseases of the genitourinary system, independently if they are inherited or acquired, and combinations thereof.

### Expression in Tumor and Cancer Cells

In some embodiments, the artificial nucleic acid molecule according to the invention may comprise UTR elements according to e-1 (TUBB4B/RPS9); b-2 (ASAH1/RPS9); c-3 (NDUFA4/COX6B1); a-1 (HSD17B4/PSMB3); c-4 (NDUFA4/NDUFA1); b-4 (HSD17B4/CASP1); d-2 (ATP5A1/CASP1); b-5 (NOSIP/COX6B1); a-2 (NDUFA4/PSMB3); b-1 (UBQLN/RPS9); a-3 (SLC7A3/PSMB3); f-4 (HSD17B4/GNAS); c-2 (NOSIP/NDUFA1); b-3 (HSD17B4/RPS9); c-5 (ATP5A1/PSMB3); a-4 (NOSIP/PSMB3); d-5 (SLC7A3/NDUFA1); or f-3 (HSD17B4/COX6B1) as defined above. Such artificial nucleic acid molecules may be particularly useful for expression of an encoded (poly-)peptide or protein of interest in a tumor or cancer cell, including a carcinoma, sarcoma, lymphoma, leukemia, germ cell tumor or blastoma cell. Accordingly, such artificial nucleic acid molecules are particularly envisaged for intra-tumoral, intramuscular, subcutaneous, intravenous, intradermal, intraperitoneal, intrapleural, intraosseous administration or injection herein. Furthermore, without wishing to imply any particular limitation, the aforementioned UTR combinations may be particularly useful for artificial nucleic acids encoding, in their at least one coding region, a therapeutic (poly-)peptide or protein, an antigenic or allergic (poly-)peptide or protein as disclosed herein, for instance a protein useful in treating a disease selected from the group consisting of a cancer or tumor disease.

### Expression in Kidney Cells

In some embodiments, the artificial nucleic acid molecule according to the invention may comprise UTR elements according to b-2 (ASAH1/RPS9); c-1 (NDUFA4/RPS9.1); e-3 (MP68/RPS9); c-4 (NDUFA4/NDUFA1); c-2 (NOSIP/NDUFA1); h-2 (RPL31/CASP1); d-2 (ATP5A1/CASP1); b-3 (HSD17B4/RPS9); a-2 (NDUFA4/PSMB3); f-4 (HSD17B4/GNAS); d-3 (SLC7A3/GNAS); g-1 (MP68/NDUFA1); c-3 (NDUFA4/COX6B1); e-5 (ATP5A1/RPS9); h-3 (RPL31/NDUFA1); a-1 (HSD17B4/PSMB3); a-5 (MP68/PSMB3); g-4 (NOSIP/CASP1); b-1 (UQBLN/RPS9); d-4 (HSD17B4/NDUFA1); or e-2 (RPL31/RPS9) as defined above. Such artificial nucleic acid molecules may be particularly useful for expression of an encoded (poly-)peptide or protein of interest in kidney cells. Accordingly, such artificial nucleic acid molecules are particularly envisaged for systemical administration, in particular intravenous, intraperitoneal, intramuscular or intratracheal administration or injection and optionally in combination with kidney-targeting elements herein. Furthermore, without wishing to imply any particular limitation, the aforementioned UTR combinations may be particularly useful for artificial nucleic acids encoding, in their at least one coding region, a therapeutic (poly-)peptide or protein, an antigenic or allergic (poly-)peptide or protein as disclosed herein, for instance a protein useful in treating a disease selected from the group consisting of genetic diseases, allergies, autoimmune diseases, infectious diseases, neoplasms, cancer, and tumor-related diseases, inflammatory diseases, diseases of the blood and blood-forming organs, endocrine, nutritional and metabolic diseases, diseases of the nervous system, diseases of the circulatory system, diseases of the respiratory system, diseases of the digestive system, diseases of the skin and subcutaneous tissue, diseases of the musculoskeletal system and connective tissue, and diseases of the genitourinary system, independently if they are inherited or acquired, and combinations thereof.

In view of the above, artificial nucleic acid molecules according to the invention may be defined as indicated above, wherein
said 5′UTR element derived from a HSD17B4 gene comprises or consists of a DNA sequence according to SEQ ID NO: 1 or a DNA sequence having, in increasing order of preference, at least 50%, 60%, 70%, 80%, 90%, 95%, 96%, 97%, 98%, or 99% sequence identity to the nucleic acid sequence according to SEQ ID NO: 1, or a fragment or a variant thereof; or an RNA sequence according to SEQ ID NO: 2, or an RNA sequence having, in increasing order of preference, at least 50%, 60%, 70%, 80%, 90%, 95%, 96%, 97%, 98%, or 99% sequence identity to the nucleic acid sequence according to SEQ ID NO: 2, or a fragment or a variant thereof;
said 5′UTR element derived from a ASAH1 gene comprises or consists of a DNA sequence according to SEQ ID NO: 3 or a DNA sequence having, in increasing order of preference, at least 50%, 60%, 70%, 80%, 90%, 95%, 96%, 97%, 98%, or 99% sequence identity to the nucleic acid sequence according to SEQ ID NO: 3, or a fragment or variant thereof; or an RNA sequence according to SEQ ID NO: 4, or an RNA sequence having, in increasing order of preference, at least 50%, 60%, 70%, 80%, 90%, 95%, 96%, 97%, 98%, or 99% sequence identity to the nucleic acid sequence according to SEQ ID NO: 4, or a fragment or a variant thereof;
said 5′UTR element derived from a ATP5A1 gene comprises or consists of a DNA sequence according to SEQ ID NO: 5, or a DNA sequence having, in increasing order of preference, at least 50%, 60%, 70%, 80%, 90%, 95%, 96%, 97%, 98%, or 99% sequence identity to the nucleic acid sequence according to SEQ ID NO: 5, or a fragment or variant thereof; or an RNA sequence according to SEQ ID NO: 6, or an RNA sequence having, in increasing order of preference, at least 50%, 60%, 70%, 80%, 90%, 95%, 96%, 97%, 98%, or 99% sequence identity to the nucleic acid sequence according to SEQ ID NO: 6, or a fragment or a variant thereof;
said 5′UTR element derived from a MP68 gene comprises or consists of a DNA sequence according to SEQ ID NO: 7, or a DNA sequence having, in increasing order of preference, at least 50%, 60%, 70%, 80%, 90%, 95%, 96%, 97%, 98%, or 99% sequence identity to the nucleic acid sequence according to SEQ ID NO: 7, or a fragment or variant thereof; or an RNA sequence according to SEQ ID NO: 8, or an RNA sequence having, in increasing order of preference, at least 50%, 60%, 70%, 80%, 90%, 95%, 96%, 97%, 98%, or 99% sequence identity to the nucleic acid sequence according to SEQ ID NO: 8, or a fragment or a variant thereof;
said 5′UTR element derived from a NDUFA4 gene comprises or consists of a DNA sequence according to SEQ ID NO: 9, or a DNA sequence having, in increasing order of preference, at least 50%, 60%, 70%, 80%, 90%, 95%, 96%, 97%, 98%, or 99% sequence identity to the nucleic acid sequence according to SEQ ID NO: 9, or a fragment or variant thereof; or an RNA sequence according to SEQ ID NO: 10, or an RNA sequence having, in increasing order of preference, at least 50%, 60%, 70%, 80%, 90%, 95%, 96%, 97%, 98%, or 99% sequence identity to the nucleic acid sequence according to SEQ ID NO: 10, or a fragment or a variant thereof;
said 5′UTR element derived from a NOSIP gene comprises or consists of a DNA sequence according to SEQ ID NO: 11, or a DNA sequence having, in increasing order of preference, at least 50%, 60%, 70%, 80%, 90%, 95%, 96%, 97%, 98%, or 99% sequence identity to the nucleic acid sequence according to SEQ ID NO: 11, or a fragment or variant thereof; or an RNA sequence according to SEQ ID NO: 12, or an RNA sequence having, in increasing order of preference, at least 50%, 60%, 70%, 80%, 90%, 95%, 96%, 97%, 98%, or 99% sequence identity to the nucleic acid sequence according to SEQ ID NO: 12, or a fragment or a variant thereof;
said 5′UTR element derived from a RPL31 gene comprises or consists of a DNA sequence according to SEQ ID NO: 13, or a DNA sequence having, in increasing order of preference, at least 50%, 60%, 70%, 80%, 90%, 95%, 96%, 97%, 98%, or 99% sequence identity to the nucleic acid sequence according to SEQ ID NO: 13, or a fragment or variant thereof; an RNA sequence according to SEQ ID NO: 14, or an RNA sequence having, in increasing order of preference, at least 50%, 60%, 70%, 80%, 90%, 95%, 96%, 97%, 98%, or 99% sequence identity to the nucleic acid sequence according to SEQ ID NO: 14, or a fragment or a variant thereof;
said 5′UTR element derived from a SLC7A3 gene comprises or consists of a DNA sequence according to SEQ ID NO: 15, or a DNA sequence having, in increasing order of preference, at least 50%, 60%, 70%, 80%, 90%, 95%, 96%, 97%, 98%, or 99% sequence identity to the nucleic acid sequence according to SEQ ID NO: 15, or a fragment or variant thereof; or an RNA sequence according to SEQ ID NO: 16, or an RNA sequence having, in increasing order of preference, at least 50%, 60%, 70%, 80%, 90%, 95%, 96%, 97%, 98%, or 99% sequence identity to the nucleic acid sequence according to SEQ ID NO: 16, or a fragment or a variant thereof;
said 5′UTR element derived from a TUBB4B gene comprises or consists of a DNA sequence according to SEQ ID NO: 17, or a DNA sequence having, in increasing order of preference, at least 50%, 60%, 70%, 80%, 90%, 95%, 96%, 97%, 98%, or 99% sequence identity to the nucleic acid sequence according to SEQ ID NO: 17, or a fragment or variant thereof; or an RNA sequence according to SEQ ID NO: 18, or an RNA sequence having, in increasing order of preference, at least 50%, 60%, 70%, 80%, 90%, 95%, 96%, 97%, 98%, or 99% sequence identity to the nucleic acid sequence according to SEQ ID NO: 18, or a fragment or a variant thereof;
said 5′UTR element derived from a UBQLN2 gene comprises or consists of a DNA sequence according to SEQ ID NO: 19, or a DNA sequence having, in increasing order of preference, at least 50%, 60%, 70%, 80%, 90%, 95%, 96%, 97%, 98%, or 99% sequence identity to the nucleic acid sequence according to SEQ ID NO: 19, or a fragment or variant thereof; or an RNA sequence according to SEQ ID NO: 20, or an RNA sequence having, in increasing order of preference, at least 50%, 60%, 70%, 80%, 90%, 95%, 96%, 97%, 98%, or 99% sequence identity to the nucleic acid sequence according to SEQ ID NO: 20, or a fragment or a variant thereof;
said 3′UTR element derived from a PSMB3 gene comprises or consists of a DNA sequence according to SEQ ID NO: 23, or a DNA sequence having, in increasing order of preference, at least 50%, 60%, 70%, 80%, 90%, 95%, 96%, 97%, 98%, or 99% sequence identity to the nucleic acid sequence according to SEQ ID NO: 23, or a fragment or variant thereof; or an RNA sequence according to SEQ ID NO: 24, or an RNA sequence having, in increasing order of preference, at least 50%, 60%, 70%, 80%, 90%, 95%, 96%, 97%, 98%, or 99% sequence identity to the nucleic acid sequence according to SEQ ID NO: 24, or a fragment or a variant thereof;
said 3′UTR element derived from a CASP1 gene comprises or consists of a DNA sequence according to SEQ ID NO: 25, or a DNA sequence having, in increasing order of preference, at least 50%, 60%, 70%, 80%, 90%, 95%, 96%, 97%, 98%, or 99% sequence identity to the nucleic acid sequence according to SEQ ID NO: 25, or a fragment or variant thereof; or an RNA sequence according to SEQ ID NO: 26, or an RNA sequence having, in increasing order of preference, at least 50%, 60%, 70%, 80%, 90%, 95%, 96%, 97%, 98%, or 99% sequence identity to the nucleic acid sequence according to SEQ ID NO: 26, or a fragment or a variant thereof;
said 3′UTR element derived from a COX6B1 gene comprises or consists of a DNA sequence according to SEQ ID NO: 27, or a DNA sequence having, in increasing order of preference, at least 50%, 60%, 70%, 80%, 90%, 95%, 96%, 97%, 98%, or 99% sequence identity to the nucleic acid sequence according to SEQ ID NO: 27, or a fragment or variant thereof; or an RNA sequence according to SEQ ID NO: 28, or an RNA sequence having, in increasing order of preference, at least 50%, 60%, 70%, 80%, 90%, 95%, 96%, 97%, 98%, or 99% sequence identity to the nucleic acid sequence according to SEQ ID NO: 28, or a fragment or a variant thereof;
said 3′UTR element derived from a GNAS gene comprises or consists of a DNA sequence according to SEQ ID NO: 29, or a DNA sequence having, in increasing order of preference, at least 50%, 60%, 70%, 80%, 90%, 95%, 96%, 97%, 98%, or 99% sequence identity to the nucleic acid sequence according to SEQ ID NO: 29, or a fragment or variant thereof; or an RNA sequence according to SEQ ID NO: 30, or an RNA sequence having, in increasing order of preference, at least 50%, 60%, 70%, 80%, 90%, 95%, 96%, 97%, 98%, or 99% sequence identity to the nucleic acid sequence according to SEQ ID NO: 30, or a fragment or a variant thereof;
said 3′UTR element derived from a NDUFA1 gene comprises or consists of a DNA sequence according to SEQ ID NO: 31, or a DNA sequence having, in increasing order of preference, at least 50%, 60%, 70%, 80%, 90%, 95%, 96%, 97%, 98%, or 99% sequence identity to the nucleic acid sequence according to SEQ ID NO: 31, or a fragment or variant thereof; or an RNA sequence according to SEQ ID NO: 32, or an RNA sequence having, in increasing order of preference, at least 50%, 60%, 70%, 80%, 90%, 95%, 96%, 97%, 98%, or 99% sequence identity to the nucleic acid sequence according to SEQ ID NO: 32, or a fragment or a variant thereof; and/or
said 3′UTR element derived from a RPS9 gene comprises or consists of a DNA sequence according to SEQ ID NO: 33, or a DNA sequence having, in increasing order of preference, at least 50%, 60%, 70%, 80%, 90%, 95%, 96%, 97%, 98%, or 99% sequence identity to the nucleic acid sequence according to SEQ ID NO: 33, or a fragment or variant thereof; or an RNA sequence according to SEQ ID NO: 34, or an RNA sequence having, in increasing order of preference, at least 50%, 60%, 70%, 80%, 90%, 95%, 96%, 97%, 98%, or 99% sequence identity to the nucleic acid sequence according to SEQ ID NO: 34, or a fragment or a variant thereof.

### Coding Region

The artificial nucleic acid according to the invention comprises at least one coding region or coding sequence operably linked to—and typically flanked by—at least one 3′-UTR element and at least one 5′-UTR element as defined herein. The terms “coding sequence” or “cds” and “coding region” are used interchangeably herein to refer to a segment or portion of a nucleic acid that encodes a (gene) product of interest. Gene products are products of gene expression and include (poly-)peptides and nucleic acids, such as (protein-)coding RNAs (such as mRNAs) and non-(protein-)coding RNAs (such as tRNAs, rRNAs, microRNAs, siRNAs). Typically, the at least one coding region of the inventive artificial nucleic acid molecule may encode at least one (poly-)peptide or protein, hereinafter referred to as “(poly-)peptide or protein of interest”. Coding regions may typically be composed of exons bounded by a start codon (such as AUG) at their 5′-end and a stop codon (such as UAG, UAA or UGA) at their 3′ end. In the artificial nucleic acid molecules of the invention, the coding region is bounded by at least one 5′-UTR element and at least one 3′-UTR element as defined herein.

(Poly-)peptides or proteins of interest generally include any (poly-)peptide or protein that can be encoded by the nucleic acid sequence of the at least one coding region, and can be expressed under suitable conditions to yield a functional (poly-)peptide or protein product. In this context, the term “functional” means “capable of exerting a desired biological function” and/or “exhibiting a desired biological property”. (Poly-)peptides or proteins of interest can have various functions and include, for instance, antibodies, enzymes, signaling proteins, receptors, receptor ligands, peptide hormones, transport proteins, structural proteins, neurotransmitters, growth regulating factors, serum proteins, carriers, drugs, immunomodulators, oncogenes, tumor suppressors, toxins, tumor antigens, and others. These proteins can be post-translationally modified to be proteins, glycoproteins, lipoproteins, phosphoproteins, etc. Further, the invention envisages any of the disclosed (poly-)peptides or proteins in their naturally occurring (wild-type) form, as well as variants, fragments and derivatives thereof. The encoded (poly-)peptides and proteins may have different effects. Without being limited thereto, coding regions encoding therapeutic, antigenic and allergenic (poly-)peptides are particularly envisaged herein.

### Therapeutic (Poly-)Peptides or Proteins

The at least one coding region of the artificial nucleic acid molecule of the invention may encode at least one “therapeutic (poly-)peptide or protein”. The term “therapeutic (poly-)peptide or protein” refers to a (poly-)peptide or protein capable of mediating a desired diagnostic, prophylactic or therapeutic effect, preferably resulting in detection, prevention, amelioration and/or healing of a disease.

Preferably, artificial nucleic acid molecules according to the invention may comprise at least one coding region encoding a therapeutic protein replacing an absent, deficient or mutated protein; a therapeutic protein beneficial for treating inherited or acquired diseases; infectious diseases, or neoplasms e.g. cancer or tumor diseases); an adjuvant or immuno-stimulating therapeutic protein; a therapeutic antibody or an antibody fragment, variant or derivative; a peptide hormone; a gene editing agent; an immune checkpoint inhibitor; a T cell receptor, or a fragment, variant or derivative T cell receptor; and/or an enzyme.

“Therapeutic (poly-)peptides or proteins “replacing an absent, deficient or mutated protein” may be selected from any (poly-)peptide or protein exhibiting the desired biological properties and/or capable of exerting the desired biological function of a wild-type protein, whose absence, deficiency or mutation causes disease. Herein, “absent” means that protein expression from its encoding gene is prevented or abolished, typically to an extent that the protein is not detectable at its target site (i.e. cellular compartment, cell type, tissue or organ) in the affected subject's body. Protein expression can be affected at a variety of levels, and the “absence” or “lack of production” of a protein in an affected patient's body may be due to mutations in the encoding gene, e.g. epigenetic alterations or sequence mutations either its open reading frame or its regulatory elements (e.g. nonsense mutations or deletions leading to the hindrance or abrogation of gene transcription), defective mRNA processing (e.g. defective mRNA splicing, maturation or export from the nucleus), protein translation deficiencies, or errors in the protein folding, translocation (i.e. failure to correctly enter the secretory pathway) or transport (i.e. failure to correctly enter its destined export pathway) process. A protein “deficiency”, i.e. reduced amount of protein detectable at its target site (i.e. cellular compartment, cell type, tissue or organ) in the affected subject's body, may be caused by the same mechanisms accounting for complete lack of protein expression as exemplified above. However, the defects leading to a protein “deficiency” may not always completely prevent or abolish protein expression from the affected gene, but rather lead to reduced expression levels (e.g. in cases where one allele is affected, and the other one functions normally). The term “mutated” encompasses both amino acid sequence variants and differences in the post-translational modification of proteins. Protein “mutants” may typically be non-functional, or mis-functional and may exhibit aberrant folding, translocation or transport properties or profiles.

Therapeutic (poly-)peptides or proteins “beneficial for treating inherited or acquired diseases such as infectious diseases, or neoplasms e.g. cancer or tumor diseases, diseases of the blood and blood-forming organs, endocrine, nutritional and metabolic diseases, diseases of the nervous system, diseases of the circulatory system, diseases of the respiratory system, diseases of the digestive system, diseases of the skin and subcutaneous tissue, diseases of the musculoskeletal system and connective tissue, and diseases of the genitourinary system, irrespective of being inherited or acquired” include any (poly-)peptides or protein whose expression is capable of preventing, ameliorating, or healing an inherited or acquired diseases. Such (poly-)peptides or proteins may in principle exert their therapeutic function by exerting any suitable biological action or function. In some embodiments, such (poly-)peptides or proteins may preferably not act by replacing an absent, deficient or mutated protein and/or by inducing an immune or allergenic response. For instance, (poly-)peptides or proteins beneficial for treating inherited or acquired diseases such as infectious diseases, or neoplasms may include particularly preferred therapeutic proteins which are inter alia beneficial in the treatment of acquired or inherited metabolic or endocrine disorders selected from (in brackets the particular disease for which the therapeutic protein is used in the treatment): Acid sphingomyelinase (Niemann-Pick disease), Adipotide (obesity), Agalsidase-beta (human galactosidase A) (Fabry disease; prevents accumulation of lipids that could lead to renal and cardiovascular complications), Alglucosidase (Pompe disease (glycogen storage disease type II)), alpha-galactosidase A (alpha-GAL A, Agalsidase alpha) (Fabry disease), alpha-glucosidase (Glycogen storage disease (GSD), Morbus Pompe), alpha-L-iduronidase (mucopolysaccharidoses (MPS), Hurler syndrome, Scheie syndrome), alpha-N-acetylglucosaminidase (Sanfilippo syndrome), Amphiregulin (cancer, metabolic disorder), Angiopoietin ((Ang1, Ang2, Ang3, Ang4, ANGPTL2, ANGPTL3, ANGPTL4, ANGPTL5, ANGPTL6, ANGPTL7) (angiogenesis, stabilize vessels), Betacellulin (metabolic disorder), Beta-glucuronidase (Sly syndrome), Bone morphogenetic protein BMPs (BMP1, BMP2, BMP3, BMP4, BMP5, BMP6, BMP7, BMP8a, BMP8b, BMP10, BMP15) (regenerative effect, bone-related conditions, chronic kidney disease (CKD)), CLN6 protein (CLN6 disease—Atypical Late Infantile, Late Onset variant, Early Juvenile, Neuronal Ceroid Lipofuscinoses (NCL)), Epidermal growth factor (EGF) (wound healing, regulation of cell growth, proliferation, and differentiation), Epigen (metabolic disorder), Epiregulin (metabolic disorder), Fibroblast Growth Factor (FGF, FGF-1, FGF-2, FGF-3, FGF-4, FGF-5, FGF-6, FGF-7, FGF-8, FGF-9, FGF-10, FGF-11, FGF-12, FGF-13, FGF-14, FGF-16, FGF-17, FGF-17, FGF-18, FGF-19, FGF-20, FGF-21, FGF-22, FGF-23) (wound healing, angiogenesis, endocrine disorders, tissue regeneration), Galsulphase (Mucopolysaccharidosis VI), Ghrelin (irritable bowel syndrome (IBS), obesity, Prader-Willi syndrome, type II diabetes mellitus), Glucocerebrosidase (Gaucher's disease), GM-CSF (regenerative effect, production of white blood cells, cancer), Heparin-binding EGF-like growth factor (HB-EGF) (wound healing, cardiac hypertrophy and heart development and function), Hepatocyte growth factor HGF (regenerative effect, wound healing), Hepcidin (iron metabolism disorders, Beta-thalassemia), Human albumin (Decreased production of albumin (hypoproteinaemia), increased loss of albumin (nephrotic syndrome), hypovolaemia, hyperbilirubinaemia), Idursulphase (Iduronate-2-sulphatase) (Mucopolysaccharidosis II (Hunter syndrome)), Integrins alphaVbeta3, alphaVbeta5 and alpha5beta1 (Bind matrix macromolecules and proteinases, angiogenesis), Iuduronate sulfatase (Hunter syndrome), Laronidase (Hurler and Hurler-Scheie forms of mucopolysaccharidosis I), N-acetylgalactosamine-4-sulfatase (rhASB; galsulfase, Arylsulfatase A (ARSA), Arylsulfatase B (ARSB)) (arylsulfatase B deficiency, Maroteaux-Lamy syndrome, mucopolysaccharidosis VI), N-acetylglucosamine-6-sulfatase (Sanfilippo syndrome), Nerve growth factor (NGF, Brain-Derived Neurotrophic Factor (BDNF), Neurotrophin-3 (NT-3), and Neurotrophin 4/5 (NT-4/5) (regenerative effect, cardiovascular diseases, coronary atherosclerosis, obesity, type 2 diabetes, metabolic syndrome, acute coronary syndromes, dementia, depression, schizophrenia, autism, Rett syndrome, anorexia nervosa, bulimia nervosa, wound healing, skin ulcers, corneal ulcers, Alzheimer's disease), Neuregulin (NRG1, NRG2, NRG3, NRG4) (metabolic disorder, schizophrenia), Neuropilin (NRP-1, NRP-2) (angiogenesis, axon guidance, cell survival, migration), Obestatin (irritable bowel syndrome (IBS), obesity, Prader-Willi syndrome, type II diabetes mellitus), Platelet Derived Growth factor (PDGF (PDFF-A, PDGF-B, PDGF-C, PDGF-D) (regenerative effect, wound healing, disorder in angiogenesis, Arteriosclerosis, Fibrosis, cancer), TGF beta receptors (endoglin, TGF-beta 1 receptor, TGF-beta 2 receptor, TGF-beta 3 receptor) (renal fibrosis, kidney disease, diabetes, ultimately end-stage renal disease (ESRD), angiogenesis), Thrombopoietin (THPO) (Megakaryocyte growth and development factor (MGDF)) (platelets disorders, platelets for donation, recovery of platelet counts after myelosuppressive chemotherapy), Transforming Growth factor (TGF (TGF-a, TGF-beta (TGFbeta1, TGFbeta2, and TGFbeta3))) (regenerative effect, wound healing, immunity, cancer, heart disease, diabetes, Marfan syndrome, Loeys-Dietz syndrome), VEGF (VEGF-A, VEGF-B, VEGF-C, VEGF-D, VEGF-E, VEGF-F und PIGF) (regenerative effect, angiogenesis, wound healing, cancer, permeability), Nesiritide (Acute decompensated congestive heart failure), Trypsin (Decubitus ulcer, varicose ulcer, debridement of eschar, dehiscent wound, sunburn, meconium ileus), adrenocorticotrophic hormone (ACTH) (“Addison's disease, Small cell carcinoma, Adrenoleukodystrophy, Congenital adrenal hyperplasia, Cushing's syndrome, Nelson's syndrome, Infantile spasms), Atrial-natriuretic peptide (ANP) (endocrine disorders), Cholecystokinin (diverse), Gastrin (hypogastrinemia), Leptin (Diabetes, hypertriglyceridemia, obesity), Oxytocin (stimulate breastfeeding, non-progression of parturition), Somatostatin (symptomatic treatment of carcinoid syndrome, acute variceal bleeding, and acromegaly, polycystic diseases of the liver and kidney, acromegaly and symptoms caused by neuroendocrine tumors), Vasopressin (antidiuretic hormone) (diabetes insipidus), Calcitonin (Postmenopausal osteoporosis, Hypercalcaemia, Paget's disease, Bone metastases, Phantom limb pain, Spinal Stenosis), Exenatide (Type 2 diabetes resistant to treatment with metformin and a sulphonylurea), Growth hormone (GH), somatotropin (Growth failure due to GH deficiency or chronic renal insufficiency, Prader-Willi syndrome, Turner syndrome, AIDS wasting or cachexia with antiviral therapy), Insulin (Diabetes mellitus, diabetic ketoacidosis, hyperkalaemia), Insulin-like growth factor 1 IGF-1 (Growth failure in children with GH gene deletion or severe primary IGF1 deficiency, neurodegenerative disease, cardiovascular diseases, heart failure), Mecasermin rinfabate, IGF-1 analog (Growth failure in children with GH gene deletion or severe primary IGF1 deficiency, neurodegenerative disease, cardiovascular diseases, heart failure), Mecasermin, IGF-1 analog (Growth failure in children with GH gene deletion or severe primary IGF1 deficiency, neurodegenerative disease, cardiovascular diseases, heart failure), Pegvisomant (Acromegaly), Pramlintide (Diabetes mellitus, in combination with insulin), Teriparatide (human parathyroid hormone residues 1-34) (Severe osteoporosis), Becaplermin (Debridement adjunct for diabetic ulcers), Dibotermin-alpha (Bone morphogenetic protein 2) (Spinal fusion surgery, bone injury repair), Histrelin acetate (gonadotropin releasing hormone; GnRH) (Precocious puberty), Octreotide (Acromegaly, symptomatic relief of VIP-secreting adenoma and metastatic carcinoid tumours), and Palifermin (keratinocyte growth factor; KGF) (Severe oral mucositis in patients undergoing chemotherapy, wound healing), or an isoform, homolog, fragment, variant or derivative of any of these proteins.

These and other proteins are understood to be therapeutic, as they are meant to treat the subject by replacing its defective endogenous production of a functional protein in sufficient amounts.

Accordingly, such therapeutic proteins are typically mammalian, in particular human proteins.

For the treatment of acquired or inherited blood disorders, diseases of the circulatory system, diseases of the respiratory system, cancer or tumour diseases, infectious diseases or immunodeficiencies, the following therapeutic proteins may be used (in brackets is the particular disease for which a use of the therapeutic protein is indicated for treatment): Alteplase (tissue plasminogen activator; tPA) (Pulmonary embolism, myocardial infarction, acute ischaemic stroke, occlusion of central venous access devices), Anistreplase (Thrombolysis), Antithrombin III (AT-III) (Hereditary AT-III deficiency, Thromboembolism), Bivalirudin (Reduce blood-clotting risk in coronary angioplasty and heparin-induced thrombocytopaenia), Darbepoetin-alpha (Treatment of anaemia in patients with chronic renal insufficiency and chronic renal failure (+/− dialysis)), Drotrecogin-alpha (activated protein C) (Severe sepsis with a high risk of death), Erythropoietin, Epoetin-alpha, erythropoetin, erthropoyetin (Anaemia of chronic disease, myleodysplasia, anaemia due to renal failure or chemotherapy, preoperative preparation), Factor IX (Haemophilia B), Factor VIIa (Haemorrhage in patients with haemophilia A or B and inhibitors to factor VIII or factor IX), Factor VIII (Haemophilia A), Lepirudin (Heparin-induced thrombocytopaenia), Protein C concentrate (Venous thrombosis, Purpura fulminans), Reteplase (deletion mutein of tPA) (Management of acute myocardial infarction, improvement of ventricular function), Streptokinase (Acute evolving transmural myocardial infarction, pulmonary embolism, deep vein thrombosis, arterial thrombosis or embolism, occlusion of arteriovenous cannula), Tenecteplase (Acute myocardial infarction), Urokinase (Pulmonary embolism), Angiostatin (Cancer), Anti-CD22 immunotoxin (Relapsed CD33+ acute myeloid leukaemia), Denileukin diftitox (Cutaneous T-cell lymphoma (CTCL)), Immunocyanin (bladder and prostate cancer), MPS (Metallopanstimulin) (Cancer), Aflibercept (Non-small cell lung cancer (NSCLC), metastatic colorectal cancer (mCRC), hormone-refractory metastatic prostate cancer, wet macular degeneration), Endostatin (Cancer, inflammatory diseases like rheumatoid arthritis as well as Crohn's disease, diabetic retinopathy, psoriasis, and endometriosis), Collagenase (Debridement of chronic dermal ulcers and severely burned areas, Dupuytren's contracture, Peyronie's disease), Human deoxy-ribonuclease I, dornase (Cystic fibrosis; decreases respiratory tract infections in selected patients with FVC greater than 40% of predicted), Hyaluronidase (Used as an adjuvant to increase the absorption and dispersion of injected drugs, particularly anaesthetics in ophthalmic surgery and certain imaging agents), Papain (Debridement of necrotic tissue or liquefication of slough in acute and chronic lesions, such as pressure ulcers, varicose and diabetic ulcers, burns, postoperative wounds, pilonidal cyst wounds, carbuncles, and other wounds), L-Asparaginase (Acute lymphocytic leukaemia, which requires exogenous asparagine for proliferation), Peg-asparaginase (Acute lymphocytic leukaemia, which requires exogenous asparagine for proliferation), Rasburicase (Paediatric patients with leukaemia, lymphoma, and solid tumours who are undergoing anticancer therapy that may cause tumour lysis syndrome), Human chorionic gonadotropin (HCG) (Assisted reproduction), Human follicle-stimulating hormone (FSH) (Assisted reproduction), Lutropin-alpha (Infertility with luteinizing hormone deficiency), Prolactin (Hypoprolactinemia, serum prolactin deficiency, ovarian dysfunction in women, anxiety, arteriogenic erectile dysfunction, premature ejaculation, oligozoospermia, asthenospermia, hypofunction of seminal vesicles, hypoandrogenism in men), alpha-1-Proteinase inhibitor (Congenital antitrypsin deficiency), Lactase (Gas, bloating, cramps and diarrhoea due to inability to digest lactose), Pancreatic enzymes (lipase, amylase, protease) (Cystic fibrosis, chronic pancreatitis, pancreatic insufficiency, post-Billroth II gastric bypass surgery, pancreatic duct obstruction, steatorrhoea, poor digestion, gas, bloating), Adenosine deaminase (pegademase bovine, PEG-ADA) (Severe combined immunodeficiency disease due to adenosine deaminase deficiency), Abatacept (Rheumatoid arthritis (especially when refractory to TNFa inhibition)), Alefacept (Plaque Psoriasis), Anakinra (Rheumatoid arthritis), Etanercept (Rheumatoid arthritis, polyarticular-course juvenile rheumatoid arthritis, psoriatic arthritis, ankylosing spondylitis, plaque psoriasis, ankylosing spondylitis), Interleukin-1 (IL-1) receptor antagonist, Anakinra (inflammation and cartilage degradation associated with rheumatoid arthritis), Thymulin (neurodegenerative diseases, rheumatism, anorexia nervosa), TNF-alpha antagonist (autoimmune disorders such as rheumatoid arthritis, ankylosing spondylitis, Crohn's disease, psoriasis, hidradenitis suppurativa, refractory asthma), Enfuvirtide (HIV-1 infection), and Thymosin alpha1 (Hepatitis B and C), or an isoform, homolog, fragment, variant or derivative of any of these proteins.

Further therapeutic (poly-)peptides or proteins may be selected from: 0ATL3, 0FC3, 0PA3, 0PD2, 4-1BBL, 5T4, 6Ckine, 707-AP, 9D7, A2M, AA, AAAS, AACT, AASS, ABAT, ABCA1, ABCA4, ABCB1, ABCB11, ABCB2, ABCB4, ABCB7, ABCC2, ABCC6, ABCC8, ABCD1, ABCD3, ABCG5, ABCG8, ABL1, ABO, ABR ACAA1, ACACA, ACADL, ACADM, ACADS, ACADVL, ACAT1, ACCPN, ACE, ACHE, ACHM3, ACHM1, ACLS, ACPI, ACTA1, ACTC, ACTN4, ACVRL1, AD2, ADA, ADAMTS13, ADAMTS2, ADFN, ADH1B, ADH1C, ADLDH3A2, ADRB2, ADRB3, ADSL, AEZ, AFA, AFD1, AFP, AGA, AGL, AGMX2, AGPS, AGS1, AGT, AGTR1, AGXT, AH02, AHCY, AHDS, AHHR, AHSG, AIC, AIED, AIH2, AIH3, AIM-2, AIPL1, AIRE, AK1, ALAD, ALAS2, ALB, HPG1, ALDH2, ALDH3A2, ALDH4A1, ALDH5A1, ALDH1A1, ALDOA, ALDOB, ALMS1, ALPL, ALPP, ALS2, ALX4, AMACR, AMBP, AMCD, AMCD1, AMCN, AMELX, AMELY, AMGL, AMH, AMHR2, AMPD3, AMPD1, AMT, ANC, ANCR, ANK1, ANOP1, AOM, AP0A4, AP0C2, AP0C3, AP3B1, APC, aPKC, APOA2, APOA1, APOB, APOC3, APOC2, APOE, APOH, APP, APRT, APS1, AQP2, AR, ARAF1, ARG1, ARHGEF12, ARMET, ARSA, ARSB, ARSC2, ARSE, ART-4, ARTC1/m, ARTS, ARVD1, ARX, AS, ASAH, ASAT, ASD1, ASL, ASMD, ASMT, ASNS, ASPA, ASS, ASSP2, ASSP5, ASSP6, AT3, ATD, ATHS, ATM, ATP2A1, ATP2A2, ATP2C1, ATP6B1, ATP7A, ATP7B, ATP8B1, ATPSK2, ATRX, ATXN1, ATXN2, ATXN3, AUTS1, AVMD, AVP, AVPR2, AVSD1, AXIN1, AXIN2, AZF2, B2M, B4GALT7, B7H4, BAGE, BAGE-1, BAX, BBS2, BBS3, BBS4, BCA225, BCAA, BCH, BCHE, BCKDHA, BCKDHB, BCL10, BCL2, BCL3, BCL5, BCL6, BCPM, BCR, BCR/ABL, BDC, BDE, BDMF, BDMR, BEST1, beta-Catenin/m, BF, BFHD, BFIC, BFLS, BFSP2, BGLAP, BGN, BHD, BHR1, BING-4, BIRC5, BJS, BLM, BLMH, BLNK, BMPR2, BPGM, BRAF, BRCA1, BRCA1/m, BRCA2, BRCA2/m, BRCD2, BRCD1, BRDT, BSCL, BSCL2, BTAA, BTD, BTK, BUB1, BWS, BZX, C0L2A1, C0L6A1, C1NH, C1QA, C1QB, C1QG, CIS, C2, C3, C4A, C4B, C5, C6, C7, C7orf2, C8A, C8B, C9, CA125, CA15-3/CA 27-29, CA195, CA19-9, CA72-4, CA2, CA242, CA50, CABYR, CACD, CACNA2D1, CACNA1A, CACNA1F, CACNA1S, CACNB2, CACNB4, CAGE, CA1, CALB3, CALCA, CALCR, CALM, CALR, CAM43, CAMEL, CAP-1, CAPN3, CARD15, CASP-5/m, CASP-8, CASP-8/m, CASR, CAT, CATM, CAV3, CB1, CBBM, CBS, CCA1, CCAL2, CCAL1, CCAT, CCL-1, CCL-11, CCL-12, CCL-13, CCL-14, CCL-15, CCL-16, CCL-17, CCL-18, CCL-19, CCL-2, CCL-20, CCL-21, CCL-22, CCL-23, CCL-24, CCL-25, CCL-27, CCL-3, CCL-4, CCL-5, CCL-7, CCL-8, CCM1, CCNB1, CCND1, CCO, CCR2, CCR5, CCT, CCV, CCZS, CD1, CD19, CD20, CD22, CD25, CD27, CD27L, cD3, CD30, CD30, CD30L, CD33, CD36, CD3E, CD3G, CD3Z, CD4, CD40, CD40L, CD44, CD44v, CD44v6, CD52, CD55, CD56, CD59, CD80, CD86, CDAN1, CDAN2, CDAN3, CDC27, CDC27/m, CDC2L1, CDH1, CDK4, CDK4/m, CDKN1C, CDKN2A, CDKN2A/m, CDKN1A, CDKN1C, CDL1, CDPD1, CDR1, CEA, CEACAM1, CEACAM5, CECR, CECR9, CEPA, CETP, CFNS, CFTR, CGF1, CHAC, CHED2, CHED1, CHEK2, CHM, CHML, CHR39C, CHRNA4, CHRNA1, CHRNB1, CHRNE, CHS, CHS1, CHST6, CHX10, CIAS1, CIDX, CKN1, CLA2, CLA3, CLA1, CLCA2, CLCN1, CLCN5, CLCNKB, CLDN16, CLP, CLN2, CLN3, CLN4, CLN5, CLN6, CLN8, C1QA, C1QB, C1QG, C1R, CLS, CMCWTD, CMDJ, CMD1A, CMD1B, CMH2, MH3, CMH6, CMKBR2, CMKBR5, CML28, CML66, CMM, CMT2B, CMT2D, CMT4A, CMT1A, CMTX2, CMTX3, C-MYC, CNA1, CND, CNGA3, CNGA1, CNGB3, CNSN, CNTF, COA-1/m, COCH, COD2, COD1, COH1, COL10A, COL2A2, COL11A2, COL17A1, COL1A1, COL1A2, COL2A1, COL3A1, COL4A3, COL4A4, COL4A5, COL4A6, COL5A1, COL5A2, COL6A1, COL6A2, COL6A3, COL7A1, COL8A2, COL9A2, COL9A3, COL11A1, COL1A2, COL23A1, COL1A1, COLQ, COMP, COMT, CORD5, CORD1, COX10, COX-2, CP, CPB2, CPO, CPP, CPS1, CPT2, CPT1A, CPX, CRAT, CRB1, CRBM, CREBBP, CRH, CRHBP, CRS, CRV, CRX, CRYAB, CRYBA1, CRYBB2, CRYGA, CRYGC, CRYGD, CSA, CSE, CSF1R, CSF2RA, CSF2RB, CSF3R, CSF1R, CST3, CSTB, CT, CT7, CT-9/BRD6, CTAA1, CTACK, CTEN, CTH, CTHM, CTLA4, CTM, CTNNB1, CTNS, CTPA, CTSB, CTSC, CTSK, CTSL, CTS1, CUBN, CVD1, CX3CL1, CXCL1, CXCL10, CXCL11, CXCL12, CXCL13, CXCL16, CXCL2, CXCL3, CXCL4, CXCL5, CXCL6, CXCL7, CXCL8, CXCL9, CYB5, CYBA, CYBB, CYBB5, CYFRA 21-1, CYLD, CYLD1, CYMD, CYP11B1, CYP11B2, CYP17, CYP17A1, CYP19, CYP19A1, CYP1A2, CYP1B1, CYP21A2, CYP27A1, CYP27B1, CYP2A6, CYP2C, CYP2C19, CYP2C9, CYP2D, CYP2D6, CYP2D7P1, CYP3A4, CYP7B1, CYPB1, CYP11B1, CYP1A1, CYP1B1, CYRAA, D40, DADI, DAM, DAM-10/MAGE-B1, DAM-6/MAGE-B2, DAX1, DAZ, DBA, DBH, DBI, DBT, DCC, DC-CK1, DCK, DCR, DCX, DDB 1, DDB2, DDIT3, DDU, DECR1, DEK-CAN, DEM, DES, DF, DFN2, DFN4, DFN6, DFNA4, DFNA5, DFNB5, DGCR, DHCR7, DHFR, DHOF, DHS, DIA1, DIAPH2, DIAPH1, DIH1, DIO1, DISCI, DKC1, DLAT, DLD, DLL3, DLX3, DMBT1, DMD, DM1, DMPK, DMWD, DNAI1, DNASE1, DNMT3B, DPEP1, DPYD, DPYS, DRD2, DRD4, DRPLA, DSCR1, DSG1, DSP, DSPP, DSS, DTDP2, DTR, DURS1, DWS, DYS, DYSF, DYT2, DYT3, DYT4, DYT2, DYT1, DYX1, EBAF, EBM, EBNA, EBP, EBR3, EBS1, ECA1, ECB2, ECE1, ECGF1, ECT, ED2, ED4, EDA, EDAR, ECA1, EDN3, EDNRB, EEC1, EEF1A1L14, EEGV1, EFEMP1, EFTUD2/m, EGFR, EGFR/Her1, EGI, EGR2, EIF2AK3, eIF4G, EKV, EI IS, ELA2, ELF2, ELF2M, ELK1, ELN, ELONG, EMD, EML1, EMMPRIN, EMX2, ENA-78, ENAM, END3, ENG, ENO1, ENPP1, ENUR2, ENUR1, EOS, EP300, EPB41, EPB42, EPCAM, EPD, EphA1, EphA2, EphA3, EphrinA2, EphrinA3, EPHX1, EPM2A, EPO, EPOR, EPX, ERBB2, ERCC2 ERCC3, ERCC4, ERCC5, ERCC6, ERVR, ESR1, ETFA, ETFB, ETFDH, ETM1, ETV6-AML1, ETV1, EVC, EVR2, EVR1, EWSR1, EXT2, EXT3, EXT1, EYA1, EYCL2, EYCL3, EYCL1, EZH2, F10, F11, F12, F13A1, F13B, F2, F5, F5F8D, F7, F8, F8C, F9, FABP2, FACL6, FAH, FANCA, FANCB, FANCC, FANCD2, FANCF, FasL, FBN2, FBN1, FBP1, FCG3RA, FCGR2A, FCGR2B, FCGR3A, FCHL, FCMD, FCP1, FDPSL5, FECH, FEO, FEOM1, FES, FGA, FGB, FGD1, FGF2, FGF23, FGF5, FGFR2, FGFR3, FGFR1, FGG, FGS1, FH, FIC1, FIH, F2, FKBP6, FLNA, FLT4, FMO3, FMO4, FMR2, FMR1, FN, FN1/m, FOXC1, FOXE1, FOXL2, FOXO1A, FPDMM, FPF, Fra-1, FRAXF, FRDA, FSHB, FSHMD1A, FSHR, FTH1, FTHL17, FTL, FTZF1, FUCA1, FUT2, FUT6, FUT1, FY, G250, G250/CAIX, G6PC, G6PD, G6PT1, G6PT2, GAA, GABRA3, GAGE-1, GAGE-2, GAGE-3, GAGE-4, GAGE-5, GAGE-6, GAGE-7b, GAGE-8, GALC, GALE, GALK1, GALNS, GALT, GAMT, GAN, GAST, GASTRIN17, GATA3, GATA, GBA, GBE, GC, GCDH, GCGR, GCH1, GCK, GCP-2, GCS1, G-CSF, GCSH, GCSL, GCY, GDEP, GDF5, GDI1, GDNF, GDXY, GFAP, GFND, GGCX, GGT1, GH2, GH1, GHR, GHRHR, GHS, GIF, GINGF, GIP, GJA3, GJA8, GJB2, GJB3, GJB6, GJB1, GK, GLA, GLB, GLB1, GLC3B, GLC1B, GLC1C, GLDC, GLI3, GLP1, GLRA1, GLUD1, GM1 (fuc-GM1), GM2A, GM-CSF, GMPR, GNAI2, GNAS, GNAT1, GNB3, GNE, GNPTA, GNRH, GNRH1, GNRHR, GNS, GnT-V, gp100, GP1BA, GP1BB, GP9, GPC3, GPD2, GPDS1, GPI, GP1BA, GPN1LW, GPNMB/m, GPSC, GPX1, GRHPR, GRK1, GROα, GROβ, GROγ, GRPR, GSE, GSM1, GSN, GSR, GSS, GTD, GTS, GUCA1A, GUCY2D, GULOP, GUSB, GUSM, GUST, GYPA, GYPC, GYS1, GYS2, HOKPP2, HOMG2, HADHA, HADHB, HAGE, HAGH, HAL, HAST-2, HB 1, HBA2, HBA1, HBB, HBBP1, HBD, HBE1, HBG2, HBG1, HBHR, HBP1, HBQ1, HBZ, HBZP, HCA, HCC-1, HCC-4, HCF2, HCG, HCL2, HCL1, HCR, HCVS, HD, HPN, HER2, HER2/NEU, HER3, HERV-K-MEL, HESX1, HEXA, HEXB, HF1, HFE, HF1, HGD, HHC2, HHC3, HHG, HK1 HLA-A, HLA-A*0201-R170I, HLA-A11/m, HLA-A2/m, HLA-DPB1 HLA-DRA, HLCS, HLXB9, HMBS, HMGA2, HMGCL, HMI, HMN2, HMOX1, HMS1 HMW-MAA, HND, HNE, HNF4A, HOAC, HOMEOBOX NKX 3.1, HOM-TES-14/SCP-1, HOM-TES-85, HOXA1 HOXD13, HP, HPC1, HPD, HPE2, HPE1, HPFH, HPFH2, HPRT1, HPS1, HPT, HPV-E6, HPV-E7, HR, HRAS, HRD, HRG, HRPT2, HRPT1, HRX, HSD11B2, HSD17B3, HSD17B4, HSD3B2, HSD3B3, HSN1, HSP70-2M, HSPG2, HST-2, HTC2, HTC1, hTERT, HTN3, HTR2C, HVBS6, HVBS1, HVEC, HV1S, HYAL1, HYR, I-309, IAB, IBGC1, IBM2, ICAM1, ICAM3, iCE, ICHQ, ICR5, ICR1, ICS 1, IDDM2, IDDM1, IDS, IDUA, IF, IFNa/b, IFNGR1, IGAD1, IGER, IGF-1R, IGF2R, IGF1, IGH, IGHC, IGHG2, IGHG1, IGHM, IGHR, IGKC, IHG1, IHH, IKBKG, IL1, IL-1 RA, IL10, IL-11, IL12, IL12RB1, IL13, IL-13Ralpha2, IL-15, IL-16, IL-17, IL18, IL-1a, IL-1alpha, IL-1b, IL-1beta, IL1RAPL1, IL2, IL24, IL-2R, IL2RA, IL2RG, IL3, IL3RA, IL4, IL4R, IL4R, IL-5, IL6, IL-7, IL7R, IL-8, IL-9, Immature laminin receptor, IMMP2L, INDX, INFGR1, INFGR2, INFalpha, IFNbeta, INFgamma, INS, INSR, INVS, IP-10, IP2, IPF1, IP1, IRF6, IRS1, ISCW, ITGA2, ITGA2B, ITGA6, ITGA7, ITGB2, ITGB3, ITGB4, ITIH1, ITM2B, IV, IVD, JAG1, JAK3, JBS, JBTS1, JMS, JPD, KAL1, KAL2, KALI, KLK2, KLK4, KCNA1, KCNE2, KCNE1, KCNH2, KCNJ1, KCNJ2, KCNJ1, KCNQ2, KCNQ3, KCNQ4, KCNQ1, KCS, KERA, KFM, KFS, KFSD, KHK, ki-67, KIAA0020, KIAA0205, KIAA0205/m, KIF1B, KIT, KK-LC-1, KLK3, KLKB1, KM-HN-1, KMS, KNG, KNO, K-RAS/m, KRAS2, KREV1, KRT1, KRT10, KRT12, KRT13, KRT14, KRT14L1, KRT14L2, KRT14L3, KRT16, KRT16L1, KRT16L2, KRT17, KRT18, KRT2A, KRT3, KRT4, KRT5, KRT6 A, KRT6B, KRT9, KRTHB1, KRTHB6, KRT1, KSA, KSS, KWE, KYNU, L0H19CR1, L1CAM, LAGE, LAGE-1, LALL, LAMA2, LAMA3, LAMB3, LAMB1, LAMC2, LAMP2, LAP, LCA5, LCAT, LCCS, LCCS 1, LCFS2, LCS1, LCT, LDHA, LDHB, LDHC, LDLR, LDLR/FUT, LEP, LEWISY, LGCR, LGGF-PBP, LGI1, LGMD2H, LGMD1A, LGMD1B, LHB, LHCGR, LHON, LHRH, LHX3, LIF, LIG1, LIMM, LIMP2, LIPA, LIPA, LIPB, LIPC, LIVIN, L1CAM, LMAN1, LMNA, LMX1B, LOLR, LOR, LOX, LPA, LPL, LPP, LQT4, LRP5, LRS 1, LSFC, LT-beta, LTBP2, LTC4S, LYL1, XCL1, LYZ, M344, MA50, MAA, MADH4, MAFD2, MAFD1, MAGE, MAGE-A1, MAGE-A10, MAGE-A12, MAGE-A2, MAGE-A3, MAGE-A4, MAGE-A6, MAGE-A9, MAGEB1, MAGE-B10, MAGE-B16, MAGE-B17, MAGE-B2, MAGE-B3, MAGE-B4, MAGE-B5, MAGE-B6, MAGE-C1, MAGE-C2, MAGE-C3, MAGE-D1, MAGE-D2, MAGE-D4, MAGE-E1, MAGE-E2, MAGE-F1, MAGE-H1, MAGEL2, MGB1, MGB2, MAN2A1, MAN2B1, MANBA, MANBB, MAOA, MAOB, MAPK8IP1, MAPT, MART-1, MART-2, MART2/m, MAT1A, MBL2, MBP, MBS1, MC1R, MC2R, MC4R, MCC, MCCC2, MCCC1, MCDR1, MCF2, MCKD, MCL1, MC1R, MCOLN1, MCOP, MCOR, MCP-1, MCP-2, MCP-3, MCP-4, MCPH2, MCPH1, MCS, M-CSF, MDB, MDCR, MDM2, MDRV, MDS 1, ME1, ME1/m, ME2, ME20, ME3, MEAX, MEB, MEC CCL-28, MECP2, MEFV, MELANA, MELAS, MEN1 MSLN, MET, MF4, MG50, MG50/PXDN, MGAT2, MGAT5, MGC1 MGCR, MGCT, MGI, MGP, MHC2TA, MHS2, MHS4, MIC2, MIC5, MIDI, MIF, MIP, MIP-5/HCC-2, MITF, MJD, MKI67, MKKS, MKS1, MLH1, MLL, MLLT2, MLLT3, MLLT7, MLLT1, MLS, MLYCD, MMA1a, MMP 11, MMVP1, MN/CA IX-Antigen, MNG1, MN1, MOC31, MOCS2, MOCS1, MOG, MORC, MOS, MOV18, MPD1, MPE, MPFD, MPI, MPIF-1, MPL, MPO, MPS3C, MPZ, MRE11A, MROS, MRP1, MRP2, MRP3, MRSD, MRX14, MRX2, MRX20, MRX3, MRX40, MRXA, MRX1, MS, MS4A2, MSD, MSH2, MSH3, MSH6, MSS, MSSE, MSX2, MSX1, MTATP6, MTC03, MTCO1, MTCYB, MTHFR, MTM1, MTMR2, MTND2, MTND4, MTND5, MTND6, MTND1, MTP, MTR, MTRNR2, MTRNR1, MTRR, MTTE, MTTG, MTTI, MTTK, MTTL2, MTTL1, MTTN, MTTP, MTTS1, MUC1, MUC2, MUC4, MUC5AC, MUM-1, MUM-1/m, MUM-2, MUM-2/m, MUM-3, MUM-3/m, MUT, mutant p21 ras, MUTYH, MVK, MX2, MXI1, MY05A, MYB, MYBPC3, MYC, MYCL2, MYH6, MYH7, MYL2, MYL3, MYMY, MYO15A, MYO1G, MYO5A, MYO7A, MYOC, Myosin/m, MYP2, MYP1, NA88-A, N-acetylglucosaminyltransferase-V, NAGA, NAGLU, NAMSD, NAPB, NAT2, NAT, NBIA1, NBS1, NCAM, NCF2, NCF1, NDN, NDP, NDUFS4, NDUFS7, NDUFS8, NDUFV1, NDUFV2, NEB, NEFH, NEM1, Neo-PAP, neo-PAP/m, NEU1, NEUROD1, NF2, NF1, NFYC/m, NGEP, NHS, NKS1, NKX2E, NM, NME1, NMP22, NMTC, NODAL, NOG, NOS3, NOTCH3, NOTCH1, NP, NPC2, NPC1, NPHL2, NPHP1, NPHS2, NPHS1, NPM/ALK, NPPA, NQO1, NR2E3, NR3C1, NR3C2, NRAS, NRAS/m, NRL, NROB1, NRTN, NSE, NSX, NTRK1, NUMA1, NXF2, NY-CO1, NY-ESO1, NY-ESO-B, NY-LU-12, ALDOA, NYS2, NYS4, NY-SAR-35, NYS1, NYX, OA3, OA1, OAP, OASD, OAT, OCA1, OCA2, OCD1, OCRL, OCRL1, OCT, ODDD, ODT1, OFC1, OFD1, OGDH, OGT, OGT/m, OPA2, OPA1, OPD1, OPEM, OPG, OPN, OPN1LW, OPN1MW, OPN1SW, OPPG, OPTB1, TTD, ORM1, ORP1, OS-9, OS-9/m, OSM LIF, OTC, OTOF, OTSC1, OXCT1, OYTES1, P15, P190 MINOR BCR-ABL, P2RY12, P3, P16, P40, P4HB, P-501, P53, P53/m, P97, PABPN1, PAFAH1B1, PAFAH1P1, PAGE-4, PAGE-5, PAH, PAI-1, PAI-2, PAK3, PAP, PAPPA, PARK2, PART-1, PATE, PAX2, PAX3, PAX6, PAX7, PAX8, PAX9, PBCA, PBCRA1, PBT, PBX1, PBXP1, PC, PCBD, PCCA, PCCB, PCK2, PCK1, PCLD, PCOS1, PCSK1, PDB1, PDCN, PDE6A, PDE6B, PDEF, PDGFB, PDGFR, PDGFRL, PDHA1, PDR, PDX1, PECAM1, PEE1, PEO1, PEPD, PEX10, PEX12, PEX13, PEX3, PEX5, PEX6, PEX7, PEX1, PF4, PFB1, PFC, PFKFB1, PFKM, PGAM2, PGD, PGK1, PGK1P1, PGL2, PGR, PGS, PHA2A, PHB, PHEX, PHGDH, PHKA2, PHKA1, PHKB, PHKG2, PHP, PHYH, PI, PI3, PIGA, PIM1-KINASE, PIN1, PIP5K1B, PITX2, PITX3, PKD2, PKD3, PKD1, PKDTS, PKHD1, PKLR, PKP1, PKU1, PLA2G2A, PLA2G7, PLAT, PLEC1, PLG, PLI, PLOD, PLP1, PMEL17, PML, PML/RARalpha, PMM2, PMP22, PMS2, PMS1, PNKD, PNLIP, POF1, POLA, POLH, POMC, PON2, PON1, PORC, POTE, POU1F1, POU3F4, POU4F3, POU1F1, PPAC, PPARG, PPCD, PPGB, PPH1, PPKB, PPMX, PPOX, PPP1R3A, PPP2R2B, PPT1, PRAME, PRB, PRB3, PRCA1, PRCC, PRD, PRDX5/m, PRF1, PRG4, PRKAR1A, PRKCA, PRKDC, PRKWNK4, PRNP, PROC, PRODH, PROM1, PROP1, PROS1, PRST, PRP8, PRPF31, PRPF8, PRPH2, PRPS2, PRPS1, PRS, PRSS7, PRSS1, PRTN3, PRX, PSA, PSAP, PSCA, PSEN2, PSEN1, PSG1, PSGR, PSM, PSMA, PSORS1, PTC, PTCH, PTCH1, PTCH2, PTEN, PTGS1, PTH, PTHR1, PTLAH, PTOS1, PTPN12, PTPNI l, PTPRK, PTPRK/m, PTS, PUJO, PVR, PVRL1, PWCR, PXE, PXMP3, PXR1, PYGL, PYGM, QDPR, RAB27A, RAD54B, RAD54L, RAG2, RAGE, RAGE-1, RAG1, RAP1, RARA, RASA1, RBAF600/m, RB1, RBP4, RBP4, RBS, RCA1, RCAS1, RCCP2, RCD1, RCV1, RDH5, RDPA, RDS, RECQL2, RECQL3, RECQL4, REG1A, REHOBE, REN, RENBP, RENS1, RET, RFX5, RFXANK, RFXAP, RGR, RHAG, RHAMM/CD168, RHD, RHO, Rip-1, RLBP1, RLN2, RLN1, RLS, RMD1, RMRP, ROM1, ROR2, RP, RP1, RP14, RP17, RP2, RP6, RP9, RPD1, RPE65, RPGR, RPGRIP1, RP1, RP10, RPS19, RPS2, RPS4X, RPS4Y, RPS6KA3, RRAS2, RS1, RSN, RSS, RU1, RU2, RUNX2, RUNXI, RWS, RYR1, S-100, SAA1, SACS, SAG, SAGE, SALL1, SARDH, SART1, SART2, SART3, SAS, SAX1, SCA2, SCA4, SCA5, SCA7, SCA8, SCA1, SCC, SCCD, SCF, SCLC1, SCN1A, SCN1B, SCN4A, SCN5A, SCNN1A, SCNN1B, SCNN1G, SCO2, SCP1, SCZD2, SCZD3, SCZD4, SCZD6, SCZD1, SDF-1alpha/beta, SDHA, SDHD, SDYS, SEDL, SERPENA7, SERPINA3, SERPINA6, SERPINA1, SERPINC1, SERPIND1, SERPINE1, SERPINF2, SERPING1, SERPINI1, SFTPA1, SFTPB, SFTPC, SFTPD, SGCA, SGCB, SGCD, SGCE, SGM1, SGSH, SGY-1, SH2D1A, SHBG, SHFM2, SHFM3, SHFM1, SHH, SHOX, SI, SIAL, SIALYL LEWISX, SIASD, S11, SIM1, SIRT2/m, SIX3, SJS1, SKP2, SLC10A2, SLC12A1, SLC12A3, SLC17A5, SLC19A2, SLC22A1L, SLC22A5, SLC25A13, SLC25A15, SLC25A20, SLC25A4, SLC25A5, SLC25A6, SLC26A2, SLC26A3, SLC26A4, SLC2A1, SLC2A2, SLC2A4, SLC3A1, SLC4A1, SLC4A4, SLC5A1, SLC5A5, SLC6A2, SLC6A3, SLC6A4, SLC7A7, SLC7A9, SLC11A1, SLOS, SMA, SMAD1, SMAL, SMARCB1, SMAX2, SMCR, SMCY, SM1, SMN2, SMN1, SMPD1, SNCA, SNRPN, SOD2, SOD3, SOD1, SOS1, SOST, SOX9, SOX10, Sp17, SPANXC, SPG23, SPG3A, SPG4, SPG5A, SPG5B, SPG6, SPG7, SPINK1, SPINK5, SPPK, SPPM, SPSMA, SPTA1, SPTB, SPTLC1, SRC, SRD5A2, SRPX, SRS, SRY, ßhCG, SSTR2, SSX1, SSX2 (HOM-MEL-40/SSX2), SSX4, ST8, STAMP-1, STAR, STARP1, STATH, STEAP, STK2, STK11, STn/KLH, STO, STOM, STS, SUOX, SURF1, SURVIVIN-2B, SYCP1, SYM1, SYN1, SYNS1, SYP, SYT/SSX, SYT-SSX-1, SYT-SSX-2, TA-90, TAAL6, TACSTD1, TACSTD2, TAG72, TAF7L, TAF1, TAGE, TAG-72, TALI, TAM, TAP2, TAP1, TAPVR1, TARC, TARP, TAT, TAZ, TBP, TBX22, TBX3, TBX5, TBXA2R, TBXAS1, TCAP, TCF2, TCF1, TCIRG1, TCL2, TCL4, TCL1A, TCN2, TCOF1, TCR, TCRA, TDD, TDFA, TDRD1, TECK, TECTA, TEK, TEL/AML1, TELAB1, TEX15, TF, TFAP2B, TFE3, TFR2, TG, TGFalpha, TGFbeta, TGFbetaI, TGFbeta1, TGFbetaR2, TGFbetaRE, TGFgamma, TGFbetaRII, TGIF, TGM-4, TGM1, TH, THAS, THBD, THC, THC2, THM, THPO, THRA, THRB, TIMM8A, TIMP2, TIMP3, TIMP1, TITF1, TKCR, TKT, TLP, TLR1, TLR10, TLR2, TLR3, TLR4, TLR4, TLR5, TLR6, TLR7, TLR8, TLR9, TLX1, TM4SF1, TM4SF2, TMC1, TMD, TMIP, TNDM, TNF, TNFRSF11A, TNFRSF1A, TNFRSF6, TNFSF5, TNFSF6, TNFalpha, TNFbeta, TNNI3, TNNT2, TOC, TOP2A, TOP1, TP53, TP63, TPA, TPBG, TPI, TPI/m, TPI1, TPM3, TPM1, TPMT, TPO, TPS, TPTA, TRA, TRAG3, TRAPPC2, TRC8, TREH, TRG, TRH, TRIM32, TRIM37, TRP1, TRP2, TRP-2/6b, TRP-2/INT2, Trp-p8, TRPS1, TS, TSC2, TSC3, TSC1, TSG101, TSHB, TSHR, TSP-180, TST, TTGA2B, TTN, TTPA, TTR, TU M2-PK, TULP1, TWIST, TYH, TYR, TYROBP, TYROBP, TYRP1, TYS, UBE2A, UBE3A, UBE1, UCHL1, UFS, UGT1A, ULR, UMPK, UMPS, UOX, UPA, UQCRC1, URO5, UROD, UPK1B, UROS, USH2A, USH3A, USH1A, USH1C, USP9Y, UV24, VBCH, VCF, VDI, VDR, VEGF, VEGFR-2, VEGFR-1, VEGFR-2/FLK-1, VHL, VIM, VMD2, VMD1, VMGLOM, VNEZ, VNF, VP, VRNI, VWF, VWS, WAS, WBS2, WFS2, WFS1, WHCR, WHN, WISP3, WMS, WRN, WS2A, WS2B, WSN, WSS, WT2, WT3, WT1, WTS, WWS, XAGE, XDH, XIC, XIST, XK, XM, XPA, XPC, XRCC9, XS, ZAP70, ZFHX1B, ZFX, ZFY, ZIC2, ZIC3, ZNF145, ZNF261, ZNF35, ZNF41, ZNF6, ZNF198, and ZWS1, or an isoform, homolog, fragment, variant or derivative of any of these proteins.

Further therapeutic (poly-)peptides or proteins may be selected from apoptotic factors or apoptosis related proteins including AIF, Apaf e.g. Apaf-1, Apaf-2, Apaf-3, oder APO-2 (L), APO-3 (L), Apopain, Bad, Bak, Bax, Bcl-2, Bcl-x[L], Bcl-x[s], bik, CAD, Calpain, Caspase e.g. Caspase-1, Caspase-2, Caspase-3, Caspase-4, Caspase-5, Caspase-6, Caspase-7, Caspase-8, Caspase-9, Caspase-10, Caspase-1 1, ced-3, ced-9, c-Jun, c-Myc, crm A, cytochrom C, CdR1, DcR1, DD, DED, DISC, DNA-PKc[S], DR3, DR4, DR5, FADD/MORT-1, FAK, Fas (Fas-ligand CD95/fas (receptor)), FLICE/MACH, FLIP, fodrin, fos, G-Actin, Gas-2, gelsolin, granzyme A/B, ICAD, ICE, JNK, lamin A/B, MAP, MCL-1, Mdm-2, MEKK-1, MORT-1, NEDD, NF-[kappa]B, NuMa, p53, PAK-2, PARP, perforin, PITSLRE, PKCdelta, pRb, presenilin, prICE, RAIDD, Ras, RIP, sphingomyelinase, thymidinkinase from herpes simplex, TRADD, TRAF2, TRAIL-R1, TRAIL-R2, TRAIL-R3, transglutaminase, et cetera, or an isoform, homolog, fragment, variant or derivative of any of these proteins.

An “adjuvant” (poly-)peptide or protein generally means any (poly-)peptide or protein capable of modifying the effect of other agents, typically other active agents that are administered simultaneously. Preferably, “adjuvant or immunostimulating” (poly-)peptides or proteins are capable potentiating or modulating a desired immune response to a (preferably co-administered) antigen. In particular, an “adjuvant or immuno-stimulating” (poly-)peptide or protein may act to accelerate, prolong, or enhance immune responses when used in combination with specific antigens. To that end, “adjuvant or immuno-stimulating” (poly-)peptides or proteins may support administration and delivery of co-administered antigens, enhance the (antigen-specific) immunostimulatory properties of co-administered antigens, and/or initiate or increase an immune response of the innate immune system, i.e. a non-specific immune response. Exemplary “adjuvant or immunostimulating (poly-)peptides or proteins” envisaged in the present invention include mammalian proteins, in particular human adjuvant proteins, which typically comprise any human protein or peptide, which is capable of eliciting an innate immune response (in a mammal), e.g. as a reaction of the binding of an exogenous TLR ligand to a TLR. More preferably, human adjuvant proteins are selected from the group consisting of proteins which are components and ligands of the signalling networks of the pattern recognition receptors including TLR, NLR and RLH, including TLR1, TLR2, TLR3, TLR4, TLR5, TLR6, TLR7, TLR8, TLR9, TLR10, TLR11; NOD1, NOD2, NOD3, NOD4, NOD5, NALP1, NALP2, NALP3, NALP4, NALP5, NALP6, NALP6, NALP7, NALP7, NALP8, NALP9, NALP10, NALP11, NALP12, NALP13, NALP14, l IPAF, NAIP, CIITA, RIG-I, MDA5 and LGP2, the signal transducers of TLR signaling including adaptor proteins including e.g. Trif and Cardif; components of the Small-GTPases signalling (RhoA, Ras, Rac1, Cdc42, Rab etc.), components of the PIP signalling (PI3K, Src-Kinases, etc.), components of the MyD88-dependent signalling (MyD88, IRAK1, IRAK2, IRAK4, TIRAP, TRAF6 etc.), components of the MyD88-independent signalling (TICAM1, TICAM2, TRAF6, TBK1, IRF3, TAK1, IRAK1 etc.); the activated kinases including e.g. Akt, MEKK1, MKK1, MKK3, MKK4, MKK6, MKK7, ERK1, ERK2, GSK3, PKC kinases, PKD kinases, GSK3 kinases, JNK, p38MAPK, TAK1, IKK, and TAK1; the activated transcription factors including e.g. NF-kappaB, c-Fos, c-Jun, c-Myc, CREB, AP-1, Elk-1, ATF2, IRF-3, IRF-7, or an isoform, homolog, fragment, variant or derivative of any of these proteins.

Adjuvant (preferably mammalian) (poly-)peptides or proteins or proteins may further be selected from the group consisting of heat shock proteins, such as HSP10, HSP60, HSP65, HSP70, HSP75 and HSP90, gp96, Fibrinogen, TypIII repeat extra domain A of fibronectin; or components of the complement system including C1q, MBL, C1r, C1s, C2b, Bb, D, MASP-1, MASP-2, C4b, C3b, C5a, C3a, C4a, C5b, C6, C7, C8, C9, CR1, CR2, CR3, CR4, C1qR, C1INH, C4 bp, MCP, DAF, H, I, P and CD59, or induced target genes including e.g. Beta-Defensin, cell surface proteins; or human adjuvant proteins including trif, flt-3 ligand, Gp96 or fibronectin, etc., or an isoform, homolog, fragment, variant or derivative of any of these proteins.

Adjuvant (preferably mammalian) (poly-)peptides or proteins or proteins may further be selected from the group consisting of cytokines which induce or enhance an innate immune response, including IL-1 alpha, IL1 beta, IL-2, IL-6, IL-7, IL-8, IL-9, IL-12, IL-13, IL-15, IL-16, IL-17, IL-18, IL-21, IL-23, TNFalpha, IFNalpha, IFNbeta, IFNgamma, GM-CSF, G-CSF, M-CSF; chemokines including IL-8, IP-10, MCP-1, MIP-1alpha, RANTES, Eotaxin, CCL21; cytokines which are released from macrophages, including IL-1, IL-6, IL-8, IL-12 and TNF-alpha; IL-1R1 and IL-1 alpha, or an isoform, homolog, fragment, variant or derivative of any of these proteins.

The term “antibody” (Ab) as used herein includes monoclonal antibodies, polyclonal antibodies, mono- and multispecific antibodies (e.g., bispecific antibodies), and antibody fragments, variants and derivatives so long as they exhibit the desired biological function, which is typically the capability of specifically binding to a target. The term “specifically binding” as used herein means that the antibody binds more readily to its intended target than to a different, non-specific target. In other words, the antibody “specifically binds” or exhibits “binding specificity” to its target if it preferentially binds or recognizes the target even in the presence of non-targets as measurable by a quantifiable assay (such as radioactive ligand binding Assays, ELISA, fluorescence based techniques (e.g. Fluorescence Polarization (FP), Fluorescence Resonance Energy Transfer (FRET)), or surface plasmon resonance). An antibody that “specifically binds” to its target may or may not exhibit cross-reactivity to (homologous) targets derived from different species.

The basic, naturally occurring antibody is a heterotetrameric glycoprotein composed of two identical light (L) chains and two identical heavy (H) chains. Some antibodies may contain additional polypeptide chains, such as the J chain in IgM and IgA antibodies. Each L chain is linked to an H chain by one covalent disulfide bond, while the two H chains are linked to each other by one or more disulfide bonds depending on the H chain isotype. Each H and L chain also comprises intrachain disulfide bridges. Each H chain comprises an N-terminal variable domain (V_{H}), followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM, having heavy chains designated α, β, ε, γ and μ, respectively. The γ and μ classes are further divided into subclasses on the basis of relatively minor differences in the CH sequence and function, e.g., humans express the following subclasses: IgG1, IgG2, IgG3, IgG4, IgA1 and IgA2.

The pairing of a V_{H} and V_{L} together forms a single antigen-binding site. The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, the V regions consist of relatively invariant stretches called framework regions (FRs) of about 15-30 amino acid residues separated by shorter regions of extreme variability called “hypervariable regions” also called “complementarity determining regions” (CDRs) that are each approximately 9-12 amino acid residues in length. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen binding site of antibodies. The constant domains are not involved directly in binding an antibody to an antigen, but exhibit various effector functions, such as participation of the antibody dependent cellular cytotoxicity (ADCC). The term “hypervariable region” (also known as “complementarity determining regions” or CDRs) when used herein refers to the amino acid residues of an antibody which are (usually three or four short regions of extreme sequence variability) within the V-region domain of an immunoglobulin which form the antigen-binding site and are the main determinants of antigen binding specificity. CDR residues may be identified based on cross-species sequence variability or crystallographic studies of antigen-antibody complexes.

The term “antibody” as used herein thus preferably refers to immunoglobulin molecules, or variants, fragments or derivatives thereof, which are capable of specifically binding to a target epitope via at least one complementarity determining region. The term includes mono-, and polyclonal antibodies, mono-, bi- and multispecific antibodies, antibodies of any isotype, including IgM, IgD, IgG, IgA and IgE antibodies, and antibodies obtained by any means, including naturally occurring antibodies, antibodies generated by immunization in a host organism, antibodies which were isolated and identified from naturally occurring antibodies or antibodies generated by immunization in a host organism and recombinantly produced by biomolecular methods known in the art, as well as chimeric antibodies, human antibodies, humanized antibodies, intrabodies, i.e. antibodies expressed in cells and optionally localized in specific cell compartments, as well as variants, fragments and derivatives of any of these antibodies.

The term “monoclonal antibody” (mab) as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to “polyclonal” antibody preparations which include different antibodies directed against different epitopes, each monoclonal antibody is directed against a single epitope on the antigen. In addition to their specificity, the monoclonal antibodies are advantageous in that they may be synthesized uncontaminated by other antibodies. The adjective “monoclonal” is not to be construed as requiring production of the antibody by any particular method. For example, the monoclonal antibodies useful in the present invention may be prepared by the hybridoma methodology first described by Kohler et al., Nature 256: 495 (1975), or they may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352: 624-628 (1991) and Marks et al., J. Mol. Biol. 222: 581-597 (1991), for example.

Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass. Chimeric antibodies include, e.g., “humanized” antibodies comprising variable domain antigen-binding sequences (partly or fully) derived from a non-human animal, e.g. a mouse or a non-human primate (e.g., Old World Monkey, Ape, etc.), and human constant region sequences, which are preferably capable of effectively mediating Fc effector functions, and/or exhibit reduced immunogenicity when introduced into the human body. “Humanized” antibodies may be prepared by creating a “chimeric” antibody (non-human Fab grafted onto human Fc) as an initial step and selective mutation of the (non-CDR) amino acids in the Fab portion of the molecule. Alternatively, “humanized” antibodies can be obtain directly by grafting appropriate “donor” CDR coding segments derived from a non-human animal onto a human antibody “acceptor” scaffold, and optionally mutating (non-CDR) amino acids for optimized binding.

An “antibody variant” or “antibody mutant” refers to an antibody comprising or consisting of an amino acid sequence wherein one or more of the amino acid residues have been modified as compared to a reference or “parent” antibody. Such antibody variants may thus exhibiting, increasing order of preference, at least about 5%, 10%, 20%, 30%, 40%, 50%, 60%, preferably at least about 70%, 80%, 85%, 86%, 87%, 88%, 89%, more preferably at least about 90%, 91%, 92%, 93%, 94%, most preferably at least about 95%, 96%, 97%, 98%, or 99% sequence identity to a reference or “parent” antibody, or to its light or heavy chain. Conceivable amino acid mutations include deletions, insertions or alterations of one or more amino acid residue(s). The mutations may be located in the constant region or in the antigen binding region (e.g., hypervariable or variable region). Conservative amino acid mutations, which change an amino acid to a different amino acid with similar biochemical properties (e.g. charge, hydrophobicity and size), may be preferred.

An “antibody fragment” comprises a portion of an intact antibody (i.e. an antibody comprising an antigen-binding site as well as a C_{L} and at least the heavy chain domains, C_{H}1, C_{H}2 and C_{H}3), preferably the antigen binding and/or the variable region of the intact antibody. Examples of antibody fragments include Fab, Fab′, F(ab′)₂ and Fv fragments; diabodies; linear antibodies, single-chain antibodies, and bi- or multispecific antibodies comprising such antibody fragments.

Papain digestion of antibodies produced two identical antigen-binding fragments, called “Fab” (fragment, antigen-binding) fragments, and a residual “Fc” (fragment, crystallisable) fragment. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen, and a pFc′ fragment. The F(ab′)₂ fragment can be split into two Fab′ fragments. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other antibody fragments and chemical fragments thereof are also known. The Fab/c or Fabc antibody fragment lacks one Fab region. Fd fragments correspond to the heavy chain portion of the Fab and contain a C-terminal constant (C_{H}1) and N-terminal variable (V_{H}) domain.

The Fc fragment comprises the carboxy-terminal portions of both H chains held together by disulphides. The effector functions of antibodies are determined by sequences in the Fc region, the region which is also recognized by Fc receptors (FcR) found on certain types of cells.

“Fv” is the minimum antibody fragment which contains a complete antigen-binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding.

The term “diabodies” (also referred to as divalent (or bivalent) single-chain variable fragments, “di-scFvs”, “bi-scFvs”) refers to antibody fragments prepared by linking two scFv fragments (see preceding paragraph), typically with short linkers (about 5-10) residues) between the V_{H} and V_{L} domains such that inter-chain but not intra-chain pairing of the V domains is achieved. Another possibility is to construct a single peptide chain with two V_{H} and two V_{L} regions (“tandem scFv). The resulting bivalent fragments, have two antigen-binding sites. Likewise, trivalent scFv trimers (also referred to as “triabodies” or “tribodies”) and tetravalent scFv tetramers (“tetrabodies”) can be produced. Di- or multivalent antibodies or antibody fragments may be monospecific, i.e. each antigen binding site may be directed against the same target. Such monospecific di- or multivalent antibodies or antibody fragments preferably exhibit high binding affinities. Alternatively, the antigen binding sites of di- or multivalent antibodies or antibody fragments may be directed against different targets, forming bi- or multispecific antibodies or antibody fragments.

“Bi- or multispecific antibodies or antibody fragments” comprise more than one specific antigen-binding region, each capable of specifically binding to a different target. “Bispecific antibodies” are typically heterodimers of two “crossover” scFv fragments in which the V_{H} and V_{L} domains of the two antibodies are present on different polypeptide chains. Bi- or multispecific antibodies may act as adaptor molecules between an effector and a respective target, thereby recruiting effectors (e.g. toxins, drugs, and cytokines or effector cells such as CTL, NK cells, macrophages, and granulocytes) to an antigen of interest, typically expressed by a target cell, such as a cancer cell. Thereby, “bi- or multispecific antibodies” preferably bring the effector molecules or cells and the desired target into close proximity and/or mediate an interaction between effector and target. Bispecific tandem di-scFvs, known as bi-specific T-cell engagers (BiTE antibody constructs) are one example of bivalent and bispecific antibodies in the context of the present invention.

The structure and properties of antibodies is well-known in the art and described, inter alia, in Janeway's Immunobiology, 9^{th} ed. (rev.), Kenneth Murphy and Casey Weaver (eds), Taylor & Francis Ltd. 2008. The term “immunoglobulin” (Ig) is used interchangeably with “antibody” herein. Exemplary antibodies may be selected from the group consisting of AAB-003; Abagovomab; Abciximab; Abituzumab; Abrilumab; Actoxumab; Adalimumab; Aducanumab; Afasevikumab; Aflibercept; Afutuzuab; Afutuzumab; Alacizumab_pegol; Alemtuzumab; Alirocumab; ALX-0061; Amatuximab; Anetumab_ravtansine; Anifrolumab; Anrukinzumab; Apolizumab; Apomab; Aquaporumab; Arcitumomab_99tc; Ascrinvacumab; Aselizuab; Atezolizumab; Atinumab; Atlizuab; Aurograb; Avelumab; Bapineuzumab; Basiliximab; Bavituximab; Begelomab; Benralizumab; Betalutin; Bevacituzuab; Bevacizumab_154-aspartic_acid; Bevacizumab_154-substitution; Bevacizumab_180-serine; Bevacizumab_180-substitution; Bevacizumab_beta; Bevacizumab; Bevacizumab-rhuMAb-VEGF; Bezlotoxumab; Bimagrumab; Bimekizumab; Bleselumab; Blinatumomab; Blinatumumab; Blontuvetmab; Blosozumab; Bococizumab; Brentuximab_vedotin; Briakinumab; Brodalumab; Brolucizumab; Brontictuzumab; BTT-1023; Burosumab; Canakinumab; Cantuzumab; Cantuzumab_mertansine; Cantuzumab_ravtansine; Caplacizumab; Carlumab; Cergutuzumab_amunaleukin; Certolizumab_pegol; Cetuximab; Citatuzumab_bogatox; Cixutumumab; Clazakizumab; Clivatuzumab_tetraxetan; Codrituzumab; Coltuximab_ravtansine; Conatumumab_CV; Conatumumab; Concizumab; Crenezumab; Crotedumab; Dacetuzumab; Dacliximab; Daclizumab; Dalotuzumab; Dapirolizumab_pegol; Daratumumab; Dectrekumab; Demcizumab; Denintuzumab_mafodotin; Denosumab; Depatuxizumab; Depatuxizumab_mafodotin; Dinutuximab_beta; Dinutuximab; Diridavumab; Domagrozumab; Drozituab; Drozitumab; Duligotumab; Duligotuzumab; Dupilumab; Durvalumab; Dusigitumab; Ecromeximab; Eculizumab; Efalizumab; Efungumab; Eldelumab; Elgemtumab; Elotuzumab; Emactuzumab; Emibetuzumab; Emicizumab; Enavatuzumab; Enfortumab; Enfortumab_vedotin; Enoblituzumab; Enokizumab; Enoticumab; Ensituximab; Entolimod; Epratuzumab; Eptacog_beta; Erlizuab; Etaracizumab; Etrolizuab; Etrolizumab; Evinacumab; Evolocumab; Exbivirumab; Farletuzumab; Fasinumab; Fezakinumab; FG-3019; Fibatuzumab; Ficlatuzumab; Figitumumab; Firivumab; Flanvotumab; Fletikumab; Fontolizumab; Foralumab; Foravirumab; Fresolimumab; Fulranumab; Futuximab; Galcanezumab; Galiximab; Ganitumab; Gantenerumab; Gemtuzumab; Gemtuzumab_ozogamicin; Gevokizumab; Girentuximab; Glembatumumab; Goilixiab; Guselkumab; HuMab-001; HuMab-005; HuMab-006; HuMab-019; HuMab-021; HuMab-025; HuMab-027; HuMab-032; HuMab-033; HuMab-035; HuMab-036; HuMab-041; HuMab-044; HuMab-049; HuMab-050; HuMab-054; HuMab-055; HuMab-059; HuMab-060; HuMab-067; HuMab-072; HuMab-084; HuMab-091; HuMab-093; HuMab-098; HuMab-100; HuMab-106; HuMab_10F8; HuMab-111; HuMab-123; HuMab-124; HuMab-125; HuMab-127; HuMab-129; HuMab-132; HuMab-143; HuMab-150; HuMab-152; HuMab-153; HuMab-159; HuMab-160; HuMab-162; HuMab-163; HuMab-166; HuMab-167; HuMab-169; HuMab-7D8; huMAb-anti-MSP10.1; huMAb-anti-MSP10.2; HUMAB-Clone_18; HUMAB-Clone_22; HuMab-L612; HuMab_LC5002-002; HuMab_LC5002-003; HuMab_LC5002-005; HuMab_LC5002-007; HuMab_LC5002-018; Ibalizumab; Ibritumomab_tiuxetan; Icrucumab; Idarucizumab; Igatuzuab; IGF-IR_HUMAB-1A; IGF-IR_HUMAB-23; IGF-IR_HUMAB-8; ImAb1; Imalumab; Imgatuzumab; Inclacumab; Indatuximab_ravtansine; Indusatumab_vedotin; Inebilizumab; Insulin_peglispro; Interferon_beta-1b; Intetumumab; Iodine_(124I)_Girentuximab; Iodine_(131I)_Derlotuxiab_biotin; Iodine_(131I)_Derlotuximab_biotin; Ipilimumab; Iratumumab; Isatuximab; Itolizumab; Ixekizumab; Labetuzumab_govitecan; Lambrolizumab; Lampalizumab; Lanadelumab; Landogrozumab; Laprituximab_emtansine; Lealesoab; Lebrikizumab; Lenercept_chainl; Lenzilumab; Lerdelimumab; Lexatumumab; Libivirumab; Lifastuzumab; Lifastuzumab_vedotin; Ligelizumab; Lilotomab; Lintuzumab; Lirilumab; Lodelcizumab; Lokivetmab; Lorvotuzumab_mertansine; Lpathomab; Lucatumumab; Lulizumab_pegol; Lumiliximab; Lumretuzumab; Lutetium_(177Lu)_lilotomab_satetraxetan; Margetuximab; Marzeptacog_alfa; Matuzumab; Mavrilimumab; MDX-1303; Mepolizumab; Metelimumab; Milatuzumab; Mirvetuximab; Modotuximab; Mogamulizumab; Monalizumab; Motavizumab; Moxetumomab_pasudotox; Muromonab-CD3; Namilumab; Naptumomab_estafenatox; Narnatumab; Natalizumab; Navicixizumab; Navivumab; Ndimab-varB; Necitumumab; Neliximab; Nemolizumab; Nesvacumab; Neuradiab; Nimotuzumab; Nivolumab; Obiltoxaximab; Obinutuzumab; Ocaratuzumab; Ocrelizumab; Ofatumumab; Olaratumab; Olizuab; Olokizumab; Omalizumab; Onartuzumab; Ontuxizumab; Opicinumab; Oportuzumab_monatox; Oreptacog_alfa; Orticumab; Otelixizumab; Otlertuzumab; Oxelumab; Ozanezumab; Ozoralizumab; Palivizumab; Pamrevlumab; Panitumumab; Pankoab; PankoMab; Panobacumab; Parsatuzumab; Pascolizumab; Pasotuxizumab; Pateclizumab; Patritumab; Pembrolizumab; Perakizumab; Pertuzuab; Pertuzumab; Pexelizumab_h5g1.1-scFv; Pexelizumab; PF-05082566; PF-05082568; Pidilizumab; Pinatuzumab_vedotin; Placulumab; Plozalizumab; Pogalizumab; Polatuzumab_vedotin; Ponezumab; Pritoxaximab; Pritumumab; Quilizumab; Racotumomab; Radretumab; Rafivirumab; Ralpancizumab; Ramucirumab; Ranibiziuab; Ranibizumab; Refanezumab; REGN2810; rhuMab_HER2(9CI); rhuMab_HER2; rhuMAb-VEGF; Rilotumumab; Rinucumab; Risankizumab; Rituximab; Rivabazumab_pegol; Robatumumab; Roledumab; Romosozumab; Rontalizuab; Rontalizumab; Rovalpituzumab_tesirine; Rovelizumab; Ruplizumab; Sacituzumab_govitecan; Samalizumab; Sarilumab; Satumomab_pendetide; Secukinumab; Seribantumab; Setoxaximab; Sifalimumab; Siltuximab; Simtuzumab; Sirukumab; Sofituzumab_vedotin; Solanezumab; Solitomab; Sonepcizumab; Stamulumab; Suptavumab; Suvizumab; Tabalumab; Tacatuzuab; Tadocizumab; Talizumab; Tamtuvetmab; Tanezumab; Tarextumab; Tefibazumab; Tenatumomab; Teneliximab; Teplizumab; Teprotumumab; Tesidolumab; Tezepelumab; ThioMAb-chMA79b-HC(A118C); ThioMab-hu10A8.v1-HC(A118C); ThioMab-hu10A8.v1-HC(V205C); ThioMab-hu10A8.v1-LC(A118C); ThioMab-hu10A8.v1-LC(V205C); ThioMAb-huMA79b.v17-HC(A118C); ThioMAb-huMA79b.v18-HC(A118C); ThioMAb-huMA79b.v28-HC(A118C); ThioMAb-huMA79b.v28-LC(V205C); Ticiliuab; Tigatuzumab; Tildrakizumab; Tisotumab_vedotin; Tocilizumab; Tosatoxumab; Tositumomab; Tovetumab; Tralokinumab; Trastuzuab; Trastuzumab_emtansine; Trastuzumab; TRC-105; Tregalizumab; Tremelimumab; Trevogrumab; Tucotuzumab_celmoleukin; Ublituximab; Ulocuplumab; Urelumab; Urtoxazumab; Ustekinumab; Vadastuximab_talirine; Vandortuzumab_vedotin; Vantictumab; Vanucizumab; Varlilumab; Vatelizumab; Vedolizumab; Veltuzumab; Vesencumab; Visilizumab; Volociximab; Vorsetuzumab; Vorsetuzumab_mafodotin; Yttrium_(90Y)_clivatuzumab_tetraxetan; Yttrium_Y_90_epratuzumab_tetraxetan; Yttrium_Y_90_epratuzumab; Zalutumumab; Zanolimumab; Zatuximab; Andecaliximab; Aprutumab; Azintuxizumab; Brazikumab; Cabiralizumab; Camrelizumab; Cosfroviximab; Crizanlizumab; Dezamizumab; Duvortuxizumab; Elezanumab; Emapalumab; Eptinezumab; Erenumab; Fremanezumab; Frunevetmab; Gatipotuzumab; Gedivumab; Gemetuzumab; Gilvetmab; Ifabotuzumab; Lacnotuzumab; Larcaviximab; Lendalizumab; Lesofavumab; Letolizumab; Losatuxizumab; Lupartumab; Lutikizumab; Oleclumab; Porgaviximab; Prezalumab; Ranevetmab; Remtolumab; Rosmantuzumab; Rozanolixizumab; Sapelizumab; Selicrelumab; Suvratoxumab; Tavolixizumab; Telisotuzumab; Telisotuzumab_vedotin; Timigutuzumab; Timolumab; Tomuzotuximab; Trastuzumab_duocarmazine; Varisacumab; Vunakizumab; Xentuzumab; anti-rabies_SO57; anti-rabies_SOJB; anti-rabies_SOJA; anti-rabies; anti-RSV_5ITB; anti-alpha-toxin_4U6V; anti-IsdB_5D1Q; anti-IsdB_5D1X; anti-IsdB_5D1Z; anti-HIV_b12; anti-HIV_2G12; anti-HIV_4E10; anti-HIV_VRC01; anti-HIV_PG9; anti-HIV_VRC07; anti-HIV_3BNC117; anti-HIV_10-1074; anti-HIV_PGT121; anti-HIV_PGDM1400; anti-HIV_N6; anti-HIV_10E8; anti-HIV_12A12; anti-HIV_12A21; anti-HIV_35022; anti-HIV_3BC176; anti-HIV_3BNC55; anti-HIV_3BNC60; anti-HIV_447-52D; anti-HIV_5H/I1-BMV-D5; anti-HIV_8ANC195; anti-HIV_cap256-176-723043/600049/531926/504134; anti-HIV_CAP256-VRC26.01/VRC26.02/VRC26.03/VRC26.04/VRC26.05/VRC26.06/VRC26.07/VRC26.08/VRC26.09/VRC26.10/VRC26.11/VRC26.12/VRC26.I1/VRC26.I2/VRC26.UCA; anti-HIV_cap256-206-252885/249183/220956/220629/200599/186347/186226/179686/173707/173339/172689/162744/146057/139519/1363 16/116098/115862/107018/098644/098135/096276/092794/086817/086446/086180/083708/079556/078657/075802/0 69097/067758/057019/055385/053187/053139/050350/046207/043389/042555/029720/028848/027652/024075/00874 8/008530; anti-HIV_cap256-119-186229/183891/183631/182676/180772/180508/180260/180173/179839/179262/178995/178455/177993/177727/176746/176241/175215/173928/173495/172882/172429/172223/171838/171587/1695 96/169523/169462/169092/168680/166385/165943/165738/164913/164167/163558/162043/161718/161675/161053/1 59499/159114/156751/155656/154420/153954/153864/153793/153462/153124/153025/152713/151794/150980/14889 5/148848/148743/148595/148490/148470/148107/147933/147434/146106/145604/143998/143441/141307/140896/14 0090/140037/139135/137881/137643/137170/136616/136206/135565/135025/133983/133917/132663/132113/131839/130626/130191/129798/128745/128593/128152/127693/126684/126056/125765/125106/124026/121783/121208/120 945/118229/118025/117418/117250/117230/116999/116558/116484/114844/114141/111917/111862/110064/109192/108793/108127/107758/107209/107184/106827/106511/106327/105486/105197/104946/103667/103385/103267/1030 11/102072/101945/101319/100871/100838/100025/100000/098890/098715/098632/097199/096189/094581/094200/0 94158/092814/092808/092573/090815/090368/089710/088555/087962/086903/086804/085910/085772/084603/08427 6/082288/080383/079333/078618/077466/076284/074680/074081/071704/071266/069667/069591/068691/068488/06 7536/065852/065457/064501/063568/063103/061027/058232/057341/056895/056402/056034/055042/054776/054539/054112/053339/052404/051123/051077/050442/049433/047532/047489/046020/044746/044740/043790/042880/042 606/042444/040328/040164/039130/038138/037868/037102/036683/036495/035375/035165/035109/033789/033641/032113/031739/030932/030740/030197/027047/026950/026279/025355/025301/025010/024631/024467/023805/0217 36/021203/020569/019432/018827/018483/018118/017782/017669/016976/015432/015281/014957/014777/014313/0 14219/013631/012924/011793/011413/011323/011233/009038/008756/008055/006949/006685/006015/005841/00582 4/005494/004949/004422/003932/003577/002155/002017/001312/001017/000594; anti-HIV_cap256-059-241099/207529/205541/188439/187234/187047/186068/182835/176659/172956/171272/168734/155838/149799/148168/1446 85/140017/137547/131908/116006/115783/114609/113952/113878/113622/109427/109081/107590/107504/099614/0 98972/097236/091487/089812/088468/088341/086533/086043/084191/082135/079417/076027/075082/072575/07192 6/069638/069165/068956/068876/067733/067450/065694/065109/065060/064001/063270/061357/059834/059313/05 7130/050520/049839/048503/045516/044188/044105/042100/040742/040554/039660/039298/037873/037633/036817/032787/032427/029390/027877/026640/026017/024100/023966/020534/019513/012963/010396/008136/006147/005 081/005006/004451/003571/003449/002712/001573/001379/001029; anti-HIV_cap256-048-165087/158861/158280/157928/157056/156422/152863/152770/150027/148246/147428/146603/145735/145116/144077/142876/140582/1393 55/139151/137672/137506/137270/135447/131966/131008/129369/128476/128270/126220/125713/123934/122673/1 22208/121552/120643/118458/118112/116469/113917/112368/112047/112029/110957/110526/109336/108152/10779 9/107384/106530/106464/106411/106306/104496/103074/100832/100188/099645/098137/097878/097510/097313/09 6626/096483/095691/095525/094783/094356/090756/089065/084986/083355/082462/082246/080752/078409/078273/078062/077798/073853/071661/071360/070955/070061/069669/069205/068882/067764/066845/065226/063717/063 150/062431/060745/060420/060014/059747/058393/058159/057127/056251/055421/054989/054759/052573/051477/051299/050815/049884/049170/048531/048259/047313/046596/044781/042599/041276/040200/039061/038515/0382 55/038177/035513/034112/033983/032688/031092/030464/030289/030261/029362/027638/027613/026627/026239/0 25518/024854/024537/021781/021758/020988/020663/020590/019765/019254/018073/016775/016069/015867/01567 3/015156/014521/014475/013798/013271/013180/012148/011870/011530/010968/010224/009749/009623/008234/00 8149/007301/007174/007079/007033/006128/005999/005394/004226/004097/003289/002601/002129/001875/001302/001203/000383; anti-HIV_cap256-038-261791/241540/235677/234314/234273/223164/220289/220020/216853/213466/213212/213120/212592/211790/209916/207938/202245/197721/196679/196118/195382/180001/178021/1771 04/171261/169090/168705/167685/158775/157318/153058/150027/146372/141868/141616/127989/118109/112226/1 05918/104487/102308/091115/090262/083260/080981/080873/074413/073153/064227/061640/059482/054000/05055 4/044256/040944/040090/032874/025899/024581/013345/011559/009634/006730/004887/004840/002181/001902/00 0976/000384; anti-HIV_048-250757/250716/250463/248153/247532/245846/244016/243682/243588/241775/237996/237730/237253/234100/230882/229473/228238/228027/227795/227770/225298/225090/224187/223055/222711/2212 09/220629/219430/216250/216133/214886/214709/214001/213230/212574/212207/209146/208206/208194/207744/2 06501/204221/204015/201240/200455/200319/197896/193813/192098/191786/188746/185937/184849/183089/18150 9/180990/177532/177426/177389/174266/172847/172845/172363/171609/170705/168381/166619/162036/160042/15 9676/159500/159421/159333/158932/155811/155464/155392/155389/154449/153379/153171/152324/146102/145984/145371/144907/142298/142277/141934/141207/140796/139893/138820/135858/134968/134312/132253/130710/128 564/126702/124521/122740/119536/116929/116577/116046/115875/115599/113988/112989/112435/111339/111055/111027/109721/109666/109196/109051/108570/108033/107279/106271/106054/104848/104638/104567/102804/1016 76/097603/097107/096871/096668/095236/094155/093219/092976/090866/090650/089009/088654/086513/086024/0 85857/084277/084245/082487/081787/081062/079639/079126/073118/070264/069426/068564/068345/067337/06718 0/063017/061885/061671/060700/060592/060300/059141/057777/056928/056131/055864/055094/054343/054193/05 2521/049037/048720/048542/047777/046841/046202/046059/043568/042713/042440/040511/039195/036935/034478/031641/029760/027970/027337/027217/026760/024800/024313/021748/020991/020340/019993/019947/017871/015 931/015920/013898/013429/012358/011158/010720/009445/006126/005652/005532/005189/005088/004023/001580; anti-HIV_119-099719/099536/098907/098555/097828/096480/095664/095212/094773/094508/093795/093732/092903/092284/091586/091023/090334/088694/088499/088298/087488/087423/087371/087279/087146/087048/0858 02/085784/085370/085276/084885/084874/084691/083793/083163/082331/082070/081512/080816/079302/079292/0 79289/078935/078702/078593/077708/076904/075862/075465/074822/074629/074500/073911/072765/072313/07228 0/071693/071353/069711/069061/068202/068063/067980/067866/067756/066859/065821/065191/064667/063791/06 2989/062286/061416/061344/060240/060184/058035/057858/057473/057090/055754/054899/054501/051867/051814/051567/051483/050913/050187/049069/048517/048470/048303/048021/047928/047384/047145/046752/046660/046 202/045790/044670/044140/042776/042581/040905/040322/039892/039764/039188/039058/038837/038396/036918/036592/036310/035618/035569/035466/035157/035121/035046/034754/034318/033780/033632/033183/030696/0300 59/029589/029448/029220/028317/028165/027147/026743/026508/025683/025614/025548/025526/023552/023092/0 22793/022395/022334/021866/021278/021183/019376/019238/018500/018318/018218/017876/017740/017128/01704 4/016644/015878/015538/015455/014425/013582/013364/012886/012249/012161/012110/012100/011651/011479/01 1232/011175/008396/007148/007029/004707/003910/002450/001552; anti-HIV_CH01/CH02/CH03/CH04/CH103/M66.6/NIH45-46/PG16/PGT122/PGT123/PGT125/PGT126/PGT127/PGT128/PGT130/PGT131/PGT135/PGT136/PGT137/PGT141/PGT142/PGT143/PGT144/PGT145/PGT151/PGT152/VRC-CH30/VRC-CH31/VRC-CH32/VRC-CH33/VRC-CH34/VRC-PG04/VRC-PG04b/VRC-PG20/VRC02/VRC03/VRC23/5CCK/5AWN/3QEG/1NOX/3QEH/2B1H/3TNM/3UJJ/3UJI/2QSC/3MLZ/3MLX/3MLW/3MLV/3MLU/3MLT/3G01/4XCY/4YBL/4R4N/4R4B/3JUY/4KG5anti-HIV-1/V3/CD4bs/V2/C38-VRC18.02/44-VRC13.02/45; anti-HIV_059-188169/183739/182376/182199/169202/155645/151619/146503/136098/105516/095709/069468/060026/053668/052864/050968/046422/045120/039932/038595/035082/029204/025235/0151 92/007060/006953/005953/003725/002618/001522/000731/000634; anti-HIV_206-314431; anti-HIV_206-247594; anti-HIV_206-116890; anti-HIV_206-072383; anti-HIV_206-037527; anti-HIV_206-009095; anti-HIV_176-503620; anti-HIV_176-478726; anti-HIV_176-245056; anti-HIV_176-164413; anti-HIV_176-094308; anti-HIV_176-065321; anti-HIV_038-221120; anti-HIV_038-197677; anti-HIV_038-196765; anti-HIV_038-186200; anti-HIV_038-126170; anti-HIV_038-108545; anti-HIV_038-107263; anti-HIV_038-104530; anti-HIV_038-099169; anti-HIV_038-075067; anti-HIV_038-072368; anti-HIV_038-068503; anti-HIV_038-068016; anti-HIV_038-063958; anti-HIV_038-033733; anti-HIV_038-030557; anti-HIV_038-024298; anti-HIV_038-011154; anti-HIV_5CIN; anti-HIV_5CIL; anti-HIV_5CIP; anti-HIV_4JKP; anti-HIV_3TNN; anti-HIV_3BQU; anti-HIV_IgG; anti-HIV_4P9M; anti-HIV_4P9H; anti-HIV_Ig; anti-HIV; anti-influenza; anti-influenza_Apo; anti-influenza-A; and anti-OX40, or a homolog, fragment, variant or derivative of any of these antibodies.

Artificial nucleic acid molecules of the invention encoding preferred antibodies may preferably comprise a coding region comprising or consisting of a nucleic acid sequence according to any one of the SEQ ID NO:1 to 61734 or respectively Table 3, Table 4, Table 5, Table 6 or Table 9 as described in international patent application PCT/EP2017/060226, in particular a nucleic acid sequence being identical or having a sequence identity of at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, preferably at least 80%, to these sequences or a fragment or variant of any of these RNA sequences. In this context, the disclosure of PCT/EP2017/060226 is also incorporated herein by reference. The person skilled in the art knows that also other (redundant) mRNA sequences can encode the proteins as shown in the above reference, therefore the mRNA sequences are not limited thereto.

Artificial nucleic acid molecules of the invention encoding preferred therapeutic proteins may preferably comprise a coding region comprising or consisting of a nucleic acid sequence according to any one of the SEQ ID NO as shown in SEQ ID NO:1 to SEQ ID NO:345916 or respectively Table I as described in U.S. application Ser. No. 15/585,561, in particular a nucleic acid sequence being identical or having a sequence identity of at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, preferably at least 80%, to these sequences or a fragment or variant of any of these RNA sequences. In this context, the disclosure of U.S. application Ser. No. 15/585,561 is also incorporated herein by reference. The person skilled in the art knows that also other (redundant) mRNA sequences can encode the proteins as shown in the above reference, therefore the mRNA sequences are not limited thereto.

Further artificial nucleic acid molecules of the invention encoding preferred therapeutic proteins may preferably comprise a coding region comprising or consisting of a nucleic acid sequence according to any one of the SEQ ID NO as shown in SEQ ID NO:1 to SEQ ID NO:345916 or respectively Table I as described in international patent application PCT/EP2017/060692, in particular a nucleic acid sequence being identical or having a sequence identity of at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, preferably at least 80%, to these sequences or a fragment or variant of any of these RNA sequences. In this context, the disclosure of international patent application PCT/EP2017/060692 is also incorporated herein by reference. The person skilled in the art knows that also other (redundant) mRNA sequences can encode the proteins as shown in the above reference, therefore the mRNA sequences are not limited thereto.

The term “peptide hormone” refers to a class of peptides or proteins that have endocrine functions in living animals. Typically, peptide hormones exert their functions by binding to receptors on the surface of target cells and transmitting signals via intracellular second messengers. Exemplary peptide hormones include Adiponectin i.e. Acrp30; Adrenocorticotropic hormone (or corticotropin) i.e. ACTH; Amylin (or Islet Amyloid Polypeptide) i.e. IAPP; Angiotensinogen and angiotensin i.e. AGT; Anti-Müllerian hormone (or Müllerian inhibiting factor or hormone) i.e. AMH; Antidiuretic hormone (or vasopressin, arginine vasopressin) i.e. ADH; Atrial-natriuretic peptide (or atriopeptin) i.e. ANP; Brain natriuretic peptide i.e. BNP; Calcitonin i.e. CT; Cholecystokinin i.e. CCK; Corticotropin-releasing hormone i.e. CRH; Cortistatin i.e. CORT; Endothelin i.e.; Enkephalin i.e.; Erythropoietin i.e. EPO; Follicle-stimulating hormone i.e. FSH; Galanin i.e. GAL; Gastric inhibitory polypeptide i.e. GIP; Gastrin i.e. GAS; Ghrelin i.e.; Glucagon i.e. GCG; Glucagon-like peptide-1 i.e. GLP1; Gonadotropin-releasing hormone i.e. GnRH; Growth hormone i.e. GH or hGH; Growth hormone-releasing hormone i.e. GHRH; Guanylin i.e. GN; Hepcidin i.e. HAMP; Human chorionic gonadotropin i.e. hCG; Human placental lactogen i.e. HPL; Inhibin i.e.; Insulin i.e. INS; Insulin-like growth factor (or somatomedin) i.e. IGF; Leptin i.e. LEP; Lipotropin i.e. LPH; Luteinizing hormone i.e. LH; Melanocyte stimulating hormone i.e. MSH or a-MSH; Motilin i.e. MLN; Orexin i.e.; Osteocalcin i.e. OCN; Oxytocin i.e. OXT; Pancreatic polypeptide i.e. Parathyroid hormone i.e. PTH; Pituitary adenylate cyclase-activating peptide i.e. PACAP; Prolactin i.e. PRL; Prolactin releasing hormone i.e. PRH; Relaxin i.e. RLN; Renin i.e.; Secretin i.e. SCT; Somatostatin i.e. SRIF; Thrombopoietin i.e. TPO; Thyroid-stimulating hormone (or thyrotropin) i.e. TSH; Thyrotropin-releasing hormone i.e. TRH; Uroguanylin i.e. UGN; or Vasoactive intestinal peptide i.e. VIP, or an isoform, homolog, fragment, variant or derivative of any of these proteins.

The term “gene editing agent” refers to (poly-)peptides or proteins that are capable of modifying (i.e. alter, induce, increase, reduce, suppress, abolish or prevent) expression of a gene. Gene expression can be modified on several levels. Gene editing agents may typically act by (a) introducing or removing epigenetic modifications, (b) altering the sequence of genes, e.g. by introducing, deleting or changing nucleic acid residues in the nucleic acid sequence of a gene of interest (c) modifying the biological function of regulatory elements operably linked to the gene of interest (d) modifying mRNA transcription, processing, splicing, maturation or export into the cytoplasm, (e) modifying mRNA translation, (f) modifying post-translational modifications, (g) modifying protein translocation or export. In a narrower sense, the term “gene editing agent” may refer to (poly-)peptides or proteins targeting the genome of a cell to modify gene expression, preferably by exerting functions (a)-(d), more preferably (a)-(c). The term “gene editing agent” as used herein thus preferably encompasses gene editing agents that cleave or alter the targeted DNA to induce mutation (e.g., via homologous directed repair or non-homologous end-joining), but also includes gene editing agents that can reduce expression in the absence of target cleavage (e.g., gene editing agents that are fused or conjugated to expression modulators such as transcriptional repressors or epigenetic modifiers that can reduce gene expression). Particular gene editing agents include: transcriptional activators, transcriptional repressors, recombinases, nucleases, DNA-binding proteins, or combinations thereof.

The present invention also relates to artificial nucleic acids, in particular RNAs, encoding CRISPR-associated proteins, and (pharmaceutical) compositions and kit-of-parts comprising the same. Said artificial nucleic acids, in particular RNAs, (pharmaceutical) compositions and kits are inter alia envisaged for use in medicine, for instance in gene therapy, and in particular in the treatment and/or prophylaxis of diseases amenable to treatment with CRISPR-associated proteins, e.g. by gene editing, knock-in, knock-out or modulating the expression of target genes of interest.

The term “CRISPR-associated protein” refers to RNA-guided endonucleases that are part of a CRISPR (Clustered Regularly Interspaced Short Palindromic Repeats) system (and their homologs, variants, fragments or derivatives), which is used by prokaryotes to confer adaptive immunity against foreign DNA elements. CRISPR-associated proteins include, without limitation, Cas9, Cpf1 (Cas12), C2c1, C2c3, C2c2, Cas13, CasX and CasY. As used herein, the term “CRISPR-associated protein” includes wild-type proteins as well as homologs, variants, fragments and derivatives thereof. Therefore, when referring to artificial nucleic acid molecules encoding Cas9, Cpf1 (Cas12), C2c1, C2c3, and C2c2, Cas13, CasX and CasY, said artificial nucleic acid molecules may encode the respective wild-type proteins, or homologs, variants, fragments and derivatives thereof.

Preferably, the at least one 5′UTR element and the at least one 3′UTR element act synergistically to increase the expression of the at least one coding sequence operably linked to said UTRs. It is envisaged herein to utilize the recited 5′-UTRs and 3′-UTRs in any useful combination. Further particularly preferred embodiments of the invention comprise the combination of the CDS of choice, i.e. a CDS selected from the group consisting of Cas9, Cpf1, CasX, CasY, and Cas13 with an UTR-combination selected from the group of HSD17B4/Gnas.1; Slc7a3.1/Gnas.1; ATP5A1/CASP.1; Ndufa4.1/PSMB3.1; HSD17B4/PSMB3.1; RPL32var/albumin7; 32L4/albumin7; HSD17B4/CASP1.1; Slc7a3.1/CASP1.1; Slc7a3.1/PSMB3.1; Nosip.1/PSMB3.1; Ndufa4.1/RPS9.1; HSD17B4/RPS9.1; ATP5A1/Gnas.1; Ndufa4.1/COX6B1.1; Ndufa4.1/Gnas.1; Ndufa4.1/Ndufa1.1; Nosip.1/Ndufa1.1; Rpl31.1/Gnas.1; TUBB4B.1/RPS9.1; and Ubqln2.1/RPS9.1.

The term “immune checkpoint inhibitor” refers to any (poly-)peptide or protein capable of inhibiting (i.e. interfering with, blocking, neutralizing, reducing, suppressing, abolishing, preventing) the biological activity of an immune checkpoint protein. Immune checkpoint proteins typically regulate T-cell activation or function and are well known in the art. Immune checkpoint proteins include, without limitation, CTLA-4, PD-1, VISTA, B7-H2, B7-H3, PD-L1 (B7-H1, CD274), B7-H4, B7-H6, 2B4, ICOS, HVEM, PD-L2 (B7-DC, CD273), CD2, CD27, CD28, CD30, CD40, CD70, CD80, CD86, CD137, CD160, CD226, CD276, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, A2aR, DR3, IDOL, IDO2, LAIR-2, LIGHT, MARCO (macrophage receptor with collagenous structure), PS (phosphatidylserine), OX-40, SLAM, TIGHT, VISTA, and/or VTCN1. Exemplary agents useful for inhibiting immune checkpoint proteins include antibodies (and antibody fragments, variants or derivatives), peptides, natural ligands (and ligand fragments, variants or derivatives), fusion proteins, that can either directly bind to (and thereby inactivate or inhibit) or indirectly inactivate or inhibit immune checkpoint proteins, e.g. by binding to, inactivating and/or inhibiting their receptors or downstream signalling molecules to block the interaction between one or more immune checkpoint proteins and their natural receptor(s) and/or to prevent inhibitory signalling mediated by binding of said immune checkpoint proteins and their natural receptor(s). Exemplary immune checkpoint inhibitors include A2AR; B7-H3 i.e. cD276; B7-H4 i.e. VTCN1; BTLA; CTLA-4; IDO i.e. Indoleamine 2,3-dioxygenase; KIR i.e. Killer-cell Immunoglobulin-like Receptor; LAG3 i.e. Lymphocyte Activation Gene-3; PD-1 i.e. Programmed Death 1 (PD-1) receptor; PD-L1, TIM-3 i.e. T-cell Immunoglobulin domain and Mucin domain 3; VISTA (protein) i.e. V-domain Ig suppressor of T cell activation; GITR, i.e. Glucocorticoid-Induced TNFR family Related gene; stimulatory checkpoint molecules i.e. CD27, CD40, CD122, OX40, GITR and CD137 or stimulatory checkpoint molecules belonging to the B7-CD28 superfamily, i.e. CD28 and ICOS, or an isoform, homolog, fragment, variant or derivative of any of these proteins.

The term “T cell receptor” or “TCR” refers to a T-cell specific protein receptor that is composed of a heterodimer of variable, disulphide-linked alpha (α) and beta ( ) chains, or of gamma and delta (γ/δ) chains, optionally forming a complex with domains for additional (co-)stimulatory signalling, such as the invariant CD3-zeta (ζ) chains and/or FcR, CD27, CD28, 4-1BB (CD137), DAP10, and/or OX40. The term “T cell receptor” includes (engineered) variants, fragments and derivatives of such naturally occurring TCRs, including chimeric antigen receptors (CARs). The term “chimeric antigen receptor (CAR)” generally refers to engineered fusion proteins comprising binding domains fused to an intracellular signalling domain capable of activating T cells. Typically, CARs are chimeric polypeptide constructs comprising at least an extracellular antigen binding domain, a transmembrane domain and a cytoplasmic signalling domain (also referred to herein as “an intracellular signalling domain”) comprising a functional signalling domain derived from a (co-)stimulatory molecule, such as the CD3-zeta chain, FcR, CD27, CD28, 4-1BB (CD137), DAP10, and/or OX40. The extracellular antigen-binding domain may typically be derived from a monoclonal antibody or a fragment, variant or derivative thereof. In particular aspects, CARs comprise fusions of single-chain variable fragments (scFv) derived from monoclonal antibodies, fused to CD3-zeta transmembrane and intracellular endodomain.

Artificial nucleic acid molecules of the invention encoding preferred sequences for the treatment of tumor or cancer diseases may preferably comprise a coding region comprising or consisting of a nucleic acid sequence according to any one of the SEQ ID NO:1 to 10071, preferably SEQ ID NO:1, 3, 5, 6, 389, or 399, or respectively Tables 1 to 12 or Tables 14-17 as described in international patent application WO2016170176A1, in particular a nucleic acid sequence being identical or having a sequence identity of at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, preferably at least 80%, to these sequences or a fragment or variant of any of these RNA sequences. In this context, the disclosure of WO2016170176A1 is also incorporated herein by reference. The person skilled in the art knows that also other (redundant) mRNA sequences can encode the proteins as shown in the above reference, therefore the mRNA sequences are not limited thereto.

Further artificial nucleic acid molecules of the invention encoding preferred sequences for the treatment of tumor or cancer diseases may preferably comprise a coding region comprising or consisting of a nucleic acid sequence according to any one of the SEQ ID NO SEQ ID NO as shown in international patent applications WO2009046974, WO2015024666, WO2009046739, WO2015024664, WO2003051401, WO2012089338, WO2013120627, WO2014127917, WO2016170176, or WO2015135558, in particular a nucleic acid sequence being identical or having a sequence identity of at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, preferably at least 80%, to these sequences or a fragment or variant of any of these RNA sequences. In this context, the disclosure of WO2009046974, WO2015024666, WO2009046739, WO2015024664, WO2003051401, WO2012089338, WO2013120627, WO2014127917, WO2016170176, or WO2015135558 is also incorporated herein by reference. The person skilled in the art knows that also other (redundant) mRNA sequences can encode the proteins as shown in the above reference, therefore the mRNA sequences are not limited thereto.

The term “enzyme” is well-known in the art and refers to (poly-)peptide and protein catalysts of chemical reactions. Enzymes include whole intact enzyme or fragments, variants or derivatives thereof. Exemplary enzymes include oxidoreductases, transferases, hydrolases, lyases, isomerases, and ligases.

Fragments, variants and derivatives of the aforementioned therapeutic proteins are also envisaged as (poly-)peptides or proteins of interest, provided that they are preferably functional and thus capable of mediating the desired biological effect or function.

### Antigenic (Poly-)Peptides or Proteins

The at least one coding region of the artificial nucleic acid molecule of the invention may encode at least one “antigenic (poly-)peptide or protein”. The term “antigenic (poly-)peptide or protein” or, shortly, “antigen” generally refers to any (poly-)peptide or protein capable, under appropriate conditions, of interacting with/being recognized by components of the immune system (such as antibodies or immune cells via their antigen receptors, e.g. B cell receptors (BCRs) or T cell receptors (TCRs)), and preferably capable of eliciting an (adaptive) immune response. The term “components of the immune system” preferably refers to immune cells, immune cell receptors and antibodies of the adaptive immune system. The “antigenic peptide or protein” preferably interacts with/is recognized by the components of the immune system via its “epitope(s)” or “antigenic determinant(s)”.

The term “epitope” or “antigenic determinant” refers to a part or fragment of an antigenic peptide or protein that recognized by the immune system. Said fragment may typically comprise from about 5 to about 20 or even more amino acids. Epitopes may be “conformational” (or “discontinuous”), i.e. composed of discontinuous sequences of the amino acids of the antigenic peptide or protein that they are derived from, but brought together in the three-dimensional structure of e.g. a MHC-complex, or “linear”, i.e. consist of a continuous sequence of amino acids of the antigenic peptides or proteins that they are derived from. The term “epitope” generally encompasses “T cell epitopes” (recognized by T cells via their T cell receptor) and “B cell epitopes” (recognized by B cells via their B cell receptor). “B cell epitopes” are typically located on the outer surface of (native) protein or peptide antigens as defined herein, and may preferably comprise or consist of between 5 to 15 amino acids, more preferably between 5 to 12 amino acids, even more preferably between 6 to 9 amino acids. “T cell epitopes” are typically recognized by T cells in a MHC-I or MHC-II bound form, i.e. as a complex formed by an antigenic protein or peptide fragment comprising the epitope, and a MHC-I or MHC-II surface molecule. “T cell epitopes” may typically have a length of about 6 to about 20 or even more amino acids, T cell epitopes presented by MHC class I molecules may preferably have a length of about 8 to about 10 amino acids, e.g. 8, 9, or 10, (or even 11, or 12 amino acids). T cell epitopes presented by MHC class II molecules may preferably have a length of about 13 or more amino acids, e.g. 13, 14, 15, 16, 17, 18, 19, 20 or even more amino acids. In the context of the present invention, the term “epitope” may in particular refer to T cell epitopes.

Thus, the term “antigenic (poly-)peptide or protein” refers to a (poly-)peptide comprising, consisting of or being capable of providing at least one (functional) epitope. Artificial nucleic acid (RNA) molecules of the invention may encode full-length antigenic (poly-)peptides or proteins, or preferably fragments thereof. Said fragments may comprise or consist of or be capable of providing (functional) epitopes of said antigenic (poly-)peptides or proteins. A “functional” epitope refers to an epitope capable of inducing a desired adaptive immune response in a subject.

Artificial nucleic acid (RNA) molecules encoding, in their at least one coding region, at least one antigenic (poly-)peptide or protein may enter the target cells (e.g. professional antigen-presenting cells (APCs), where the at least one antigenic (poly-)peptide or protein is expressed, processed and presented to immune cells (e.g. T cells) on an MHC molecule, preferably resulting in an antigen-specific immune response (e.g. cell-mediated immunity or formation of antibodies). Alternatively, artificial nucleic acid (RNA) molecules encoding, in their at least one coding region, at least one antigenic (poly-)peptide or protein may enter the target cells (e.g. muscle cells, dermal cells) where the at least one antigenic (poly-)peptide or protein is expressed and for instance secreted by the target cell to the extracellular environment, where it encounters cells of the immune system (e.g. B cells, macrophages) and preferably induces an antigen-specific immune response (e.g. formation of antibodies).

When referring to an artificial nucleic acid (RNA) molecule encoding “at least one antigenic peptide or protein” herein, it is envisaged that said artificial nucleic acid (RNA) molecule may encode one or more full-length antigenic (poly-)peptide(s) or protein(s), or one or more fragment(s), in particular a (functional) epitope(s), of said antigenic (poly-)peptide or protein. Said full-length antigenic (poly-)peptide(s) or protein(s), or its fragment(s), preferably comprises, consists of or is capable of providing at least one (functional) epitope, i.e. said antigenic (poly-)peptide(s) or protein(s) or its fragment(s) preferably either comprise(s) or consist(s) of a native epitope (preferably recognized by B cells) or is capable of being processed and presented by an MHC-I or MHC-II molecule to provide a MHC-bound epitope (preferably recognized by T cells).

The choice of particular antigenic (poly-)peptides or proteins generally depends on the disease to be treated or prevented. In general, the artificial nucleic acid (RNA) molecule, may encode any antigenic (poly-)peptide or protein associated with a disease amenable to treatment by inducing an immune response against said antigen (e.g. cancer, infections).

Preferably, artificial nucleic acid molecules according to the invention may comprise at least one coding region encoding a tumor antigen, a pathogenic antigen, an autoantigen, an alloantigen, or an allergenic antigen.

The term “tumor antigen” refers to antigenic (poly-)peptides or proteins derived from or associated with a (preferably malignant) tumor or a cancer disease. As used herein, the terms “cancer” and “tumor” are used interchangeably to refer to a neoplasm characterized by the uncontrolled and usually rapid proliferation of cells that tend to invade surrounding tissue and to metastasize to distant body sites. The term encompasses benign and malignant neoplasms. Malignancy in cancers is typically characterized by anaplasia, invasiveness, and metastasis; whereas benign malignancies typically have none of those properties. The terms “cancer” and “tumor” in particular refer to neoplasms characterized by tumor growth, but also to cancers of blood and lymphatic system. A “tumor antigen” is typically derived from a tumor/cancer cell, preferably a mammalian tumor/cancer cell, and may be located in or on the surface of a tumor cell derived from a mammalian, preferably from a human, tumor, such as a systemic or a solid tumor. “Tumor antigens” generally include tumor-specific antigens (TSAs) and tumor-associated-antigens (TAAs). TSAs typically result from a tumor specific mutation and are specifically expressed by tumor cells. TAAs, which are more common, are usually presented by both tumor and “normal” (healthy, non-tumor) cells.

The protein or polypeptide may comprise or consist of a tumour antigen, a fragment, variant or derivative of a tumour antigen. Such nucleic acid molecules are particularly useful for therapeutic purposes, particularly genetic vaccination. Preferably, the tumour antigen may be selected from the group comprising a melanocyte-specific antigen, a cancer-testis antigen or a tumour-specific antigen, preferably a CT-X antigen, a non-X CT-antigen, a binding partner for a CT-X antigen or a binding partner for a non-X CT-antigen or a tumour-specific antigen, more preferably a CT-X antigen, a binding partner for a non-X CT-antigen or a tumour-specific antigen or a fragment, variant or derivative of said tumour antigen; and wherein each of the nucleic acid sequences encodes a different peptide or protein; and wherein at least one of the nucleic acid sequences encodes for 5T4, 707-AP, 9D7, AFP, AlbZIP HPG1, alpha-5-beta-1-integrin, alpha-5-beta-6-integrin, alpha-actinin-4/m, alpha-methylacyl-coenzyme A racemase, A T-4, ARTC1/m, B7H4, BAGE-1, BCL-2, bcr/abl, beta-catenin/m, BING-4, BRCAI/m, BRCA2/m, CA 1 5-3/CA 27-29, CA 19-9, CA72-4, CA125, calreticulin, CAMEL, CASP-8/m, cathepsin B, cathepsin L, CD19, CD20, CD22, CD25, CDE30, CD33, CD4, CD52, CD55, CD56, CD80, CDC27/m, CDK4/m, CDKN2A/m, CEA, CLCA2, CML28, CML66, COA-1/m, coactosin-like protein, collage XXIII, COX-2, CT-9/BRD6, Cten, cyclin B1, cyclin D1, cyp-B, CYPB1, DAM-10, DAM-6, DEK-CAN, EFTUD2/m, EGFR, ELF2/m, EMMPRIN, EpCam, EphA2, EphA3, ErbB3, ETV6-AML1, EZH2, FGF-5, FN, Frau-1, G250, GAGE-1, GAGE-2, GAGE-3, GAGE-4, GAGE-5, GAGE-6, GAGE7b, GAGE-8, GDEP, GnT-V, gp100, GPC3, GPNMB/m, HAGE, HAST-2, hepsin, Her2/neu, HERV-K-MEL, HLA-A*0201-R1 7I, HLA-A1 1/m, HLA-A2/m, HNE, homeobox NKX3.1, HOM-TES-14/SCP-1, HOM-TES-85, HPV-E6, HPV-E7, HSP70-2M, HST-2, hTERT, iCE, IGF-1 R, IL-13Ra2, IL-2R, IL-5, immature laminin receptor, kallikrein-2, kallikrein-4, i67, KIAA0205, KIAA0205/m, KK-LC-1, K-Ras/m, LAGE-A1, LDLR-FUT, MAGE-A1, MAGE-A2, MAGE-A3, MAGE-A4, MAGE-A6, MAGE-A9, MAGE-A10, MAGE-A12, MAGE-B1, MAGE-B2, MAGE-B3, MAGE-B4, MAGE-B5, MAGE-B6, MAGE-B10, MAGE-B1 6, MAGE-B1 7, MAGE-C1, MAGE-C2, MAGE-C3, MAGE-D1, MAGE-D2, MAGE-D4, MAGE-E1, MAGE-E2, MAGE-F1, MAGE-H I, MAGEL2, mammaglobin A, MART-1/melan-A, MART-2, MART-2/m, matrix protein 22, MC1 R, M-CSF, ME 1/m, mesothelin, MG50/PXDN, MMP1 1, MN/CA IX-antigen, MRP-3, MUC-1, MUC-2, MUM-1/m, MUM-2/m, MUM-3/m, myosin class l/m, NA88-A, N-acetylgl ucosaminy transferase-V, Neo-PAP, Neo-PAP/m, NFYC/m, NGEP, NMP22, NPM/ALK, N-Ras/m, NSE, NY-ESO-1, NY-ESO-B, OA1, OFA-iLRP, OGT, OGT/m, OS-9, OS-9/m, osteocalcin, osteopontin, pi 5, p190 minor bcr-abl, p53, p53/m, PAGE-4, PAI-1, PAI-2, PAP, PART-1, PATE, PDEF, Pim-1-Kinase, Pin-1, Pml/PARalpha, POTE, PRAME, PRDX5/m, prostein, proteinase-3, PSA, PSCA, PSGR, PSM, PSMA, PTPRK/m, RAGE-1, RBAF600/m, RHAMM/CD1 68, RU1, RU2, S-100, SAGE, SART-1, SART-2, SART-3, SCC, SIRT2/m, Sp1 7, SSX-1, SSX-2/HOM-MEL-40, SSX-4, STAMP-1, STEAP-1, survivin, survivin-2B, SYT-SSX-1, SYT-SSX-2, TA-90, TAG-72, TARP, TEL-AML1, TGFbeta, TGFbetaRII, TGM-4, TPI/m, TRAG-3, TRG, TRP-1, TRP-2/6b, TRP/INT2, TRP-p8, tyrosinase, UPA, VEGFR1, VEGFR-2/FLK-1, WT1 and a immunoglobulin idiotype of a lymphoid blood cell or a T cell receptor idiotype of a lymphoid blood cell, or a homolog, fragment, variant or derivative of any of these tumor antigens; preferably survivin or a homologue thereof, an antigen from the MAGE-family or a binding partner thereof or a fragment, variant or derivative of said tumour antigen.

Particularly preferred in this context are the tumour antigens NY-ESO-1, 5T4, MAGE-C1, MAGE-C2, Survivin, Muc-1, PSA, PSMA, PSCA, STEAP and PAP, or homologs, fragments, variants or derivatives of any of these tumor antigens.

The term “pathogenic antigen” refers to antigenic (poly-)peptides or proteins derived from or associated with pathogens, i.e. viruses, microorganisms, or other substances causing infection and typically disease, including, besides viruses, bacteria, protozoa or fungi. In particular, such “pathogenic antigens” may be capable of eliciting an immune response in a subject, preferably a mammalian subject, more preferably a human. Typically, pathogenic antigens may be surface antigens, e.g. (poly-)peptides or proteins (or fragments of proteins, e.g. the exterior portion of a surface antigen) located at the surface of the pathogen (e.g. its capsid, plasma membrane or cell wall).

Accordingly, in some preferred embodiments, the artificial nucleic acid (RNA) molecule may encode in its at least one coding region at least one pathogenic antigen selected from a bacterial, viral, fungal or protozoal antigen. The encoded (poly-)peptide or protein may consist or comprise of a pathogenic antigen or a fragment, variant or derivative thereof.

Pathogenic antigens may preferably be selected from antigens derived from the pathogens Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo haemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli 01 57:H7, 01 1 1 and O1 04:H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Henclra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis, or an isoform, homolog, fragment, variant or derivative of any of these proteins.

Further preferred pathogenic antigens may be derived from Influenza virus, respiratory syncytial virus (RSV), Herpes simplex virus (HSV), human Papilloma virus (HPV), Human immunodeficiency virus (HIV), Plasmodium, Staphylococcus aureus, Dengue virus, Chlamydia trachomatis, Cytomegalovirus (CMV), Hepatitis B virus (HBV), Mycobacterium tuberculosis, Rabies virus, and Yellow Fever Virus, or an isoform, homolog, fragment, variant or derivative of any of these proteins.

Further preferred pathogenic antigens may be derived from Agrobacterium tumefaciens, Ajellomyces dermatitidis ATCC 60636, Alphapapillomavirus 10, Andes orthohantavirus, Andes virus CHI-7913, Aspergillus terreus NIH2624, Avian hepatitis E virus, Babesia microti, Bacillus anthracis, Bacteria, Betacoronavirus England 1, Blattella germanica, Bordetella pertussis, Borna disease virus Giessen strain He/80, Borrelia burgdorferi B31, Borrelia burgdorferi CA12, Borrelia burgdorferi N40, Borrelia burgdorferi ZS7, Borrelia garinii IP90, Borrelia hermsii, Borreliella afzelii, Borreliella burgdorferi, Borreliella garinii, Bos taurus, Brucella melitensis, Brugia malayi, Bundibugyo ebolavirus, Burkholderia pseudomallei, Burkholderia pseudomallei K96243, Campylobacter jejuni, Campylobacter upsaliensis, Candida albicans, Cavia porcellus, Chikungunya virus, Chikungunya virus MY/08/065, Chikungunya virus Singapore/11/2008, Chikungunya virus strain LR2006_OPY1 IMT/Reunion Island/2006, Chikungunya virus strain S27-African prototype, Chlamydia pneumoniae, Chlamydia trachomatis, Chlamydia trachomatis Serovar D, Chlamydiae, Clostridioides difficile, Clostridium difficile BI/NAP1/027, Clostridium tetani, Convict Creek 107 virus, Corynebacterium diphtheriae, Cowpox virus (Brighton Red) White-pock, Coxsackievirus A16, Coxsackievirus A9, Coxsackievirus B1, Coxsackievirus B2, Coxsackievirus B3, Coxsackievirus B4, Crimean-Congo hemorrhagic fever orthonairovirus, Cryptosporidium parvum, Dengue virus, Dengue virus 1, Dengue virus 1 Nauru/West Pac/1974, Dengue virus 1 PVP159, Dengue virus 1 Singapore/S275/1990, Dengue virus 2, Dengue virus 2 D2/SG/05K4155DK1/2005, Dengue virus 2 Jamaica/1409/1983, Dengue virus 2 Puerto Rico/PR159-S1/1969, Dengue virus 2 strain 43, Dengue virus 2 Thailand/16681/84, Dengue virus 2 Thailand/NGS-C/1944, Dengue virus 3, Dengue virus 4, Dengue virus 4 Dominica/814669/1981, Dengue virus 4 Thailand/0348/1991, Dengue virus type 1 Hawaii, Ebola virus—Mayinga, Zaire, 1976, Ebolavirus, Echinococcus granulosus, Echinococcus multilocularis, Echovirus E11, Echovirus E9, Ehrlichia canis str. Jake, Ehrlichia chaffeensis, Ehrlichia chaffeensis str. Arkansas, Entamoeba histolytica, Entamoeba histolytica YS-27, Enterococcus faecium, Enterovirus A, Enterovirus A71, Enterovirus C, Escherichia coli, Fasciola gigantica, Fasciola hepatica, Four Corners hantavirus, Francisella tularensis, Francisella tularensis subsp. holarctica LVS, Francisella tularensis subsp. tularensis SCHU S4, Gambierdiscus toxicus, GB virus C, Glossina morsitans morsitans, Gnathostoma binucleatum, Gp160, H1N1 subtype, H5N1 subtype, Haemophilus influenzae NTHi 1128, Haemophilus influenzae Serotype B, Haemophilus influenzae Subtype 1H, Hantaan orthohantavirus, Hantaan virus 76-118, HBV genotype D, Helicobacter pylori, Helicobacter pylori 26695, Heligmosomoides polygyrus, Hepatitis B virus, Hepatitis B virus adr4, Hepatitis B virus ayw/France/Tiollais/1979, Hepatitis B virus genotype D, Hepatitis B virus subtype adr, Hepatitis B virus subtype adw, Hepatitis B virus subtype adw2, Hepatitis B virus subtype adyw, Hepatitis B virus subtype AYR, Hepatitis B virus subtype ayw, Hepatitis C virus, Hepatitis C virus (isolate 1), Hepatitis C virus (isolate BK), Hepatitis C virus (isolate Con1), Hepatitis C virus (isolate Glasgow), Hepatitis C virus (isolate H), Hepatitis C virus (isolate H77), Hepatitis C virus (isolate HC-G9), Hepatitis C virus (isolate HCV-K3a/650), Hepatitis C virus (isolate Japanese), Hepatitis C virus (isolate JK049), Hepatitis C virus (isolate NZL1), Hepatitis C virus (isolate Taiwan), Hepatitis C virus genotype 1, Hepatitis C virus genotype 2, Hepatitis C virus genotype 3, Hepatitis C virus genotype 4, Hepatitis C virus genotype 5, Hepatitis C virus genotype 6, Hepatitis C virus HCT18, Hepatitis C virus HCV-KF, Hepatitis C virus isolate HC-J1, Hepatitis C virus isolate HC-J6, Hepatitis C virus isolate HC-J8, Hepatitis C virus JFH-1, Hepatitis C virus subtype 1a, Hepatitis C virus subtype 1a Chiron Corp., Hepatitis C virus subtype 1b, Hepatitis C virus subtype 1b AD78, Hepatitis C virus subtype 1b isolate BE-11, Hepatitis C virus subtype 1b JK1, Hepatitis C virus subtype 2a, Hepatitis C virus subtype 2b, Hepatitis C virus subtype 3a, Hepatitis C virus subtype 5a, Hepatitis C virus subtype 6a, Hepatitis delta virus, Hepatitis delta virus TW2667, Hepatitis E virus, Hepatitis E virus (strain Burma), Hepatitis E virus (strain Mexico), Hepatitis E virus SAR-55, Hepatitis E virus type 3 Kernow-C1, Hepatitis E virus type 4 JAK-Sai, Hepatovirus A, Heron hepatitis B virus, Herpes simplex virus (type 1/strain 17), Herpesviridae, HIV-1 CRF01_AE, HIV-1 group O, HIV-1 M:A, HIV-1 M:B, HIV-1 M:B_89.6, HIV-1 M:B_HXB2R, HIV-1 M:B_MN, HIV-1 M:C, HIV-1 M:CRF01_AE, HIV-1 M:G, HIV-1 O_ANT70, Human adenovirus 11, Human adenovirus 2, Human adenovirus 40, Human adenovirus 5, Human alphaherpesvirus 1, Human alphaherpesvirus 2, Human alphaherpesvirus 3, Human betaherpesvirus 5, Human betaherpesvirus 6B, Human bocavirus 1, Human bocavirus 2, Human bocavirus 3, Human coronavirus 229E, Human coronavirus OC43, Human endogenous retrovirus, Human endogenous retrovirus H, Human endogenous retrovirus K, Human enterovirus 71 Subgenogroup C4, Human gammaherpesvirus 4, Human gammaherpesvirus 8, Human hepatitis A virus Hu/Australia/HM175/1976, Human herpesvirus 1 strain KOS, Human herpesvirus 2 strain 333, Human herpesvirus 2 strain HG52, Human herpesvirus 3 H-551, Human herpesvirus 3 strain Oka vaccine, Human herpesvirus 4 strain B95-8, Human herpesvirus 4 type 1, Human herpesvirus 4 type 2, Human herpesvirus 5 strain AD169, Human herpesvirus 5 strain Towne, Human herpesvirus 6 (strain Uganda-1102), Human herpesvirus 7 strain JI, Human immunodeficiency virus 1, Human immunodeficiency virus 2, Human immunodeficiency virus type 1 (isolate YU2), Human immunodeficiency virus type 1 (JRCSF ISOLATE), Human immunodeficiency virus type 1 (NEW YORK-5 ISOLATE), Human immunodeficiency virus type 1 (SF162 ISOLATE), Human immunodeficiency virus type 1 (SF33 ISOLATE), Human immunodeficiency virus type 1 BH10, Human metapneumovirus, Human orthopneumovirus, Human papillomavirus, Human papillomavirus type 11, Human papillomavirus type 16, Human papillomavirus type 18, Human papillomavirus type 29, Human papillomavirus type 31, Human papillomavirus type 33, Human papillomavirus type 35, Human papillomavirus type 39, Human papillomavirus type 44, Human papillomavirus type 45, Human papillomavirus type 51, Human papillomavirus type 52, Human papillomavirus type 58, Human papillomavirus type 59, Human papillomavirus type 6, Human papillomavirus type 68, Human papillomavirus type 6b, Human papillomavirus type 73, Human parainfluenza 3 virus (strain NIH 47885), Human parechovirus 1, Human parvovirus 4, Human parvovirus B19, Human poliovirus 1, Human poliovirus 1 Mahoney, Human poliovirus 3, Human polyomavirus 1, Human respiratory syncytial virus (strain RSB1734), Human respiratory syncytial virus (strain RSB6190), Human respiratory syncytial virus (strain RSB6256), Human respiratory syncytial virus (strain RSB642), Human respiratory syncytial virus (subgroup B/strain 18537), Human respiratory syncytial virus A, Human respiratory syncytial virus A strain Long, Human respiratory syncytial virus A2, Human respiratory syncytial virus S2, Human respirovirus 3, Human rhinovirus A89, Human rotavirus A, Human T-cell lymphotrophic virus type 1 (Caribbean isolate), Human T-cell lymphotrophic virus type 1 (isolate MT-2), Human T-cell lymphotrophic virus type 1 (strain ATK), Human T-cell lymphotropic virus type 1 (african isolate), Human T-lymphotropic virus 1, Human T-lymphotropic virus 2, Influenza A virus, Influenza A virus (A/Anhui/1/2005(H5N1)), Influenza A virus (A/Anhui/PA-1/2013(H7N9)), Influenza A virus (A/Argentina/3779/94(H3N2)), Influenza A virus (A/Auckland/1/2009(H1N1)), Influenza A virus (A/Bar-headed Goose/Qinghai/61/05(H5N1)), Influenza A virus (A/Brevig Mission/1/1918(H1N1)), Influenza A virus (A/California/04/2009(H1N1)), Influenza A virus (A/California/07/2009(H1N1)), Influenza A virus (A/California/08/2009(H1N1)), Influenza A virus (A/California/10/1978(H1N1)), Influenza A virus (A/Christchurch/2/1988(H3N2)), Influenza A virus (A/Cordoba/3278/96(H3N2)), Influenza A virus (A/France/75/97(H3N2)), Influenza A virus (A/Fujian/411/2002(H3N2)), Influenza A virus (A/Hong Kong/01/2009(H1N1)), Influenza A virus (A/Hong Kong/1/1968(H3N2)), Influenza A virus (A/Indonesia/CDC699/2006(H5N1)), Influenza A virus (A/Iran/1/1957(H2N2)), Influenza A virus (A/Memphis/13/1978(H1N1)), Influenza A virus (A/Memphis/4/1980(H3N2)), Influenza A virus (A/Nanchang/58/1993(H3N2)), Influenza A virus (A/New York/232/2004(H3N2)), Influenza A virus (A/New_York/15/94(H3N2)), Influenza A virus (A/New_York/17/94(H3N2)), Influenza A virus (A/Ohio/3/95(H3N2)), Influenza A virus (A/Otago/5/2005(H1N1)), Influenza A virus (A/Puerto Rico/8/1934(H1N1)), Influenza A virus (A/Shangdong/5/94(H3N2)), Influenza A virus (A/Solomon Islands/3/2006 (Egg passage)(H1N1)), Influenza A virus (A/South Carolina/1/1918(H1N1)), Influenza A virus (A/swine/Hong Kong/126/1982(H3N2)), Influenza A virus (A/swine/Iowa/15/1930(H1N1)), Influenza A virus (A/Sydney/05/97-like(H3N2)), Influenza A virus (A/Texas/1/1977(H3N2)), Influenza A virus (A/Udorn/307/1972(H3N2)), Influenza A virus (A/Uruguay/716/2007(H3N2)), Influenza A virus (A/USSR/26/1985(H3N2)), Influenza A virus (A/Viet Nam/1203/2004(H5N1)), Influenza A virus (A/Vietnam/1194/2004(H5N1)), Influenza A virus (A/Wellington/75/2006(H1N1)), Influenza A virus (A/Wilson-Smith/1933(H1N1)), Influenza A virus (A/Wuhan/359/1995(H3N2)), Influenza A virus (STRAIN A/EQUINE/NEW MARKET/76), Influenza B virus, Japanese encephalitis virus, Japanese encephalitis virus strain Nakayama, Japanese encephalitis virus Vellore P20778, JC polyomavirus, Junin mammarenavirus, Klebsiella pneumoniae, Kumlinge virus, Lake Victoria marburgvirus—Popp, Lassa mammarenavirus, Lassa virus Josiah, Leishmania, Leishmania aethiopica, Leishmania braziliensis, Leishmania braziliensis MHOM/BR/75/M2904, Leishmania chagasi, Leishmania donovani, Leishmania infantum, Leishmania major, Leishmania major strain Friedlin, Leishmania panamensis, Leishmania pifanoi, Leptospira interrogans, Leptospira interrogans serovar Australis, Leptospira interrogans serovar Copenhageni, Leptospira interrogans serovar Copenhageni str. Fiocruz 1-130, Leptospira interrogans serovar Lai, Leptospira interrogans serovar Lai str. HY-1, Leptospira interrogans serovar Pomona, Little cherry virus 1, Lymphocytic choriomeningitis mammarenavirus, Measles morbillivirus, Measles virus strain Edmonston, Merkel cell polyomavirus, Mobala mammarenavirus, Modified Vaccinia Ankara virus, Moraxella catarrhalis O35E, Mupapillomavirus 1, Mus musculus, Mycobacterium, Mycobacterium abscessus, Mycobacterium avium, Mycobacterium avium serovar 8, Mycobacterium avium subsp. paratuberculosis, Mycobacterium bovis AN5, Mycobacterium bovis BCG, Mycobacterium bovis BCG str. Pasteur 1173P2, Mycobacterium fortuitum subsp. fortuitum, Mycobacterium gilvum, Mycobacterium intracellulare, Mycobacterium kansasii, Mycobacterium leprae, Mycobacterium leprae TN, Mycobacterium marinum, Mycobacterium neoaurum, Mycobacterium phlei, Mycobacterium smegmatis, Mycobacterium tuberculosis, Mycobacterium tuberculosis CDC1551, Mycobacterium tuberculosis H37Ra, Mycobacterium tuberculosis H37Rv, Mycobacterium ulcerans, Mycoplasma pneumoniae, Mycoplasma pneumoniae FH, Mycoplasma pneumoniae M129, Necator americanus, Neisseria gonorrhoeae, Neisseria meningitidis serogroup B H44/76, Nipah henipavirus, Norovirus genogroup 2 Camberwell 1890, Onchocerca volvulus, Orientia tsutsugamushi, Oryctolagus cuniculus, Pan troglodytes, Paracoccidioides brasiliensis, Paracoccidioides brasiliensis B339, Plasmodium falciparum, Plasmodium falciparum 3D7, Plasmodium falciparum 7G8, Plasmodium falciparum FC27/Papua New Guinea, Plasmodium falciparum FCR-3/Gambia, Plasmodium falciparum isolate WELLCOME, Plasmodium falciparum K1, Plasmodium falciparum LE5, Plasmodium falciparum Mad20/Papua New Guinea, Plasmodium falciparum NF54, Plasmodium falciparum Palo Alto/Uganda, Plasmodium falciparum RO-33, Plasmodium reichenowi, Plasmodium vivax, Plasmodium vivax NK, Plasmodium vivax Sal-1, Plasmodium vivax strain Belem, Plasmodium vivax-like sp., Porphyromonas gingivalis, Porphyromonas gingivalis 381, Porphyromonas gingivalis OMZ 409, Prevotella sp. oral taxon 472 str. F0295, Pseudomonas aeruginosa, Puumala orthohantavirus, Puumala virus (strain Umea/hu), Puumala virus sotkamo/v-2969/81, Pythium insidiosum, Ravn-Ravn, Kenya, 1987, Respiratory syncytial virus, Rhodococcus fascians, Rhodococcus hoagii, Rubella virus, Rubella virus strain M33, Rubella virus strain Therien, Rubella virus vaccine strain RA27/3, Saccharomyces cerevisiae, Saimiriine gammaherpesvirus 2, Salmonella enterica subsp. enterica serovar Typhi, Salmonella ‘group A’, Salmonella ‘group D’, Salmonella sp. ‘group B’, Sapporo rat virus, SARS coronavirus, SARS coronavirus BJ01, SARS coronavirus TJF, SARS coronavirus Tor2, SARS coronavirus Urbani, Schistosoma, Schistosoma japonicum, Schistosoma mansoni, Schistosoma mansoni Puerto Rico, Sin Nombre orthohantavirus, Sindbis virus, Staphylococcus aureus, Staphylococcus aureus subsp. aureus COL, Staphylococcus aureus subsp. aureus MRSA252, Streptococcus, Streptococcus mutans, Streptococcus mutans MT 8148, Streptococcus oralis, Streptococcus pneumoniae, Streptococcus pyogenes, Streptococcus pyogenes serotype M24, Streptococcus pyogenes serotype M3 D58, Streptococcus pyogenes serotype M5, Streptococcus pyogenes serotype M6, Streptococcus sp. ‘group A’, Taenia crassiceps, Taenia saginata, Taenia solium, Tick-borne encephalitis virus, Toxocara canis, Toxoplasma gondii, Toxoplasma gondii ME49, Toxoplasma gondii RH, Toxoplasma gondii type I, Toxoplasma gondii type II, Toxoplasma gondii type III, Toxoplasma gondii VEG, Treponema pallidum, Treponema pallidum subsp. pallidum str. Nichols, Trichomonas vaginalis, Triticum aestivum, Trypanosoma brucei brucei, Trypanosoma brucei gambiense, Trypanosoma cruzi, Trypanosoma cruzi Dm28c, Trypanosoma cruzi strain CL Brener, Vaccinia virus, Vesicular stomatitis virus, Vibrio cholerae, West Nile virus, West Nile virus NY-99, Wuchereria bancrofti, Yellow fever virus 17D/Tiantan, Yersinia enterocolitica, Zaire ebolavirus, Zika virus, or an isoform, homolog, fragment, variant or derivative of any of these proteins.

Artificial nucleic acid molecules of the invention encoding preferred influenza-derived pathogenic antigens may preferably comprise a coding region comprising or consisting of a nucleic acid sequence according to any one of the SEQ ID NOs as shown in FIG. 1, FIG. 2, FIG. 3 or FIG. 4 or respectively Table 1, Table 2, Table 3 or Table 4 of international patent application PCT/EP2017/060663, or a fragment or variant of any of these sequences, in particular a nucleic acid sequence having a sequence identity of at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, preferably at least 80% to any of these sequences. In this context, the disclosure of PCT/EP2017/060663 is incorporated herein by reference.

Artificial nucleic acid molecules of the invention encoding further preferred influenza-derived pathogenic antigens may preferably comprise a coding region comprising or consisting of a nucleic acid sequence according to any one of the SEQ ID NOs as shown in FIG. 20, FIG. 21, FIG. 22, or FIG. 23 or respectively Table 1, Table 2, Table 3 or Table 4 of international patent application PCT/EP2017/064066, or a fragment or variant of any of these sequences, in particular a nucleic acid sequence having a sequence identity of at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, preferably at least 80% to any of these sequences. In this context, the disclosure of PCT/EP2017/064066 is incorporated herein by reference.

Artificial nucleic acid molecules of the invention encoding preferred rabies virus-derived pathogenic antigens may preferably comprise a coding region comprising or consisting of a nucleic acid sequence according to SEQ ID NO: 24 or SEQ ID NO: 25 of international patent application WO 2015/024665 A1, or a fragment or variant of any of these sequences, in particular a nucleic acid sequence having a sequence identity of at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, preferably at least 80% to any of these sequences. In this context, the disclosure of WO 2015/024665 A1 is incorporated herein by reference.

Artificial nucleic acid molecules of the invention encoding further preferred rabies virus-derived pathogenic antigens may preferably comprise a coding region comprising or consisting of a nucleic acid sequence according to SEQ ID NO: 24 or Table 5 of international patent application PCT/EP2017/064066, or a fragment or variant of any of these sequences, in particular a nucleic acid sequence having a sequence identity of at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, preferably at least 80% to any of these sequences. In this context, the disclosure of PCT/EP2017/064066 is incorporated herein by reference.

Artificial nucleic acid molecules of the invention encoding preferred RSV-derived pathogenic antigens may preferably comprise a coding region comprising or consisting of a nucleic acid sequence according to any one of SEQ ID NOs: 31 to 35 of international patent application WO 2015/024668 A2, or a fragment or variant of any of these sequences, in particular a nucleic acid sequence having a sequence identity of at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, preferably at least 80% to any of these sequences. In this context, the disclosure of WO 2015/024668 A2 is incorporated herein by reference.

Artificial nucleic acid molecules of the invention encoding preferred Ebola or Marburgvirus-derived pathogenic antigens may preferably comprise a coding region comprising or consisting of a nucleic acid sequence according to any one of SEQ ID NOs: 20 to 233 of international patent application WO 2016/097065 A1, or a fragment or variant of any of these sequences, in particular a nucleic acid sequence having a sequence identity of at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, preferably at least 80% to any of these sequences. In this context, the disclosure of WO 2016/097065 A1 is incorporated herein by reference.

Artificial nucleic acid molecules of the invention encoding preferred Zikavirus-derived pathogenic antigens may preferably comprise a coding region comprising or consisting of a nucleic acid sequence according to any one of SEQ ID NOs: 1 to 11759 or Table 1, Table 1A, Table 2, Table 2A, Table 3, Table 3A, Table 4, Table 4A, Table 5, Table 5A, Table 6, Table 6A, Table 7, Table 8, or Table 14 of international patent application WO 2017/140905 A1, or a fragment or variant of any of these sequences, in particular a nucleic acid sequence having a sequence identity of at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, preferably at least 80% to any of these sequences. In this context, the disclosure of WO 2017/140905 A1 is incorporated herein by reference.

Artificial nucleic acid molecules of the invention encoding preferred Norovirus-derived pathogenic antigens may preferably comprise a coding region comprising or consisting of a nucleic acid sequence according to any one of SEQ ID NOs: 1 to 39746 or Table 1 of international patent application PCT/EP2017/060673, or a fragment or variant of any of these sequences, in particular a nucleic acid sequence having a sequence identity of at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, preferably at least 80% to any of these sequences. In this context, the disclosure of PCT/EP2017/060673 is incorporated herein by reference.

Artificial nucleic acid molecules of the invention encoding preferred Rotavirus-derived pathogenic antigens may preferably comprise a coding region comprising or consisting of a nucleic acid sequence according to any one of SEQ ID NOs: 1 to 3593 or Tables 1-20 of international patent application WO 2017/081110 A1, or a fragment or variant of any of these sequences, in particular a nucleic acid sequence having a sequence identity of at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, preferably at least 80% to any of these sequences. In this context, the disclosure of WO 2017/081110 A1 is incorporated herein by reference.

The term “autoantigen” refers to an endogenous “self-” antigen that—despite being a normal body constituent—induces an autoimmune reaction in the host. In the context of the present invention, autoantigens are preferably of human origin. The provision of an artificial nucleic acid (RNA) molecule encoding an antigenic (poly-)peptide or protein derived from an autoantigen can, for instance, be used to induce immune tolerance towards said autoantigen. Exemplary autoantigens in the context of the present invention include, without limitation, autoantigen derived or selected from 60 kDa chaperonin 2, Lipoprotein LpqH, Melanoma antigen recognized by T-cells 1, MHC class I polypeptide-related sequence A, Parent Protein, Structural polyprotein, Tyrosinase, Myelin proteolipid protein, Epstein-Barr nuclear antigen 1, Envelope glycoprotein GP350, Genome polyprotein, Collagen alpha-1(II) chain, Aggrecan core protein, Melanocyte-stimulating hormone receptor, Acetylcholine receptor subunit alpha, 60 kDa heat shock protein, mitochondrial, Histone H4, Myosin-11, Glutamate decarboxylase 2, 60 kDa chaperonin, PqqC-like protein, Thymosin beta-10, Myelin basic protein, Epstein-Barr nuclear antigen 4, Melanocyte protein PMEL, HLA class II histocompatibility antigen, DQ beta 1 chain, Latent membrane protein 2, Integrin beta-3, Nucleoprotein, 60S ribosomal protein L10, Protein BOLF1, 60S acidic ribosomal protein P2, Latent membrane protein 1, Collagen alpha-2(VI) chain, Exodeoxyribonuclease V, Gamma, Trans-activator protein BZLF1, S-arrestin, HLA class I histocompatibility antigen, A-3 alpha chain, Protein CT_579, Matrin-3, Envelope glycoprotein B, ATP-dependent zinc metalloprotease FtsH, U1 small nuclear ribonucleoprotein 70 kDa, CD48 antigen, Tubulin beta chain, Actin, cytoplasmic 1, Epstein-Barr nuclear antigen 3, NEDD4 family-interacting protein 1, 60S ribosomal protein L28, Immediate-early protein 2, Insulin, isoform 2, Keratin, type II cytoskeletal 3, Matrix protein 1, Histone H2A.Z, mRNA export factor ICP27 homolog, Small nuclear ribonucleoprotein-associated proteins B and B′, Large cysteine-rich periplasmic protein OmcB, Smoothelin, Small nuclear ribonucleoprotein Sm D1, Acetylcholine receptor subunit epsilon, Invasin repeat family phosphatase, Alpha-crystallin B chain, HLA class II histocompatibility antigen, DRB1-13 beta chain, HLA class II histocompatibility antigen, DRB1-4 beta chain, Dihydrolipoyllysine-residue acetyltransferase component of pyruvate dehydrogenase complex, mitochondrial, Keratin, type I cytoskeletal 18, Epstein-Barr nuclear antigen 6, Protein Tax-1, Vimentin, Keratin, type I cytoskeletal 16, Keratin, type I cytoskeletal 10, HLA class I histocompatibility antigen, B-27 alpha chain, Thyroglobulin, Acetylcholine receptor subunit gamma, Chaperone protein DnaK, Protein U24, Na(+)-translocating NADH-quinone reductase subunit A, 65 kDa phosphoprotein, Probable ATP-dependent Clp protease ATP-binding subunit, Probable outer membrane protein PmpC, Heat shock 70 kDa protein 1B, Hemagglutinin, Tetanus toxin, Enolase, Ras-associated and pleckstrin homology domains-containing protein 1, Keratin, type II cytoskeletal 7, Myosin-9, Histone H1-like protein Hc1, Envelope glycoprotein gp160, Urease subunit beta, Vasoactive intestinal polypeptide receptor 1, Viral interleukin-10 homolog, Histone H3.3, Replication protein A 32 kDa subunit, Probable outer membrane protein PmpD, Insulin-2, L-dopachrome tautomerase, Keratin, type I cytoskeletal 9, Envelope glycoprotein H, DNA polymerase catalytic subunit, Beta-2-glycoprotein 1, Envelope glycoprotein gp62, Serum albumin, Major DNA-binding protein, HLA class I histocompatibility antigen, A-2 alpha chain, Myeloblastin, POTE ankyrin domain family member I, Protein E7, Predicted Efflux Protein, Replication and transcription activator, Gag-Pro-Pol polyprotein, Capsid protein VP26, Major capsid protein, Apoptosis regulator BHRF1, Epstein-Barr nuclear antigen 2, HLA class I histocompatibility antigen, B-7 alpha chain, Calreticulin, Gamma-secretase C-terminal fragment 59, Insulin, Glucose-6-phosphatase 2, Islet amyloid polypeptide, Receptor-type tyrosine-protein phosphatase N2, Receptor-type tyrosine-protein phosphatase-like N, Islet cell autoantigen 1, Bos d 6, Glutamate decarboxylase 1, 60S ribosomal protein L29, 28S ribosomal protein S31, mitochondrial, HLA class II histocompatibility antigen, DRB1-16 beta chain, Collagen alpha-3(IV) chain, Glucose-6-phosphatase, Glucose-6-phosphatase 3, Collagen alpha-5(IV) chain, Protein Nef, Glial fibrillary acidic protein, Fibrillin-1, Tenascin, Stromelysin-1, Interstitial collagenase, Calpain-2 catalytic subunit, Chondroitin sulfate proteoglycan 4, Fibrinogen beta chain, Chaperone protein DnaJ, Chitinase-3-like protein 1, Matrix metalloproteinase-16, DNA topoisomerase 1, Follistatin-related protein 1, Ig gamma-1 chain C region, Ig gamma-3 chain C region, Collagen alpha-2(XI) chain, Desmoglein-3, Fibrinogen alpha chain, Filaggrin, T-cell receptor beta chain V region CTL-L17, T-cell receptor beta-1 chain C region, Ig heavy chain V-I region EU, Collagen alpha-1(IV) chain, HLA class I histocompatibility antigen, Cw-7 alpha chain, HLA class I histocompatibility antigen, B-35 alpha chain, HLA class I histocompatibility antigen, B-38 alpha chain, High mobility group protein B2, Ig heavy chain V-II region ARH-77, HLA class II histocompatibility antigen, DR beta 4 chain, Ig kappa chain C region, Alpha-enolase, Lysosomal-associated transmembrane protein 5, HLA class I histocompatibility antigen, B-52 alpha chain, Heterogeneous nuclear ribonucleoproteins A2/B1, T-cell receptor beta chain V region YT35, Ig gamma-4 chain C region, T-cell receptor beta-2 chain C region, DnaJ homolog subfamily B member 2, DnaJ homolog subfamily A member i, Ig kappa chain V-IV region Len, Ig heavy chain V-II region OU, Ig kappa chain V-IV region B17, 2′, 3′-cyclic-nucleotide 3′-phosphodiesterase, Ig heavy chain V-II region MCE, Ig kappa chain V-III region HIC, Ig heavy chain V-II region COR, Myelin-oligodendrocyte glycoprotein, Ig kappa chain V-II region RPMI 6410, Ig kappa chain V-II region GM607, Immunoglobulin lambda-like polypeptide 5, Ig heavy chain V-II region WAH, Biotin-protein ligase, Oligodendrocyte-myelin glycoprotein, Transaldolase, DNA helicase/primase complex-associated protein, Interferon beta, Myelin-associated oligodendrocyte basic protein, Myelin-associated glycoprotein, Fusion glycoprotein F0, Myelin protein P0, Ig lambda chain V-II region MGC, DNA primase, Minor capsid protein L2, Myelin P2 protein, Peripheral myelin protein 22, Retinol-binding protein 3, Butyrophilin subfamily 1 member A1, Alkaline nuclease, Claudin-11, N-acetylmuramoyl-L-alanine amidase CwlH, GTPase Der, Possible transposase, ABC transporter, ATP-binding protein, putative, Collagen alpha-2(IV) chain, Calpastatin, Ig kappa chain V-III region SIE, E3 ubiquitin-protein ligase TRIM68, Glutamate receptor ionotropic, NMDA 2A, Spectrin alpha chain, non-erythrocytic 1, Lupus La protein, Complement C1q subcomponent subunit A, U1 small nuclear ribonucleoprotein A, 60 kDa SS-A/Ro ribonucleoprotein, DNA repair protein XRCC4, Histone H3-like centromeric protein A, Histone H1.4, Putative HTLV-1-related endogenous sequence, HLA class II histocompatibility antigen, DRB1-3 chain, HLA class II histocompatibility antigen, DRB1-1 beta chain, Small nuclear ribonucleoprotein Sm D3, Tumor necrosis factor receptor superfamily member 6, Phosphomannomutase/phosphoglucomutase, Tripartite terminase subunit UL15, Proteasome subunit beta type-3, Proliferating cell nuclear antigen, Inner capsid protein sigma-2, Histone H2B type 1, E3 ubiquitin-protein ligase TRIM21, DNA-directed RNA polymerase II subunit RPB1, X-ray repair cross-complementing protein 6, U1 small nuclear ribonucleoprotein C, Caspase-8, 60S ribosomal protein L7, 5-hydroxytryptamine receptor 4, Small nuclear ribonucleoprotein-associated protein N, Exportin-1, 60S acidic ribosomal protein P0, Neurofilament heavy polypeptide, putative env, T-cell receptor alpha chain C region, T-cell receptor alpha chain V region CTL-L17, RNA polymerase sigma factor SigA, Small nuclear ribonucleoprotein Sm D2, Immunoglobulin iota chain, Ig kappa chain V-III region WOL, Histone H2B type 1-F/J/L, High mobility group protein B1, X-ray repair cross-complementing protein 5, Muscarinic acetylcholine receptor M3, Major viral transcription factor ICP4, Voltage-dependent P/Q-type calcium channel subunit alpha-1A, Heat shock protein HSP 90-beta, DNA topoisomerase 2-beta, Histone H3.1, Tumor necrosis factor ligand superfamily member 6, Phospho-N-acetylmuramoyl-pentapeptide-transferase, Hemoglobin subunit alpha, Apolipoprotein E, CD99 antigen, ATP synthase subunit beta, mitochondrial, Acetylcholine receptor subunit delta, Acyl-CoA dehydrogenase family member 10, KN motif and ankyrin repeat domain-containing protein 3, SAM and SH3 domain-containing protein 1, Elongation factor 1-alpha 1, GTP-binding nuclear protein Ran, Myosin-7, Sal-like protein 1, IgGFc-binding protein, E3 ubiquitin-protein ligase SIAH1, Muscleblind-like protein 2, Annexin A1, Protein PET117 homolog, mitochondrial, Nuclear ubiquitous casein and cyclin-dependent kinase substrate 1, Pleiotropic regulator 1, NADH dehydrogenase [ubiquinone] 1 alpha subcomplex subunit 3, Guanine nucleotide-binding protein G(o) subunit alpha, Microtubule-associated protein 1B, L-serine dehydratase/L-threonine deaminase, Centromere protein J, SH3 and multiple ankyrin repeat domains protein 3, Fumarate hydratase, mitochondrial, Cofilin-1, Rho GTPase-activating protein 9, Phosphatidate cytidylyltransferase 1, Neurofilament light polypeptide, Calsyntenin-1, GPI transamidase component PIG-T, Perilipin-3, Protein unc-13 homolog D, WD40 repeat-containing protein SMU1, Neurofilament medium polypeptide, Protein S100-B, Carboxypeptidase E, Neurexin-2-beta, NAD-dependent protein deacetylase sirtuin-2, Tripartite motif-containing protein 40, Neurexin-1-beta, Annexin A11, Hemoglobin subunit beta, Glyceraldehyde-3-phosphate dehydrogenase, Histidine triad nucleotide-binding protein 3, ATP synthase subunit e, mitochondrial, 10 kDa heat shock protein, mitochondrial, Cellular tumor antigen p53, Leukocyte-associated immunoglobulin-like receptor 1, Tubulin alpha-1B chain, Splicing factor, proline- and glutamine-rich, Olfactory receptor 10A4, Histone H2B type 2-F, Calmodulin, RNA-binding protein Raly, Phosphoinositide-3-kinase-interacting protein 1, Alpha-2-macroglobulin, Glycogen phosphorylase, brain form, THO complex subunit 4, Neuroblast differentiation-associated protein AHNAK, Phosphoserine aminotransferase, Mitochondrial folate transporter/carrier, Sentrin-specific protease 3, Cytosolic Fe—S cluster assembly factor NUBP2, Histone deacetylase 7, Serine/threonine-protein phosphatase 2A 55 kDa regulatory subunit B alpha isoform, Serine/threonine-protein phosphatase 2A regulatory subunit B” subunit alpha, Gelsolin, Insulin-like growth factor II, Tight junction protein ZO-1, Hsc70-interacting protein, FXYD domain-containing ion transport regulator 6, AP-1 complex subunit mu-1, Syntenin-1, NADH dehydrogenase [ubiquinone] iron-sulfur protein 7, mitochondrial, Low-density lipoprotein receptor, LIM domain transcription factor LMO4, Spectrin beta chain, non-erythrocytic 1, ATP-binding cassette sub-family A member 2, NADH dehydrogenase [ubiquinone] 1 subunit C2, SPARC-like protein 1, Electron transfer flavoprotein subunit alpha, mitochondrial, Glutamate dehydrogenase 1, mitochondrial, Complexin-2, Protein-serine O-palmitoleoyltransferase porcupine, Plexin domain-containing protein 2, Threonine synthase-like 2, Testican-2, C—X—C chemokine receptor type 1, Arachidonate 5-lipoxygenase-activating protein, Neuroguidin, Fatty acid 2-hydroxylase, Nuclear factor 1 X-type, LanC-like protein 1, Glutamine synthetase, Lysosome-associated membrane glycoprotein 1, Apolipoprotein A-I, Alpha-adducin, Guanine nucleotide-binding protein G(I)/G(S)/G(T) subunit beta-3, Integral membrane protein GPR137B, Ubiquilin-1, Aldose reductase, Clathrin light chain B, V-type proton ATPase subunit F, Apolipoprotein D, 40S ribosomal protein SA, Bcl-2-associated transcription factor 1, Phosphatidate cytidylyltransferase 2, ATP synthase-coupling factor 6, mitochondrial, Receptor tyrosine-protein kinase erbB-2, Echinoderm microtubule-associated protein-like 5, Phosphatidylethanolamine-binding protein 1, Myc box-dependent-interacting protein 1, Membrane-associated phosphatidylinositol transfer protein 1, 40S ribosomal protein S29, Small acidic protein, Galectin-3-binding protein, Fatty acid synthase, Baculoviral IAP repeat-containing protein 5, Septin-2, cAMP-dependent protein kinase type II-alpha regulatory subunit, Reelin, Apoptosis facilitator Bcl-2-like protein 14, Staphylococcal nuclease domain-containing protein 1, Methyl-CpG-binding domain protein 2, Transformation/transcription domain-associated protein, Transcription factor HES-1, Protein transport protein Sec23B, Paralemmin-2, C—C motif chemokine 15, Sodium/potassium-transporting ATPase subunit alpha-1, Stathmin, Heterogeneous nuclear ribonucleoprotein L-like, Nodal modulator 3, Interferon-induced GTP-binding protein Mx2, Integrin alpha-D, Low-density lipoprotein receptor-related protein 5-like protein, Macrophage migration inhibitory factor, Ferritin light chain, Dihydropyrimidinase-related protein 2, Neuronal membrane glycoprotein M6-b, ATP-binding cassette sub-family A member 5, Synaptosomal-associated protein 25, Insulin-like growth factor I, Ankyrin repeat domain-containing protein 29, Protein spinster homolog 3, Peflin, Contactin-1, Microfibril-associated glycoprotein 3, von Willebrand factor, Small nuclear ribonucleoprotein G, Interleukin-12 receptor subunit beta-1, Epoxide hydrolase 1, Cytochrome b-c1 complex subunit 10, Monoglyceride lipase, Serotransferrin, Alpha-synuclein, Cytosolic non-specific dipeptidase, Transgelin-2, Testisin, Fms-related tyrosine kinase 3 ligand, Noelin-2, Serine/threonine-protein kinase DCLK1, Interferon alpha-2, Acetylcholine receptor subunit beta, Histone H2A type 1, Beta-2 adrenergic receptor, Putrescine aminotransferase, Interferon alpha-1/13, Protein NEDD1, DnaJ homolog subfamily B member 1, Tubulin beta-6 chain, Non-histone chromosomal protein HMG-17, Polyprotein, Exosome component 10, Natural cytotoxicity triggering receptor 3 ligand 1, Gag polyprotein, Band 3 anion transport protein, Protease, Histidine-tRNA ligase, cytoplasmic, Collagen alpha-1(XVII) chain, Envoplakin, Histone H2B type 1-C/E/F/G/I, Diaminopimelate decarboxylase, Histone H2B type 2-E, Cytochrome P450 2D6, Dihydrolipoyllysine-residue succinyltransferase component of 2-oxoglutarate dehydrogenase complex, Histone H2B type 1-H, Thyroid peroxidase, Proline-rich transmembrane protein 2, Periplakin, Integrin alpha-6, Dystonin, Desmoplakin, Histone H2B type 1-J, Histone H2B type 1-B, 6,7-dimethyl-8-ribityllumazine synthase, Thyrotropin receptor, Integrin alpha-IIb, Nuclear pore membrane glycoprotein 210, Protein U2, DST protein, Plectin, Sll0397 protein, Bos d 10, Outer capsid protein VP4, 5,6-dihydroxyindole-2-carboxylic acid oxidase, O-phosphoseryl-tRNA(Sec) selenium transferase, ATP-dependent Clp protease proteolytic subunit, Lymphocyte activation gene 3 protein, Phosphoprotein 85, L1 protein, Actin, alpha skeletal muscle, Dihydrolipoyl dehydrogenase, Dihydrolipoyllysine-residue succinyltransferase component of 2-oxoglutarate dehydrogenase complex, mitochondrial, Liver carboxylesterase 1, Dihydrolipoyllysine-residue acetyltransferase component of pyruvate dehydrogenase complex, Acetyltransferase component of pyruvate dehydrogenase complex, Pyruvate dehydrogenase protein X component, mitochondrial, Dihydrolipoamide acetyltransferase, Protein disulfide-isomerase A3, Flotillin-2, Beta-galactosidase, TSHR protein, Lipoamide acyltransferase component of branched-chain alpha-keto acid dehydrogenase complex, mitochondrial, Nuclear autoantigen Sp-100, Desmoglein-1, Glucagon receptor, Membrane glycoprotein US8, Sodium/iodide cotransporter, ORF2, Capsid protein, Uncharacterized protein LF3, Formimidoyltransferase-cyclodeaminase, Core-capsid bridging protein, Neurovirulence factor ICP34.5, Probable RNA-binding protein, Cholesterol side-chain cleavage enzyme, mitochondrial, Histone H1.0, Non-histone chromosomal protein HMG-14, Histone H5, 60S acidic ribosomal protein P1, Pyruvate dehydrogenase E1 component subunit alpha, somatic form, mitochondrial, Leiomodin-1, Uncharacterized protein RP382, Uncharacterized protein U95, (Type IV) pilus assembly protein PilB, 2-succinylbenzoate-CoA ligase, TAZ protein, Tafazzin, Putative lactose-specific phosphotransferase system (PTS), IIBC component, Claudin-17, Pericentriolar material 1 protein, Yop proteins translocation protein L, Laminin subunit alpha-1, A disintegrin and metalloproteinase with thrombospondin motifs 13, Keratin, type I cytoskeletal 14, Coagulation factor VIII, Keratin, type I cytoskeletal 17, Neutrophil defensin 1, Ig alpha-1 chain C region, BRCA1-associated RING domain protein 1, Trinucleotide repeat-containing gene 6A protein, Thrombopoietin, Plasminogen-binding protein PgbA, Steroid 17-alpha-hydroxylase/17,20 lyase, Nucleolar RNA helicase 2, Histone H2B type 1-N, Steroid 21-hydroxylase, UreB, Melanin-concentrating hormone receptor 1, Blood group Rh(CE) polypeptide, HLA class II histocompatibility antigen, DP beta 1 chain, Platelet glycoprotein Ib alpha chain, Muscarinic acetylcholine receptor M1, Outer capsid glycoprotein VP7, Fibronectin, HLA class I histocompatibility antigen, B-8 alpha chain, AhpC, Cytoskeleton-associated protein 5, Sucrase-isomaltase, intestinal, Leukotriene B4 receptor 2, Glutathione peroxidase 2, Collagen alpha-1(VII) chain, Nucleosome assembly protein 1-like 4, Alanine-tRNA ligase, cytoplasmic, Extracellular calcium-sensing receptor, Major centromere autoantigen B, Large tegument protein deneddylase, Blood group Rh(D) polypeptide, Kininogen-1, Peroxiredoxin-2, Ezrin, DNA replication and repair protein RecF, Keratin, type II cytoskeletal 6C, Trigger factor, Serpin B5, Heat shock protein beta-1, Protein-arginine deiminase type-4, Potassium-transporting ATPase alpha chain 1, Potassium-transporting ATPase subunit beta, Forkhead box protein E3, Condensin-2 complex subunit D3, Myotonin-protein kinase, Zinc transporter 8, ABC transporter, substrate-binding protein, putative, Aquaporin-4, Cartilage intermediate layer protein 1, HLA class II histocompatibility antigen, DR beta 5 chain, Small nuclear ribonucleoprotein F, Small nuclear ribonucleoprotein E, Ig kappa chain V-V region L7, Ig heavy chain Mem5, Ig heavy chain V-III region J606, Hemoglobin subunit delta, Collagen alpha-1(XV) chain, 78 kDa glucose-regulated protein, 60S ribosomal protein L22, Alpha-1-acid glycoprotein 1, Malate dehydrogenase, mitochondrial, 60S ribosomal protein L8, Serine protease HTRA2, mitochondrial, 60S ribosomal protein L23a, Complement C3, Collagen alpha-1(XII) chain, Angiotensinogen, Protein S100-A9, Annexin A2, Alpha-actinin-4, HLA class II histocompatibility antigen, DQ alpha 1 chain, Apolipoprotein A-IV, Actin, aortic smooth muscle, HLA class II histocompatibility antigen, DP alpha 1 chain, Creatine kinase B-type, HLA class II histocompatibility antigen, DR beta 3 chain, Histone H1x, Heterogeneous nuclear ribonucleoprotein U-like protein 2, Basement membrane-specific heparan sulfate proteoglycan core protein, Cadherin-5, 40S ribosomal protein S13, Alpha-1-antitrypsin, Multimerin-2, Centromere protein F, 40S ribosomal protein S18, 40S ribosomal protein S25, Na(+)/H(+) exchange regulatory cofactor NHE-RF1, Actin, cytoplasmic 2, Hemoglobin subunit gamma-1, Hemoglobin subunit gamma-2, Protein NipSnap homolog 3A, Cathepsin D, 1-phosphatidylinositol 4,5-bisphosphate phosphodiesterase epsilon-1, 40S ribosomal protein S17, Apolipoprotein B-100, Histone H2B type 1-K, Collagen alpha-1(I) chain, Collagen alpha-2(I) chain, 3-hydroxyacyl-CoA dehydrogenase type-2, 60S ribosomal protein L27, Histone H1.2, Nidogen-2, Cadherin-1, 60S ribosomal protein L27a, HLA class II histocompatibility antigen, DR alpha chain, Dipeptidyl peptidase 1, Ubiquitin-40S ribosomal protein S27a, Citrate synthase, mitochondrial, Taxi-binding protein 1, Myeloperoxidase, Plexin domain-containing protein 1, Glycogen synthase, [Pyruvate dehydrogenase [acetyl-transferring]]-phosphatase 1, mitochondrial, Phorbol-12-myristate-13-acetate-induced protein 1, Peroxiredoxin-5, mitochondrial, 14-3-3 protein zeta/delta, ATP synthase subunit d, mitochondrial, Vitronectin, Lipopolysaccharide-binding protein, Ig heavy chain V-III region GAL, Protein CREG1, 60S ribosomal protein L6, Stabilin-1, Plasma protease C1 inhibitor, Ig kappa chain V-III region VG, Inter-alpha-trypsin inhibitor heavy chain H4, Alpha-1B-glycoprotein, Tartrate-resistant acid phosphatase type 5, Sulfhydryl oxidase 1, Complement component C6, Glycogen phosphorylase, muscle form, SH3 domain-binding glutamic acid-rich-like protein 3, Transforming protein RhoA, Albumin, isoform CRA_k, V-type proton ATPase subunit G 1, Flavin reductase (NADPH), Heat shock cognate 71 kDa protein, Lipoprotein lipase, Plasminogen, Annexin, Syntaxin-7, Transmembrane glycoprotein NMB, Coagulation factor XIII A chain, Apolipoprotein A-II, N-acetylglucosamine-6-sulfatase, Complement C1q subcomponent subunit B, Protein S100-A10, Microfibril-associated glycoprotein 4, 72 kDa type IV collagenase, Collagen alpha-1(XI) chain, Cathepsin B, Palmitoyl-protein thioesterase 1, Macrosialin, Histone H1.1, Histone H1.5, Fibromodulin, Thrombospondin-1, Rho GDP-dissociation inhibitor 2, Alpha-galactosidase A, Superoxide dismutase [Cu—Zn], HLA class I histocompatibility antigen, alpha chain E, Phosphatidylcholine-sterol acyltransferase, Legumain, Low affinity immunoglobulin gamma Fc region receptor II-c, Fructose-bisphosphate aldolase A, Cytochrome c oxidase subunit 8A, mitochondrial, Pyruvate kinase PKM, Endoglin, Target of Nesh-SH3, Cytochrome c oxidase subunit 5A, mitochondrial, EGF-containing fibulin-like extracellular matrix protein 2, Epididymal secretory protein E1, Cathepsin S, Annexin A5, Allograft inflammatory factor 1, Decorin, Complement Cis subcomponent, Low affinity immunoglobulin gamma Fc region receptor II-b, Leucine-rich alpha-2-glycoprotein, Lysosomal alpha-glucosidase, Disintegrin and metalloproteinase domain-containing protein 9, Transthyretin, Malate dehydrogenase, cytoplasmic, Filamin-A, Retinoic acid receptor responder protein 1, T-cell surface glycoprotein CD4, Procollagen-lysine, 2-oxoglutarate 5-dioxygenase 1, Fibrinogen gamma chain, Collagen alpha-2(V) chain, Cystatin-B, Lysosomal protective protein, Granulins, Collagen alpha-1(XIV) chain, C-reactive protein, Beta-1,4-galactosyltransferase 1, Prolow-density lipoprotein receptor-related protein 1, Ig heavy chain V-III region 23, Phosphoglycerate kinase 1, Alpha-2-antiplasmin, V-set and immunoglobulin domain-containing protein 4, Probable serine carboxypeptidase CPVL, NEDD8, Ganglioside GM2 activator, Clusterin, Alpha-2-HS-glycoprotein, HLA class I histocompatibility antigen, B-37 alpha chain, Adenosine deaminase CECR1, HLA class II histocompatibility antigen, DRB1-11 beta chain, Monocyte differentiation antigen CD14, Erythrocyte band 7 integral membrane protein, Profilin-1, E3 ubiquitin-protein ligase TRIM9, Tripartite motif-containing protein 67, TNF receptor-associated factor 1, Alpha-crystallin A chain, Mitotic checkpoint serine/threonine-protein kinase BUB1, TATA-binding protein-associated factor 2N, Cyclin-F, Centromere protein C, Apoptosis regulator Bcl-2, 2-oxoisovalerate dehydrogenase subunit beta, mitochondrial, Coilin, Nucleoplasmin-3, Homeobox protein Hox-A1, Serine/threonine-protein kinase Chk1, Mitotic checkpoint protein BUB3, Deoxyribonuclease-1, rRNA 2′-O-methyltransferase fibrillarin, Histone H1.3, DNA-directed RNA polymerase III subunit RPC1, DNA-directed RNA polymerase III subunit RPC2, Centromere-associated protein E, Kinesin-like protein KIF11, Histone H4-like protein type G, Tyrosine 3-monooxygenase, ABC transporter, permease/ATP-binding protein, Translation initiation factor IF-1, Protein FAN, Reticulon-4 receptor, Myeloid cell nuclear differentiation antigen, Glucose-6-phosphate isomerase, High affinity immunoglobulin gamma Fc receptor I, Tryptophan 5-hydroxylase 1, Tryptophan 5-hydroxylase 2, Secretory phospholipase A2 receptor, Aquaporin TIP4-1, Histone H2B type F—S, Histone H2AX, Histone H2A type 1-C, ATP-sensitive inward rectifier potassium channel 10, pVII, hypothetical protein TTV27_gp4, hypothetical protein TTV25_gp2, Alpha-1D adrenergic receptor, Alpha-1B adrenergic receptor, Packaging protein 3, hypothetical protein TTV14_gp2, KRR1 small subunit processome component homolog, Bestrophin-4, Alpha-2C adrenergic receptor, Uncharacterized ORF3 protein, Retinoic acid receptor beta, Retinoic acid receptor alpha, B-cell lymphoma 3 protein, Carbohydrate sulfotransferase 8, Harmonin, Prolactin-releasing peptide receptor, Sphingosine 1-phosphate receptor 1, Acyl-CoA-binding domain-containing protein 5, ORF1, hypothetical protein TTMV3_gp2, Mitochondrial import inner membrane translocase subunit Tim17-B, hypothetical protein TTV2_gp2, Absent in melanoma 1 protein, hypothetical protein TTV28_gp1, hypothetical protein TTV26_gp2, hypothetical protein TTV4_gp2, hypothetical protein TTV28_gp4, Mesencephalic astrocyte-derived neurotrophic factor, hypothetical protein TTMV7_gp2, hypothetical protein TTV19_gp2, pORF1, Pre-histone-like nucleoprotein, hypothetical protein TTV8_gp4, hypothetical protein TTV16_gp2, hypothetical protein TTV15_gp2, ORF2/4 protein, P2X purinoceptor 2, membrane glycoprotein E3 CR1-beta, D(2) dopamine receptor, Toll-like receptor 9, Phosphatidylcholine transfer protein, Transcription factor HIVEP2, Probable peptidylarginine deiminase, 60S ribosomal protein L9, Integrin beta-4, Keratin, type II cytoskeletal 1, Chromogranin-A, Histone H3.1t, Voltage-dependent L-type calcium channel subunit alpha-1D, Heat shock 70 kDa protein 1-like, ABC transporter related, UDP-N-acetylglucosamine pyrophosphorylase, Protein GREB1, Aldo/keto reductase, Component of the TOM (Translocase of outer membrane) complex, Excinuclease ABC C subunit domain protein, Phosphoenolpyruvate carboxylase, Arylacetamide deacetylase-like 4, Dynein heavy chain 10, axonemal, Putative Uracil-DNA glycosylase, Spore germination protein PE, Teneurin-1, Putative dehydrogenase, Polysaccharide biosynthesis protein, VCBS, Glutamate/aspartate transport system permease protein GltK, Noggin, Sclerostin, HLA class I histocompatibility antigen, A-30 alpha chain, HLA class I histocompatibility antigen, A-69 alpha chain, HLA class I histocompatibility antigen, B-15 alpha chain, Glutamate receptor ionotropic, NMDA 1, NarH, 40S ribosomal protein S21, Ceruloplasmin, 3-hydroxy-3-methylglutaryl-coenzyme A reductase, 60S ribosomal protein L30, HLA class II histocompatibility antigen gamma chain, HLA class I histocompatibility antigen, Cw-6 alpha chain, HLA class I histocompatibility antigen, Cw-16 alpha chain, Lysosomal alpha-mannosidase, Heat shock protein HSP 90-alpha, Histone H3.2, Histone H2A.J, Voltage-dependent T-type calcium channel subunit alpha-1G, Syncytin-1, Cathelicidin antimicrobial peptide, Tubulin beta-3 chain, Stress-70 protein, mitochondrial, Probable 1,4-alpha-glucan branching enzyme Rv3031, Nuclease-sensitive element-binding protein 1, Complement factor H-related protein 1, Glutaredoxin-1, Gamma-enolase, Platelet-derived growth factor receptor alpha, Collagen alpha-1(VIII) chain, Matrix metalloproteinase-25, Interferon regulatory factor 5, Cytochrome c oxidase subunit 7C, mitochondrial, Heat shock-related 70 kDa protein 2, Cysteine-rich protein 1, NADH dehydrogenase [ubiquinone] flavoprotein 2, mitochondrial, Glutathione S-transferase P, HLA class I histocompatibility antigen, A-68 alpha chain, HLA class II histocompatibility antigen, DM beta chain, Fructose-bisphosphate aldolase C, Beta-2-microglobulin, Cytochrome c oxidase subunit 5B, mitochondrial, Heat shock 70 kDa protein 13, ATP synthase protein 8, 60S ribosomal protein L13a, TRNA nucleotidyltransferase family enzyme, Ferredoxin-dependent glutamate synthase 2, Alkaline phosphatase, tissue-nonspecific isozyme, SLAM family member 5, Slit homolog 3 protein, Transforming growth factor-beta-induced protein ig-h3, Mannose-binding protein C, Calpain-1 catalytic subunit, Actin, gamma-enteric smooth muscle, Creatine kinase M-type, Protein THEM6, Histone-lysine N-methyltransferase ASH1L, C2 calcium-dependent domain-containing protein 4A, Ras association domain-containing protein 10, Hepatocyte cell adhesion molecule, ADAMTS-like protein 5, HLA class II histocompatibility antigen, DRB1-15 beta chain, Anoctamin-2, Phosphoglycerate mutase 1, Por secretion system protein porV (Pg27, lptO), Beta-enolase, Receptor antigen A, 3-oxoacyl-[acyl-carrier-protein] synthase 2, Putative heat shock protein HSP 90-beta 2, Radixin, Tubulin beta-1 chain, Vacuolar protein sorting-associated protein 26A, Serine/threonine-protein phosphatase 5, Catalase, Transketolase, Protein S100-A1, Alpha-centractin, Tubulin beta-4A chain, Beta-centractin, Probable phosphoglycerate mutase 4, Beta-actin-like protein 2, Tubulin beta-4B chain, Phosphoglycerate mutase 2, Alpha-internexin, Tubulin beta-2A chain, Dihydropyrimidinase-related protein 3, Putative heat shock protein HSP 90-beta-3, Fructose-bisphosphate aldolase B, Protein P, Endoplasmin, ATP synthase subunit 0, mitochondrial, Heat shock 70 kDa protein 6, Glyceraldehyde-3-phosphate dehydrogenase, testis-specific, Nascent polypeptide-associated complex subunit alpha-2, Carbonic anhydrase 2, Annexin A6, E3 ubiquitin-protein ligase RNF13, Myeloid-derived growth factor, Tyrosine-protein phosphatase non-receptor type substrate 1, Laminin subunit gamma-1, Trichohyalin, Thrombospondin-2, Sialoadhesin, GTPase IMAP family member 1, C4b-binding protein alpha chain, Voltage-dependent anion-selective channel protein 1, Hemopexin, Complement C5, FYVE, RhoGEF and PH domain-containing protein 2, Haptoglobin, Cytochrome P450 1B1, Titin, Myeloma-overexpressed gene 2 protein, Adipocyte enhancer-binding protein 1, Protein-glutamine gamma-glutamyltransferase 2, Protein Trim21, ADAMTS-like protein 3, N-alpha-acetyltransferase 16, NatA auxiliary subunit, Transforming growth factor beta-1, Elastin, Protein disulfide-isomerase AS, Plastin-2, Leukocyte immunoglobulin-like receptor subfamily B member 1, Histamine H2 receptor, Elongation factor 2, Caveolin-1, Ig gamma-2 chain C region, Immunoglobulin superfamily containing leucine-rich repeat protein, 40S ribosomal protein S9, Prolyl 4-hydroxylase subunit alpha-1, Endoplasmic reticulum-Golgi intermediate compartment protein 1, Tetranectin, Serine protease HTRA1, Heterogeneous nuclear ribonucleoprotein A1, Phosducin-like protein 3, Ig lambda chain V-VI region EB4, Fibronectin type III domain-containing protein 1, Keratin, type II cytoskeletal 2 epidermal, Ferritin heavy chain, Y-box-binding protein 3, Complement C4-B, HLA class I histocompatibility antigen, Cw-15 alpha chain, HLA class I histocompatibility antigen, B-42 alpha chain, Collagen alpha-i(V) chain, HLA class I histocompatibility antigen, B-73 alpha chain, Integral membrane protein 2B, Lysosome-associated membrane glycoprotein 3, Proteoglycan 4, Ribosomal protein S6 kinase alpha-6, Metalloproteinase inhibitor 2, HLA class II histocompatibility antigen, DRB1-12 beta chain, ATP-sensitive inward rectifier potassium channel 15, Vitamin D-binding protein, Osteopontin, Deoxynucleotidyltransferase terminal-interacting protein 2, Olfactory receptor 5K4, Myosin light chain kinase 2, skeletal/cardiac muscle, Non-POU domain-containing octamer-binding protein, Ubiquilin-2, HLA class I histocompatibility antigen, B-51 alpha chain, Minor histocompatibility antigen H13, Glycophorin-C, Eosinophil cationic protein, SWI/SNF complex subunit SMARCC2, Macrophage mannose receptor 1, tRNA-splicing ligase RtcB homolog, Reticulocalbin-2, Heterogeneous nuclear ribonucleoprotein L, 40S ribosomal protein S30, Collagen alpha-3(VI) chain, Matrix metalloproteinase-14, Antithrombin-III, 60S ribosomal protein L10a, Retinol-binding protein 4, Heterogeneous nuclear ribonucleoprotein R, Lithostathine-1-alpha, Ret finger protein-like 2, Zinc-alpha-2-glycoprotein, Carboxypeptidase Q, HLA class I histocompatibility antigen, B-56 alpha chain, Chondroadherin, Cysteine-rich protein 2, Prosaposin, Complement component C9, Apolipoprotein C-II, Protocadherin-16, Leukocyte immunoglobulin-like receptor subfamily B member 4, Galactokinase, Complement factor H, Uncharacterized protein YEL014C, Glycerophosphocholine phosphodiesterase GPCPD1, Echinoderm microtubule-associated protein-like 6, or an isoform, homolog, fragment, variant or derivative of any of these proteins.

The term “alloantigen” (also referred to as “allogeneic antigen” or “isoantigen”) refers to an antigen existing in alternative (allelic) forms in a species, and can therefore induce alloimmunity (or isoimmunity) in members of the same species, e.g. upon blood transfusion, tissue or organ transplantation, or sometimes pregnancy. Typical allogeneic antigens include histocompatibility antigens and blood group antigens. In the context of the present invention, alloantigens are preferably of human origin. Artificial nucleic acid (RNA) molecules encoding antigenic (poly-)peptides or proteins derived from alloantigens can, for instance, be used to induce immune tolerance towards said alloantigen.

Exemplary allogeneic antigens in the context of the present invention include, without limitation, allogeneic antigens derived or selected from UDP-glucuronosyltransferase 2B17 precursor, MHC class I antigen HLA-A2, Coagulation factor VIII precursor, coagulation factor VIII, Thrombopoietin precursor (Megakaryocyte colony-stimulating factor) (Myeloproliferative leukemia virus oncogene ligand) (C-mpl ligand) (ML) (Megakaryocyte growth and development factor) (MGDF), Integrin beta-3, histocompatibility (minor) HA-1, SMCY, thymosin beta-4, Y-chromosomal, Histone demethylase UTY, HLA class II histocompatibility antigen, DP(W2) beta chain, lysine-specific demethylase 5D isoform 1, myosin-Ig, Probable ubiquitin carboxyl-terminal hydrolase FAF-Y, Pro-cathepsin H, DRB1, MHC DR beta DRw13 variant, HLA class II histocompatibility antigen, DRB1-15 beta chain, HLA class II histocompatibility antigen, DRB1-1 beta chain precursor, Minor histocompatibility protein HMSD variant form, HLA-DR3, Chain B, Hla-Dr1 (Dra, Drb1 0101) Human Class Ii Histocompatibility Protein (Extracellular Domain) Complexed With Endogenous Peptide, MHC classII HLA-DRB1, MHC class I HLA-A, human leukocyte antigen B, RAS protein activator like-3, anoctamin-9, ATP-dependent RNA helicase DDX3Y, Protocadherin-11 Y-linked, KIAA0020, platelet glycoprotein IIIa leucine-33 form-specific antibody light chain variable region, dead box, Y isoform, ATP-dependent RNA helicase DDX3X isoform 2, HLA-DRB1 protein, truncated integrin beta 3, glycoprotein IIIa, platelet membrane glycoprotein IIb, Carbonic anhydrase 1, HLA class I histocompatibility antigen, A-11 alpha chain precursor, HLA-A11 antigen A11.2, HLA class I histocompatibility antigen, A-68 alpha chain, MHC HLA-B51, MHC class I antigen HLA-A30, HLA class I histocompatibility antigen, A-1 alpha chain precursor variant, HLA class I histocompatibility antigen B-57, MHC class I antigen, MHC class II antigen, MHC HLA-DR-beta cell surface glycoprotein, DR7 beta-chain glycoprotein, MHC DR-beta, lymphocyte antigen, collagen type V alpha 1, collagen alpha-2(V) chain preproprotein, sp110 nuclear body protein isoform d, integrin, alpha 2b (platelet glycoprotein IIb of IIb/IIIa complex, antigen CD41), isoform CRA_c, 40S ribosomal protein S4, Y isoform 1, uncharacterized protein KIAA1551, factor VIII, UDP-glucuronosyltransferase 2B17, HLA class I histocompatibility antigen, A-2 alpha chain, Thrombopoietin, Minor histocompatibility protein HA-1, Lysine-specific demethylase 5D, HLA class II histocompatibility antigen, DP beta 1 chain, Unconventional myosin-Ig, HLA class II histocompatibility antigen, DRB1-13 beta chain, HLA class II histocompatibility antigen, DRB1-1 beta chain, HLA class II histocompatibility antigen, DRB1-3 chain, HLA class I histocompatibility antigen, B-46 alpha chain, Pumilio homolog 3, ATP-dependent RNA helicase DDX3X, Integrin alpha-IIb, HLA class I histocompatibility antigen, A-11 alpha chain, HLA class I histocompatibility antigen, B-51 alpha chain, HLA class I histocompatibility antigen, A-30 alpha chain, HLA class I histocompatibility antigen, A-1 alpha chain, HLA class I histocompatibility antigen, B-57 alpha chain, HLA class I histocompatibility antigen, B-40 alpha chain, HLA class II histocompatibility antigen, DRB1-7 beta chain, HLA class II histocompatibility antigen, DRB1-12 beta chain, Collagen alpha-1(V) chain, Collagen alpha-2(V) chain, Sp110 nuclear body protein, or an isoform, homolog, fragment, variant or derivative of any of these proteins.

### Allergenic (Poly-)Peptides or Proteins

The at least one coding region of the artificial nucleic acid molecule of the invention may encode at least one “allergenic (poly-)peptide or protein”. The term “allergenic (poly-)peptide or protein” or “allergen” refers to (poly-)peptides or proteins capable of inducing an allergic reaction, i.e. a pathological immunological reaction characterized by an altered bodily reactivity (such as hypersensitivity), upon exposure to a subject. Typically, “allergens” are implicated in “atopy”, i.e. adverse immunological reactions involving immunoglobulin E (IgE). The term “allergen” thus typically means a substance (here: a (poly-)peptide or protein) that is involved in atopy and induces IgE antibodies. Typical allergens envisaged herein include proteinaceous Crustacea-derived allergens, insect-derived allergens, mammalian allergens, mollusk-derived allergens, plant allergens and fungal allergens.

Exemplary allergens in the context of the present invention include, without limitation, allergens derived or selected from from Allergen Pen n 18, Antigen Name, Ara h 2.01 allergen, Melanoma antigen recognized by T-cells 1, Non-specific lipid-transfer protein precursor (LTP) (Allergen Mal d 3), ovalbumin, Parvalbumin beta, Pollen allergen Lol p VA precursor, Pollen allergen Phl p 5b precursor, pru p 1, Pollen allergen Phl p 5a, Der p 1 allergen precursor, Pollen allergen KBG 60 precursor, major allergen Tur c1—Turbo cornutus, Mite group 2 allergen Lep d 2 precursor, Lep D 2 precursor, Major latex allergen Hev b 5, major allergen Cor a 1.0401, Major pollen allergen Art v 1 precursor, Major pollen allergen Bet v 1-A, Beta-lactoglobulin precursor, Alpha-amylase inhibitor 0.28 precursor (CIII) (WMAI-1), group V allergen Phl p 5.0203 precursor, Polygalacturonase precursor, pollen allergen Phl pI, Der f 2 allergen, Probable non-specific lipid-transfer protein 2 precursor, Venom allergen 5 precursor, Pollen allergen Phl p 1 precursor, group V allergen, Chain A, Crystal Structure Of The Calcium-Binding Pollen Allergen Phl P 7 (Polcalcin) At 1.75 Angstroem, Tri r 2 allergen, Pathogenesis-related protein precursor, Globin CTT-III precursor, Major allergen Alt a 1, 13S globulin seed storage protein 3 precursor (Legumin-like protein 3) (Allergen Fag e 1), Lit v 1 tropomyosin, Rubber elongation factor protein, Ovomucoid precursor, Small rubber particle protein, Mag3, Allergen Ara h 1, clone P41B precursor, 13S globulin seed storage protein 1 precursor (Legumin-like protein 1), Pollen allergen Lol p 1 precursor, Major pollen allergen Jun a 1 precursor, Sugi basic protein precursor, profilin, Globin CTT-IV precursor, alkaline serine protease, Glycinin, Conglutin-7 precursor, 2S protein 1, Globin CTT-VI precursor, Ribonuclease mitogillin precursor, Major pollen allergen Cyn d 1, Melanocyte-stimulating hormone receptor, P34 probable thiol protease precursor, Vicilin-like protein, Major allergen Equ c 1 precursor, major allergen Bet v 1, Major allergen Can f 1 precursor, Bd 30K (34 kDa maturing seed protein), Major pollen allergen, Major pollen allergen Hol I 1 precursor, Kappa-casein precursor, major allergen Dau c 1/1, Stress-induced protein SAM22, Major allergen Api g 1, Glycinin G2 precursor, allergen Arah3/Arah4, Der f 1 allergen, Peptidase 1 precursor (Mite group 1 allergen Eur m 1) (Allergen Eur m I), Oryzin precursor, alpha S1 casein, Major pollen allergen Cha o 1 precursor, Non-specific lipid-transfer protein 1, collagen, type I, alpha 2, Der P 1, Peptidase 1 precursor (Major mite fecal allergen Der p 1) (Allergen Der p I), pollen allergen Bet v 1, Phospholipase A2 precursor, Mite group 2 allergen Der p 2, Allergen Mag, Major urinary protein precursor, Major allergen I polypeptide chain 2 precursor, Pen a 1 allergen, Fag e 1, Serum albumin precursor, Pollen allergen Amb a 3, putative alpha-amylase inhibitor 0.28, Albumin seed storage protein, 2S sulfur-rich seed storage protein precursor (Allergen Ber e 1), seed storage protein SSP2, Pro-hevein precursor, pollen allergen, Der p 2 allergen precursor, 2S seed storage protein 1 precursor, prohevein, 2s albumin, major allergen I, polypeptide chain 1, Major allergen I polypeptide chain 1 precursor, Cry j IB precursor, Mite group 2 allergen Der f 2 precursor, beta-casein precursor, Lep D 2 allergen precursor, Allergen Cry j 2 (Pollen allergen), KIAA1224 protein, Hydrophobic seed protein, Allergen Bos d 2 precursor, Allergen II, Mite group 2 allergen Der p 2 precursor, Mite allergen Blo t 5, Peptidase 1 precursor (Major mite fecal allergen Der f 1) (Allergen Der f I), Par j, Can f I, Pollen allergen Lol p 2-A (Lol p II-A), Paramyosin, Alpha-S2-casein precursor, P34 probable thiol protease, beta-lactoglobulin, major allergen Phl p 5, Chain A, Structure Of Erythrocruorin In Different Ligand States Refined At 1.4 Angstroms Resolution, Globin CTT-VIII, Major allergen Asp f 2 precursor, tropomyosin, core protein [Hepatitis B virus], Omega gliadin storage protein, Alpha/beta-gliadin A-V, group 14 allergen protein, Pollen allergen Amb a 1.1 precursor, Glycinin G1 precursor, Pollen allergen Amb a 2 precursor, Cry j 1 precursor, allergen Ziz m 1, Glycine-rich cell wall structural protein 1.8 precursor, Putative pectate lyase 17 precursor, pectate lyase, Pectate lyase precursor, Probable pectate lyase 18 precursor, major allergen beta-lactoglobulin, Major allergen Mal d 1, Alpha-S1-casein precursor, 2S seed storage protein 1, plectrovirus spv1-r8a2b orf 14 transmembrane protein, allergen I/a, Allergen Cr-PI, Probable non-specific lipid-transfer protein 1, Cr-PII allergen, melanoma antigen gp100, Alpha-lactalbumin precursor, Chain A, Anomalous Substructure Of Alpha-Lactalbumin, Pilosulin-1 precursor (Major allergen Myr p 1) (Myr p I), Pollen allergen Lol p 3 (Lol p III), Lipocalin 1 (tear prealbumin), Major pollen allergen Cup a 1, Melanocyte protein Pmel 17 precursor, major house dust allergen, Non-specific lipid-transfer protein 1 (LTP 1) (Major allergen Pru d 3), Non-specific lipid-transfer protein 1 (LTP 1) (Major allergen Pru ar 3), Pollen allergen Lol p 1, alpha-gliadin, Cr-PII, albumin, Alpha-S1-casein, major allergen I, Ribonuclease mitogillin, beta-casein, UA3-recognized allergen, 2S sulfur-rich seed storage protein 1, unnamed protein product, Polygalacturonase, Major allergen Pru av 1, Der p 1 allergen, lyase allergen, Major pollen allergen Bet v 1-F/I, Gamma-gliadin precursor, 5-hydroxytryptamine receptor 2C (5-HT-2C) (Serotonin receptor 2C) (5-HT2C) (5-HTR2C) (5HT-1C), omega-5 gliadin, Enolase 1 (2-phosphoglycerate dehydratase) (2-phospho-D-glycerate hydro-lyase), Probable non-specific lipid-transfer protein, Allergen Sin a 1, Glutenin, low molecular weight subunit precursor, Major Peanut Allergen Ara H 1, mal d 3, Eukaryotic translation initiation factor 3 subunit D, tyrosinase-related protein-2, PC4 and SFRS1-interacting protein, RAD51-like 1 isoform 1, Antimicrobial peptide 2, Proteasome subunit alpha type-3, Neurofilament heavy polypeptide (NF—H) (Neurofilament triplet H protein) (200 kDa neurofilament protein), Superoxide dismutase, Major pollen allergen Cor a 1 isoforms 5, 6, 11 and 16, cherry-allergen PRUA1, Allergen Asp f 4 precursor, Chain A, Tertiary Structure Of The Major House Dust Mite Allergen Der P 2, Nmr, 10 Structures, RNA-binding protein NOB1, Dermatan-sulfate epimerase precursor, Squamous cell carcinoma antigen recognized by T-cells 3, Peptidyl-prolyl cis-trans isomerase B precursor, Probable glycosidase crf1, Chain A, Birch Pollen Profilin, Profilin-1, avenin precursor (clone pAv122)—oat, gamma 3 avenin, coeliac immunoreactive protein 2, CIP-2, prolamin 2 {N-terminal}, avenin gamma-3—small naked oat (fragment), major pollen allergen Ole e 1, Cytochrome P450 3A1, Ole e 1 protein, Ole e 1.0102 protein, Der f 2, GroEL-like chaperonin, major allergen Arah1, manganese superoxide dismutase, beta-1,3-glucanase-like protein, Ara h 1 allergen, Major allergen Alt a 1 precursor, Bla g 4 allergen, Per a 4 allergen variant 1, Lyc e 2.0101, pectate lyase 2, allergen, hypothetical protein, Probable pectate lyase P59, Pollen allergen Amb a 1.4, Patatin-2-Kuras 1, calcium-binding protein, vicilin seed storage protein, major allergenic protein Mal f4, pel protein, ripening-related pectate lyase, pectate lyase/Amb allergen, Bet v 4, Polcalcin Bet v 4, Mite allergen Der f 6, Allergen Alt a 2, Extracellular elastinolytic metalloproteinase, pectate lyase-like protein, Pectate lyase E, Profilin-2, Venom allergen 5, Cucumisin, Putative peroxiredoxin, putative pectate lyase precursor, Serum albumin, pollen allergen Phl p 11, serine (or cysteine) proteinase inhibitor, clade B (ovalbumin), member 3, Allergen Bla g 4 precursor (Bla g IV), Allergen Pen n 13, Hyaluronidase A, pectate lyase homolog, putative allergen Cup a 1, Major pollen allergen Jun v 1, putative allergen jun o 1, Pollen allergen Amb a 1.2, Probable pectate lyase 13, P8 protein, Cytochrome c, Glucan endo-1,3-beta-glucosidase, basic vacuolar isoform, 13S globulin, beta-1,3-glucanase, beta-1, 3-glucananse, Glutenin, high molecular weight subunit DX5 precursor, X-type HMW glutenin, Glutenin, high molecular weight subunit DX5, high-molecular-weight glutenin subunit 1Dx2.1, high molecular weight glutenin subunit, 11S globulin-like protein, seed storage protein, alpha-L-Fucp-(1->3)-[alpha-D-Manp-(1->6)-[beta-D-Xylp-(1->2)]-beta-D-Manp-(1->4)-beta-D-GcpNAc-(1->4)]-D-GlcpNAc, beta casein B, type 1 non-specific lipid transfer protein precursor, Fas AMA, Caspase-8 precursor, H antigen glycoprotein, H antigen gl, Heat shock protein HSP 90-beta, dihydrolipoamide S-acetyltransferase (E2 component of pyruvate dehydrogenase complex), isoform CRA_a, Group V allergen Phl p 5.0103 precursor, Phl p6 allergen precursor, Group V allergen Phl p 5, Major pollen allergen Phl p 4 precursor, Pollen allergen Phl p V, Phl p 3 allergen, Pollen allergen Phl pI precursor, Chain A, Crystal Structure Of Phl P 1, A Major Timothy Grass Pollen Allergen, Pollen allergen Phl p 4, Profilin-3, Profilin-2/4, Pollen allergen Phl p 2, Phl p6 IgE binding fragment, Phlp5, Chain N, Crystal Structure Of Phl P 6, A Major Timothy Grass Pollen Allergen Co-Crystallized With Zinc, group V allergen Phl p 5.0206 precursor, allergenic protein, Major allergen Ani s 1, allergen Ana o 2, ENSP-like protein, BW 16 kDa allergen, alpha2(I) collagen, collagen a2(I), type 1 collagen alpha 2, Cyn d 1, Major pollen allergen Aln g 1 (Allergen Aln g I), allergen Len c 1.0101, galactomannan, Aspartic protease Bla g 2, alcohol dehydrogenase, lipid transfer protein precursor, alpha/beta gliadin precursor, Der f 7 allergen, Der p 7 allergen polypeptide, non-specific lipid transfer protein, Major allergen I polypeptide chain 1, prunin 1 precursor, prunin 2 precursor, 11S legumin protein, Ara h 7 allergen precursor, vicilin-like protein precursor, allergen Arah6, parvalbumin like 2, parvalbumin like 1, casein kappa, Ribosomal biogenesis protein LAS1L, Pen c 1, SchS21 protein, Inactive hyaluronidase B, Mup1 protein, Macrophage migration inhibitory factor, Eukaryotic translation initiation factor 2 subunit 3, CR2/CD21/C3d/Epstein-Barr virus receptor precursor, DNA topoisomerase 2-alpha, pollen allergen Cyn d 23, major allergen Bla g 1.02, pectin methylesterase allergenic protein, major allergen Pha a 5 isoform, 2S albumin seed storage protein, aldehyde dehydrogenase (NAD+), pollen allergen Poa p 5, Bla g 1.02 variant allergen, partial, Major pollen allergen Lol p 5b, allergen Bla g 6.0301, protein disulfide isomerase, putative mannitol dehydrogenase, pollen allergen Lol p 4, Aspartic protease pep1, enolase, IgE-binding protein, Minor allergen Alt a 5, HDM allergen, Chain A, Crystal Structure Of An Mbp-Der P 7 Fusion Protein, allergen Bla g 6.0201, major allergen Bla g 1.0101, alpha-amylase, minor allergen, ribosomal protein P2, metalloprotease (MEP), autophagic serine protease Alp2, allergenic isoflavone reductase-like protein Bet v 6.0102, Chain A, Crystal Structure Of The Complex Of Antibody And The Allergen Bla G 2, minor allergen, thioredoxin TrxA, enolase, allergen Cla h 6, glutathione-S-transferase, molecular chaperone and allergen Mod-E/Hsp90/Hsp1, major allergen Asp F2, Mite allergen Der p 3, Chain B, Crystal Structure Of Aspergillus Fumigatus Mnsod, Glutathione S-transferase (GST class-sigma) (Major allergen Bla g 5), Minor allergen Cla h 7, unknown protein, allergenic cerato-platanin Asp F13, art v 2 allergen, Polcalcin Aln g 4, major allergen and cytotoxin AspF1, pollen allergen Que a 1 isoform, trypsin-like serine protease, Mite group 6 allergen Der p 6, allergen Asp F7, cell wall protein PhiA, 60 kDa allergen Der f 18p, hsp70, Sal k 3 pollen allergen, acidic ribosomal protein P2, Chain B, Crystal Structure Of The Nadp-Dependent Mannitol Dehydrogenase From Cladosporium herbarum., Art v 3.0301 allergen precursor, 60S ribosomal protein L3, Der p 20 allergen, Pollen allergen Sal k 1, Per a 6 allergen, gelsolin-like allergen Der f 16, Chain A, Structural Characterization Of The Tetrameric Form Of The Major Cat Allergen Fel D 1, Glutathione S-transferase, Fel d 4 allergen, Major pollen allergen Dac g 4, Group I allergen Ant o I (Form 1), pollen, allergen Bla g 6.0101, cystatin, Mite allergen Der p 5, allergen Fra e 1, allergen Asp F4, major antigen-like protein, PR5 allergen Cup s 3.1 precursor, heat shock protein, allergen precursor, arginine esterase precursor, Sal k 4 pollen allergen, 60S acidic ribosomal protein P1, pollen allergen Jun o 4, Polcalcin Cyn d 7, group I pollen allergen, peptidyl-prolyl cis-trans isomerase/cyclophilin, putative, profilin 2, pollen allergen Cyn d 15, Der f 13 allergen, Can f 2, peroxisomal-like protein, peptidylprolyl isomerase (cyclophilin), MHC class II antigen, BETV4 protein, Major pollen allergen Pla l 1, peptidase, MPA3 allergen, plantain pollen major allergen, Pla l 1.0103, major allergen Bla g 1.0101, partial, Pollen allergen Amb p 5a, Der f 16 allergen, Pollen allergen Dac g 2, IgE-binding protein C-terminal fragment (148 AA), Pollen allergen Dac g 3, PPIase, rAsp f 9, Mite allergen Der p 7, thioredoxin, hydrolase, Major pollen allergen Pha a 1, Der p 13 allergen, Chain B, X-Ray Structure Of Der P 2, The Major House Dust Mite Allergen, oleosin 3, Peptidyl-prolyl cis-trans isomerase, Chain A, Crystal Structure Of A Major House Dust Mite Allergen, Derf 2, Chain A, Crystal Structure Of Major Allergens, Bla G 4 From Cockroaches, Amb a 1-like protein, D-type LMW glutenin subunit, Glutathione S-transferase 2, acidic Cyn d 1 isoallergen isoform 4 precursor, albumin seed storage protein precursor, tyrosine 3-monooxygenase isoform b, N-glycoprotein, FAD-linked oxidoreductase BG60, Blo t 21 allergen, Ubiquitin D, Nucleoporin Nup37, Non-POU domain-containing octamer-binding protein, Transcription elongation factor SPT5, Major allergen Mal d 1 (Ypr10 protein), Serpin-Z2B, Pas n 1 allergen precursor, arginine kinase, Lit v 3 allergen myosin light chain, sarcoplasmic calcium-binding protein, alpha subunit of beta conglycinin, prunin, allergen Cry j 2, Plexin-A4, Non-specific lipid-transfer protein, Low molecular weight glutenin subunit precursor, gamma-gliadin, friend of GATA-1, Wilms tumor protein, Ubiquitin-conjugating enzyme E2 C, Fatty acid synthase, Histone H4, Fructose-bisphosphate aldolase A, oxidoreductase, lactoglobulin beta, immunoglobulin gamma 3 heavy chain constant region, Phlp5 precursor, dust mite allergen precursor, heat shock protein 70, Major allergen I polypeptide chain 2, alpha-lactalbumin precursor protein, 30 kDa pollen allergen, group 5 allergen precursor, group 1 allergen Dac g 1.01 precursor, uncharacterized protein, unknown Timothy grass protein, kappa-casein, alpha-S1 casein, SXP/RAL-2 family protein, Lipocalin-1 precursor, alpha purothionin, major allergen Bet v 1.01A, P2 protein, Osmotin, Major Peanut Allergen Ara H 2, Der f 3 allergen, Conglutin, Ara h 6 allergen, Cathelicidin antimicrobial peptide, cholinesterase, Per a 2 allergen, Submaxillary gland androgen-regulated protein 3B, chitinase, partial, allergen Can f 4 precursor, Can f 4 variant allergen precursor, nascent polypeptide-associated complex subunit alpha-2, Polcalcin Phl p 7 (Calcium-binding pollen allergen Phl p 7) (P7), Der p II allergen, main allergen Ara h1, allergen Ara h 2.02, fatty acid binding protein, glutamate receptor, glycinin A3B4 subunit, profilin isoallergen 2, Pollen allergen Amb p 5b, calcium-binding protein isoallergen 2, calcium-binding protein isoallergen 1, cysteine protease, profilin isoallergen 1, ragweed homologue of Art v 1 precursor, Amb p 5, ragweed homologue of Art v 1 (isoform 1), partial, antigen E, putative pectate lyase precursor, partial, Pollen allergen Amb a 5, Amb p V allergen, hemocyanin subunit 6, major pollen allergen Cha o 2, trichohyalin, aspartyl endopeptidase, NCRA10, allergen bla g 8, vitellogenin, NCRA3, NCRA4, allergen Bla g 3 isoform 2 precursor, partial, NCRA2, NCRA13, NCRA8, NCRA1, Bla g 11, receptor for activated protein kinase C-like, NCRA5, NCRA14, triosephosphate isomerase, NCRA12, NCRA7, NCRA11, trypsin, triosephosphate isomerase, partial, NCRA6, structural protein, NCRA15, NCRA9, NCRA16, Der f 4 allergen, Der f 5 allergen, Phl p6 allergen, Der f Gal d 2 allergen, Derp_19830, glucosylceramidase, carboxypeptidase, Der f 8 allergen, partial, fructose bisphosphate aldolase, ATP synthase, Der f Alt a 10 allergen, glutamine synthetase, Derp_c23425, myosin, Der f 8 allergen, LytFM, Der f 11 allergen, serine protease, glutathione transferase mu, triose-phosphate isomerase, ubiquinol-cytochrome c reductase binding protein-like protein, ferritin, isomerase, filamin C, Der p 5, Mag44, partial, venom, muscle specific protein, Der f 5.02 allergen, Mag44, Derp_c21462, group 18 allergen protein, Derf_c9409, napin-type 2S albumin 1 precursor, napin-type 2S albumin 3, isoflavone reductase-like protein CJP-6, Pectate lyase 1, allergen Cry j 2, partial, Major allergen Dau c 1, Filamin-C, putative, Pis v 5.0101 allergen 11S globulin precursor, Pis v 5, 48-kDa glycoprotein precursor, vicilin, or a homolog, fragment, variant or derivative of any of these allergens.

### Reporter Proteins

The at least one coding region of the artificial nucleic acid (RNA) molecule of the invention may encode at least one “reporter (poly-)peptide or protein”.

The term “reporter (poly-)peptide or protein” refers to a (poly-)peptide or protein that is expressed from a reporter gene. Reporter (poly-)peptides or proteins are typically heterologous to the expression system used. Their presence and/or functionality can be preferably readily detected, visualized and/or measured (e.g. by fluorescence, spectroscopy, luminometry, etc.).

Exemplary reporter (poly-)peptides or proteins include beta-galactosidase (encoded by the bacterial gene IacZ); luciferase; chloramphenyl acetyltransferase (CAT); GUS (beta-glucuronidase); alkaline phosphatase; green fluorescent protein (GFP) and its variants and derivatives, such as enhanced Green Fluorescent Proteins (eGFP), CFP, YFP, GFP+; alkaline phosphatase or secreted alkaline phosphatase; peroxidase, beta-xylosidase; XylE (catechol dioxygenase); TreA (trehalase); Discosoma sp. red fluorescent protein (dsRED) and its variants and derivatives, such as mCherry; HcRed; AmCyan; ZsGreen; ZsYellow; AsRed; and other bioluminescent and fluorescent proteins. The term “luciferase” refers to a class of oxidative enzymes that are capable of producing bioluminescence. Many luciferases are known in the art, for example firefly luciferase (for example from the firefly Photinus pyralis), Renilla luciferase (Renilla reniformis), Metridia luciferase (MetLuc, derived from the marine copepod Metridia longa), Aequorea luciferase, Dinoflagellate luciferase, or Gaussia luciferase (Gluc) or an isoform, homolog, fragment, variant or derivative of any of these proteins.

### Additional Domains, Tags, Linkers, Sequences or Elements

The at least one coding region of the inventive artificial nucleic acid molecule may encode, preferably in addition to the at least one (poly-)peptide or protein of interest, further (poly-)peptide domains, tags, linkers, sequences or elements. It is envisioned that the nucleic acid sequences encoding said additional domains, tags, linkers, sequences or elements are operably linked in frame to the region encoding the (poly-)peptide or protein of interest, such that expression of the coding sequence preferably yields a fusion product (or: derivative) of the (poly-)peptide or protein of interest coupled to the additional domain(s), tag(s), linker(s), sequence(s) or element(s).

For example, the nucleic acid sequences encoding further (poly-)peptide domains, tags, linkers, sequences or elements is preferably in-frame with the nucleic acid sequence encoding the (poly-)peptide or protein of interest. Codon usage may be adapted to the host envisaged for expressing the artificial nucleic acid (RNA) molecule of the invention.

Preferably, the at least one coding region of the artificial nucleic acid molecule of the invention may further encode at least one (a) effector domain; (b) peptide or protein tag; (c) localization signal or sequence; (d) nuclear localization signal (NLS); (e) signal peptide; (f) peptide linker; (g) secretory signal peptide (SSP), (h) multimerization element including dimerization, trimerization, tetramerization or oligomerization elements; (i) virus like particle (VLP) forming element; (j) transmembrane element; (k) dendritic cell targeting element; (l) immunological adjuvant element; (m) element promoting antigen presentation; (n) 2A peptide; (o) element that extends protein half-life; and/or (p) element for post-translational modification (e.g. glycosylation).

### Effector Domains

The term “effector domain” refers to (poly-)peptides or protein domains conferring biological effector functions, typically by interacting with a target, e.g. enzymatic activity, target (e.g. ligand, receptor, protein, nucleic acid, hormone, neurotransmitter small organic molecule) binding, signal transduction, immunostimulation, and the like.

Effector domains may suitably be (additionally) encoded by artificial nucleic acid (RNA) molecules encoding any (poly-)peptide or protein of interest as disclosed herein. Effector domains fused to or inserted into (poly-)peptides or proteins of interest may advantageously impart an additional biological function or activity on said (poly-)peptide or protein. When encoded in combination with a (poly-)peptide or protein of interest, effector domains may be placed at at the N-terminus, C-terminus and/or within of the (poly-)peptide or protein of interest, or combinations thereof. Different effector domains may be combined. On nucleic acid level, the coding sequence for such effector domain is typically placed in frame (i.e. in the same reading frame), 3′ to, 5′ to or within the coding sequence for the (poly-)peptide or protein of interest, or combinations thereof.

### Peptide or Protein Tag

“Peptide or protein tags” are short amino acid sequences introduced into (poly-)peptides or proteins of interest to confer a desired biological functionality or property. Typically, “peptide tags” may be used for detection, purification, separation or the addition of certain desired biological properties or functionalities.

Peptide or protein tags may thus be deployed for different purposes. Almost all peptide tags can be used to enable detection of a (poly-)peptide or protein of interest through Western blot, ELISA, ChIP, immunocytochemistry, immunohistochemistry, and fluorescence measurement. Most protein or peptide tags can be utilized for purification of (poly-)peptides or proteins of interest. Some tags can be explored to extend the biological protein half-lives or increasing solubility of (poly-)peptides and proteins of interest, or help to localize a (poly-)peptide or protein to a cellular compartment.

Protein or peptide tags may suitably be (additionally) encoded by artificial nucleic acid (RNA) molecules encoding any (poly-)peptide or protein of interest as disclosed herein. Protein or peptide tags fused to or inserted into (poly-)peptides or proteins of interest may advantageously enable, e.g., the detection, purification or separation of said (poly-)peptide or protein. When encoded in combination with a (poly-)peptide or protein of interest, protein or peptide tags may be placed at at the N-terminus, C-terminus and/or within of the (poly-)peptide or protein of interest, or combinations thereof. Different protein or peptide tags may be combined. Protein or peptide tags may be repeated and for instance expressed in a tandem or triplet. On nucleic acid level, the coding sequence for such protein or peptide tags is typically placed in frame (i.e. in the same reading frame), 3′ to, 5′ to or within the coding sequence for the (poly-)peptide or protein of interest, or combinations thereof.

Protein and peptide tags may be classified based on their (primary) function. Exemplary protein and peptide tags envisaged in the context of the present invention include, without limitation, tags selected from the following groups. Affinity tags enable the purification of (poly-)peptides or proteins of interest and include, without limitation, chitin binding protein (CBP), maltose binding protein (MBP), Strep-tag, glutathione-S-transferase (GST) and poly(His) tags typically comprising six tandem histidine residues which form a nickel-binding structure. Solubilisation tags assist in proper folding and prevent precipitating of (poly-)peptides or proteins of interest and include thioredoxin (TRX) and poly(NANP). MBP- and GST-tags may be utilized as solubilisation tags as well. Chromatography tags alter the chromatographic properties of proteins or (poly-)peptides of interest and enable their separation via chromatographic techniques. Typically, chromatography tags consist of polyanionic amino acids, such as the FLAG-tag (which may typically comprise the amino acid sequence N-DYKDDDDK-C(SEQ ID NO:378). Epitope tags are short peptide sequences capable of binding to high-affinity antibodies, e.g. in western blotting, immunofluorescence or immunoprecipitation, but may also be used for purification of (poly-)peptides or proteins of interest. Epitope tags may be derived from pathogenic antigens, such as viruses, and include, without limitation, V5-tags (which may typically contain a short amino acid sequence GKPIPNPLLGLDST derived from the P/V proteins of paramyxovirus SV5), Myc-tags (which may typically contain a 10 amino acid segment of human proto-oncogene Myc (EQKLISEEDL (SEQ ID NO:379), HA-tags (which may typically comprise a short segment YPYDVPDYA (SEQ ID NO:380) from human influenza hemagglutinin protein) and NE-tags. Fluorescence tags like GFP and its variants and derivatives (e.g. mfGFP, EGFP) may be used for the detection of (poly-)peptides or proteins (either by direct visual readout, or by binding to anti-GFP antibodies) or as reporters. Protein tags may allow specific enzymatic modification (such as biotinylation by biotin ligase) or chemical modification (such as reaction with FlAsH-EDT2 for fluorescence imaging). Tags like thioredoxin, poly(NANP), can increase protein solubility, while others can help localize a target protein to a desired cellular compartment. Further tags include ABDz1-tag, Adenylate kinase (AK-tag), Calmodulin-binding peptide, CusF, Fh8, HaloTag, Heparin-binding peptide (HB-tag), Ketosteroid isomerase (KSI), Inntag, PA(NZ-1), Poly-Arg tag, Poly-Lys tag, S-tag and SUMO. Peptide or protein tags may be combined or repeated. After purification, protein or peptide tags may sometimes be removed by specific proteolysis (e.g. by TEV protease, Thrombin, Factor Xa or Enteropeptidase).

### Nuclear Localization Signal or Sequence (NLS)

A “nuclear localization signal” or “nuclear localization sequence” (NLS) is an amino acid sequence capable of targeting a (poly-)peptide or protein of interest to the nucleus—in other words, a nuclear localization signal “tags” a (poly-)peptide or protein of interest for nuclear import. Generally, proteins gain entry into the nucleus through the nuclear envelope. The nuclear envelope consists of concentric membranes, the outer and the inner membrane. The inner and outer membranes connect at multiple sites, forming channels between the cytoplasm and the nucleoplasm. These channels are occupied by nuclear pore complexes (NPCs), complex multiprotein structures that mediate the transport across the nuclear membrane.

Nuclear localization signals may suitably be (additionally) encoded by artificial nucleic acid (RNA) molecules encoding any (poly-)peptide or protein of interest as disclosed herein. Nuclear localization signals fused to or inserted into (poly-)peptides or proteins of interest may advantageously promote importin (aka karyopherin) binding and/or nuclear import of said (poly-)peptide or protein. Without wishing to be bound by specific theory, NLS may be particular useful when fused to or inserted into therapeutic (poly-)peptides or proteins that are intended for nuclear targeting, e.g. gene editing agents, transcriptional inducers or repressors. However, an NLS may be encoded with any other (poly-)peptide or protein disclosed herein as well. When encoded in combination with a (poly-)peptide or protein of interest, such nuclear localization signals may be placed at at the N-terminus, C-terminus and/or within the (poly-)peptide or protein of interest, or combinations thereof. It is also envisaged that the artificial nucleic acid (RNA) molecule may encode two or more NLS fused/inserted (in)to the encoded (poly-)peptide or protein of interest. On nucleic acid level, the coding sequence for such nuclear localization signal is typically placed in frame (i.e. in the same reading frame), 3′ to or 5′ to or within the coding sequence for the (poly-)peptide or protein of interest, or combinations thereof.

Typically, a “NLS” may comprise or consist of one or more short sequences of positively charged lysines or arginines, which are preferably exposed on the protein surface. A variety of NLS sequences are known in the art. Exemplary NLS sequences that may be selected for use with the present invention include, without limitation, the following. The best characterized transport signal is the classical NLS (cNLS) for nuclear protein import, which consists of either one (monopartite) or two (bipartite) stretches of basic amino acids. Typically, the monopartite motif is characterized by a cluster of basic residues preceded by a helix-breaking residue. Similarly, the bipartite motif consists of two clusters of basic residues separated by 9-12 residues. Monopartite cNLSs are exemplified by the SV40 large T antigen NLS (¹²⁶PKKKRRV¹³² (SEQ ID NO: 381) and bipartite cNLSs are exemplified by the nucleoplasmin NLS (¹⁵⁵KRPAATKKAGQAKKKK¹⁷⁰ (SEQ ID NO: 382). Consecutive residues from the N-terminal lysine of the monopartite NLS are referred to as P1, P2, etc. Monopartite cNLS typically require a lysine in the P1 position, followed by basic residues in positions P2 and P4 to yield a loose consensus sequence of K(K/R)X(K/R) (SEQ ID NO: 384) (Lange et al. J Biol Chem. 2007 Feb. 23; 282(8): 5101-5105).

### Signal Peptide

The term “signal peptide” (sometimes referred to as secretory signal peptide or SSP, signal sequence, leader sequence or leader peptide) refers to a typically short peptide (usually 16-30 amino acids long) that is usually present at the N-terminus of newly synthesized proteins destined towards the secretory pathway. These proteins include those that reside either inside certain organelles (the endoplasmic reticulum, golgi or endosomes), secreted from the cell, or inserted into most cellular membranes. In eukaryotic cells, signal peptides are typically cleaved from the nascent polypeptide chain immediately after it has been translocated into the membrane of the endoplasmic reticulum. The translocation occurs co-translationally and is dependent on a cytoplasmic protein-RNA complex (signal recognition particle, SRP). Protein folding and certain post-translational modifications (e.g. glycosylation) typically occur within the ER. Subsequently, the protein is typically transported into Golgi vesicles and secreted.

Signal peptides may suitably be (additionally) encoded by artificial nucleic acid (RNA) molecules encoding any (poly-)peptide or protein of interest as disclosed herein. Signal peptides fused to or inserted into (poly-)peptides or proteins of interest may advantageously mediate the transport of said (poly-)peptide or protein of interest (in)to a defined cellular compartment, e.g. the cell surface, the endoplasmic reticulum (ER) or the endosomal-lysosomal compartment. Preferably, signal peptides may be introduced into (poly-)peptide or protein of interest to promote secretion of said (poly-)peptides or proteins. In particular in case of artificial nucleic acids encoding antigenic (poly-)peptides or proteins are fused to a signal peptide, proper secretion may aid in triggering an immune response against said antigen, as its release and distribution preferably mimics a naturally occurring viral infection and ensures that professional antigen-presenting cells (APCs) are exposed to the encoded antigens. However, signal peptides may be usefully combined with any other (poly-)peptide or protein disclosed herein as well. When encoded in combination with a (poly-)peptide or protein of interest, such signal peptides may be placed at at the N-terminus, C-terminus and/or within the (poly-)peptide or protein of interest, preferably at its N-Terminus. On nucleic acid level, the coding sequence for such signal peptide is typically placed in frame (i.e. in the same reading frame), 5′ or 3′ or within the coding sequence for the (poly-)peptide or protein of interest, or combinations thereof, preferably 3′ to said coding sequence.

Signal peptides may typically exhibit a tripartite structure, consisting of a hydrophobic core region flanked by an n- and c-region. Typically, the n-region is one to five amino acids in length and comprises mostly positively charged amino acids. The c-region, which is located between the hydrophobic core region and the signal peptidase cleavage site, typically consists of three to seven polar, but mostly uncharged, amino acids. A specific pattern of amino acids (conforming to the so-called “(3,1)-rule”) is found near the cleavage site: the amino acid residues at positions 3 and 1 (relative to the cleavage site) are typically small and neutral.

Exemplary signal peptides envisaged in the context of the present invention include, without being limited thereto, signal sequences of classical or non-classical MHC-molecules (e.g. signal sequences of MHC I and II molecules, e.g. of the MHC class I molecule HLA-A*0201), signal sequences of cytokines or immunoglobulins, signal sequences of the invariant chain of immunoglobulins or antibodies, signal sequences of Lamp1, Tapasin, Erp57, Calretikulin, Calnexin, PLAT, EPO or albumin and further membrane associated proteins or of proteins associated with the endoplasmic reticulum (ER) or the endosomal-lysosomal compartment. Most preferably, signal sequences may be derived from (human) HLA-A2, (human) PLAT, (human) sEPO, (human) ALB, (human) IgE-leader, (human) CD5, (human) IL2, (human) CTRB2, (human) IgG-HC, (human) Ig-HC, (human) Ig-LC, GpLuc, (human) Igkappa or a fragment or variant of any of the aforementioned proteins, in particular HLA-A2, HsPLAT, sHsEPO, HsALB, HsPLAT(aa1-21), HsPLAT(aa1-22), IgE-leader, HsCD5(aa1-24), HsIL2(aa1-20), HsCTRB2(aa1-18), IgG-HC(aa1-19), Ig-HC(aa1-19), Ig-LC(aa1-19), GpLuc(1-17) or MmIgkappa.

Particular signal peptides and nucleic acid sequences encoding the same envisaged for use in the present invention are inter alia disclosed in WO 2017/081082 A2, which is incorporated by reference in its entirety herein.

### Peptide Linkers

A “peptide linker” or “spacer” is a short amino acid sequences joining domains, portions or parts of (poly-)peptides or proteins of interest as disclosed herein, for instance of multidomain-proteins or fusion proteins. The (poly-)peptides or proteins, or domains, portions or parts thereof are preferably functional, i.e. fulfil a specific biological function.

Peptide linkers may suitably be (additionally) encoded by artificial nucleic acid (RNA) molecules encoding any (poly-)peptide or protein of interest as disclosed herein. Peptide linkers may be inserted into (poly-)peptides or proteins of interest may advantageously ensure proper folding, flexibility and function of the (poly-)peptides or proteins of interest, or domains, portions or parts thereof. When encoded in combination with a (poly-)peptide or protein of interest, such signal peptides are typically placed between said (poly-)peptides or proteins, or their domains, portions or parts. On nucleic acid level, the coding sequence for such peptide linker is typically placed in frame (i.e. in the same reading frame), 5′ to, 3′ to or within the coding sequence(s) encoding (poly-)peptides or proteins, domains, portions or parts thereof.

Peptide linkers are typically short (comprising 1-150 amino acids, preferably 1-50 amino acids, more preferably 1 to 20 amino acids) and may preferably be composed of small, non-polar (e.g. Gly) or polar (e.g. Ser or Thr) amino acids. Peptide linkers are generally known in the art and may be classified into three types: flexible linkers, rigid linkers, and cleavable linkers. Flexible linkers are usually applied when joined (poly-)peptides or proteins, or domains, portions or parts thereof require a certain degree of movement, flexibility and/or interaction. Flexible linkers are generally rich in small, non-polar (e.g. Gly) or polar (e.g. Ser or Thr) amino acids to provide good flexibility and solubility, and support the mobility of the joined (poly-)peptides or proteins, or domains, portions or parts thereof. Exemplary flexible linker arm sequences typically contain about 4 to about 10 glycine residues. The incorporation of Ser or Thr may maintain the stability of the linker in aqueous solutions by forming hydrogen bonds with water molecules, and therefore reduces unfavorable interactions between the linker and the protein moieties.

The most commonly used flexible linkers have sequences consisting primarily of stretches of Gly and Ser residues (“GS” linker). For instance, the linker may have the following sequence: GS, GSG, SGG, SG, GGS, SGS, GSS, and SSG. The same sequence may be repeated multiple times (e.g. two, three, four, five or six times) to create a longer linker. It is also conceivable to introduce a single amino acid residue such as S or G as a peptide linker. An example of the most widely used flexible linker has the sequence of (G-G-G-G-S)ₙ (SEQ ID NO: 383). By adjusting the copy number “n”, the length of this GS linker can be optimized to achieve appropriate separation and/or flexibility of the joined (poly-)peptides or proteins, or domains, portions or parts thereof, or to maintain necessary inter-domain interactions. Aside from GS linkers, many other flexible linkers are known in the art. These flexible linkers are also rich in small or polar amino acids such as Gly and Ser, but may contain additional amino acids such as Thr and Ala to maintain flexibility, as well as polar amino acids such as Lys and Glu to improve solubility. Rigid linkers may be employed to ensure separation of the joined (poly-)peptides or proteins, or domains, portions or parts thereof and reduce interference or sterical hindrance. Cleavable linkers, on the other hand, may be introduced to release free functional (poly-)peptides or proteins, or domains, portions or parts thereof in vivo. For instance, the cleavable linkers may be Arg-Arg or Lys-Lys that is sensitive to cleavage with an enzyme such as cathepsin or trypsin. Peptide linkers may or may not be non-immunogenic (i.e. capable of triggering an immune response). Chen et al. Adv Drug Deliv Rev. 2013 Oct. 15; 65(10): 1357-1369 reviews the most commonly used peptide linkers and their applications, and is incorporated herein by reference in its entirety. Particular peptide linkers of interest and nucleic acid sequences encoding the same are inter alia disclosed in WO 2017/081082 A2, WO 2017/WO 2002/014478 A2, WO 2001/008636 A2, WO 2013/171505 A2, WO 2008/017517 A1 and WO 1997/047648 A1, which are incorporated by reference in their entirety as well.

### Multimerization Element

The term “multimerization element” or “multimerization domain” refers to (poly-)peptides or proteins capable of inducing or promoting the multimerization of (poly-)peptides or proteins of interest. The term includes oligomerization elements, tetramerization elements, trimerization elements or dimerization elements.

Multimerization elements may for instance suitably be (additionally) encoded by artificial nucleic acid (RNA) molecules encoding antigenic (poly-)peptides or proteins. Multimerization elements inserted into or fused to antigenic (poly-)peptides or proteins of interest may advantageously mediate the formation of multimeric antigen-complexes or antigenic nanoparticles, which are preferably capable of inducing, promoting or potentiating immune responses to said antigen. Thereby, multimerization elements may be used to mimic a “natural” infection with a pathogen (e.g., virus) exhibiting a plurality of antigens adjacent to each other (e.g., hemagglutinin (HA) antigen of the influenza virus). However, multimerization elements may be usefully combined with any other (poly-)peptide or protein of interest as well. When encoded in combination with a (poly-)peptide or protein of interest, such multimerization element can be placed at its N-Terminus, or the C-Terminus, or both. On nucleic acid level, the coding sequence for such multimerization element is typically placed in frame (i.e. in the same reading frame), 5′ or 3′ to the coding sequence for the (poly-)peptide or protein of interest.

When used in combination with a polypeptide or protein of interest in the context of the present invention, such multimerization element can be placed at the N-terminus, C-terminus and/or within the (poly-)peptide or protein of interest. On nucleic acid level, the coding sequence for such multimerization element is typically placed in frame (i.e. in the same reading frame), 5′ or 3′ to the coding sequence for the polypeptide or protein of interest.

Exemplary dimerization elements may be selected from e.g. dimerization elements/domains of heat shock proteins, immunoglobulin Fc domains and leucine zippers (dimerization domains of the basic region leucine zipper class of transcription factors). Exemplary trimerization and tetramerization elements may be selected from e.g. engineered leucine zippers (engineered a-helical coiled coil peptide that adopt a parallel trimeric state), fibritin foldon domain from enterobacteria phage T4, GCN4pll, CCN4-pLI, and p53. Exemplary oligomerization elements may be selected from e.g. ferritin, surfactant D, oligomerization domains of phosphoproteins of paramyxoviruses, complement inhibitor C4 binding protein (C4 bp) oligomerization domains, Viral infectivity factor (Vif) oligomerization domain, sterile alpha motif (SAM) domain, and von Wil lebrand factor type D domain.

Ferritin forms oligomers and is a highly conserved protein found in all animals, bacteria, and plants. Ferritin is a protein that spontaneously forms nanoparticles of 24 identical subunits. Ferritin-antigen fusion constructs potentially form oligomeric aggregates or “clusters” of antigens that may enhance the immune response. Surfactant D protein (SPD) is a hydrophilic glycoprotein that spontaneously self-assembles to form oligomers. An SPD-antigen fusion constructs may form oligomeric aggregates or “clusters” of antigens that may enhance the immune response. Phosphoprotein of paramyxoviruses (negative sense RNA viruses) functions as a transcriptional transactivator of the viral polymerase. Oligomerization of the phosphoprotein is critical for viral genome replication. A phosphoprotein-antigen fusion constructs may form oligomeric aggregates or “clusters” of antigens that may enhance the immune response. Complement inhibitor C4 binding Protein (C4 bp) may also be used as a fusion partner to generate oligomeric antigen aggregates. The C-terminal domain of C4 bp (57 amino acid residues in humans and 54 amino acid residues in mice) is both necessary and sufficient for the oligomerization of C4 bp or other polypeptides fused to it. A C4 bp-antigen fusion constructs may form oligomeric aggregates or “clusters” of antigens that may enhance the immune response. Viral infectivity factor (Vif) multimerization domain has been shown to form oligomers both in vitro and in vivo. The oligomerization of Vif involves a sequence mapping between residues 151 to 164 in the C-terminal domain, the 161 PPLP 164 motif (for human HIV-1, TPKKIKPPLP). A Vif-antigen fusion constructs may form oligomeric aggregates or “clusters” of antigens that may enhance the immune response.

The sterile alpha motif (SAM) domain is a protein interaction module present in a wide variety of proteins involved in many biological processes. The SAM domain that spreads over around 70 residues is found in diverse eukaryotic organisms. SAM domains have been shown to homo- and hetero-oligomerise, forming multiple self-association oligomeric architectures. A SAM-antigen fusion constructs may form oligomeric aggregates or “clusters” of antigens that may enhance the immune response. von Willebrand factor (vWF) contains several type D domains: D1 and D2 are present within the N-terminal propeptide whereas the remaining D domains are required for oligomerization. The vWF domain is found in various plasma proteins: complement factors B, C2, C3 and CR4; the Integrins (I-domains); collagen types VI, VII, XII and XIV; and other extracellular proteins. A vWF-antigen fusion constructs may form oligomeric aggregates or “clusters” of antigens that may enhance the immune response.

Particular multimerization elements and nucleic acid sequences encoding the same envisaged for use in the present invention are inter alia disclosed in WO 2017/081082 A2, which is incorporated by reference in its entirety herein.

### Virus-Like Particle Forming Element

The term “virus-like particle forming element” or “VLP-forming element” refers to (poly-)peptides or proteins capable of assembling into non-replicative and/or non-infective virus-like particles structurally resembling a virus particle. VLPs are essentially devoid of infectious and/or replicative viral genome or genome function. Typically, a VLP lacks all or part of the replicative and infectious components of the viral genome.

VLP-forming elements are typically viral or phage structural proteins (i.e. envelope proteins or capsid proteins) which preferably comprise repetitive high density displays of antigens forming conformational epitopes that can elicit strong adaptive immune responses.

VLP-forming elements may for instance suitably be (additionally) encoded by artificial nucleic acid (RNA) molecules encoding antigenic (poly-)peptides or proteins, but can, however, be usefully combined with any other (poly-)peptide or protein of interest as well. VLP-forming elements inserted into or fused to (poly-)peptides or proteins of interest may for instance be used to promote or improve antigen clustering and immunogenicity of an antigenic (poly-)peptide or protein of interest. When encoded in combination with a (poly-)peptide or protein of interest, such VLP-forming element can be placed at the N-terminus, C-terminus and/or within the (poly-)peptide or proteins of interest. On nucleic acid level, the coding sequence for such VLP-forming element is typically placed in frame (i.e. in the same reading frame), 5′ to, 3′ to or within the coding sequence for the (poly-)peptide or protein of interest.

Exemplary VLP-forming elements may be derived from RNA bacteriophages, bacteriophages, Hepatitis B virus (HBV), preferably its capsid protein or its envelope protein, measles virus, Sindbis virus, rotavirus, foot-and-mouth-disease virus, Norwalk virus, Alphavirus, retrovirus, preferably its GAG protein, retrotransposon Ty, preferably the protein pi, human Papilloma virus, Polyoma virus, Tobacco mosaic virus, Flock House Virus, cowpea mosaic virus (CPMV), cowpea chlorotic mottle virus (CCMV), or Sobemovirus. Particular VLP-forming elements and nucleic acid sequences encoding the same envisaged for use in the present invention are inter alia disclosed in WO 2017/081082 A2, which is incorporated by reference in its entirety herein.

### Transmembrane Elements

“Transmembrane elements” or“membrane spanning polypeptide elements” (also referred to as “transmembrane domains” or “TM”) are present in proteins that are integrated or anchored in cellular plasma membranes. Transmembrane elements thus preferably comprise or consist of a sequence of amino acid residues capable of spanning and, thereby, preferably anchoring a fused (poly-)peptide or protein in a phospholipid membrane. A transmembrane element may comprise at least about 15 amino acid residues, preferably at least 18, 20, 22, 24, 25, 30, 35 or 40 amino acid residues. Typical transmembrane elements are about 20±5 amino acids in length. The amino acid residues constituting the transmembrane element are preferably selected from non-polar, primarily hydrophobic amino acids. Preferably, at least 50%, 60%, 70%, 80%, 90%, 95% or more of the amino acids of a transmembrane element may be hydrophobic, e.g., leucines, isoleucines, tyrosines, or tryptophans. Transmembrane elements may in particular include a series of conserved serine, threonine, and tyrosine residues. Typical transmembrane elements are alpha-helical transmembrane elements. Transmembrane elements may comprise single hydrophobic alpha helices or beta barrel structures; whereas hydrophobic alpha helices are usually present in proteins that are present in membrane anchored proteins (e.g., seven transmembrane domain receptors), beta-barrel structures are often present in proteins that generate pores or channels.

Transmembrane elements may for instance suitably be (additionally) encoded by artificial nucleic acid (RNA) molecules encoding antigenic (poly-)peptides or proteins, but can, however, be usefully combined with any other (poly-)peptide or protein of interest as well. TM elements fused to or inserted into (poly-)peptides or proteins of interest may advantageously anchor said (poly-)peptide or protein in the cell plasma membrane. In case of antigenic (poly-)peptides or proteins, such anchoring may promote antigen clustering, preferably resulting in enhanced immune responses. However, TM elements may be combined with any other (poly-)peptide or protein as well. When encoded in combination with a (poly-)peptide or protein of interest, such transmembrane element can be placed at at the N-terminus, C-terminus and/or within of the (poly-)peptide or protein of interest. On nucleic acid level, the coding sequence for such transmembrane element is typically placed in frame (i.e. in the same reading frame), 5′ to, 3′ or within the coding sequence for the (poly-)peptide or protein of interest.

Exemplary transmembrane elements may be selected from the transmembrane domain of Hemagglutinin (HA) of Influenza virus, Env of HIV-1, EIAV (equine infectious anemia virus), MLV (murine leukemia virus), mouse mammary tumor virus, G protein of VSV (vesicular stomatitis virus), Rabies virus, or a transmembrane element of a seven transmembrane domain receptor. Particular transmembrane elements and nucleic acid sequences encoding the same envisaged for use in the present invention are inter alia disclosed in WO 2017/081082 A2, which is incorporated by reference in its entirety herein.

### Dendritic Cell Targeting Elements

The term “dendritic cell targeting element” refers to a (poly-)peptide or protein capable of targeting to dendritic cells (CDs). Dendritic cells (DCs), the most potent antigen presenting cells (APCs), link the innate immune response to the adaptive immune response. They bind and internalize pathogens/antigens and display fragments of the antigen on their membrane (via MHC molecules) to stimulate T-cell responses against those pathogens/antigens.

Dendritic cell targeting elements may for instance suitably be (additionally) encoded by artificial nucleic acid (RNA) molecules encoding antigenic (poly-)peptides or proteins, to target antigens to DCs in order to stimulate and induce effective immune responses. However, dendritic cell targeting elements can be usefully combined with any other (poly-)peptide or protein of interest as well. When used in combination with a polypeptide or protein of interest in the context of the present invention, such dendritic cell targeting element can be placed at the N-terminus, C-terminus and/or within the (poly-)peptide or protein of interest. On nucleic acid level, the coding sequence for such dendritic cell element is typically placed in frame (i.e. in the same reading frame), 5′ or 3′ to the coding sequence for the (poly-)peptide or protein of interest.

Dendritic cell targeting elements include (poly-)peptides and proteins (e.g., antibody fragments, receptor ligands) preferably capable of interacting with or binding to DC surface receptors, such as C-type lectins (mannose receptors (e.g., MR1, DEC-205 (CD205)), CD206, DC-SIGN (CD209), Clec9a, DCIR, Lox-1, MGL, MGL-2, Clec12A, Dectin-1, Dectin-2, langerin (CD207)), scavenger receptors, F4/80 receptors (EMR1), DC-STAMP, receptors for the Fc portion of antibodies (Fc receptors), toll-like receptors (e.g., TLR2, 5, 7, 8, 9) and complement receptors (e.g., CR1, CR2).

Exemplary dendritic cell targeting elements may be selected from anti-DC-SIGN antibodies, CD1.1 c specific single chain fragments (scFv), DEC205-specific single chain fragments (scFv), soluble PD-1, chemokine (C motif) ligand XCL1, CD40 ligand, human IgG1, murine IgG2a, anti Celec 9A, anti MHCII scFv. Particular dendritic cell targeting elements and nucleic acid sequences encoding the same envisaged for use in the present invention are inter alia disclosed in WO 2017/081082 A2 as well as in Apostolopoulos et al. J Drug Deliv. 2013; 2013:869718 and Kastenmûller et al. Nat Rev Immunol. 2014 October; 14(10):705-11, all of which are incorporated by reference in their entirety herein.

### Immunological Adjuvant Element

The term “immunological adjuvant elements”, or “adjuvant elements”, refers to (poly-)peptides or proteins that enhance the immune response, e.g. by triggering a danger response (e.g., damage-associated molecular pattern molecules (DAMPs)), activating the complement system (e.g., peptides/proteins involved in the classical complement pathway, the alternative complement pathway, and the lectin pathway) or triggering an innate immune response (e.g., pathogen-associated molecular pattern molecules, PAMPs).

Immunological adjuvant elements may for instance suitably be (additionally) encoded by artificial nucleic acid (RNA) molecules encoding antigenic (poly-)peptides or proteins, to enhance immune responses to the encoded antigens. However, immunological adjuvant elements can be usefully combined with any other (poly-)peptide or protein of interest as well. When used in combination with a polypeptide or protein of interest in the context of the present invention, immunological adjuvant elements can be placed at the N-terminus, C-terminus and/or within the (poly-)peptide or protein of interest. On nucleic acid level, the coding sequence for such immunologic adjuvant element is typically placed in frame (i.e. in the same reading frame), 5′ to, 3′ to or within the coding sequence for the (poly-)peptide or protein of interest.

Exemplary immunological adjuvant elements may be selected from heat shock proteins (e.g., HSP60, HSP70, gp96), flagellin FliC, high mobility group box 1 proteins (e.g., HMGN1), extra domain A of fibronectin (EDA), C3 protein fragments (e.g. C3d), transferrin, β-defensin, or any other peptide/protein PAMP-receptor (PRs) ligand, DAMP or element that activates the complement system. Particular immunological adjuvant elements and nucleic acid sequences encoding the same envisaged for use in the present invention are inter alia disclosed in WO 2017/081082 A2, which is incorporated by reference in its entirety herein.

### Elements Promoting Antigen Presentation

The term “element promoting antigen presentation” refers to (poly-)peptides or proteins that are capable of mediating of promoting entry into the lysosomal/proteasomal or exosomal pathway and/or loading and presentation of processed (poly-)peptides or proteins onto major histocompatibility complex (MHC) molecules (MHC-I or MHC-II) and presentation in an MHC-bound form on the cell surface.

Elements promoting antigen presentation may for instance suitably be (additionally) encoded by artificial nucleic acid (RNA) molecules encoding antigenic (poly-)peptides or proteins, to enhance processing and MHC-presentation of the encoded antigens. However, elements promoting antigen presentation can be usefully combined with any other (poly-)peptide or protein of interest as well. When used in combination with a (poly-)peptide or protein of interest, elements promoting antigen presentation can be placed at the N-terminus, C-terminus and/or within said (poly-)peptide or protein of interest, or combinations thereof. On nucleic acid level, the coding sequence for such elements promoting antigen presentation is typically placed in frame (i.e. in the same reading frame), 5′ to, 3′ to or within the coding sequence for the (poly-)peptide or protein of interest.

Exemplary elements promoting antigen presentation may be selected from MHC invariant chain (li), invariant chain (li) lysosome targeting signal, sorting signal of the lysosomal-associated membrane protein LAMP-1, lysosomal integral membrane protein-II (LIMP-II) and C1C2 Lactadherin domain. Particular elements promoting antigen presentation and nucleic acid sequences encoding the same envisaged for use in the present invention are inter alia disclosed in WO 2017/081082 A2, which is incorporated by reference in its entirety herein.

### 2A Peptides

Viral “2A peptides” (also referred to as “self-cleaving” peptides) are (poly-)peptides or proteins which allow the expression of multiple proteins from a single open reading frame. The terms “2A peptide” and “2A element” are used interchangeably herein. The mechanism by the 2A sequence for generating two proteins from one transcript is by ribosome skipping—a normal peptide bond is impaired at 2A, resulting in two discontinuous protein fragments from one translation event.

2A peptides may for instance suitably be (additionally) encoded by artificial nucleic acid (RNA) molecules encoding (poly-)peptides or proteins that require cleavage. For instance, 2A peptides may be inserted into polypeptide fusions between two or more two antigenic (poly-)peptides, or between a protein of interest and a signal peptide. The coding sequence for such a 2A peptide is typically located in between the (poly-)peptide or protein encoding sequences. Self-cleavage of the 2A peptide preferably yields at least one separate (poly-)peptide or protein of interest (e.g. a protein of interest without its signal peptide, or two antigenic (poly-)peptides or proteins of interest). 2A peptides may also suitably be encoded by artificial nucleic acid (RNA) molecules encoding multi-chain (poly-)peptides or proteins of interest, such as antibodies. Such artificial nucleic acid (RNA) molecules may comprise, for instance, two coding sequences encoding two antibody chains separated by a nucleic acid sequence encoding a 2A peptide.

When used in combination with a polypeptide or protein of interest in the context of the present invention, 2A peptides can be placed at the N-terminus, C-terminus and/or within the (poly-)peptide or protein of interest, or combinations thereof. On nucleic acid level, the coding sequence for such 2A peptide is typically placed in frame (i.e. in the same reading frame), 5′ to, 3′ to or within the coding sequence for the (poly-)peptide or protein of interest.

Exemplary 2A peptides may be derived from foot-and-mouth diseases virus, from equine rhinitis A virus, Thosea asigna virus, Porcine teschovirus-1. Particular 2A peptides and nucleic acid sequences encoding the same envisaged for use in the present invention are inter alia disclosed in WO 2017/081082 A2, which is incorporated by reference in its entirety herein.

### Isoforms, Homologs, Variants, Fragments and Derivatives

Each of the (poly-)peptides and proteins of interest and, where applicable, each additional tag, sequence, linker, element or domain disclosed herein also includes isoforms, homologs, variants, fragments and derivatives thereof. Thus, artificial nucleic acid (RNA) molecules of the invention may encode in their at least one coding region, at least one therapeutic, antigenic or allergenic (poly-)peptide or protein, and optionally at least one additional tag, sequence, linker, element or domain as disclosed herein, or an isoform, homolog, variant, fragment or derivative thereof. Such isoforms, homologs, variants, fragments and derivatives are preferably functional, i.e. exhibit the same desired biological properties, and/or capable of exerting the same desired biological function as the respective reference (poly-)peptide, protein, tag, sequence, linker, element or domain. For example, isoforms, homologs, variants, fragments and derivatives of therapeutic (poly-)peptides or proteins are preferably capable of mediating the desired therapeutic effect. Isoforms, homologs, variants, fragments and derivatives of antigenic or allergenic (poly-)peptides or proteins are preferably capable of mediating the desired antigenic or allergenic effect, i.e. more preferably of inducing an immune response or allergenic response.

The term “isoform” refers to post-translational modification (PTM) variants of (poly-)peptides, proteins or amino acid sequences as disclosed herein. PTMs may result in covalent or non-covalent modifications of a given protein. Common post-translational modifications include glycosylation, phosphorylation, ubiquitinylation, S-nitrosylation, methylation, N-acetylation, lipidation, disulfide bond formation, sulfation, acylation, deamination etc. Different PTMs may result, e.g., in different chemistries, activities, localizations, interactions or conformations.

The term “homolog” encompasses “orthologs” and “paralogs”. “Orthologs” are (poly-)peptides or proteins or amino acid sequences encoded by genes in different species that evolved from a common ancestral gene by speciation. “Paralogs” are genes produced via gene duplication within a genome.

The term “variant” in the context of (poly-)peptides, proteins or amino acid sequences refers to “(amino acid) sequence variants”, i.e. (poly-)peptides, proteins or amino acid sequences with at least one amino acid mutation as compared to a reference (or “parent”) amino acid sequence. Amino acid mutations include amino acid substitutions, insertions or deletions. The term (amino acid) “substitution” may refers to conservative or non-conservative amino acid substitutions. In some embodiments, it may be preferred that a “variant” essentially comprises conservative amino acid substitutions, wherein amino acids, originating from the same class, are exchanged for one another. In particular, these are amino acids having aliphatic side chains, positively or negatively charged side chains, aromatic groups in the side chains or amino acids, the side chains of which can form hydrogen bridges, e.g. side chains which have a hydroxyl function. By conservative constitution, e.g. an amino acid having a polar side chain may be replaced by another amino acid having a corresponding polar side chain, or, for example, an amino acid characterized by a hydrophobic side chain may be substituted by another amino acid having a corresponding hydrophobic side chain (e.g. serine (threonine) by threonine (serine) or leucine (isoleucine) by isoleucine (leucine)).

Preferably, the term “variant” as used herein includes naturally occurring variants, such as prepeptides, preproproteins, proproteins, that have been subjected to post-translational proteolytic processing (this may involve removal of the N-terminal methionine, signal peptide, and/or the conversion of an inactive or non-functional protein to an active or functional one), transcript variants, as well as naturally occurring and engineered mutant (poly-)peptides, proteins and amino acid sequences. The terms “transcript variants” or “splice variants” refer to variants of (poly-)peptides, proteins or amino acid sequences produced from messenger RNAs that are initially transcribed from the same gene, but are subsequently subjected to alternative (or differential) splicing, where particular exons of a gene may be included within or excluded from the final, processed messenger RNA (mRNA). A “variant” as defined herein may be derived from, isolated from, related to, based on or homologous to the reference (poly-)peptide, protein or amino acid sequence. A “variant” (poly-)peptide, protein or amino acid sequence may preferably have a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an amino acid sequence of the respective reference (poly-)peptide, protein or amino acid sequence.

The term “fragment” in the context of (poly-)peptides, proteins or amino acid sequences refers to (poly-)peptides, proteins or amino acid sequences which consist of a continuous subsequence of the full-length amino acid sequence of a reference (or “parent”) (poly-)peptide, proteins or amino acid sequences. The “fragment” is, with regard to its amino acid sequence, N-terminally, C-terminally and/or intrasequentially truncated as compared to the reference amino acid sequence. Such truncation may occur either on the amino acid level or on the nucleic acid level, respectively. In other words, a “fragment” may typically consist of a shorter portion of a full-length amino acid sequence and thus preferably consists of an amino acid sequence that is identical to the corresponding stretch within a full-length reference amino acid sequence. The term includes naturally occurring fragments (such as fragments resulting from naturally occurring in vivo protease activity) as well as engineered fragments. Fragments may be derived from naturally occurring (poly-)peptides, proteins or amino acid sequences as disclosed herein, or from isoforms, homologs or variants thereof.

A “fragment” may comprise at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of respective reference amino acid sequences.

It may be preferred that “fragments” consists of a continuous stretch of amino acids corresponding to a continuous amino acid stretch in the reference amino acid sequence, wherein the fragment corresponds to at least 20%, preferably at least 30%, more preferably at least 40%, more preferably at least 50%, even more preferably at least 60%, even more preferably at least 70%, and most preferably at least 80% of the total (i.e. full-length) reference amino acid sequence. A sequence identity indicated with respect to a “fragment” may preferably refer to the full-length reference amino acid sequence. A (poly-)peptide, protein or amino acid sequence “fragment” may preferably have an amino acid sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with the reference amino acid sequence.

The term “derivative” in the context of (poly-)peptides, proteins or amino acid sequences refers to modifications of a reference or “parent” (poly-)peptide, protein or amino acid sequence including or lacking an additional biological property or functionality. For instance, (poly-)peptide or protein “derivatives” may be modified through the introduction or removal of domains that confer a particular biological functionality, such as the capability of binding to a (further) target, or an enzymatic activity. Other modifications may modulate the pharmacokinetic/pharmacodynamics properties, such as stability, biological half-life, bioavailability, absorption; distribution and/or reduced clearance. “Derivatives” may be prepared by introducing or deleting amino acid sequences post-translationally or on a nucleic acid sequence level (cf. using standard genetic engineering techniques (cf. Sambrook J et al., 2012 (4th ed.), Molecular cloning: a laboratory manual. Cold Spring Harbor Laboratory, Cold Spring Harbor, New York). A “derivative” may be derived from, i.e. correspond to a modified full-length wild-type (poly-)peptide, protein or amino acid sequence, or an isoform, homolog, fragment or variant thereof. The term “derivatives” further include (poly-)peptides, proteins or amino acid sequences that are chemically modified or modifiable after translation, e.g. by PEGylation or PASylation.

According to some embodiments, the particularly preferred that if, in addition to the (poly-)peptide or protein of interest, a further (poly-)peptide or protein is encoded by the at least one coding sequence as defined herein—the encoded peptide or protein is preferably no histone protein, no reporter protein (e.g. Luciferase, GFP and its variants (such as eGFP, RFP or BFP), and/or no marker or selection protein, including alpha-globin, galactokinase and Xanthine:Guanine phosphoribosyl transferase (GPT), hypoxanthine-guanine phosphoribosyltransferase (HGPRT), beta-galactosidase, galactokinase, alkaline phosphatase, secreted embryonic alkaline phosphatase (SEAP) or a resistance gene (such as a resistance gene against neomycin, puromycin, hygromycin and zeocin). In preferred embodiments, the artificial nucleic acid (RNA) molecule, does not encode a reporter gene or a marker gene. In preferred embodiments, the artificial nucleic acid (RNA) molecule, does not encode luciferase. In other embodiments, the artificial nucleic acid (RNA) molecule, does not encode GFP or a variant thereof.

### Nucleic Acid Sequences

The artificial nucleic acid (RNA) molecule of the invention may encode any desired (poly-)peptide or protein disclosed herein. Specifically, said artificial nucleic acid (RNA) molecule may comprise at least one coding region encoding a (poly-)peptide or protein comprising or consisting of an amino acid sequence according to any one of SEQ ID NOs: 42-45, or a homolog, variant, fragment or derivative thereof, preferably having an amino acid sequence having, in increasing order of preference, at least 50%, 60%, 70%, 80%, 90%, 95%, 96%, 97%, 98%, or 99% sequence identity to the amino acid sequence according to any one of SEQ ID NOs: 42-45, or a variant or fragment of any of these sequences.

Accordingly, the artificial nucleic acid (RNA) molecule of the invention may preferably comprise or consist of a nucleic acid sequence according to any one of SEQ ID NOs: 46-49; or a nucleic acid sequence having, in increasing order of preference, at least 50%, 60%, 70%, 80%, 90%, 95%, 96%, 97%, 98%, or 99% sequence identity to the any one of said nucleic acid sequences.

The present invention envisages the beneficial combination of coding regions encoding (poly-)peptides or proteins of interest operably linked to UTR elements as defined herein, in order to preferably increase the expression of said encoded proteins. Preferably, said artificial nucleic acids may thus comprise or consist of a nucleic acid sequence according to any one of SEQ ID NOs: 50-368, or a (functional) variant, fragment or derivative thereof, in particular nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### Nucleic Acid Molecules and RNAs

The terms “nucleic acid”, “nucleic acid molecule” or “artificial nucleic acid molecule” means any DNA- or RNA-molecule and is used synonymous with polynucleotide. Where ever herein reference is made to a nucleic acid or nucleic acid sequence encoding a particular protein and/or peptide, said nucleic acid or nucleic acid sequence, respectively, preferably also comprises regulatory sequences allowing in a suitable host, e.g. a human being, its expression, i.e. transcription and/or translation of the nucleic acid sequence encoding the particular protein or peptide.

The inventive artificial nucleic acid molecule may be a DNA or preferably be an RNA. It will be understood that the term “RNA” refers to ribonucleic acid molecules characterized by the specific succession of their nucleotides joined to form said molecules (i.e. their RNA sequence). The term “RNA” may thus be used to refer to RNA molecules or RNA sequences as will be readily understood by the skilled person in the respective context. For instance, the term “RNA” as used in the context of the invention preferably refers to an RNA molecule (said molecule being characterized, inter alia, by its particular RNA sequence). In the context of the sequence modifications disclosed herein, the term “RNA” will be understood to relate to (modified) RNA sequences, but typically also includes the resulting RNA molecules (which are modified with regard to their RNA sequence). In preferred embodiments, the RNA may be an mRNA, a viral RNA, a self-replicating RNA or a replicon RNA, preferably an mRNA.

### Mono-, Bi- or Multicistronic RNAs

In preferred embodiments, the artificial nucleic acid (RNA) molecule, of the invention may be mono-, bi-, or multicistronic. Bi- or multicistronic RNAs typically comprise two (bicistronic) or more (multicistronic) open reading frames (ORF).

An open reading frame in this context is a sequence of codons that is translatable into a peptide or protein. The coding sequences in a bi- or multicistronic artificial nucleic acid (RNA) molecule, may encode the same or, preferably, distinct (poly-)peptides or proteins of interest. In this context, “distinct” (poly-)peptides or proteins means (poly-)peptides or proteins being encoded by different genes, having a different amino acid sequence, exhibiting different biochemical or biological properties, having different biological functions and/or being derived from different species. In other words, coding sequences encoding two or more “distinct” (poly-)peptides or proteins, may for instance encode: (a) protein A and protein B, wherein A and B are derived from gene A′ and B′, respectively, or (b) human protein A and mouse protein A, or (c) protein A and protein A′, wherein protein A′ is a variant, fragment or derivative of A, and optionally exhibits a different amino acid sequence and/or different biochemical or biological properties as compared to A.

Bi- or even multicistronic artificial nucleic acid (RNA) molecules, may encode, for example, two or more, i.e. at least two, three, four, five, six or more (preferably distinct) (poly-)peptides or proteins of interest.

In some embodiments, the coding sequences encoding two or more (preferably distinct) (poly-)peptides or proteins of interest, may be separated in the bi- or multicistronic artificial nucleic acid (RNA) molecule, by at least one IRES (internal ribosomal entry site) sequence. The term “IRES” (internal ribosomal entry site) refers to an RNA sequence that allows for translation initiation. An IRES can function as a sole ribosome binding site, but it can also serve to provide a bi- or even multicistronic artificial nucleic acid (RNA) molecule which encodes several (preferably distinct) (poly-)peptides or proteins of interest (or homologs, variants, fragments or derivatives thereof), which are to be translated by the ribosomes independently of one another. Examples of IRES sequences, which can be used according to the invention, are those derived from picornaviruses (e.g. FMDV), pestiviruses (CFFV), polioviruses (PV), encephalomyocarditis viruses (ECMV), foot and mouth disease viruses (FMDV), hepatitis C viruses (HCV), classical swine fever viruses (CSFV), mouse leukoma virus (MLV), simian immunodeficiency viruses (SIV) or cricket paralysis viruses (CrPV).

According to further embodiments the at least one coding sequence of the artificial nucleic acid (RNA) molecule, of the invention may encode at least two, three, four, five, six, seven, eight and more, preferably distinct, (poly-)peptides or proteins of interest linked with or without an amino acid linker sequence, wherein said linker sequence may comprise rigid linkers, flexible linkers, cleavable linkers (e.g., self-cleaving peptides) or a combination thereof.

Preferably, the artificial nucleic acid (RNA) molecule, comprises a length of about 50 to about 20000, or 100 to about 20000 nucleotides, preferably of about 250 to about 20000 nucleotides, more preferably of about 500 to about 10000, even more preferably of about 500 to about 5000.

The artificial nucleic acid (RNA) molecule, of the invention may further be single stranded or double stranded. When provided as a double stranded RNA or DNA, the artificial nucleic acid molecule preferably comprises a sense and a corresponding antisense strand.

### Nucleic Acid Modifications

Artificial nucleic acid molecules, preferably RNAs, of the invention, may be provided in the form of modified nucleic acids. Suitable nucleic acid modifications envisaged in the context of the present invention are described below.

According to preferred embodiments, the at least one artificial nucleic acid (RNA) molecule, of the invention may be “modified”, i.e. comprise at least one modification as defined herein. Said modification may preferably be a sequence modification, or a (chemical) nucleobase modification as described herein. A “modification” as defined herein preferably leads to a stabilization of said artificial nucleic acid (RNA) molecule. More preferably, the invention thus provides a “stabilized” artificial nucleic acid (RNA) molecule. According to preferred embodiments, the artificial nucleic acid (RNA) molecule, of the invention may thus be provided as a “stabilized” artificial nucleic acid (RNA) molecule, in particular mRNA, i.e. which is essentially resistant to in vivo degradation (e.g. by an exo- or endo-nuclease).

### Nucleobase Modifications

Artificial nucleic acid molecules of the invention may be modified in their nucleotides, more specifically in the phosphate backbone, the sugar moiety or the nucleobases. In other words, the present invention envisages that a “modified” artificial nucleic acid (RNA) molecule, may contain nucleotide/nucleoside analogues/modifications (modified nucleotides or nucleosides), e.g. backbone modifications, sugar modifications or nucleobase modifications.

### Phosphate Backbone Modifications

Artificial nucleic acid molecules of the invention may comprise backbone modifications, i.e. nucleotides that are modified in their phosphate backbone. The term “backbone modification” refers to chemical modifications of the nucleotides' phosphate backbone, which may stabilize the backbone-modified nucleic acid molecule. A “backbone modification” is therefore understood as a modification, in which phosphates of the backbone of the nucleotides contained in said artificial nucleic acid (RNA) molecule, are chemically modified.

The phosphate groups of the backbone can be modified by replacing one or more of the oxygen atoms with a different substituent. Further, the modified nucleotides can include the full replacement of an unmodified phosphate moiety with a modified phosphate as described herein.

Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulphur. The phosphate linker can also be modified by the replacement of a linking oxygen with nitrogen (bridged phosphoroamidates), sulphur (bridged phosphorothioates) and carbon (bridged methylene-phosphonates).

Preferably, “backbone-modified” artificial nucleic acid molecules, preferably RNAs, may comprise phosphorothioate-modified backbones, wherein preferably at least one of the phosphate oxygens contained in the phosphate backbone is replaced by a sulphur atom. Further suitable phosphate backbone modifications include the incorporation of non-ionic phosphate analogues, such as, for example, alkyl and aryl phosphonates, in which the charged phosphonate oxygen is replaced by an alkyl or aryl group, or phosphodiesters and alkylphosphotriesters, in which the charged oxygen residue is present in alkylated form. Such backbone modifications typically include, without limitation, modifications from the group consisting of methylphosphonates, phosphoramidates and phosphorothioates (e.g. cytidine-5′-O-(1-thiophosphate)).

### Sugar Modifications:

Artificial nucleic acid molecules of the invention may comprise sugar modifications, i.e. nucleotides that are modified in their sugar moiety. The term “sugar modification” refers to chemical modifications of the nucleotides' sugar moiety. A “sugar modification” is therefore understood as a chemical modification of the sugar of the nucleotides of the artificial nucleic acid (RNA) molecule.

For example, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g., R═H, alkyl, cycloalkyl, aryl, aralkyl, heteroaryl or sugar); polyethyleneglycols (PEG), —O(CH₂CH₂O)nCH₂CH₂OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy.

“Deoxy” modifications include hydrogen, amino (e.g. NH₂; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O.

Modified sugar moieties may contain one or more carbons that possess the opposite stereochemical configuration as compared to the stereochemical configuration of the corresponding carbon in ribose. Thus, a sugar-modified artificial nucleic acid (RNA) molecule, may include nucleotides containing, for instance, arabinose as the sugar.

### Nucleobase Modifications:

Artificial nucleic acid molecules of the invention may comprise nucleobase modifications, i.e. nucleotides that are modified in their nucleobase moiety. The term “nucleobase modification” refers to chemical modifications of the nucleotides' nucleobase moiety. A “nucleobase modification” is therefore understood as a chemical modification of the nucleobase of the nucleotides of the artificial nucleic acid (RNA) molecule. Suitable nucleotides or nucleosides that are modified in their nucleobase moiety (also referred to as “nucleoside analogous” or “nucleotide analogues”) may advantageously increase the stability of the artificial nucleic acid (RNA) molecule and/or enhance the expression of a (poly-)peptide or protein encoded by its at least one coding region.

Examples of nucleobases found in RNA include, but are not limited to, adenine, guanine, cytosine and uracil. For example, the nucleotides described herein can be chemically modified on the major groove face. In some embodiments, the major groove chemical modifications can include an amino group, a thiol group, an alkyl group, or a halo group.

When referring to preferred “nucleoside modifications (nucleoside analogues)” below, the respective modified nucleotides (nucleotide analogues) are equally envisaged, and vice versa.

In some embodiments, the nucleotide analogues/modifications are selected from nucleobase modifications, which are preferably selected from 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-Iodo-2′-deoxycytidine-5′-triphosphate, 5-iodouridine-5′-triphosphate, 5-Iodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate.

In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the nucleotide can be modified on the major groove face and can include replacing hydrogen on C-5 of uracil with a methyl group or a halo group. In specific embodiments, a modified nucleoside is 5′-O-(1-thiophosphate)-adenosine, 5′-O-(1-thiophosphate)-cytidine, 5′-O-(1-thiophosphate)-guanosine, 5′-O-(1-thiophosphate)-uridine or 5′-O-(1-thiophosphate)-pseudouridine.

In some embodiments, the modified artificial nucleic acid (RNA) molecule, of the invention may comprise nucleoside modifications selected from 6-aza-cytidine, 2-thio-cytidine, a-thio-cytidine, Pseudo-iso-cytidine, 5-aminoallyl-uridine, 5-iodo-uridine, N1-methyl-pseudouridine, 5,6-dihydrouridine, a-thio-uridine, 4-thio-uridine, 6-aza-uridine, 5-hydroxy-uridine, deoxy-thymidine, 5-methyl-uridine, Pyrrolo-cytidine, inosine, a-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, a-thio-adenosine, 8-azido-adenosine, 7-deaza-adenosine.

In some embodiments, a modified artificial nucleic acid (RNA) molecule (or any other nucleic acid, in particular RNA, as defined herein) does not comprise any of the chemical modifications as described herein. Such modified artificial nucleic acids, may nevertheless comprise a lipid modification or a sequence modification as described below.

### Lipid Modifications

According to further embodiments, artificial nucleic acid molecules (RNAs) of the invention may contain at least one lipid modification.

Such a “lipid-modified” artificial nucleic acid molecule (RNA), of the invention typically comprises (i) an artificial nucleic acid molecule (RNA), as defined herein, (ii) at least one linker covalently linked to said artificial nucleic acid molecule (RNA), (iii) at least one lipid covalently linked to the respective linker.

Alternatively, the “lipid-modified” artificial nucleic acid molecule (RNA), may comprise at least one artificial nucleic acid molecule (RNA) and at least one (bifunctional) lipid covalently linked (without a linker) with said artificial nucleic acid molecule (RNA).

Alternatively, the “lipid-modified” artificial nucleic acid molecule (RNA) may comprise (i) an artificial nucleic acid molecule (RNA), (ii) at least one linker covalently linked to said artificial nucleic acid molecule (RNA), and (iii) at least one lipid covalently linked to the respective linker, and further (iv) at least one (bifunctional) lipid covalently linked (without a linker) to said artificial nucleic acid molecule (RNA).

In this context, it is particularly preferred that the lipid modification is present at the terminal ends of a linear artificial nucleic acid molecule (RNA).

### Sequence Modifications

According to preferred embodiments, the artificial nucleic acid molecule (RNA, preferably mRNA) of the invention, is “sequence-modified”, i.e. comprises at least one sequence modification as described below. Without wishing to be bound by specific theory, such sequence modifications may increase stability and/or enhance expression of the inventive artificial nucleic acid molecules (RNAs).

### G/C Content Modification

According to preferred embodiments, the artificial nucleic acid (RNA) molecule, more preferably mRNA, of the invention may be modified, and thus stabilized, by modifying its guanosine/cytosine (G/C) content, preferably by modifying the G/C content of the at least one coding sequence. In other words, the artificial nucleic acid molecule (RNA) may preferably be G/C modified, i.e. preferably comprise G/C modified (coding) sequence.

A “G/C-modified” nucleic acid (RNA) sequence typically refers to a nucleic acid (RNA) comprising a nucleic acid (RNA) sequence that is based on a modified wild-type nucleic acid (RNA) sequence and comprises an altered number of guanosine and/or cytosine nucleotides as compared to said wild-type nucleic acid (RNA) sequence. Such an altered number of G/C nucleotides may be generated by substituting codons containing adenosine or thymidine nucleotides by “synonymous” codons containing guanosine or cytosine nucleotides. Accordingly, the codon substitutions preferably do not alter the encoded amino acid residues, but exclusively alter the G/C content of the nucleic acid (RNA).

In a particularly preferred embodiment of the present invention, the G/C content of the coding sequence of the artificial nucleic acid molecule (RNA) of the invention is modified, particularly increased, compared to the G/C content of the coding sequence of the respective wild-type, i.e. unmodified nucleic acid (RNA). The amino acid sequence encoded by the inventive artificial nucleic acid molecule (RNA) is preferably not modified as compared to the amino acid sequence encoded by the respective wild-type nucleic acid (RNA).

The provision of “G/C modified” nucleic acid molecules (RNAs) is based on the finding that nuclei acid (RNA) sequences having an increased G (guanosine)/C (cytosine) content are generally more stable than nucleic acid (RNA) sequences having an increased A (adenosine)/U (uracil) content.

According to the invention, the codons of the inventive artificial nucleic acid molecule (RNA) are therefore varied as compared to the respective wild-type nucleic acid (RNA), while retaining the translated amino acid sequence, such that they include an increased amount of G/C nucleotides.

In respect to the fact that several codons code for one and the same amino acid (so-called degeneration of the genetic code), the most favourable codons for the stability can be determined (so-called alternative codon usage). Depending on the amino acid to be encoded by the inventive artificial nucleic acid molecule (RNA), there are various possibilities for modification its nucleic acid sequence, compared to its wild-type sequence. In the case of amino acids, which are encoded by codons, which contain exclusively G or C nucleotides, no modification of the codon is necessary.

Thus, the codons for Pro (CCC or CCG), Arg (CGC or CGG), Ala (GCC or GCG) and Gly (GGC or GGG) require no modification, since no A or U is present. In contrast, codons which contain A and/or U nucleotides can be modified by substitution of other codons, which code for the same amino acids but contain no A and/or U. Examples of these are: the codons for Pro can be modified from CCU or CCA to CCC or CCG; the codons for Arg can be modified from CGU or CGA or AGA or AGG to CGC or CGG; the codons for Ala can be modified from GCU or GCA to GCC or GCG; the codons for Gly can be modified from GGU or GGA to GGC or GGG. In other cases, although A or U nucleotides cannot be eliminated from the codons, it is however possible to decrease the A and U content by using codons which contain a lower content of A and/or U nucleotides. Examples of these are: the codons for Phe can be modified from UUU to UUC; the codons for Leu can be modified from UUA, UUG, CUU or CUA to CUC or CUG; the codons for Ser can be modified from UCU or UCA or AGU to UCC, UCG or AGC; the codon for Tyr can be modified from UAU to UAC; the codon for Cys can be modified from UGU to UGC; the codon for His can be modified from CAU to CAC; the codon for Gln can be modified from CAA to CAG; the codons for Ile can be modified from AUU or AUA to AUC; the codons for Thr can be modified from ACU or ACA to ACC or ACG; the codon for Asn can be modified from AAU to AAC; the codon for Lys can be modified from AAA to AAG; the codons for Val can be modified from GUU or GUA to GUC or GUG; the codon for Asp can be modified from GAU to GAC; the codon for Glu can be modified from GAA to GAG; the stop codon UAA can be modified to UAG or UGA. In the case of the codons for Met (AUG) and Trp (UGG), on the other hand, there is no possibility of sequence modification. The substitutions listed above can be used either individually or in all possible combinations to increase the G/C content of the inventive artificial nucleic acid sequence, preferably RNA sequence (or any other nucleic acid sequence as defined herein) compared to its particular wild-type nucleic acid sequence (i.e. the original sequence). Thus, for example, all codons for Thr occurring in the wild-type sequence can be modified to ACC (or ACG). Preferably, however, for example, combinations of the above substitution possibilities are used:

substitution of all codons coding for Thr in the original sequence (wild-type RNA) to ACC (or ACG) and
substitution of all codons originally coding for Ser to UCC (or UCG or AGC); substitution of all codons coding for Ile in the original sequence to AUC and
substitution of all codons originally coding for Lys to AAG and
substitution of all codons originally coding for Tyr to UAC; substitution of all codons coding for Val in the original sequence to GUC (or GUG) and
substitution of all codons originally coding for Glu to GAG and
substitution of all codons originally coding for Ala to GCC (or GCG) and
substitution of all codons originally coding for Arg to CGC (or CGG); substitution of all codons coding for Val in the original sequence to GUC (or GUG) and
substitution of all codons originally coding for Glu to GAG and
substitution of all codons originally coding for Ala to GCC (or GCG) and
substitution of all codons originally coding for Gly to GGC (or GGG) and
substitution of all codons originally coding for Asn to AAC; substitution of all codons coding for Val in the original sequence to GUC (or GUG) and
substitution of all codons originally coding for Phe to UUC and
substitution of all codons originally coding for Cys to UGC and
substitution of all codons originally coding for Leu to CUG (or CUC) and
substitution of all codons originally coding for Gln to CAG and
substitution of all codons originally coding for Pro to CCC (or CCG); etc.

Preferably, the G/C content of the coding sequence of the artificial nucleic acid molecule (RNA) of the invention may be increased by at least 7%, more preferably by at least 15%, particularly preferably by at least 20%, compared to the G/C content of the coding sequence of the wild-type nucleic acid (RNA) coding for the same (poly-)peptide or protein of interest.

According to preferred embodiments, at least 5%, 10%, 20%, 30%, 40%, 50%, 60%, more preferably at least 70%, even more preferably at least 80% and most preferably at least 90%, 95% or even 100% of the substitutable codons in the region coding for a (poly-)peptide or protein of interest, or the whole sequence of the wild type nucleic acid (RNA) sequence may be substituted, thereby increasing the G/C content of the resulting “G/C modified” sequence.

In this context, it is particularly preferable to increase the G/C content of the artificial nucleic acid molecule (RNA), preferably of its at least one coding sequence, to the maximum (i.e. 100% of the substitutable codons) as compared to the wild-type nucleic acid (RNA) sequence.

### Substitution of Rare Codons

Another preferred modification of the artificial nucleic acid molecule (RNA) is based on the finding that the translation efficiency is also determined by a different frequency in the occurrence of tRNAs in cells. Thus, if so-called “rare codons” are present in the artificial nucleic acid molecule (RNA) to an increased extent, the corresponding modified nucleic acid (RNA) sequence is translated less effectively than a nucleic acid (RNA) sequence comprising codons coding for relatively “frequent” tRNAs.

In some preferred embodiments, in modified artificial nucleic acid molecules (RNAs) of the invention, the coding region is thus modified compared to the coding region of the corresponding wild-type nucleic acid (RNA), such that at least one codon of the wild-type sequence, which codes for a tRNA which is relatively rare in the cell, is exchanged for a codon, which codes for a tRNA which is relatively frequent in the cell and carries the same amino acid as the relatively rare tRNA.

Thereby, the sequences of the artificial nucleic acid molecule (RNA) of the invention is modified such that codons for which frequently occurring tRNAs are available are inserted.

Thereby, all codons of the wild-type nucleic acid (RNA) sequence, which code for a tRNA which is relatively rare in the cell, can in each case be exchanged for a codon, which codes for a tRNA which is relatively frequent in the cell and which, in each case, carries the same amino acid as the relatively rare tRNA. The frequency of specific tRNAs in the cell is well-known to the skilled person; cf. e.g. Akashi, Curr. Opin. Genet. Dev. 2001, 11(6): 660-666. Codons recruiting the most frequent tRNA for a given amino acid (e.g. Gly) in the (human) cell, are particularly preferred.

According to the invention, it is particularly preferable to combine a modified (preferably increased, more preferably maximized) G/C with the use of “frequent” codons as described above, without modifying the amino acid sequence encoded by the coding sequence of said artificial nucleic acid molecule (RNA). Such “combined” modifications preferably result in an increased translation efficacy and stabilization of the resulting, modified artificial nucleic acid molecule (RNA).

Modified artificial nucleic acid molecules (RNAs) exhibiting the sequence modifications described herein (e.g., increased G/C content and exchange of tRNAs) can be provided with the aid of computer programs as explained in WO 02/098443, the disclosure content of which is included in its full scope in the present invention. Using this computer program, the nucleotide sequence of any desired nucleic acid, in particular RNA, can be modified in silico obtain modified artificial nucleic acid molecules (RNAs) with a nucleic acid (RNA) sequence exhibiting a maximum G/C content in combination with codons recruiting frequent tRNAs, while encoding the same (non-modified) amino acid sequence as a respective wild-type nucleic acid (RNA) sequence.

Alternatively, it is also possible to modify either the G/C content or the codon usage individually as compared to a reference sequence. The source code in Visual Basic 6.0 (development environment used: Microsoft Visual Studio Enterprise 6.0 with Servicepack 3) is also described in WO 02/098443.

### A/U Content Modification

According to further preferred embodiments, the A/U content at or near the ribosome binding site of the artificial nucleic acid molecule (RNA) of the invention is increased compared to the A/U content at or near the ribosome binding site of a respective wild-type nucleic acid (RNA). Increasing the A/U content around the ribosome binding site may preferably enhance ribosomal binding efficacy. Effective ribosome binding the ribosome binding site (Kozak sequence) preferably facilitates efficient translation of the artificial nucleic acid molecule (RNA).

### DSE Modifications

According to further preferred embodiments, the artificial nucleic acid molecule (RNA) may be modified with respect to potentially destabilizing sequence elements. Particularly, the coding sequence and/or the 5′ and/or 3′ untranslated region of said artificial nucleic acid molecule (RNA) may be modified compared to the respective wild-type nucleic acid (RNA) by removing any destabilizing sequence elements (DSEs), while the encoded amino acid sequence of the modified artificial nucleic acid molecule (RNA) is preferably not being modified compared to its respective wild-type nucleic acid (RNA).

Eukaryotic RNAs may comprise destabilizing sequence elements (DSE), which may draw signal proteins mediating enzymatic degradation of the nucleic acid molecule (RNA) in vivo. Exemplary DSEs include AU-rich sequences (AURES), which occur in 3′-UTRs of numerous unstable RNAs (Caput et al., Proc. Natl. Acad. Sci. USA 1986, 83: 1670 to 1674). Also encompassed by the term are sequence motifs, which are recognized by possible endonucleases, e.g. the sequence GAACAAG, which is contained in the 3′-UTR segment of the gene encoding the transferrin receptor (Binder et al., EMBO J. 1994, 13: 1969 to 1980).

By removing or substantially removing such DSEs from the nucleic acid sequence of the artificial nucleic acid molecule (RNA) of the invention, in particular from its coding region and/or its 3′- and/or 5′-UTR elements, the artificial nucleic acid molecule (RNA) is preferably stabilized.

The artificial nucleic acid molecule (RNA) of the invention may therefore be modified as compared to a respective wild-type nucleic acid (RNA) such that said artificial nucleic acid molecule (RNA) is devoid of destabilizing sequence elements (DSEs).

### Sequences Adapted to Human Codon Usage:

A further preferred modification of the artificial nucleic acid (RNA) molecule of the invention is based on the finding that codons encoding the same amino acid typically occur at different frequencies.

According to further preferred embodiments, in the modified artificial nucleic acid molecule (RNA), the coding sequence is modified compared to the corresponding region of the respective wild-type nucleic acid (RNA) such that the frequency of the codons encoding the same amino acid corresponds to the naturally occurring frequency of that codon according to the human codon usage as e.g. shown in Table 2.

For example, the coding sequence of a wild-type nucleic acid molecule (RNA) may be adapted in a way that the codon “GCC” (for Ala) is used with a frequency of 0.40, the codon “GCT” (for Ala) is used with a frequency of 0.28, the codon “GCA” (for Ala) is used with a frequency of 0.22 and the codon “GCG” (for Ala) is used with a frequency of 0.10 etc. (see Table 2).

TABLE 2
Human codon usage table

| Amino acid | codon | fraction | /1000 |
|---|---|---|---|
| Ala | GCG | 0.10 | 7.4 |
| Ala | GCA | 0.22 | 15.8 |
| Ala | GCT | 0.28 | 18.5 |
| Ala | GCC* | 0.40 | 27.7 |
| Cys | TGT | 0.42 | 10.6 |
| Cys | TGC* | 0.58 | 12.6 |
| Asp | GAT | 0.44 | 21.8 |
| Asp | GAC* | 0.56 | 25.1 |
| Glu | GAG* | 0.59 | 39.6 |
| Glu | GAA | 0.41 | 29.0 |
| Phe | TTT | 0.43 | 17.6 |
| Phe | TTC* | 0.57 | 20.3 |
| Gly | GGG | 0.23 | 16.5 |
| Gly | GGA | 0.26 | 16.5 |
| Gly | GGT | 0.18 | 10.8 |
| Gly | GGC* | 0.33 | 22.2 |
| His | CAT | 0.41 | 10.9 |
| His | CAC* | 0.59 | 15.1 |
| Ile | ATA | 0.14 | 7.5 |
| Ile | ATT | 0.35 | 16.0 |
| Ile | ATC* | 0.52 | 20.8 |
| Lys | AAG* | 0.60 | 31.9 |
| Lys | AAA | 0.40 | 24.4 |
| Leu | TTG | 0.12 | 12.9 |
| Leu | TTA | 0.06 | 7.7 |
| Leu | CTG* | 0.43 | 39.6 |
| Leu | CTA | 0.07 | 7.2 |
| Leu | CTT | 0.12 | 13.2 |
| Leu | CTC | 0.20 | 19.6 |
| Met | ATG* | 1 | 22.0 |
| Asn | AAT | 0.44 | 17.0 |
| Asn | AAC* | 0.56 | 19.1 |
| Pro | CCG | 0.11 | 6.9 |
| Pro | CCA | 0.27 | 16.9 |
| Pro | CCT | 0.29 | 17.5 |
| Pro | CCC* | 0.33 | 19.8 |
| Gln | CAG* | 0.73 | 34.2 |
| Gln | CAA | 0.27 | 12.3 |
| Arg | AGG | 0.22 | 12.0 |
| Arg | AGA* | 0.21 | 12.1 |
| Arg | CGG | 0.19 | 11.4 |
| Arg | CGA | 0.10 | 6.2 |
| Arg | CGT | 0.09 | 4.5 |
| Arg | CGC | 0.19 | 10.4 |
| Ser | AGT | 0.14 | 12.1 |
| Ser | AGC* | 0.25 | 19.5 |
| Ser | TCG | 0.06 | 4.4 |
| Ser | TCA | 0.15 | 12.2 |
| Ser | TCT | 0.18 | 15.2 |
| Ser | TCC | 0.23 | 17.7 |
| Thr | ACG | 0.12 | 6.1 |
| Thr | ACA | 0.27 | 15.1 |
| Thr | ACT | 0.23 | 13.1 |
| Thr | ACC* | 0.38 | 18.9 |
| Val | GTG* | 0.48 | 28.1 |
| Val | GTA | 0.10 | 7.1 |
| Val | GTT | 0.17 | 11.0 |
| Val | GTC | 0.25 | 14.5 |
| Trp | TGG* | 1 | 13.2 |
| Tyr | TAT | 0.42 | 12.2 |
| Tyr | TAC* | 0.58 | 15.3 |
| Stop | TGA* | 0.61 | 1.6 |
| Stop | TAG | 0.17 | 0.8 |
| Stop | TAA | 0.22 | 1.0 |

*most frequent codon

### Codon-Optimized Sequences:

As described above, in preferred embodiments of the present invention, all codons of the wild-type nucleic acid sequence which code for a relatively rare tRNA may be exchanged for a codon which codes for a relatively frequent tRNA carrying the same amino acid as the relatively rare tRNA.

It is particularly preferred that the most frequent codons are used for each encoded amino acid (see Table 2, most frequent codons are marked with asterisks). Such an optimization procedure increases the codon adaptation index (CAI) and ultimately maximises the CAI. In the context of the invention, nucleic acid (RNA) sequences with increased or maximized CAI are typically referred to as “codon-optimized” and/or “CAI increased” and/or “maximized” nucleic acid (RNA) sequences. According to preferred embodiments, the artificial nucleic acid molecule (RNA) of the invention comprises at least one coding sequence, wherein the coding sequence is “codon-optimized” as described herein. More preferably, the codon adaptation index (CAI) of the at least one coding sequence may be at least 0.5, at least 0.8, at least 0.9 or at least 0.95. Most preferably, the codon adaptation index (CAI) of the at least one coding sequence may be 1.

For example, the coding sequence of a wild-type nucleic acid molecule (RNA) may be adapted in a way that the most frequent (human) codon is always used for each encoded amino acid, e.g. “GCC” for Ala or “TGC” for Cys.

### C-Optimized Sequences:

According to preferred embodiments, the artificial nucleic acid molecule (RNA) is modified by altering, preferably increasing, the cytosine (C) content of its nucleic acid (RNA) sequence, in particular in its at least one coding sequence.

In preferred embodiments, the C content of the coding sequence of the artificial nucleic acid molecule (RNA) of the invention is modified, preferably increased, compared to the C content of the coding sequence of the respective wild-type (unmodified) nucleic acid (RNA). The amino acid sequence encoded by the at least one coding sequence of the artificial nucleic acid molecule (RNA) of the invention is preferably not modified as compared to the amino acid sequence encoded by the respective wild-type nucleic acid (RNA).

In preferred embodiments, said modified artificial nucleic acid molecule (RNA) may be modified such that at least 10%, 20%, 30%, 40%, 50%, 60%, 70% or 80%, or at least 90% of the theoretically possible maximum cytosine-content or even a maximum cytosine-content is achieved.

In further preferred embodiments, at least 10%, 20%, 30%, 40%, 50%, 60%, 70%, 80%, 90% or even 100% of the codons of the wild-type nucleic acid (RNA) sequence, which are “cytosine content optimizable” are replaced by codons having a higher cytosine-content than the ones present in the wild type sequence.

In further preferred embodiments, some of the codons of the wild type coding sequence may additionally be modified such that a codon for a relatively rare tRNA in the cell is exchanged by a codon for a relatively frequent tRNA in the cell, provided that the substituted codon for a relatively frequent tRNA carries the same amino acid as the relatively rare tRNA of the original wild-type codon. Preferably, all of the codons for a relatively rare tRNA may be replaced by a codon for a relatively frequent tRNA in the cell, except codons encoding amino acids, which are exclusively encoded by codons not containing any cytosine, or except for glutamine (Gln), which is encoded by two codons each containing the same number of cytosines.

In further preferred embodiments of the present invention, the modified artificial nucleic acid molecule (RNA) may be modified such that at least 80%, or at least 90% of the theoretically possible maximum cytosine-content or even a maximum cytosine-content is achieved by means of codons, which code for relatively frequent tRNAs in the cell, wherein the amino acid sequence encoded by the at least one coding region remains unchanged.

Due to the natural degeneracy of the genetic code, more than one codon may encode a particular amino acid. Accordingly, 18 out of 20 naturally occurring amino acids are encoded by more than one codon (with Tryp and Met being an exception), e.g. by 2 codons (e.g. Cys, Asp, Glu), by three codons (e.g. Ile), by 4 codons (e.g. Al, Gly, Pro) or by 6 codons (e.g. Leu, Arg, Ser). However, not all codons encoding the same amino acid are utilized with the same frequency under in vivo conditions. Depending on each single organism, a typical codon usage profile is established.

The term “cytosine content-optimizable codon” refers to codons, which exhibit a lower content of cytosines than other codons encoding the same amino acid. Accordingly, any wild-type codon, which may be replaced by another codon encoding the same amino acid and exhibiting a higher number of cytosines within that codon, is considered to be cytosine-optimizable (C-optimizable). Any such substitution of a C-optimizable wild-type codon by the specific C-optimized codon within a wild type coding sequence increases its overall C-content and reflects a C-enriched modified nucleic acid (RNA) sequence.

According to some preferred embodiments, the artificial nucleic acid (RNA) molecule of the invention, and in particular its at least one coding sequence, comprises or consists of a C-maximized sequence containing C-optimized codons for all potentially C-optimizable codons. Accordingly, 100% or all of the theoretically replaceable C-optimizable codons may preferably be replaced by C-optimized codons over the entire length of the coding sequence.

In this context, cytosine-content optimizable codons are codons, which contain a lower number of cytosines than other codons coding for the same amino acid.

Any of the codons GCG, GCA, GCU codes for the amino acid Ala, which may be exchanged by the codon GCC encoding the same amino acid, and/or

the codon UGU that codes for Cys may be exchanged by the codon UGC encoding the same amino acid, and/or
the codon GAU which codes for Asp may be exchanged by the codon GAC encoding the same amino acid, and/or
the codon that UUU that codes for Phe may be exchanged for the codon UUC encoding the same amino acid, and/or
any of the codons GGG, GGA, GGU that code Gly may be exchanged by the codon GGC encoding the same amino acid, and/or
the codon CAU that codes for His may be exchanged by the codon CAC encoding the same amino acid, and/or
any of the codons AUA, AUU that code for Ile may be exchanged by the codon AUC, and/or
any of the codons UUG, UUA, CUG, CUA, CUU coding for Leu may be exchanged by the codon CUC encoding the same amino acid, and/or
the codon AAU that codes for Asn may be exchanged by the codon AAC encoding the same amino acid, and/or
any of the codons CCG, CCA, CCU coding for Pro may be exchanged by the codon CCC encoding the same amino acid, and/or
any of the codons AGG, AGA, CGG, CGA, CGU coding for Arg may be exchanged by the codon CGC encoding the same amino acid, and/or
any of the codons AGU, AGC, UCG, UCA, UCU coding for Ser may be exchanged by the codon UCC encoding the same amino acid, and/or
any of the codons ACG, ACA, ACU coding for Thr may be exchanged by the codon ACC encoding the same amino acid, and/or
any of the codons GUG, GUA, GUU coding for Val may be exchanged by the codon GUC encoding the same amino acid, and/or
the codon UAU coding for Tyr may be exchanged by the codon UAC encoding the same amino acid.

In any of the above instances, the number of cytosines is increased by 1 per exchanged codon. Exchange of all non C-optimized codons (corresponding to C-optimizable codons) of the coding sequence results in a “C-maximized” coding sequence. In the context of the invention, at least 70%, preferably at least 80%, more preferably at least 90%, of the non C-optimized codons within the at least one coding sequence of the artificial nucleic acid (RNA) molecule of the invention may be replaced by “C-optimized” codons.

It may be preferred that for some amino acids the percentage of C-optimizable codons replaced by C-optimized codons is less than 70%, while for other amino acids the percentage of replaced codons may be higher than 70% to meet the overall percentage of C-optimization of at least 70% of all C-optimizable wild type codons of the coding sequence.

Preferably, in a “C-optimized” artificial nucleic acid (RNA) molecule, at least 50% of the C-optimizable wild type codons for any given amino acid may be replaced by “C-optimized” codons, e.g. any modified C-enriched nucleic acid (RNA) molecule preferably contains at least 50% C-optimized codons at C-optimizable wild type codon positions encoding any one of the above mentioned amino acids Ala, Cys, Asp, Phe, Gly, His, Ile, Leu, Asn, Pro, Arg, Ser, Thr, Val and Tyr, preferably at least 60%.

In this context, codons encoding amino acids, which are not cytosine content-optimizable and which are, however, encoded by at least two codons, may be used without any further selection process. However, the codon of the wild type sequence that codes for a relatively rare tRNA in the cell, e.g. a human cell, may be exchanged for a codon that codes for a relatively frequent tRNA in the cell, wherein both code for the same amino acid.

Accordingly, the relatively rare codon GAA coding for Glu may be exchanged by the relative frequent codon GAG coding for the same amino acid, and/or

the relatively rare codon AAA coding for Lys may be exchanged by the relative frequent codon AAG coding for the same amino acid, and/or
the relatively rare codon CAA coding for Gln may be exchanged for the relative frequent codon CAG encoding the same amino acid.

In this context, the amino acids Met (AUG) and Trp (UGG), which are encoded by only one codon each, remain unchanged. Stop codons are not cytosine-content optimized, however, the relatively rare stop codons amber, ochre (UAA, UAG) may be exchanged by the relatively frequent stop codon opal (UGA).

The single substitutions listed above may be used individually as well as in all possible combinations in order to optimize the cytosine-content of the modified artificial nucleic acid molecule (RNA), compared to a respective wild-type nucleic acid (RNA) sequence.

Accordingly, the at least one coding sequence as defined herein may be modified compared to the coding sequence of the respective wild type nucleic acid (RNA) sequence, in such a way that codons are exchanged for C-optimized codons comprising additional cytosines and encoding the same amino acid, i.e. the encoded amino acid sequence is preferably not modified as compared to the encoded wild-type amino acid sequence.

According to particularly preferred embodiments, the inventive artificial nucleic acid (RNA) molecule comprises (in addition to the 5′ UTR and 3′ UTR element specified herein) at least one coding sequence as defined herein, wherein (a) the G/C content of the at least one coding sequence of said artificial nucleic acid (RNA) molecule is increased compared to the G/C content of the coding sequence of the corresponding wild-type nucleic acid (RNA), and/or (b) wherein the C content of the at least one coding sequence of said artificial nucleic acid molecule (RNA), is increased compared to the C content of the coding sequence of the corresponding wild-type nucleic acid (RNA), and/or (c) wherein the codons in the at least one coding sequence of said artificial nucleic acid (RNA) molecule are adapted to human codon usage, wherein the codon adaptation index (CAI) is preferably increased or maximized in the at least one coding sequence of said artificial nucleic acid (RNA) molecule, and wherein the amino acid sequence encoded by said artificial nucleic acid (RNA) molecule is preferably not being modified compared to the amino acid sequence encoded by the corresponding wild-type nucleic acid (RNA).

### Modified Nucleic Acid Sequences

The sequence modifications indicated above can in general be applied to any of the nucleic acid (RNA) sequences described herein, and are particularly envisaged to be applied to the coding sequences comprising or consisting of nucleic acid sequences encoding (poly-)peptides or proteins of interest as defined herein. The modifications (including chemical modifications, lipid modifications and sequence modifications) may, if suitable or necessary, be combined with each other in any combination, provided that the combined modifications do not interfere with each other, and preferably provided that the encoded (poly-)peptide or protein of interest is preferably functional, i.e. exhibits a desired biological property or exerts a desired biological function.

Accordingly, in preferred embodiments, artificial nucleic acid (RNA) molecules of the invention comprise at least one coding sequence encoding a (poly-)peptide or protein of interest, wherein said coding sequence has been modified as described above.

Therefore, in some preferred embodiments, artificial nucleic acid (RNA) molecules according to the invention comprise at least one 5′ UTR element as defined herein, at least one 3′ UTR element as defined herein and a coding sequence encoding a (poly-)peptide or protein of interest, wherein said artificial nucleic acid (RNA) molecule comprises or consists of a nucleic acid sequence according to SEQ ID NO: 50-368 or a variant, fragment or derivative of any one of said sequences, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### 5′ Cap

According to further preferred embodiments of the invention, a modified artificial nucleic acid (RNA) molecule, is modified by the addition of a so-called “5′-Cap”, which may preferably stabilize said artificial nucleic acid (RNA) molecule.

A “5′-Cap” is an entity, typically a modified nucleotide entity, which generally “caps” the 5′-end of a mature mRNA. A 5′-cap may typically be formed by a modified nucleotide, particularly by a derivative of a guanine nucleotide. Preferably, the 5′-cap is linked to the 5′-terminus via a 5′-5′-triphosphate linkage. A 5′-cap may be methylated, e.g. m7GpppN, wherein N is the terminal 5′ nucleotide of the nucleic acid carrying the 5′-cap, typically the 5′-end of an mRNA. m7GpppN is the 5′-cap structure, which naturally occurs in mRNA transcribed by polymerase II and is therefore preferably not considered a “modification” comprised in a modified mRNA in this context. Accordingly, a“modified” artificial nucleic acid (RNA) molecule (or any other nucleic acid, in particular RNA, as defined herein) may comprise a m7GpppN as 5′-cap, but additionally said modified artificial nucleic acid (RNA) molecule (or other nucleic acid) typically comprises at least one further modification as defined herein.

Further examples of 5′cap structures include glyceryl, inverted deoxy abasic residue (moiety), 4′, 5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′, 4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety. These modified 5′-cap structures are regarded as at least one modification in this context.

Particularly preferred modified 5′-cap structures are cap1 (methylation of the ribose of the adjacent nucleotide of m7G), cap2 (additional methylation of the ribose of the 2nd nucleotide downstream of the m7G), cap3 (additional methylation of the ribose of the 3rd nucleotide downstream of the m7G), cap4 (methylation of the ribose of the 4th nucleotide downstream of the m7G), ARCA (anti-reverse cap analogue, modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

According to preferred embodiments, the artificial nucleic acid comprises a methyl group at the 2′-O position of the ribose-2′-O position of the first nucleotide adjacent to the cap structure at the 5′ end of the RNA (cap-1). Typically, methylation may be accomplished by the action of Cap 2′-O-Methyltransferase, utilizing m7GpppN capped artificial nucleic acids (preferably RNA) as a substrate and S-adenosylmethionine (SAM) as a methyl donor to methylate capped RNA (cap-0) resulting in the cap-1 structure. The cap-1 structure has been reported to enhance mRNA translation efficiency and hence may help improving expression efficacy of the inventive artificial nucleic acid, preferably RNA, described herein.

### Poly(A)

According to further preferred embodiments, the artificial nucleic acid (RNA) molecule, of the invention may contain a poly(A) sequence.

The term “poly(A) sequence”, also called “poly(A) tail” or “3′-poly(A) tail” means a sequence of adenosine nucleotides, e.g., of up to about 400 adenosine nucleotides, e.g. from about 20 to about 400, preferably from about 50 to about 400, more preferably from about 50 to about 300, even more preferably from about 50 to about 250, most preferably from about 60 to about 250 adenosine nucleotides. As used herein, a “poly(A) sequence” may also comprise about 10 to 200 adenosine nucleotides, preferably about 10 to 100 adenosine nucleotides, more preferably about 40 to 80 adenosine nucleotides or even more preferably about 50 to 70 adenosine nucleotides. A “poly(A) sequence” is typically located at the 3′end of an RNA, in particular a mRNA.

Accordingly, in further preferred embodiments, the artificial nucleic acid (RNA) molecule, of the invention may contain at its 3′ terminus a poly(A) tail of typically about 10 to 200 adenosine nucleotides, preferably about 10 to 100 adenosine nucleotides, more preferably about 40 to 80 adenosine nucleotides or even more preferably about 50 to 70 adenosine nucleotides.

The poly(A) sequence in the artificial nucleic acid (RNA) molecule may preferably originate from a DNA template by RNA in vitro transcription. Alternatively, the poly(A) sequence may also be obtained in vitro by common methods of chemical-synthesis without being necessarily transcribed from a DNA template.

Moreover, “poly(A) sequences”, or “poly(A) tails” may be generated by enzymatic polyadenylation of the artificial nucleic acid (RNA) molecule using commercially available polyadenylation kits and corresponding protocols known in the art. Polyadenylation is typically understood to be the addition of a poly(A) sequence to a nucleic acid (RNA) molecule, e.g. to a premature mRNA. Polyadenylation may be induced by a so-called polyadenylation signal. This signal is preferably located within a stretch of nucleotides at the 3′-end of the nucleic acid (RNA) sequence to be polyadenylated. A polyadenylation signal typically comprises a hexamer consisting of adenine and uracil/thymine nucleotides, preferably the hexamer sequence AAUAAA. Other sequences, preferably hexamer sequences, are also conceivable. Polyadenylation may for instance occur during processing of a pre-mRNA (also called premature-mRNA). Typically, RNA maturation (from pre-mRNA to mature mRNA) comprises a step of polyadenylation.

Accordingly, the artificial nucleic acid (RNA) molecule of the invention may comprise a polyadenylation signal which conveys polyadenylation to a (transcribed) RNA by specific protein factors (e.g. cleavage and polyadenylation specificity factor (CPSF), cleavage stimulation factor (CstF), cleavage factors I and II (CF I and CF II), poly(A) polymerase (PAP)).

In this context, a consensus polyadenylation signal is preferred comprising the NN(U/T)ANA consensus sequence. In a particularly preferred aspect, the polyadenylation signal comprises one of the following sequences: AA(U/T)AAA or A(U/T)(U/T)AAA (wherein uridine is usually present in RNA and thymidine is usually present in DNA).

### Poly(C)

According to some embodiments, the artificial nucleic acid (RNA) molecule, may contain a poly(C) tail on the 3′ terminus of typically about 10 to 200 cytosine nucleotides, preferably about 10 to 100 cytosine nucleotides, more preferably about 20 to 70 cytosine nucleotides or even more preferably about 20 to 60 or even 10 to 40 cytosine nucleotides.

### Histone Stem-Loop (Histone SL or HSL)

According to some embodiments, the artificial nucleic acid (RNA) molecule may comprise a histone stem-loop sequence/structure. Such histone stem-loop sequences are preferably selected from histone stem-loop sequences as disclosed in WO 2012/019780, the disclosure of which is incorporated herewith by reference.

A histone stem-loop sequence, suitable to be used within the present invention, is preferably selected from at least one of the following formulae (I) or (II):

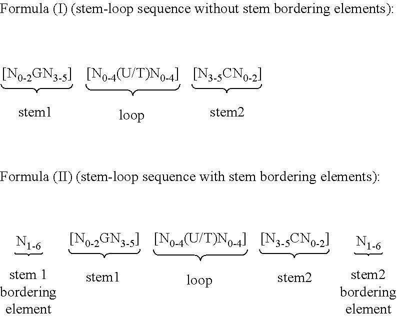

wherein:

| | |
|---|---|
| stem1 or stem2 bordering | is a consecutive sequence of 1 to 6, preferably of 2 to 6, more |
| elements N₁₋₆ | preferably of 2 to 5, even more preferably of 3 to 5, most preferably |
| | of 4 to 5 or 5N, wherein each N is independently from another selected |
| | from a nucleotide selected from A, U, T, G and C, or a nucleotide |
| | analogue thereof; |
| stem1 [N₀₋₂GN₃₋₅] | is reverse complementary or partially reverse complementary with |
| | element stem2, and is a consecutive sequence between of 5 to 7 |
| | nucleotides; |
| | wherein N₀₋₂ is a consecutive sequence of 0 to 2, preferably of 0 to 1, |
| | more preferably of 1N, wherein each N is independently from another |
| | selected from a nucleotide selected from A, U, T, G and C or a |
| | nucleotide analogue thereof; |
| | wherein N₃₋₅ is a consecutive sequence of 3 to 5, preferably of 4 to 5, |
| | more preferably of 4N, wherein each N is independently from another |
| | selected from a nucleotide selected from A, U, T, G and C or a |
| | nucleotide analogue thereof, and |
| | wherein G is guanosine or an analogue thereof, and may be optionally |
| | replaced by a cytidine or an analogue thereof, provided that its |
| | complementary nucleotide cytidine in stem2 is replaced by guanosine; |
| loop | is located between elements stem1 and stem2, and is a consecutive |
| sequence [N₀₋₄(U/T)N₀₋₄] | sequence of 3 to 5 nucleotides, more preferably of 4 nucleotides; |
| | wherein each N₀₋₄ is independent from another a consecutive |
| | sequence of 0 to 4, preferably of 1 to 3, more preferably of 1 to 2N, |
| | wherein each N is independently from another selected from a |
| | nucleotide selected from A, U, T, G and C or a nucleotide analogue |
| | thereof; and |
| | wherein U/T represents uridine, or optionally thymidine; |
| stem2 [N₃₋₅CN₀₋₂] | is reverse complementary or partially reverse complementary with |
| | element stem1, and is a consecutive sequence between of 5 to 7 |
| | nucleotides; |
| | wherein N₃₋₅ is a consecutive sequence of 3 to 5, preferably of 4 to 5, |
| | more preferably of 4N, wherein each N is independently from another |
| | selected from a nucleotide selected from A, U, T, G and C or a |
| | nucleotide analogue thereof; |
| | wherein N₀₋₂ is a consecutive sequence of 0 to 2, preferably of 0 to 1, |
| | more preferably of 1N, wherein each N is independently from another |
| | selected from a nucleotide selected from A, U, T, G or C or a nucleotide |
| | analogue thereof; and |
| | wherein C is cytidine or an analogue thereof, and may be optionally |
| | replaced by a guanosine or an analogue thereof provided that its |
| | complementary nucleoside guanosine in stem1 is replaced by cytidine; |

wherein
stem1 and stem2 are capable of base pairing with each other forming a reverse complementary sequence, wherein base pairing may occur between stem1 and stem2, e.g. by Watson-Crick base pairing of nucleotides A and U/T or G and C or by non-Watson-Crick base pairing e.g. wobble base pairing, reverse Watson-Crick base pairing, Hoogsteen base pairing, reverse Hoogsteen base pairing or are capable of base pairing with each other forming a partially reverse complementary sequence, wherein an incomplete base pairing may occur between stem1 and stem2, on the basis that one or more bases in one stem do not have a complementary base in the reverse complementary sequence of the other stem.

According to further embodiments, the artificial nucleic acid (RNA) molecule of the invention may comprise at least one histone stem-loop sequence according to at least one of the following specific formulae (Ia) or (IIa):

formula (Ia) (stem-loop sequence without stem bordering elements):

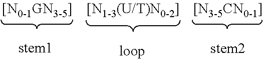

formula (IIa) (stem-loop sequence with stem bordering elements):

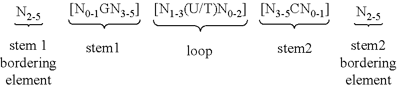

wherein:
N, C, G, T and U are as defined above.

According to further embodiments, the artificial nucleic acid (RNA) molecule of the invention may comprise at least one histone stem-loop sequence according to at least one of the following specific formulae (Ib) or (IIb):

formula (Ib) (stem-loop sequence without stem bordering elements):

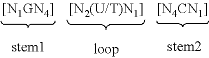

formula (IIb) (stem-loop sequence with stem bordering elements):

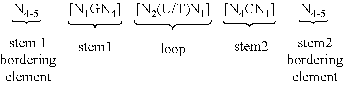

wherein:
N, C, G, T and U are as defined above.

A particularly preferred histone stem-loop sequence is the sequence CAAAGGCTCTTTTCAGAGCCACCA (SEQ ID NO: 37) or more preferably the corresponding RNA sequence CAAAGGCUCUUUUCAGAGCCACCA (SEQ ID NO: 38).

### Constructs

The artificial nucleic acid (RNA) molecule of the invention, which comprises at least one 5′ UTR element, at least one 3′ UTR element and optionally at least one coding sequence as defined herein, may optionally further comprise at least one histone stem-loop, poly(A) and/or poly(C) sequence. The elements may occur therein in any order from 5′ to 3′ along the sequence of the artificial nucleic acid (RNA) molecule.

In addition, the artificial nucleic acid (RNA) molecule of the invention may comprise further elements as described herein, such as a stabilizing sequence as defined herein (e.g. derived from the UTR of a globin gene), IRES sequences, etc. Each of the elements may also be repeated in the artificial nucleic acid (RNA) molecule, of the invention at least once (particularly in di- or multicistronic constructs), e.g. twice or more. As an example, the individual elements may be present in the artificial nucleic acid (RNA) molecule, preferably RNA, of the invention in the following order:

5′-coding sequence-histone stem-loop-poly(A)/(C) sequence-3′; or
5′-coding sequence-poly(A)/(C) sequence-histone stem-loop-3′; or
5′-coding sequence-histone stem-loop-polyadenylation signal-3′; or
5′-coding sequence-polyadenylation signal-histone stem-loop-3′; or
5′-coding sequence-histone stem-loop-histone stem-loop-poly(A)/(C) sequence-3′; or
5′-coding sequence-histone stem-loop-histone stem-loop-polyadenylation signal-3′; or
5′-coding sequence-stabilizing sequence-poly(A)/(C) sequence-histone stem-loop-3′; or
5′-coding sequence-stabilizing sequence-poly(A)/(C) sequence-poly(A)/(C) sequence-histone stem-loop-3′; etc.

According to further embodiments, the artificial nucleic acid (RNA) molecule of the invention may optionally further comprises at least one of the following structural elements: a histone-stem-loop structure, preferably a histone-stem-loop in its 3′ untranslated region; a 5′-cap structure; a poly-A tail; and/or a poly(C) sequence.

Specifically, artificial nucleic acid (RNA) molecules of to the invention may comprise preferably in 5′ to 3′ direction, the following elements:
a) a 5′-CAP structure, preferably m7GpppN or Cap1
b) a 5′-UTR element, which comprises or consists of a nucleic acid sequence, which is derived from a 5′-UTR as defined herein, preferably comprising a nucleic acid sequence corresponding to the nucleic acid sequence according to SEQ ID NO: 1-22 or a homolog, fragment or variant thereof;
c) at least one coding sequence as defined herein;
d) a 3′-UTR element, which comprises or consists of a nucleic acid sequence, which is derived from a 3′-UTR as defined herein, preferably comprising a nucleic acid sequence corresponding to the nucleic acid sequence according to SEQ ID NO: 23-36, or a homolog, a fragment or a variant thereof,
e) optionally a poly(A) tail, preferably consisting of 10 to 1000, 10 to 500, 10 to 300 10 to 200, 10 to 100, 40 to 80 or 50 to 70 adenosine nucleotides,
f) optionally a poly(C) tail, preferably consisting of 10 to 200, 10 to 100, 20 to 70, 20 to 60 or 10 to 40 cytosine nucleotides, and
g) optionally a histone stem-loop.

Preferred artificial nucleic acid constructs are discussed in detail below.

### HSD17B4-Derived 5′ UTR Element and PSMB3-Derived 3′ UTR Element:

In some preferred embodiments, artificial nucleic acid (RNA) molecules of the invention comprise at least one 5′ UTR element derived from a 5′UTR of a HSD17B4 gene, or from a homolog, fragment, variant or derivative thereof and at least one 3′ UTR element derived from a 3′UTR of a GNAS gene, or from a homolog, fragment, variant or derivative thereof; wherein said artificial nucleic acid (RNA) molecule preferably comprises or consists of a nucleic acid sequence according to any one of SEQ ID NOs: 54-60, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### NDUFA4-Derived 5′ UTR Element and PSMB3-Derived 3′UTR Element:

In some preferred embodiments, artificial nucleic acid (RNA) molecules of the invention comprise at least one 5′ UTR element derived from a 5′UTR of a NDUFA4 gene, or from a homolog, fragment, variant or derivative thereof and at least one 3′ UTR element derived from a 3′UTR of a PSMB3 gene, or from a homolog, fragment, variant or derivative thereof; wherein said artificial nucleic acid (RNA) molecule preferably comprises or consists of a nucleic acid sequence according to any one of SEQ ID NOs: 188-193, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### SLC7A3-Derived 5′ UTR Element and PSMB3-Derived 3′ UTR Element:

In some preferred embodiments, artificial nucleic acid (RNA) molecules of the invention comprise at least one 5′ UTR element derived from a 5′UTR of a SLC7A3 gene, or from a homolog, fragment, variant or derivative thereof and at least one 3′ UTR element derived from a 3′UTR of a PSMB3 gene, or from a homolog, fragment, variant or derivative thereof; wherein said artificial nucleic acid (RNA) molecule preferably comprises or consists of a nucleic acid sequence according to any one of SEQ ID NOs: 313-319, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### NOSIP-Derived 5′ UTR Element and PSMB3-Derived 3′ UTR Element:

In some preferred embodiments, artificial nucleic acid (RNA) molecules of the invention comprise at least one 5′ UTR element derived from a 5′UTR of a NOSIP gene, or from a homolog, fragment, variant or derivative thereof and at least one 3′ UTR element derived from a 3′UTR of a PSMB3 gene, or from a homolog, fragment, variant or derivative thereof; wherein said artificial nucleic acid (RNA) molecule preferably comprises or consists of a nucleic acid sequence according to any one of SEQ ID NOs: 229-235, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### NOSIP-Derived 5′ UTR Element and GNAS-Derived 3′ UTR Element:

In some preferred embodiments, artificial nucleic acid (RNA) molecules of the invention comprise at least one 5′ UTR element derived from a 5′UTR of a NOSIP gene, or from a homolog, fragment, variant or derivative thereof and at least one 3′ UTR element derived from a 3′UTR of a GNAS gene, or from a homolog, fragment, variant or derivative thereof; wherein said artificial nucleic acid (RNA) molecule preferably comprises or consists of a nucleic acid sequence according to any one of SEQ ID NOs: 250-256, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### MP68-Derived 5′ UTR Element and PSMB3-Derived 3′ UTR Element:

In some preferred embodiments, artificial nucleic acid (RNA) molecules of the invention comprise at least one 5′ UTR element derived from a 5′UTR of a MP68 gene, or from a homolog, fragment, variant or derivative thereof and at least one 3′ UTR element derived from a 3′UTR of a PSMB3 gene, or from a homolog, fragment, variant or derivative thereof; wherein said artificial nucleic acid (RNA) molecule preferably comprises or consists of a nucleic acid sequence according to any one of SEQ ID NOs: 145-151, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### MP68-Derived 5′ UTR Element and CASP1-Derived 3′ UTR Element:

In some preferred embodiments, artificial nucleic acid (RNA) molecules of the invention comprise at least one 5′ UTR element derived from a 5′UTR of a MP68 gene, or from a homolog, fragment, variant or derivative thereof and at least one 3′ UTR element derived from a 3′UTR of a CASP1 gene, or from a homolog, fragment, variant or derivative thereof; wherein said artificial nucleic acid (RNA) molecule preferably comprises or consists of a nucleic acid sequence according to any one of SEQ ID NOs: 152-158, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### MP68-Derived 5′ UTR Element and GNAS-Derived 3′ UTR Element:

In some preferred embodiments, artificial nucleic acid (RNA) molecules of the invention comprise at least one 5′ UTR element derived from a 5′UTR of a MP68 gene, or from a homolog, fragment, variant or derivative thereof and at least one 3′ UTR element derived from a 3′UTR of a GNAS gene, or from a homolog, fragment, variant or derivative thereof; wherein said artificial nucleic acid (RNA) molecule preferably comprises or consists of a nucleic acid sequence according to any one of SEQ ID NOs: 166-172, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### UBQLN2-Derived 5′ UTR Element and RPS9-Derived 3′ UTR Element:

In some preferred embodiments, artificial nucleic acid (RNA) molecules of the invention comprise at least one 5′ UTR element derived from a 5′UTR of a UBQLN2 gene, or from a homolog, fragment, variant or derivative thereof and at least one 3′ UTR element derived from a 3′UTR of a RPS9 gene, or from a homolog, fragment, variant or derivative thereof; wherein said artificial nucleic acid (RNA) molecule preferably comprises or consists of a nucleic acid sequence according to any oen of SEQ ID NOs: 362-368, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### ASAH1-Derived 5′ UTR Element and RPS9-Derived 3′ UTR Element:

In some preferred embodiments, artificial nucleic acid (RNA) molecules of the invention comprise at least one 5′ UTR element derived from a 5′UTR of a ASAH1 gene, or from a homolog, fragment, variant or derivative thereof and at least one 3′ UTR element derived from a 3′UTR of a RPS9 gene, or from a homolog, fragment, variant or derivative thereof; wherein said artificial nucleic acid (RNA) molecule preferably comprises or consists of a nucleic acid sequence according to any one of SEQ ID NOs: 96-102, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### HSD17B4-Derived 5′ UTR Element and RPS9-Derived 3′ UTR Element:

In some preferred embodiments, artificial nucleic acid (RNA) molecules of the invention comprise at least one 5′ UTR element derived from a 5′UTR of a HSD17B4 gene, or from a homolog, fragment, variant or derivative thereof and at least one 3′ UTR element derived from a 3′UTR of a RPS9 gene, or from a homolog, fragment, variant or derivative thereof; wherein said artificial nucleic acid (RNA) molecule preferably comprises or consists of a nucleic acid sequence according to any one of SEQ ID NOs: 89-95, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### HSD17B4-Derived 5′ UTR Element and CASP1-Derived 3′ UTR Element:

In some preferred embodiments, artificial nucleic acid (RNA) molecules of the invention comprise at least one 5′ UTR element derived from a 5′UTR of a HSD17B4 gene, or from a homolog, fragment, variant or derivative thereof and at least one 3′ UTR element derived from a 3′UTR of a CASP1 gene, or from a homolog, fragment, variant or derivative thereof; wherein said artificial nucleic acid (RNA) molecule preferably comprises or consists of a nucleic acid sequence according to any one of SEQ ID NOs: 61-67, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### NOSIP-Derived 5′ UTR Element and COX6B1-Derived 3′ UTR Element:

In some preferred embodiments, artificial nucleic acid (RNA) molecules of the invention comprise at least one 5′ UTR element derived from a 5′UTR of a NOSIP gene, or from a homolog, fragment, variant or derivative thereof and at least one 3′ UTR element derived from a 3′UTR of a COX6B1 gene, or from a homolog, fragment, variant or derivative thereof; wherein said artificial nucleic acid (RNA) molecule preferably comprises or consists of a nucleic acid sequence according to any one of SEQ ID Nos: 243-249, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### NDUFA4-Derived 5′ UTR Element and RPS9-Derived 3′ UTR Element:

In some preferred embodiments, artificial nucleic acids according to the invention comprise at least one 5′ UTR element derived from a 5′UTR of a NDUFA4 gene, or from a homolog, fragment, variant or derivative thereof and at least one 3′ UTR element derived from a 3′UTR of a RPS9 gene, or from a homolog, fragment, variant or derivative thereof, wherein said artificial nucleic acid comprises or consists of a nucleic acid sequence according to any one of SEQ ID NOs: 222-228, or a homolog, variant, fragment or derivative thereof, in particular a nucleic acid sequence in having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### NOSIP-Derived 5′ UTR Element and NDUFA1-Derived 3′ UTR Element:

In some preferred embodiments, artificial nucleic acid (RNA) molecules of the invention comprise at least one 5′ UTR element derived from a 5′UTR of a NOSIP gene, or from a homolog, fragment, variant or derivative thereof and at least one 3′ UTR element derived from a 3′UTR of a NDUFA1 gene, or from a homolog, fragment, variant or derivative thereof; wherein said artificial nucleic acid (RNA) molecule preferably comprises or consists of a nucleic acid sequence according to any one of SEQ ID NOs: 257-263, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### NDUFA4-Derived 5′ UTR Element and COX6B1-Derived 3′ UTR Element:

In some preferred embodiments, artificial nucleic acid (RNA) molecules of the invention comprise at least one 5′ UTR element derived from a 5′UTR of a NDUFA4 gene, or from a homolog, fragment, variant or derivative thereof and at least one 3′ UTR element derived from a 3′UTR of a COX6B1 gene, or from a homolog, fragment, variant or derivative thereof; wherein said artificial nucleic acid (RNA) molecule preferably comprises or consists of a nucleic acid sequence according to any one of SEQ ID NOs: 201-207, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### NDUFA4-Derived 5′ UTR Element and NDUFA1-Derived 3′ UTR Element:

In some preferred embodiments, artificial nucleic acid (RNA) molecules of the invention comprise at least one 5′ UTR element derived from a 5′UTR of a NDUFA4 gene, or from a homolog, fragment, variant or derivative thereof and at least one 3′ UTR element derived from a 3′UTR of a NDUFA1 gene, or from a homolog, fragment, variant or derivative thereof; wherein said artificial nucleic acid (RNA) molecule preferably comprises or consists of a nucleic acid sequence according to any one of SEQ ID NOs: 215-221, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### ATP5A1-Derived 5′ UTR Element and CASP1-Derived 3′ UTR Element:

In some preferred embodiments, artificial nucleic acid (RNA) molecules of the invention comprise at least one 5′ UTR element derived from a 5′UTR of a ATP5A1 gene, or from a homolog, fragment, variant or derivative thereof and at least one 3′ UTR element derived from a 3′UTR of a CASP1 gene, or from a homolog, fragment, variant or derivative thereof; wherein said artificial nucleic acid (RNA) molecule preferably comprises or consists of a nucleic acid sequence according to any one of SEQ ID NOs: 110-116, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### SLC7A3-Derived 5′ UTR Element and GNAS-Derived 3′ UTR Element:

In some preferred embodiments, artificial nucleic acid (RNA) molecules of the invention comprise at least one 5′ UTR element derived from a 5′UTR of a SLC7A3 gene, or from a homolog, fragment, variant or derivative thereof and at least one 3′ UTR element derived from a 3′UTR of a GNAS gene, or from a homolog, fragment, variant or derivative thereof; wherein said artificial nucleic acid (RNA) molecule preferably comprises or consists of a nucleic acid sequence according to any one of SEQ ID NOs: 334-340, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### HSD17B4-Derived 5′ UTR Element and NDUFA1-Derived 3′ UTR Element:

In some preferred embodiments, artificial nucleic acid (RNA) molecules of the invention comprise at least one 5′ UTR element derived from a 5′UTR of a HSD17B4 gene, or from a homolog, fragment, variant or derivative thereof and at least one 3′ UTR element derived from a 3′UTR of a NDUFA1 gene, or from a homolog, fragment, variant or derivative thereof; wherein said artificial nucleic acid (RNA) molecule preferably comprises or consists of a nucleic acid sequence according to any one of SEQ ID NOs: 82-88, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### SLC7A3-Derived 5′ UTR Element and NDUFA1-Derived 3′ UTR Element:

In some preferred embodiments, artificial nucleic acid (RNA) molecules of the invention comprise at least one 5′ UTR element derived from a 5′UTR of a SLC7A3 gene, or from a homolog, fragment, variant or derivative thereof and at least one 3′ UTR element derived from a 3′UTR of a NDUFA1 gene, or from a homolog, fragment, variant or derivative thereof; wherein said artificial nucleic acid (RNA) molecule preferably comprises or consists of a nucleic acid sequence according to any one of SEQ ID NOs: 341-347, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### SLC7A3-Derived 5′ UTR Element and RPS9-Derived 3′ UTR Element:

In some preferred embodiments, artificial nucleic acid (RNA) molecules of the invention comprise at least one 5′ UTR element derived from a 5′UTR of a SLC7A3 gene, or from a homolog, fragment, variant or derivative thereof and at least one 3′ UTR element derived from a 3′UTR of a RPS9 gene, or from a homolog, fragment, variant or derivative thereof; wherein said artificial nucleic acid (RNA) molecule preferably comprises or consists of a nucleic acid sequence according to any one of SEQ ID NOs: 348-354, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### TUBB4B-Derived 5′ UTR Element and RPS9-Derived 3′ UTR Element:

In some preferred embodiments, artificial nucleic acid (RNA) molecules of the invention comprise at least one 5′ UTR element derived from a 5′UTR of a TUBB4B gene, or from a homolog, fragment, variant or derivative thereof and at least one 3′ UTR element derived from a 3′UTR of a RPS9 gene, or from a homolog, fragment, variant or derivative thereof; wherein said artificial nucleic acid (RNA) molecule preferably comprises or consists of a nucleic acid sequence according to any one of SEQ ID NOs: 355-361, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### RPL31-Derived 5′ UTR Element and RPS9-Derived 3′ UTR Element:

In some preferred embodiments, artificial nucleic acid (RNA) molecules of the invention comprise at least one 5′ UTR element derived from a 5′UTR of a RPL31 gene, or from a homolog, fragment, variant or derivative thereof and at least one 3′ UTR element derived from a 3′UTR of a RPS9 gene, or from a homolog, fragment, variant or derivative thereof; wherein said artificial nucleic acid (RNA) molecule preferably comprises or consists of a nucleic acid sequence according to any one of SEQ ID NOs: 306-312, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### MP68-Derived 5′ UTR Element and RPS9-Derived 3′ UTR Element:

In some preferred embodiments, artificial nucleic acid (RNA) molecules of the invention comprise at least one 5′ UTR element derived from a 5′UTR of a MP68 gene, or from a homolog, fragment, variant or derivative thereof and at least one 3′ UTR element derived from a 3′UTR of a RPS9 gene, or from a homolog, fragment, variant or derivative thereof; wherein said artificial nucleic acid (RNA) molecule preferably comprises or consists of a nucleic acid sequence according to any one of SEQ ID NOs: 180-187, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### NOSIP-Derived 5′ UTR Element and RPS9-Derived 3′ UTR Element:

In some preferred embodiments, artificial nucleic acid (RNA) molecules of the invention comprise at least one 5′ UTR element derived from a 5′UTR of a NOSIP gene, or from a homolog, fragment, variant or derivative thereof and at least one 3′ UTR element derived from a 3′UTR of a RPS9 gene, or from a homolog, fragment, variant or derivative thereof; wherein said artificial nucleic acid (RNA) molecule preferably comprises or consists of a nucleic acid sequence according to any one of SEQ ID NOs: 264-270, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### ATP5A1-Derived 5′ UTR Element and RPS9-Derived 3′ UTR Element:

In some preferred embodiments, artificial nucleic acid (RNA) molecules of the invention comprise at least one 5′ UTR element derived from a 5′UTR of a ATP5A1 gene, or from a homolog, fragment, variant or derivative thereof and at least one 3′ UTR element derived from a 3′UTR of a RPS9 gene, or from a homolog, fragment, variant or derivative thereof; wherein said artificial nucleic acid (RNA) molecule preferably comprises or consists of a nucleic acid sequence according to any one of SEQ ID NOs: 138-144, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### ATP5A1-Derived 5′ UTR Element and COX6B1-Derived 3′ UTR Element:

In some preferred embodiments, artificial nucleic acid (RNA) molecules of the invention comprise at least one 5′ UTR element derived from a 5′UTR of a ATP5A1 gene, or from a homolog, fragment, variant or derivative thereof and at least one 3′ UTR element derived from a 3′UTR of a COX6B1 gene, or from a homolog, fragment, variant or derivative thereof; wherein said artificial nucleic acid (RNA) molecule preferably comprises or consists of a nucleic acid sequence according to any one of SEQ ID NOs: 117-123, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### ATP5A1-Derived 5′ UTR Element and GNAS1-Derived 3′ UTR Element:

In some preferred embodiments, artificial nucleic acid (RNA) molecules of the invention comprise at least one 5′ UTR element derived from a 5′UTR of a ATP5A1 gene, or from a homolog, fragment, variant or derivative thereof and at least one 3′ UTR element derived from a 3′UTR of a GNAS1 gene, or from a homolog, fragment, variant or derivative thereof; wherein said artificial nucleic acid (RNA) molecule preferably comprises or consists of a nucleic acid sequence according to any one of SEQ ID NOs: 124-130, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### ATP5A1-Derived 5′ UTR Element and NDUFA1-Derived 3′ UTR Element:

In some preferred embodiments, artificial nucleic acid (RNA) molecules of the invention comprise at least one 5′ UTR element derived from a 5′UTR of a ATP5A1 gene, or from a homolog, fragment, variant or derivative thereof and at least one 3′ UTR element derived from a 3′UTR of a NDUFA1 gene, or from a homolog, fragment, variant or derivative thereof; wherein said artificial nucleic acid (RNA) molecule preferably comprises or consists of a nucleic acid sequence according to any one of SEQ ID NOs: 131-137, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### ATP5A1-Derived 5′ UTR Element and PSMB3-Derived 3′ UTR Element:

In some preferred embodiments, artificial nucleic acid (RNA) molecules of the invention comprise at least one 5′ UTR element derived from a 5′UTR of a ATP5A1 gene, or from a homolog, fragment, variant or derivative thereof and at least one 3′ UTR element derived from a 3′UTR of a PSMB3 gene, or from a homolog, fragment, variant or derivative thereof; wherein said artificial nucleic acid (RNA) molecule preferably comprises or consists of a nucleic acid sequence according to any one of SEQ ID NOs: 103-109, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### HSD17B4-Derived 5′ UTR Element and COX6B1-Derived 3′ UTR Element:

In some preferred embodiments, artificial nucleic acid (RNA) molecules of the invention comprise at least one 5′ UTR element derived from a 5′UTR of a HSD17B4 gene, or from a homolog, fragment, variant or derivative thereof and at least one 3′ UTR element derived from a 3′UTR of a COX6B1 gene, or from a homolog, fragment, variant or derivative thereof; wherein said artificial nucleic acid (RNA) molecule preferably comprises or consists of a nucleic acid sequence according to any one of SEQ ID NOs: 68-74, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### HSD17B4-Derived 5′ UTR Element and GNAS1-Derived 3′ UTR Element:

In some preferred embodiments, artificial nucleic acid (RNA) molecules of the invention comprise at least one 5′ UTR element derived from a 5′UTR of a HSD17B4 gene, or from a homolog, fragment, variant or derivative thereof and at least one 3′ UTR element derived from a 3′UTR of a GNAS1 gene, or from a homolog, fragment, variant or derivative thereof; wherein said artificial nucleic acid (RNA) molecule preferably comprises or consists of a nucleic acid sequence according to any one of SEQ ID NOs: 75-81, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### MP68-Derived 5′ UTR Element and COX6B1-Derived 3′ UTR Element:

In some preferred embodiments, artificial nucleic acid (RNA) molecules of the invention comprise at least one 5′ UTR element derived from a 5′UTR of a MP68 gene, or from a homolog, fragment, variant or derivative thereof and at least one 3′ UTR element derived from a 3′UTR of a COX6B1 gene, or from a homolog, fragment, variant or derivative thereof; wherein said artificial nucleic acid (RNA) molecule preferably comprises or consists of a nucleic acid sequence according to any one of SEQ ID NOs: 159-165, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### MP68-Derived 5′ UTR Element and NDUFA1-Derived 3′ UTR Element:

In some preferred embodiments, artificial nucleic acid (RNA) molecules of the invention comprise at least one 5′ UTR element derived from a 5′UTR of a MP68 gene, or from a homolog, fragment, variant or derivative thereof and at least one 3′ UTR element derived from a 3′UTR of a NDUFA1 gene, or from a homolog, fragment, variant or derivative thereof; wherein said artificial nucleic acid (RNA) molecule preferably comprises or consists of a nucleic acid sequence according to any one of SEQ ID NOs: 173-179, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### NDUFA4-Derived 5′ UTR Element and CASP1-Derived 3′ UTR Element:

In some preferred embodiments, artificial nucleic acid (RNA) molecules of the invention comprise at least one 5′ UTR element derived from a 5′UTR of a NDUFA4 gene, or from a homolog, fragment, variant or derivative thereof and at least one 3′ UTR element derived from a 3′UTR of a CASP1 gene, or from a homolog, fragment, variant or derivative thereof; wherein said artificial nucleic acid (RNA) molecule preferably comprises or consists of a nucleic acid sequence according to any one of SEQ ID NOs: 194-200, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### NDUFA4-Derived 5′ UTR Element and GNAS1-Derived 3′ UTR Element:

In some preferred embodiments, artificial nucleic acid (RNA) molecules of the invention comprise at least one 5′ UTR element derived from a 5′UTR of a NDUFA4 gene, or from a homolog, fragment, variant or derivative thereof and at least one 3′ UTR element derived from a 3′UTR of a GNAS1 gene, or from a homolog, fragment, variant or derivative thereof; wherein said artificial nucleic acid (RNA) molecule preferably comprises or consists of a nucleic acid sequence according to any one of SEQ ID NOs: 208-214, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### NOSIP-Derived 5′ UTR Element and CASP1-Derived 3′ UTR Element:

In some preferred embodiments, artificial nucleic acid (RNA) molecules of the invention comprise at least one 5′ UTR element derived from a 5′UTR of a NOSIP gene, or from a homolog, fragment, variant or derivative thereof and at least one 3′ UTR element derived from a 3′UTR of a CASP1 gene, or from a homolog, fragment, variant or derivative thereof; wherein said artificial nucleic acid (RNA) molecule preferably comprises or consists of a nucleic acid sequence according to any one of SEQ ID NOs: 236-242, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### RPL31-Derived 5′ UTR Element and CASP1-Derived 3′ UTR Element:

In some preferred embodiments, artificial nucleic acid (RNA) molecules of the invention comprise at least one 5′ UTR element derived from a 5′UTR of a RPL31 gene, or from a homolog, fragment, variant or derivative thereof and at least one 3′ UTR element derived from a 3′UTR of a CASP1 gene, or from a homolog, fragment, variant or derivative thereof; wherein said artificial nucleic acid (RNA) molecule preferably comprises or consists of a nucleic acid sequence according to any one of SEQ ID NOs: 278-284, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### RPL31-Derived 5′ UTR Element and COX6B1-Derived 3′ UTR Element:

In some preferred embodiments, artificial nucleic acid (RNA) molecules of the invention comprise at least one 5′ UTR element derived from a 5′UTR of a RPL31 gene, or from a homolog, fragment, variant or derivative thereof and at least one 3′ UTR element derived from a 3′UTR of a COX6B1 gene, or from a homolog, fragment, variant or derivative thereof; wherein said artificial nucleic acid (RNA) molecule preferably comprises or consists of a nucleic acid sequence according to any one of SEQ ID NOs: 285-291, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### RPL31-Derived 5′ UTR Element and GNAS1-Derived 3′ UTR Element:

In some preferred embodiments, artificial nucleic acid (RNA) molecules of the invention comprise at least one 5′ UTR element derived from a 5′UTR of a RPL31 gene, or from a homolog, fragment, variant or derivative thereof and at least one 3′ UTR element derived from a 3′UTR of a GNAS1 gene, or from a homolog, fragment, variant or derivative thereof; wherein said artificial nucleic acid (RNA) molecule preferably comprises or consists of a nucleic acid sequence according to any one fo SEQ ID NOs: 292-298, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### RPL31-Derived 5′ UTR Element and NDUFA1-Derived 3′ UTR Element:

In some preferred embodiments, artificial nucleic acid (RNA) molecules of the invention comprise at least one 5′ UTR element derived from a 5′UTR of a RPL31 gene, or from a homolog, fragment, variant or derivative thereof and at least one 3′ UTR element derived from a 3′UTR of a NDUFA1 gene, or from a homolog, fragment, variant or derivative thereof; wherein said artificial nucleic acid (RNA) molecule preferably comprises or consists of a nucleic acid sequence according to any one of SEQ ID NOs: 299-305, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### SLC7A3-Derived 5′ UTR Element and CASP1-Derived 3′ UTR Element:

In some preferred embodiments, artificial nucleic acid (RNA) molecules of the invention comprise at least one 5′ UTR element derived from a 5′UTR of a SLC7A3 gene, or from a homolog, fragment, variant or derivative thereof and at least one 3′ UTR element derived from a 3′UTR of a CASP1 gene, or from a homolog, fragment, variant or derivative thereof; wherein said artificial nucleic acid (RNA) molecule preferably comprises or consists of a nucleic acid sequence according to any one of SEQ ID NOs: 320-326, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### SLC7A3-Derived 5′ UTR Element and COX6B1-Derived 3′ UTR Element:

In some preferred embodiments, artificial nucleic acid (RNA) molecules of the invention comprise at least one 5′ UTR element derived from a 5′UTR of a SLC7A3 gene, or from a homolog, fragment, variant or derivative thereof and at least one 3′ UTR element derived from a 3′UTR of a COX6B1 gene, or from a homolog, fragment, variant or derivative thereof; wherein said artificial nucleic acid (RNA) molecule preferably comprises or consists of a nucleic acid sequence according to any one of SEQ ID NOs: 327-333, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### RPL31-Derived 5′ UTR Element and PSMB3-Derived 3′ UTR Element:

In some preferred embodiments, artificial nucleic acid (RNA) molecules of the invention comprise at least one 5′ UTR element derived from a 5′UTR of a RPL31 gene, or from a homolog, fragment, variant or derivative thereof and at least one 3′ UTR element derived from a 3′UTR of a PSMB3 gene, or from a homolog, fragment, variant or derivative thereof; wherein said artificial nucleic acid (RNA) molecule preferably comprises or consists of a nucleic acid sequence according to any one of SEQ ID NOs: 271-277, or a homolog, variant, fragment or derivative thereof, in particular a nucleic acid sequence having, in increasing order of preference, at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, sequence identity to any of these sequences.

### Complexation

In preferred embodiments, at least one artificial nucleic acid (RNA) molecule of the invention may be provided in a complexed form, i.e. complexed or associated with one or more (poly-)cationic compounds, preferably with (poly-)cationic polymers, (poly-)cationic peptides or proteins, e.g. protamine, (poly-)cationic polysaccharides and/or (poly-)cationic lipids. In this context, the terms “complexed” or “associated” refer to the essentially stable combination of the at least one artificial nucleic acid (RNA) molecule with one or more of the aforementioned compounds into larger complexes or assemblies, typically without covalent binding.

### Lipids

According to preferred embodiments, the artificial nucleic acid (RNA) molecule of the invention, is complexed or associated with lipids (in particular cationic and/or neutral lipids) to form one or more liposomes, lipoplexes, lipid nanoparticles, or nanoliposomes.

Therefore, in some embodiments, the artificial nucleic acid (RNA) molecule of the invention may be provided in the form of a lipid-based formulation, in particular in the form of liposomes, lipoplexes, and/or lipid nanoparticles comprising said artificial nucleic acid (RNA) molecule.

### Lipid Nanoparticles

According to some preferred embodiments, the artificial nucleic acid (RNA) molecule of the invention, is complexed or associated with lipids (in particular cationic and/or neutral lipids) to form one or more lipid nanoparticles.

Preferably, lipid nanoparticles (LNPs) may comprise: (a) at least one artificial nucleic acid (RNA) molecule of the invention, (b) a cationic lipid, (c) an aggregation reducing agent (such as polyethylene glycol (PEG) lipid or PEG-modified lipid), (d) optionally a non-cationic lipid (such as a neutral lipid), and (e) optionally, a sterol.

In some embodiments, LNPs may comprise, in addition to the at least one artificial nucleic acid (RNA) molecule of the invention, (i) at least one cationic lipid; (ii) a neutral lipid; (iii) a sterol, e.g., cholesterol; and (iv) a PEG-lipid, in a molar ratio of about 20-60% cationic lipid: 5-25% neutral lipid: 25-55% sterol; 0.5-15% PEG-lipid.

In some embodiments, the artificial nucleic acid (RNA) molecule of the invention may be formulated in an aminoalcohol lipidoid. Aminoalcohol lipidoids which may be used in the present invention may be prepared by the methods described in U.S. Pat. No. 8,450,298, herein incorporated by reference in its entirety.

### (i) Cationic Lipids

LNPs may include any cationic lipid suitable for forming a lipid nanoparticle. Preferably, the cationic lipid carries a net positive charge at about physiological pH.

The cationic lipid may be an amino lipid. As used herein, the term “amino lipid” is meant to include those lipids having one or two fatty acid or fatty alkyl chains and an amino head group (including an alkylamino or dialkylamino group) that may be protonated to form a cationic lipid at physiological pH.

The cationic lipid may be, for example, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-dimethylamino)butanoate (MC3), 1,1′-(2-(4-(2-((2-(bis(2-hydroxydodecyl)amino)ethyl) (2-hydroxydodecyl)amino)ethyl)piperazin-1-yl) ethylazanediyl)didodecan-2-ol (C₁₂₋₂₀₀), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), (6Z,9Z,28Z,31Z)-heptatriaconta-6,9,28,31-tetraen-19-yl-4-(dimethylamino)butanoate (DLin-M-C3-DMA), 3-((6Z,9Z,28Z,31Z)-heptatriaconta-6,9,28,3-1-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), or any combination of any of the foregoing.

Other suitable cationic lipids include, but are not limited to, N,N-distearyl-N,N-dimethylammonium bromide (DDAB), 3P—(N—(N′,N′-dimethylaminoethane)-carbamoyl)cholesterol (DC-Chol), N—(I-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), l,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and LIPOFECTAMINE (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. No. 8,158,601; and Love et al, PNAS, 107(5), 1864-69, 2010.

Other suitable amino lipids include those having alternative fatty acid groups and other dialkylamino groups, including those in which the alkyl substituents are different (e.g., N-ethyl-N-methylamino-, and N-propyl-N-ethylamino-). In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C₁₄ to C₂₂ may be used. Other scaffolds can also be used to separate the amino group and the fatty acid or fatty alkyl portion of the amino lipid.

In a further preferred embodiment, the LNP comprises the cationic lipid with formula (III) according to the patent application PCT/EP2017/064066. In this context, the disclosure of PCT/EP2017/064066 is also incorporated herein by reference.

In some embodiments, amino or cationic lipids have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the invention.

In some embodiments, the protonatable lipids have a pKa of the protonatable group in the range of about 4 to about 11, e.g., a pKa of about 5 to about 7.

LNPs may include two or more cationic lipids. The cationic lipids may be selected to contribute different advantageous properties. For example, cationic lipids that differ in properties such as amine pKa, chemical stability, half-life in circulation, half-life in tissue, net accumulation in tissue, or toxicity may be used in the LNP. In particular, the cationic lipids may be chosen so that the properties of the mixed-LNP are more desirable than the properties of a single-LNP of individual lipids.

In some embodiments, the cationic lipid is present in a ratio of from about 20 mol % to about 70 or 75 mol % or from about 45 to about 65 mol % or about 20, 25, 30, 35, 40, 45, 50, 55, 60, 65, or about 70 mol % of the total lipid present in the LNP. In further embodiments, the LNPs comprise from about 25% to about 75% on a molar basis of cationic lipid, e.g., from about 20 to about 70%, from about 35 to about 65%, from about 45 to about 65%, about 60%, about 50% or about 40% on a molar basis (based upon 100% total moles of lipid in the lipid nanoparticle). In some embodiments, the ratio of cationic lipid to nucleic acid is from about 3 to about 15, such as from about 5 to about 13 or from about 7 to about 11.

In some embodiments, the liposome may have a molar ratio of nitrogen atoms in the cationic lipid to the phosphates in the RNA (N:P ratio) of between 1:1 and 20:1 as described in International Publication No. WO 2013/006825 A1, herein incorporated by reference in its entirety. In other embodiments, the liposome may have an N:P ratio of greater than 20:1 or less than 1:1.

### (ii) Neutral and Non-Cationic Lipids

The “non-cationic lipid” may be a neutral lipid, an anionic lipid, or an amphipathic lipid.

Neutral lipids may be any of a number of lipid species which exist either in an uncharged or neutral zwitterionic form at physiological pH. Such lipids include, for example, diacylphosphatidylcholine, diacylphosphatidylethanolamine, ceramide, sphingomyelin, dihydrosphingomyelin, cephalin, and cerebrosides. The selection of neutral lipids for use in the LNPs described herein is generally guided by consideration of, e.g., LNP size and stability of the LNP in the bloodstream. Preferably, the neutral lipid may be a lipid having two acyl groups (e.g., diacylphosphatidylcholine and diacylphosphatidylethanolamine).

In some embodiments, the neutral lipids contain saturated fatty acids with carbon chain lengths in the range of C₁₀ to C₂₀. In other embodiments, neutral lipids with mono or diunsaturated fatty acids with carbon chain lengths in the range of C₁₀ to C₂₀ are used. Additionally, neutral lipids having mixtures of saturated and unsaturated fatty acid chains can be used.

Suitable neutral lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), dimyristoyl phosphatidylcholine (DMPC), distearoyl-phosphatidyl-ethanolamine (DSPE), SM, 16-0-monomethyl PE, 16-O-dimethyl PE, 18-1-trans-PE, 1-stearoyl-2-oleoyl-phosphatidyethanolamine (SOPE), cholesterol, or a mixture thereof. Anionic lipids suitable for use in LNPs include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

“Amphipathic lipid” means any suitable material, wherein the hydrophobic portion of a lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleoylphosphatidylcholine. Other phosphorus-lacking compounds, such as sphingolipids, glycosphingolipid families, diacylglycerols, and beta-acyloxyacids, can also be used.

In some embodiments, the non-cationic lipid may be present in a ratio of from about 5 mol % to about 90 mol %, about 5 mol % to about 10 mol %, about 5, 10, 15, 20, 25, 30, 35, 40, 45, 50, 55, 60, 65, 70, 75, 80, 85, or about 90 mol % of the total lipid present in the LNP.

In some embodiments, LNPs comprise from about 0% to about 15 or 45% on a molar basis of neutral lipid, e.g., from about 3 to about 12% or from about 5 to about 10%. For instance, LNPs may include about 15%, about 10%, about 7.5%, or about 7.1% of neutral lipid on a molar basis (based upon 100% total moles of lipid in the LNP).

(iii) Sterols

The sterol may preferably be cholesterol.

The sterol may be present in a ratio of about 10 mol % to about 60 mol % or about 25 mol % to about 40 mol % of the LNP. In some embodiments, the sterol is present in a ratio of about 10, 15, 20, 25, 30, 35, 40, 45, 50, 55, or about 60 mol % of the total lipid present in the LNP. In other embodiments, LNPs comprise from about 5% to about 50% on a molar basis of the sterol, e.g., about 15% to about 45%, about 20% to about 40%, about 48%, about 40%, about 38.5%, about 35%, about 34.4%, about 31.5% or about 31% on a molar basis (based upon 100% total moles of lipid in the LNP).

### (iv) Aggregation Reducing Agents

The aggregation reducing agent may be a lipid capable of reducing aggregation.

Examples of such lipids include, but are not limited to, polyethylene glycol (PEG)-modified lipids, monosialoganglioside Gml, and polyamide oligomers (PAO) such as those described in U.S. Pat. No. 6,320,017, which is incorporated by reference in its entirety. Other compounds with uncharged, hydrophilic, steric-barrier moieties, which prevent aggregation during formulation, like PEG, Gml or ATTA, can also be coupled to lipids. ATTA-lipids are described, e.g., in U.S. Pat. No. 6,320,017, and PEG-lipid conjugates are described, e.g., in U.S. Pat. Nos. 5,820,873, 5,534,499 and 5,885,613, each of which is incorporated by reference in its entirety.

The aggregation reducing agent may be, for example, selected from a polyethyleneglycol (PEG)-lipid including, without limitation, a PEG-diacylglycerol (DAG), a PEG-dialkylglycerol, a PEG-dialkyloxypropyl (DAA), a PEG-phospholipid, a PEG-ceramide (Cer), or a mixture thereof (such as PEG-Cerl4 or PEG-Cer20). The PEG-DAA conjugate may be, for example, a PEG-dilauryloxypropyl (C₁₂), a PEG-dimyristyloxypropyl (C14), a PEG-dipalmityloxypropyl (C₁₆), or a PEG-distearyloxypropyl (C₁₈). Other pegylated-lipids include, but are not limited to, polyethylene glycol-didimyristoyl glycerol (C₁₄-PEG or PEG-_{C14}, where PEG has an average molecular weight of 2000 Da) (PEG-DMG); (R)-2,3-bis(octadecyloxy)propyl-1-(methoxypoly(ethyleneglycol)2000)propylcarbamate) (PEG-DSG); PEG-carbamoyl-1,2-dimyristyloxypropylamine, in which PEG has an average molecular weight of 2000 Da (PEG-cDMA); N-Acetylgalactosamine-((R)-2,3-bis(octadecyloxy)propyl-1-(methoxypoly(ethyleneglycol)2000)propylcarbamate)) (GalNAc-PEG-DSG); mPEG (mw2000)-diastearoylphosphatidyl-ethanolamine (PEG-DSPE); and polyethylene glycol-dipalmitoylglycerol (PEG-DPG).

In some embodiments, the aggregation reducing agent is PEG-DMG. In other embodiments, the aggregation reducing agent is PEG-c-DMA.

In further preferred embodiments, the LNP comprises PEG-lipid alternatives, are PEG-less, and/or comprise phosphatidylcholine (PC) replacement lipids (e.g. oleic acid or analogs thereof).

In further preferred embodiments, the LNP comprises the aggregation reducing agent with formula (IV) according to the patent application PCT/EP2017/064066.

### LNP Composition

The composition of LNPs may be influenced by, inter alia, the selection of the cationic lipid component, the degree of cationic lipid saturation, the nature of the PEGylation, the ratio of all components and biophysical parameters such as its size. In one example by Semple et al. (Semple et al. Nature Biotech. 2010 28: 172-176; herein incorporated by reference in its entirety), the LNP composition was composed of 57.1% cationic lipid, 7.1% dipalmitoylphosphatidylcholine, 34.3% cholesterol, and 1.4% PEG-c-DMA (Basha et al. Mol Ther. 2011 19:2186-2200; herein incorporated by reference in its entirety).

In some embodiments, LNPs may comprise from about 35 to about 45% cationic lipid, from about 40% to about 50% cationic lipid, from about 50% to about 60% cationic lipid and/or from about 55% to about 65% cationic lipid. In some embodiments, the ratio of lipid to nucleic acid may range from about 5:1 to about 20:1, from about 10:1 to about 25:1, from about 15:1 to about 30:1 and/or at least 30:1.

The average molecular weight of the PEG moiety in the PEG-modified lipids can range from about 500 to about 8,000 Daltons (e.g., from about 1,000 to about 4,000 Daltons). In one preferred embodiment, the average molecular weight of the PEG moiety is about 2,000 Daltons.

The concentration of the aggregation reducing agent may range from about 0.1 to about 15 mol %, per 100% total moles of lipid in the LNP. In some embodiments, LNPs include less than about 3, 2, or 1 mole percent of PEG or PEG-modified lipid, based on the total moles of lipid in the LNP. In further embodiments, LNPs comprise from about 0.1% to about 20% of the PEG-modified lipid on a molar basis, e.g., about 0.5 to about 10%, about 0.5 to about 5%, about 10%, about 5%, about 3.5%, about 1.5%, about 0.5%, or about 0.3% on a molar basis (based on 100% total moles of lipids in the LNP).

Different LNPs having varying molar ratios of cationic lipid, non-cationic (or neutral) lipid, sterol (e.g., cholesterol), and aggregation reducing agent (such as a PEG-modified lipid) on a molar basis (based upon the total moles of lipid in the lipid nanoparticles) as depicted in Table 3 below. In preferred embodiments, the lipid nanoparticle formulation of the invention consists essentially of a lipid mixture in molar ratios of about 20-70% cationic lipid:5-45% neutral lipid:20-55% cholesterol, 0.5-15% PEG-modified lipid, more preferably in molar ratios of about 20-60% cationic lipid:5-25% neutral lipid:25-55% cholesterol:0.5-15% PEG-modified lipid.

TABLE 3
Lipid-based formulations

| | Molar ratio of Lipids | | | |
|---|---|---|---|---|
| | (based upon 100% total moles of lipid in the lipid nanoparticle) | | | |
| | | Non-Cationic | | Aggregation |
| | Cationic | (or Neutral) | | Reducing Agent |
| # | Lipid | Lipid | Sterol | (e.g., PEG-lipid) |
| 1 | from about 35% | from about 3% | from about 15% | from about 0.1% |
| | to about 65% | to about 12% | to about 45% | to about 10% |
| | | or 15% | | (preferably from |
| | | | | about 0.5% to |
| | | | | about 2% or 3% |
| 2 | from about 20% | from about 5% | from about 20% | from about 0.1% |
| | to about 70% | to about 45% | to about 55% | to about 10% |
| | | | | (preferably from |
| | | | | about 0.5% to |
| | | | | about 2% or 3% |
| 3 | from about 45% | from about 5% | from about 5% | from about 0.1% |
| | to about 65% | to about 10% | to about 45% | to about 3% |
| 4 | from about 20% | from about 5% | from about 25% | from about 0.1% |
| | to about 60% | to about 25% | to about 40% | to about 5% |
| | | | | (preferably from |
| | | | | about 0.1% to |
| | | | | about 3%) |
| 5 | about 40% | about 10% | from about 25% | about 10% |
| | | | to about 55% | |
| 6 | about 35% | about 15% | | about 10% |
| 7 | about 52% | about 13% | | about 5% |
| 8 | about 50% | about 10% | | about 1.5% |

In some embodiments, LNPs may occur as liposomes or lipoplexes as described in further detail below.

### LNP Size

In some embodiments, LNPs have a median diameter size of from about 50 nm to about 300 nm, such as from about 50 nm to about 250 nm, for example, from about 50 nm to about 200 nm.

In some embodiments, smaller LNPs may be used. Such particles may comprise a diameter from below 0.1 um up to 100 nm such as, but not limited to, less than 0.1 um, less than 1.0 um, less than 5 um, less than 10 um, less than 15 um, less than 20 um, less than 25 um, less than 30 um, less than 35 um, less than 40 um, less than 50 um, less than 55 um, less than 60 um, less than 65 um, less than 70 um, less than 75 um, less than 80 um, less than 85 um, less than 90 um, less than 95 um, less than 100 um, less than 125 um, less than 150 um, less than 175 um, less than 200 um, less than 225 um, less than 250 um, less than 275 um, less than 300 um, less than 325 um, less than 350 um, less than 375 um, less than 400 um, less than 425 um, less than 450 um, less than 475 um, less than 500 um, less than 525 um, less than 550 um, less than 575 um, less than 600 um, less than 625 um, less than 650 um, less than 675 um, less than 700 um, less than 725 um, less than 750 um, less than 775 um, less than 800 um, less than 825 um, less than 850 um, less than 875 um, less than 900 um, less than 925 um, less than 950 um, less than 975 um, In another embodiment, nucleic acids may be delivered using smaller LNPs which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nM, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm.

In some embodiments, the LNP have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In other embodiments, LNPs have a single mode particle size distribution (i.e., they are not bi- or poly-modal).

### Other Components

LNPs may further comprise one or more lipids and/or other components in addition to those mentioned above.

Other lipids may be included in the liposome compositions for a variety of purposes, such as to prevent lipid oxidation or to attach ligands onto the liposome surface. Any of a number of lipids may be present in LNPs, including amphipathic, neutral, cationic, and anionic lipids. Such lipids can be used alone or in combination.

Additional components that may be present in a LNP include bilayer stabilizing components such as polyamide oligomers (see, e.g., U.S. Pat. No. 6,320,017, which is incorporated by reference in its entirety), peptides, proteins, and detergents.

### Liposomes

In some embodiments, artificial nucleic acid (RNA) molecules of the invention are formulated as liposomes.

Cationic lipid-based liposomes are able to complex with negatively charged nucleic acids (e.g. RNAs) via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the nucleic acid is then released from the endosome/carrier into the cytoplasm. Liposomes have long been perceived as drug delivery vehicles because of their superior biocompatibility, given that liposomes are basically analogs of biological membranes, and can be prepared from both natural and synthetic phospholipids (Int J Nanomedicine. 2014; 9: 1833-1843).

Liposomes may typically consist of a lipid bilayer that can be composed of cationic, anionic, or neutral (phospho)lipids and cholesterol, which encloses an aqueous core. Both the lipid bilayer and the aqueous space can incorporate hydrophobic or hydrophilic compounds, respectively. Liposomes may have one or more lipid membranes. Liposomes may be single-layered, referred to as unilamellar, or multi-layered, referred to as multilamellar.

Liposome characteristics and behaviour in vivo can be modified by addition of a hydrophilic polymer coating, e.g. polyethylene glycol (PEG), to the liposome surface to confer steric stabilization. Furthermore, liposomes may be used for specific targeting by attaching ligands (e.g., antibodies, peptides, and carbohydrates) to its surface or to the terminal end of the attached PEG chains (Front Pharmacol. 2015 Dec. 1; 6:286).

Liposomes may typically present as spherical vesicles and may range in size from 20 nm to a few microns.

Liposomes may be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, the contents of each of which are herein incorporated by reference in its entirety. At least one artificial nucleic acid (RNA) molecule of the invention may be encapsulated by the liposome and/or may be contained in an aqueous core which may then be encapsulated by the liposome (see International Pub. Nos. WO2012031046, WO2012031043, WO2012030901 and WO2012006378 and US Patent Publication No. US20130189351, US20130195969 and US20130202684; the contents of each of which are herein incorporated by reference in their entirety).

In some embodiments, the artificial nucleic acid (RNA) molecule of the invention may be formulated in liposomes such as, but not limited to, DiLa2 liposomes (Marina Biotech, Bothell, Wash.), SMARTICLES® (Marina Biotech, Bothell, Wash.), neutral DOPC (l,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

### Lipoplexes

In some embodiments, artificial nucleic acid (RNA) molecules of the invention are formulated as lipoplexes, i.e. cationic lipid bilayers sandwiched between nucleic acid layers.

Cationic lipids, such as DOTAP, (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids to form nanoparticles by electrostatic interaction, providing high in vitro transfection efficiency.

### Nanoliposomes

In some embodiments, artificial nucleic acid (RNA) molecules of the invention are formulated as neutral lipid-based nanoliposomes such as 1,2-dioleoyl-sn-glycero-3-phosphatidylcholine (DOPC)-based nanoliposomes (Adv Drug Deliv Rev. 2014 February; 66: 110-116.).

### Emulsions

In some embodiments, artificial nucleic acid (RNA) molecules of the invention are formulated as emulsions. In another embodiment, said artificial nucleic acid (RNA) molecules are formulated in a cationic oil-in-water emulsion where the emulsion particle comprises an oil core and a cationic lipid which can interact with the nucleic acid(s) anchoring the molecule to the emulsion particle (see International Pub. No. WO2012006380; herein incorporated by reference in its entirety). In some embodiments, said artificial nucleic acid (RNA) molecules are formulated in a water-in-oil emulsion comprising a continuous hydrophobic phase in which the hydrophilic phase is dispersed. As a non-limiting example, the emulsion may be made by the methods described in International Publication No. WO201087791, the contents of which are herein incorporated by reference in its entirety.

### (Poly-)Cationic Compounds and Carriers

In preferred embodiments, artificial nucleic acid (RNA) molecules of the invention are complexed or associated with a cationic or polycationic compound (“(poly-)cationic compound”) and/or a polymeric carrier.

The term “(poly-)cationic compound” typically refers to a charged molecule, which is positively charged (cation) at a pH value typically from 1 to 9, preferably at a pH value of or below 9 (e.g. from 5 to 9), of or below 8 (e.g. from 5 to 8), of or below 7 (e.g. from 5 to 7), most preferably at a physiological pH, e.g. from 7.3 to 7.4.

Accordingly, a “(poly-)cationic compound” may be any positively charged compound or polymer, preferably a cationic peptide or protein, which is positively charged under physiological conditions, particularly under physiological conditions in vivo. A “(poly-)cationic peptide or protein” may contain at least one positively charged amino acid, or more than one positively charged amino acid, e.g. selected from Arg, His, Lys or Orn.

### (Poly-)Cationic Amino Acids, Peptides and Proteins

(Poly-)cationic compounds being particularly preferred agents for complexation or association of artificial nucleic acid (RNA) molecules of the invention include protamine, nucleoline, spermine or spermidine, or other cationic peptides or proteins, such as poly-L-lysine (PLL), poly-arginine, basic polypeptides, cell penetrating peptides (CPPs), including HIV-binding peptides, HIV-1 Tat (HIV), Tat-derived peptides, Penetratin, VP22 derived or analog peptides, HSV VP22 (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides (particularly from Drosophila antennapedia), pAntp, pIsl, FGF, Lactoferrin, Transportan, Buforin-2, Bac715-24, SynB, SynB(1), pVEC, hCT-derived peptides, SAP, or histones.

Preferably, the artificial nucleic acid (RNA) molecule of the invention may be complexed with one or more (poly-)cations, preferably with protamine or oligofectamine (discussed below), most preferably with protamine.

Further preferred (poly-)cationic proteins or peptides may be selected from the following proteins or peptides according to the following formula (III):

(Arg)ₗ;(Lys)ₘ;(His)ₙ;(Orn)ₒ;(Xaa)ₓ, (formula (III))

wherein l+m+n+o+x=8-15, and l, m, n or o independently of each other may be any number selected from 0, 1, 2, 3, 4, 5, 6, 7, 8, 9, 10, 11, 12, 13, 14 or 15, provided that the overall content of Arg, Lys, His and Orn represents at least 50% of all amino acids of the oligopeptide; and Xaa may be any amino acid selected from native (=naturally occurring) or non-native amino acids except of Arg, Lys, His or Orn; and x may be any number selected from 0, 1, 2, 3 or 4, provided, that the overall content of Xₐₐ does not exceed 50% of all amino acids of the oligopeptide. Particularly preferred cationic peptides in this context are e.g. Arg₇, Arg₈, Arg₉, H₃R₉, R₉H₃, H₃R₉H₃, YSSR₉SSY, (RKH)₄, Y(RKH)₂R, etc. In this context, the disclosure of WO 2009/030481 is incorporated herewith by reference.

### (Poly-)Cationic Polysaccharides

Further preferred (poly-)cationic compounds for complexation of or association with artificial nucleic acid (RNA) molecules of the invention include (poly-)cationic polysaccharides, e.g. chitosan, polybrene, cationic polymers, e.g. polyethyleneimine (PEI).

### (Poly-)Cationic Lipids

Further preferred (poly-)cationic compounds for complexation of or association with artificial nucleic acid (RNA) molecules of the invention include (poly-)cationic lipids, e.g. DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS: Dioctadecylamidoglicylspermin, DIMRI: Dimyristo-oxypropyl dimethyl hydroxyethyl ammonium bromide, DOTAP: dioleoyloxy-3-(trimethylammonio)propane, DC-6-14: O,O-ditetradecanoyl-N-(alpha-trimethylammonioacetyl)diethanolamine chloride, CLIP1: rac-[(2,3-dioctadecyloxypropyl)(2-hydroxyethyl)]-dimethylammonium chloride, CLIP6: rac-[2(2,3-dihexadecyloxypropyl-oxymethyloxy)ethyl]trimethylammonium, CLIP9: rac-[2(2,3-dihexadecyloxypropyl-oxysuccinyloxy)ethyl]-trimethylammonium, or oligofectamine.

### (Poly-)Cationic Polymers

Further preferred (poly-)cationic compounds for complexation of or association with artificial nucleic acid (RNA) molecules of the invention include (poly-)cationic polymers, e.g. modified polyaminoacids, such as beta-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified amidoamines such as pAMAM (poly(amidoamine)), etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., or blockpolymers consisting of a combination of one or more cationic blocks (e.g. selected from a cationic polymer as mentioned above) and of one or more hydrophilic or hydrophobic blocks (e.g. polyethyleneglycole).

### Polymeric Carriers

According to preferred embodiments, artificial nucleic acid (RNA) molecules of the invention may be complexed or associated with a polymeric carrier.

A “polymeric carrier” used according to the invention may be a polymeric carrier formed by disulfide-crosslinked cationic components. The disulfide-crosslinked cationic components may be the same or different from each other. The polymeric carrier may also contain further components.

It may be particularly preferred that the polymeric carrier used according to the present invention comprises mixtures of cationic peptides, proteins or polymers and optionally further components as defined herein, which are crosslinked by disulfide bonds as described herein. In this context, the disclosure of WO 2012/013326 is incorporated herewith by reference.

In this context, the cationic components, which form basis for the polymeric carrier by disulfide-crosslinkage, are typically selected from any suitable (poly-)cationic peptide, protein or polymer suitable for this purpose, particular any (poly-)cationic peptide, protein or polymer capable of complexing, and thereby preferably condensing, the artificial nucleic acid (RNA) molecule of the invention. The (poly-)cationic peptide, protein or polymer, may preferably be a linear molecule, however, branched (poly-)cationic peptides, proteins or polymers may also be used.

Every disulfide-crosslinking (poly-)cationic protein, peptide or polymer of the polymeric carrier, which may be used to complex the artificial nucleic acid (RNA) molecules typically contains at least one —SH moiety, most preferably at least one cysteine residue or any further chemical group exhibiting an —SH moiety, capable of forming a disulfide linkage upon condensation with at least one further (poly-)cationic protein, peptide or polymer as cationic component of the polymeric carrier as mentioned herein.

As defined above, the polymeric carrier, which may be used to complex the artificial nucleic acid (RNA) molecule of the invention may be formed by disulfide-crosslinked cationic (or polycationic) components. Preferably, such (poly-)cationic peptides or proteins or polymers of the polymeric carrier, which comprise or are additionally modified to comprise at least one —SH moiety, are selected from, proteins, peptides and polymers as defined herein.

In some embodiments, the polymeric carrier may be selected from a polymeric carrier molecule according to formula (IV):

L-P¹—S—[S—P²—S]—S—P³-L formula (IV)

wherein,
P¹ and P³ are different or identical to each other and represent a linear or branched hydrophilic polymer chain, each P¹ and P³ exhibiting at least one —SH-moiety, capable to form a disulfide linkage upon condensation with component P², or alternatively with (AA), (AA)ₓ, or [(AA)ₓ]_{z} if such components are used as a linker between P¹ and P² or P³ and P²) and/or with further components (e.g. (AA), (AA)ₓ, [(AA)ₓ]_{z} or L), the linear or branched hydrophilic polymer chain selected independent from each other from polyethylene glycol (PEG), poly-N-(2-hydroxypropyl)methacrylamide, poly-2-(methacryloyloxy)ethyl phosphorylcholines, poly(hydroxyalkyl L-asparagine), poly(2-(methacryloyloxy)ethyl phosphorylcholine), hydroxyethylstarch or poly(hydroxyalkyl L-glutamine), wherein the hydrophilic polymer chain exhibits a molecular weight of about 1 kDa to about 100 kDa, preferably of about 2 kDa to about 25 kDa; or more preferably of about 2 kDa to about 10 kDa, e.g. about 5 kDa to about 25 kDa or 5 kDa to about 10 kDa;
P² is a (poly-)cationic peptide or protein, e.g. as defined above for the polymeric carrier formed by disulfide-crosslinked cationic components, and preferably having a length of about 3 to about 100 amino acids, more preferably having a length of about 3 to about 50 amino acids, even more preferably having a length of about 3 to about 25 amino acids, e.g. a length of about 3 to 10, 5 to 15, 10 to 20 or 15 to 25 amino acids, more preferably a length of about 5 to about 20 and even more preferably a length of about 10 to about 20; or is a (poly-)cationic polymer, e.g. as defined above for the polymeric carrier formed by disulfide-crosslinked cationic
components, typically having a molecular weight of about 0.5 kDa to about 30 kDa, including a molecular weight of about 1 kDa to about 20 kDa, even more preferably of about 1.5 kDa to about 10 kDa, or having a molecular weight of about 0.5 kDa to about 100 kDa, including a molecular weight of about 10 kDa to about 50 kDa, even more preferably of about 10 kDa to about 30 kDa;
each P² exhibiting at least two —SH-moieties, capable to form a disulfide linkage upon condensation with further components P² or component(s) P¹ and/or P³ or alternatively with further components (e.g. (AA), (AA)ₓ, or [(AA)ₓ]_{z}); —S—S— is a (reversible) disulfide bond (the brackets are omitted for better readability), wherein S preferably represents sulphur or a —SH carrying moiety, which has formed a (reversible) disulfide bond. The (reversible) disulfide bond is preferably formed by condensation of —SH-moieties of either components P¹ and P², P² and P², or P² and P³, or optionally of further components as defined herein (e.g. L, (AA), (AA)ₓ, [(AA)ₓ]_{z}, etc); The —SH-moiety may be part of the structure of these components or added by a modification as defined below;
L is an optional ligand, which may be present or not, and may be selected independent from the other from RGD, Transferrin, Folate, a signal peptide or signal sequence, a localization signal or sequence, a nuclear localization signal or sequence (NLS), an antibody, a cell penetrating peptide, (e.g. TAT or KALA), a ligand of a receptor (e.g. cytokines, hormones, growth factors etc), small molecules (e.g. carbohydrates like mannose or galactose or synthetic ligands), small molecule agonists, inhibitors or antagonists of receptors (e.g. RGD peptidomimetic analogues), or any further protein as defined herein, etc.;
n is an integer, typically selected from a range of about 1 to 50, preferably from a range of about 1, 2 or 3 to 30, more preferably from a range of about 1, 2, 3, 4, or 5 to 25, or a range of about 1, 2, 3, 4, or 5 to 20, or a range of about 1, 2, 3, 4, or 5 to 15, or a range of about 1, 2, 3, 4, or 5 to 10, including e.g. a range of about 4 to 9, 4 to 10, 3 to 20, 4 to 20, 5 to 20, or 10 to 20, or a range of about 3 to 15, 4 to 15, 5 to 15, or 10 to 15, or a range of about 6 to 11 or 7 to 10. Most preferably, n is in a range of about 1, 2, 3, 4, or 5 to 10, more preferably in a range of about 1, 2, 3, or 4 to 9, in a range of about 1, 2, 3, or 4 to 8, or in a range of about 1, 2, or 3 to 7.

In this context, the disclosure of WO 2011/026641 is incorporated herewith by reference. Each of hydrophilic polymers P¹ and P³ typically exhibits at least one —SH-moiety, wherein the at least one —SH-moiety is capable to form a disulfide linkage upon reaction with component P² or with component (AA) or (AA)ₓ, if used as linker between P¹ and P² or P³ and P² as defined below and optionally with a further component, e.g. L and/or (AA) or (AA)ₓ, e.g. if two or more —SH-moieties are contained. The following subformulae “P¹—S—S—P²” and “P²—S—S—P³” within generic formula (IV) above (the brackets are omitted for better readability), wherein any of S, P¹ and P³ are as defined herein, typically represent a situation, wherein one-SH-moiety of hydrophilic polymers P¹ and P³ was condensed with one —SH-moiety of component P² of generic formula (IV) above, wherein both sulphurs of these —SH-moieties form a disulfide bond —S—S— as defined herein in formula (IV). These —SH-moieties are typically provided by each of the hydrophilic polymers P¹ and P³, e.g. via an internal cysteine or any further (modified) amino acid or compound which carries a —SH moiety. Accordingly, the subformulae “P¹—S—S—P²” and “P²—S—S—P³” may also be written as “P¹-Cys-Cys-P²” and “P²-Cys-Cys-P³”, if the —SH— moiety is provided by a cysteine, wherein the term Cys-Cys represents two cysteines coupled via a disulfide bond, not via a peptide bond. In this case, the term “—S—S—” in these formulae may also be written as “—S-Cys”, as “-Cys-S” or as “-Cys-Cys-”. In this context, the term “-Cys-Cys-” does not represent a peptide bond but a linkage of two cysteines via their —SH-moieties to form a disulfide bond. Accordingly, the term “-Cys-Cys-” also may be understood generally as “-(Cys-S)—(S-Cys)-”, wherein in this specific case S indicates the sulphur of the —SH-moiety of cysteine. Likewise, the terms “—S-Cys” and “—Cys-S” indicate a disulfide bond between a —SH containing moiety and a cysteine, which may also be written as “—S—(S-Cys)” and “-(Cys-S)—S”. Alternatively, the hydrophilic polymers P¹ and P³ may be modified with a —SH moiety, preferably via a chemical reaction with a compound carrying a —SH moiety, such that each of the hydrophilic polymers P¹ and P³ carries at least one such —SH moiety. Such a compound carrying a —SH moiety may be e.g. an (additional) cysteine or any further (modified) amino acid, which carries a —SH moiety. Such a compound may also be any non-amino compound or moiety, which contains or allows to introduce a —SH moiety into hydrophilic polymers P¹ and P³ as defined herein. Such non-amino compounds may be attached to the hydrophilic polymers P¹ and P³ of formula (IV) of the polymeric carrier according to the present invention via chemical reactions or binding of compounds, e.g. by binding of a 3-thio propionic acid or thioimolane, by amide formation (e.g. carboxylic acids, sulphonic acids, amines, etc), by Michael addition (e.g maleinimide moieties, α,β-unsaturated carbonyls, etc), by click chemistry (e.g. azides or alkines), by alkene/alkine methatesis (e.g. alkenes or alkines), imine or hydrozone formation (aldehydes or ketons, hydrazins, hydroxylamins, amines), complexation reactions (avidin, biotin, protein G) or components which allow Sₙ-type substitution reactions (e.g halogenalkans, thiols, alcohols, amines, hydrazines, hydrazides, sulphonic acid esters, oxyphosphonium salts) or other chemical moieties which can be utilized in the attachment of further components. A particularly preferred PEG derivate in this context is alpha-Methoxy-omega-mercapto poly(ethylene glycol). In each case, the SH-moiety, e.g. of a cysteine or of any further (modified) amino acid or compound, may be present at the terminal ends or internally at any position of hydrophilic polymers P¹ and P³. As defined herein, each of hydrophilic polymers P¹ and P³ typically exhibits at least one —SH-moiety preferably at one terminal end, but may also contain two or even more —SH-moieties, which may be used to additionally attach further components as defined herein, preferably further functional peptides or proteins e.g. a ligand, an amino acid component (AA) or (AA)ₓ, antibodies, cell penetrating peptides or enhancer peptides (e.g. TAT, KALA), etc.

### Weight Ratio and N/P Ratio

In some embodiments of the invention, the artificial nucleic acid (RNA) molecule is associated with or complexed with a (poly-)cationic compound or a polymeric carrier, optionally in a weight ratio selected from a range of about 6:1 (w/w) to about 0.25:1 (w/w), more preferably from about 5:1 (w/w) to about 0.5:1 (w/w), even more preferably of about 4:1 (w/w) to about 1:1 (w/w) or of about 3:1 (w/w) to about 1:1 (w/w), and most preferably a ratio of about 3:1 (w/w) to about 2:1 (w/w) of nucleic acid to (poly-)cationic compound and/or polymeric carrier; or optionally in a nitrogen/phosphate (N/P) ratio of nucleic acid (RNA) to (poly-)cationic compound and/or polymeric carrier in the range of about 0.1-10, preferably in a range of about 0.3-4 or 0.3-1, and most preferably in a range of about 0.5-1 or 0.7-1, and even most preferably in a range of about 0.3-0.9 or 0.5-0.9. More preferably, the N/P ratio of the at least one artificial nucleic acid (RNA) molecule to the one or more polycations is in the range of about 0.1 to 10, including a range of about 0.3 to 4, of about 0.5 to 2, of about 0.7 to 2 and of about 0.7 to 1.5.

The artificial nucleic acid (RNA) molecule of the invention may also be associated with a vehicle, transfection or complexation agent for increasing the transfection efficiency of said artificial nucleic acid (RNA) molecule.

In this context, the artificial nucleic acid (RNA) molecule may preferably be complexed at least partially with a (poly-)cationic compound and/or a polymeric carrier, preferably cationic proteins or peptides. In this context, the disclosure of WO 2010/037539 and WO 2012/113513 is incorporated herewith by reference. “Partially” means that only a part of said artificial nucleic acid (RNA) molecule is complexed with a (poly-)cationic compound and/or polymeric carrier, while the rest of said artificial nucleic acid (RNA) molecule is present in uncomplexed (“free) form.

Preferably, the molar ratio of the complexed artificial nucleic acid (RNA) molecule, to the free artificial nucleic acid (RNA) molecule may be selected from a molar ratio of about 0.001:1 to about 1:0.001, including a ratio of about 1:1. More preferably the ratio of complexed artificial nucleic acid (RNA) molecule to free artificial nucleic acid (RNA) molecule may be selected from a range of about 5:1 (w/w) to about 1:10 (w/w), more preferably from a range of about 4:1 (w/w) to about 1:8 (w/w), even more preferably from a range of about 3:1 (w/w) to about 1:5 (w/w) or 1:3 (w/w), and most preferably from a ratio of about 1:1 (w/w).

The complexed artificial nucleic acid (RNA) molecule of the invention is preferably prepared according to a first step by complexing the artificial nucleic acid (RNA) molecule with a (poly-)cationic compound and/or with a polymeric carrier, preferably as defined herein, in a specific ratio to form a stable complex. In this context, it is highly preferable, that no free (poly-)cationic compound or polymeric carrier or only a negligibly small amount thereof remains in the fraction of the complexed artificial nucleic acid (RNA) molecule after complexing said artificial nucleic acid (RNA) molecule. Accordingly, the ratio of the artificial nucleic acid (RNA) molecule and the (poly-)cationic compound and/or the polymeric carrier in the fraction of the complexed artificial nucleic acid (RNA) molecule is typically selected in a range so that the artificial nucleic acid (RNA) molecule is entirely complexed and no free (poly-)cationic compound or polymeric carrier or only a negligibly small amount thereof remains in said fraction.

Preferably, the ratio of the artificial nucleic acid (RNA) molecule to the (poly-)cationic compound and/or the polymeric carrier, preferably as defined herein, is selected from a range of about 6:1 (w/w) to about 0,25:1 (w/w), more preferably from about 5:1 (w/w) to about 0,5:1 (w/w), even more preferably of about 4:1 (w/w) to about 1:1 (w/w) or of about 3:1 (w/w) to about 1:1 (w/w), and most preferably a ratio of about 3:1 (w/w) to about 2:1 (w/w).

Alternatively, the ratio of the artificial nucleic acid (RNA) molecule to the (poly-)cationic compound and/or the polymeric carrier may also be calculated on the basis of the nitrogen/phosphate ratio (N/P-ratio) of the entire complex. In the context of the present invention, an N/P-ratio is preferably in the range of about 0.1-10, preferably in a range of about 0.3-4 and most preferably in a range of about 0.5-2 or 0.7-2 regarding the ratio of artificial nucleic acid (RNA) molecule to (poly-)cationic compound and/or polymeric carrier, preferably as defined herein, in the complex, and most preferably in a range of about 0.7-1,5, 0.5-1 or 0.7-1, and even most preferably in a range of about 0.3-0.9 or 0.5-0.9, preferably provided that the (poly-)cationic compound in the complex is a (poly-)cationic protein or peptide and/or the polymeric carrier as defined above.

In other embodiments, artificial nucleic acid (RNA) molecule is provided and used in free or naked form without being associated with any further vehicle, transfection or complexation agent.

### Targeted Delivery

In some embodiments, artificial nucleic acid (RNA) molecules of the invention (or (pharmaceutical) compositions or kits comprising the same) are adapted for targeted delivery to organs, tissues or cells or interest. “Targeted delivery” typically involves the use of targeting elements which specifically enhance translocation of the artificial nucleic acid (RNA) molecule to specific tissues or cells.

Such (proteinaceous) targeting elements may either be encoded by the artificial nucleic acid (RNA) molecule, preferably in frame with the coding sequence encoding the desired therapeutic, antigenic, allergenic or reporter protein such that said protein is expressed as a fusion protein comprising said proteinaceous targeting element. Alternatively, said (proteinaceous or non-proteinaceous) targeting element may be present in, form part of or be associated with (poly-)cationic compounds or carriers complexing said artificial nucleic acid (RNA) molecules, and/or may be resent in, form part of or be associated with lipids enclosing or complexing said artificial nucleic acid (RNA) molecules as liposomes, lipid nanoparticles, lipoplexes, and the like.

A “target” is a specific organ, tissue, or cell for which uptake of the artificial nucleic acid (RNA) molecule and preferably expression of the encoded (poly-)peptide or protein of interest is intended. “Uptake” means the translocation of the artificial nucleic acid (RNA) molecule from the extracellular to intracellular compartments. This can involve receptor mediated processes, fusion with cell membranes, endocytosis, potocytosis, pinocytosis or other translocation mechanisms. The artificial nucleic acid (RNA) molecule may be taken up by itself or as part of a complex.

As a non-limiting example, (poly-)cationic compounds, carriers, liposomes or lipid nanoparticles associated with or complexing the inventive artificial nuclei acid (RNA) molecules may be endowed with targeting elements or -functionalities. Additionally or alternatively, the artificial nucleic acid (RNA) molecule may encode (poly-)peptides or proteins carrying, preferably via covalent linkages, targeting elements. Targeting elements may be selected from proteins (e.g., glycoproteins, or peptides, e.g., molecules having a specific affinity for a co-ligand, or antibodies e.g., an antibody, that binds to a specified cell type such as a epithelial cell, keratinocyte or the like), hormones and hormone receptors, non-peptidic species, such as lipids, lectins, carbohydrates, vitamins, cofactors, multivalent lactose, multivalent galactose, N-acetyl-galactosamine, N-acetyl-gulucosamine multivalent mannose, multivalent fucose, or aptamers, and any ligand capable of targeting an artificial nucleic acid (RNA) molecule to a site of interest, such as an organ, tissue or cell.

In some embodiments, the artificial nucleic acid (RNA) molecules, or (pharmaceutical) compositions or kits comprising the same, are adapted for targeting (in)to the liver. Such artificial nucleic acid (RNA) molecules or (pharmaceutical) compositions or kits may be particularly suited for treatment, prevention, post-exposure prophylaxis or attenuation of a disease selected from the group consisting of genetic diseases, allergies, autoimmune diseases, infectious diseases, neoplasms, cancer and tumor-related diseases, inflammatory diseases, diseases of the blood and blood-forming organs, endocrine, nutritional and metabolic diseases, diseases of the nervous system, inherited diseases, diseases of the circulatory system, diseases of the respiratory system, diseases of the digestive system, diseases of the skin and subcutaneous tissue, diseases of the musculoskeletal system and connective tissue, and diseases of the genitourinary system independently if they are inherited or acquired and combinations thereof. In some embodiments, artificial nucleic acid (RNA) molecules adapted for liver-targeting comprise UTR elements according to a-2 (NDUFA4/PSMB3); a-5 (MP68/PSMB3); c-1 (NDUFA4/RPS9); a-1 (HSD17B4/PSMB3); e-3 (MP68/RPS9); e-4 (NOSIP/RPS9); a-4 (NOSIP/PSMB3); e-2 (RPL31/RPS9); e-5 (ATP5A1/RPS9); d-4 (HSD17B4/NUDFA1); b-5 (NOSIP/COX6B1); a-3 (SLC7A3/PSMB3); b-1 (UBQLN2/RPS9); b-2 (ASAH1/RPS9); b-4 (HSD17B4/CASP1); e-6 (ATP5A1/COX6B1); b-3 (HSD17B4/RPS9); g-5 (RPL31/CASP1); h-1 (RPL31/COX6B1); and/or c-5 (ATP5A1/PSMB3) as defined above. Such artificial nucleic acid (RNA) molecules or particles comprising such RNA molecules may for instance comprise targeting elements or modifications selected from the group consisting of galactose or lactose (targeting the asialoglycoprotein-receptor); apolipoprotein E; mannose; fucose; hyaluran; mannose-6-phosphate; lactose; mannose; Vitamin-A; galactosamine, GalNac and antibodies or fragments targeting synaptophysin as described by Poelstra et al. (J Control Release 161:188-197, 2012) or Mishra et al. (Biomed Res Int. 2013:382184, 2013).

In some embodiments, the artificial nucleic acid (RNA) molecules, or (pharmaceutical) compositions or kits comprising the same, are adapted for targeting to the skin. In some embodiments, such artificial nucleic acid (RNA) molecules comprise UTR elements according to a-2 (NDUFA4/PSMB3); a-5 (MP68/PSMB3); c-1 (NDUFA4/RPS9); a-1 (HSD17B4/PSMB3); e-3 (MP68/RPS9); e-4 (NOSIP/RPS9); a-4 (NOSIP/PSMB3); e-2 (RPL31/RPS9); e-5 (ATP5A1/RPS9); d-4 (HSD17B4/NUDFA1); b-5 (NOSIP/COX6B1); a-3 (SLC7A3/PSMB3); b-1 (UBQLN2/RPS9); b-2 (ASAH1/RPS9); b-4 (HSD17B4/CASP1); e-6 (ATP5A1/COX6B1); b-3 (HSD17B4/RPS9); g-5 (RPL31/CASP1); h-1 (RPL31/COX6B1); and/or c-5 (ATP5A1/PSMB3) as defined above. Such artificial nucleic acid (RNA) molecules or particles comprising such RNA molecules may for instance comprise targeting elements as described herein below.

In some embodiments, the artificial nucleic acid (RNA) molecules, or (pharmaceutical) compositions or kits comprising the same, are adapted for targeting to the muscle. In some embodiments, such artificial nucleic acid (RNA) molecules comprise UTR elements according to a-2 (NDUFA4/PSMB3); a-5 (MP68/PSMB3); c-1 (NDUFA4/RPS9); a-1 (HSD17B4/PSMB3); e-3 (MP68/RPS9); e-4 (NOSIP/RPS9); a-4 (NOSIP/PSMB3); e-2 (RPL31/RPS9); e-5 (ATP5A1/RPS9); d-4 (HSD17B4/NUDFA1); b-5 (NOSIP/COX6B1); a-3 (SLC7A3/PSMB3); b-1 (UBQLN2/RPS9); b-2 (ASAH1/RPS9); b-4 (HSD17B4/CASP1); e-6 (ATP5A1/COX6B1); b-3 (HSD17B4/RPS9); g-5 (RPL31/CASP1); h-1 (RPL31/COX6B1); and/or c-5 (ATP5A1/PSMB3) as defined above. Such artificial nucleic acid (RNA) molecules or particles comprising such RNA molecules may for instance comprise targeting elements as described herein below.

Suitable targeting elements for use in connection with the present invention include: lectins, glycoproteins, lipids and proteins, e.g., antibodies. In particular, targeting elements may be selected from a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Further targeting elements may be selected from proteins, e.g., glycoproteins, or peptides, e.g., molecules having a specific affinity for a co-ligand, or antibodies e.g., capable of binding to a specified cell type such as a liver, tumor, muscle, skin or kidney cell. Further targeting elements may be selected from hormones and hormone receptors. Further targeting elements may be selected from lipids, lectins, carbohydrates, vitamins, cofactors, multivalent lactose, multivalent galactose, N-acetyl-galactosamine, N-acetyl-gulucosamine multivalent mannose, multivalent fucose, or aptamers. Targeting elements may bind to any suitable ligand selected from, e.g. a lipopolysaccharide, or an activator of p38 MAP kinase.

Further targeting elements may be selected from ligands capable of targeting a specific receptor. Examples include, without limitation, folate, GalNAc, galactose, mannose, mannose-6P, apatamers, integrin receptor ligands, chemokine receptor ligands, transferrin, biotin, serotonin receptor ligands, PSMA, endothelin, GCPII, somatostatin, (KKEEE)3K, LDL, and HDL ligands. Further targeting elements may be selected from aptamers. The aptamer may be unmodified or may have any combination of modifications disclosed herein.

### (Pharmaceutical) Composition and Vaccines

In a further aspect, the present invention provides a composition comprising the artificial nucleic acid (RNA) molecule of the invention, and preferably at least one pharmaceutically acceptable carrier and/or excipient. According to preferred embodiments, the composition is provided as a pharmaceutical composition. According to further preferred embodiments, the (pharmaceutical) composition may be provided as a vaccine. A “vaccine” is typically understood to be a prophylactic or therapeutic material providing at least one antigen, preferably an antigenic peptide or protein. “Providing at least on antigen” means, for example, that the vaccine comprises the antigen or that the vaccine comprises a molecule that, e.g., codes for the antigen. Accordingly, it is particularly envisaged herein that the inventive vaccine comprises at least one artificial nucleic acid (RNA) molecule encoding at least one antigenic (poly-)peptide or protein as defined herein, which may, for instance, be derived from a tumor antigen, a bacterial, viral, fungal or protozoal antigen, an autoantigen, an allergen, or an allogenic antigen, and preferably induces an immune response towards the respective antigen when it is expressed and presented to the immune system. However, artificial nucleic acid (RNA) molecules encoding non-antigenic (poly-)peptides or proteins of interest may also be used in the inventive vaccine.

The (pharmaceutical) composition or vaccine of the invention preferably comprises at least one, preferably a plurality of at least two artificial nucleic acid (RNA) molecules as described herein. Said plurality of at least two artificial nucleic acid (RNA) molecules may be monocistronic, bicistronic or multicistronic as described herein. Each of the artificial nucleic acid (RNA) molecules in the (pharmaceutical) composition or vaccine may encode at least one, or a plurality of at least two (identical or different) (poly-)peptides or proteins of interest. The artificial nucleic acid (RNA) molecules may be provided in the (pharmaceutical) composition or vaccine in “complexed” or “free” form as described above, or a mixture thereof. The (pharmaceutical) composition or vaccine may further comprise at least one additional active agent useful for treatment of the disease or condition that is subject to therapy with the artificial nucleic acid (RNA) molecule, or (pharmaceutical) composition or vaccine comprising the same.

### Pharmaceutically Acceptable Excipients and Carriers

Preferably, the (pharmaceutical) composition or vaccine according to the invention comprises at least one pharmaceutically acceptable carrier and/or excipient. The term “pharmaceutically acceptable” refers to a compound or agent that is compatible with the one or more active agent(s) (here: artificial nucleic acid (RNA) molecule and optionally additional active agent) and does not interfere with and/or substantially reduce its/their pharmaceutical effect. Pharmaceutically acceptable carriers and excipients preferably have sufficiently high purity and sufficiently low toxicity to make them suitable for administration to a subject to be treated.

### Excipients

Pharmaceutically acceptable excipients can exhibit different functional roles and include, without limitation, diluents, fillers, bulking agents, carriers, disintegrants, binders, lubricants, glidants, coatings, solvents and co-solvents, buffering agents, preservatives, adjuvants, anti-oxidants, wetting agents, anti-foaming agents, thickening agents, sweetening agents, flavouring agents and humectants.

For (pharmaceutical) compositions in liquid form, useful pharmaceutically acceptable carriers and excipients include solvents, diluents, or carriers such as (pyrogen-free) water, (isotonic) saline solutions such phosphate or citrate buffered saline, fixed oils, vegetable oils, such as, for example, groundnut oil, cottonseed oil, sesame oil, olive oil, corn oil, ethanol, polyols (for example, glycerol, propylene glycol, polyetheylene glycol, and the like); lecithin; surfactants; preservatives such as benzyl alcohol, parabens, chlorobutanol, phenol, ascorbic acid, thimerosal, and the like; isotonic agents such as sugars, polyalcohols such as manitol, sorbitol, or sodium chloride; aluminium monostearate or gelatine; antioxidants such as ascorbic acid or sodium bisulphite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. Buffers may be hypertonic, isotonic or hypotonic with reference to the specific reference medium, i.e. the buffer may have a higher, identical or lower salt content with reference to the specific reference medium, wherein preferably such concentrations of the aforementioned salts may be used, which do not lead to damage of cells due to osmosis or other concentration effects. Reference media are e.g. liquids occurring in “in vivo” methods, such as blood, lymph, cytosolic liquids, or other body liquids, or e.g. liquids, which may be used as reference media in “in vitro” methods, such as common buffers or liquids. Such common buffers or liquids are known to a skilled person.

Ringer solution or Ringer-Lactate solution are particularly preferred as a liquid carrier.

For (pharmaceutical) compositions in (semi-)solid form, useful pharmaceutically acceptable carriers and excipients include binders such as microcrystalline cellulose, gum tragacanth or gelatine; starch or lactose; sugars, such as, for example, lactose, glucose and sucrose; starches, such as, for example, corn starch or potato starch; cellulose and its derivatives, such as, for example, sodium carboxymethylcellulose, ethylcellulose, cellulose acetate; disintegrants such as alginic acid; lubricants such as magnesium stearate; glidants such as stearic acid, magnesium stearate; calcium sulphate, colloidal silicon dioxide and the like; sweetening agents such as sucrose or saccharin; and/or flavouring agents such as peppermint, methyl salicylate, or orange flavouring.

### Formulations

Suitable pharmaceutically acceptable carriers and excipients may typically be chosen based on the desired formulation of the (pharmaceutical) composition.

Liquid (pharmaceutical) compositions administered via injection and in particular via i.v. injection should be sterile and stable under the conditions of manufacture and storage. Such compositions are typically formulated as parenterally acceptable aqueous solutions that are pyrogen-free, have suitable pH, are isotonic and maintain stability of the active ingredient(s). Particularly useful pharmaceutically acceptable carriers and excipients for liquid (pharmaceutical) compositions according to the invention include water, typically pyrogen-free water; isotonic saline or buffered (aqueous) solutions, e.g phosphate, citrate etc. buffered solutions. Particularly for injection of the inventive (pharmaceutical) compositions, water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, preferably at least 3 mM of a potassium salt.

According to preferred embodiments, the sodium, calcium and, optionally, potassium salts may occur in the form of their halogenides, e.g. chlorides, iodides, or bromides, in the form of their hydroxides, carbonates, hydrogen carbonates, or sulphates, etc. Without being limited thereto, examples of sodium salts include e.g. NaCl, NaI, NaBr, Na₂CO₃, NaHCO₃, Na₂SO₄, examples of the optional potassium salts include e.g. KCl, KI, KBr, K₂CO₃, KHCO₃, K₂SO₄, and examples of calcium salts include e.g. CaCl₂, CaI₂, CaBr₂, CaCO₃, CaSO₄, Ca(OH)₂. Furthermore, organic anions of the aforementioned cations may be contained in the buffer.

According to preferred embodiments, the buffer suitable for injection purposes as defined above, may contain salts selected from sodium chloride (NaCl), calcium chloride (CaCl₂) and optionally potassium chloride (KCl), wherein further anions may be present additional to the chlorides. CaCl₂ can also be replaced by another salt like KCl. Typically, the salts in the injection buffer are present in a concentration of at least 50 mM sodium chloride (NaCl), at least 3 mM potassium chloride (KCl) and at least 0.01 mM calcium chloride (CaCl₂). The injection buffer may be hypertonic, isotonic or hypotonic with reference to the specific reference medium, i.e. the buffer may have a higher, identical or lower salt content with reference to the specific reference medium, wherein preferably such concentrations of the afore mentioned salts may be used, which do not lead to damage of cells due to osmosis or other concentration effects. Reference media are e.g. in “in vivo” methods occurring liquids such as blood, lymph, cytosolic liquids, or other body liquids, or e.g. liquids, which may be used as reference media in “in vitro” methods, such as common buffers or liquids.

Such common buffers or liquids are known to a skilled person. Ringer-Lactate solution is particularly preferred as a liquid basis.

(Pharmaceutical) compositions for topical administration can be formulated as creams, ointments, gels, pastes or powders, using suitable liquid and/or (semi-)solid excipients or carriers as described elsewhere herein. (Pharmaceutical) compositions for oral administration can be formulated as tablets, capsules, liquids, powders or in a sustained release format, using suitable liquid and/or (semi-)solid excipients or carriers as described elsewhere herein.

According to some preferred embodiments, the inventive (pharmaceutical) composition or vaccine is administered parenterally, in particular via intradermal or intramuscular injection, orally, nasally, pulmonary, by inhalation, topically, rectally, buccally, vaginally, or via an implanted reservoir, and is provided in liquid or lyophilized formulations for parenteral administration as discussed elsewhere herein. Parenteral formulations are typically stored in vials, IV bags, ampoules, cartridges, or prefilled syringes and can be administered as injections, inhalants, or aerosols, with injections being preferred.

According to preferred embodiments, (pharmaceutical) compositions or vaccine of the invention may comprise artificial nucleic acid (RNA) molecules of the invention complexed with lipids, preferably in the form of lipid nanoparticles, liposomes, lipoplexes or emulsions as described elsewhere herein.

According to further preferred embodiments, the (pharmaceutical) composition or vaccine is provided in lyophilized form. Preferably, the lyophilized (pharmaceutical) composition or vaccine is reconstituted in a suitable buffer, advantageously based on an aqueous carrier, prior to administration, e.g. Ringer-Lactate solution, which is preferred, Ringer solution, a phosphate buffer solution. In some embodiments, the (pharmaceutical) composition or vaccine of the invention contains at least two, three, four, five, six or more different artificial nucleic acid (RNA) molecules as defined herein, which may be provided separately in lyophilized form (optionally together with at least one further additive) and which may be reconstituted separately in a suitable buffer (such as Ringer-Lactate solution) prior to their use so as to allow individual administration of each of said artificial nucleic acid (RNA) molecules.

### Adjuvants

According to preferred embodiments, the (pharmaceutical) composition or vaccine of the invention may further comprise at least one adjuvant.

An “adjuvant” or “adjuvant component” in the broadest sense is typically a pharmacological and/or immunological agent that may modify, e.g. enhance, the effect of other active agents, e.g. therapeutic agents or vaccines. In this context, an “adjuvant” may be understood as any compound, which is suitable to support administration and delivery of inventive (pharmaceutical) composition. Specifically, an adjuvant may preferably enhance the immunostimulatory properties of the (pharmaceutical) composition or vaccine to which it is added. Furthermore, such adjuvants may, without being bound thereto, initiate or increase an immune response of the innate immune system, i.e. a non-specific immune response.

“Adjuvants” typically do not elicit an adaptive immune response. Insofar, “adjuvants” do not qualify as antigens. In other words, when administered, the inventive (pharmaceutical) composition or vaccine typically initiates an adaptive immune response due to an antigenic peptide or protein, which is encoded by the at least one coding sequence of the artificial nucleic acid (RNA) molecule contained in said (pharmaceutical) composition or vaccine. Additionally, an adjuvant present in the (pharmaceutical) composition or vaccine may generate an (supportive) innate immune response.

Suitable adjuvants may be selected from any adjuvant known to a skilled person and suitable for the present case, i.e. supporting the induction of an immune response in a mammal, and include, without limitation, TDM, MDP, muramyl dipeptide, pluronics, alum solution, aluminium hydroxide, ADJUMER™ (polyphosphazene); aluminium phosphate gel; glucans from algae; algammulin; aluminium hydroxide gel (alum); highly protein-adsorbing aluminium hydroxide gel; low viscosity aluminium hydroxide gel; AF or SPT (emulsion of squalane (5%), Tween 80 (0.2%), Pluronic L121 (1.25%), phosphate-buffered saline, pH 7.4); AVRIDINE™ (propanediamine); BAY R1005™ ((N-(2-deoxy-2-L-leucylamino-b-D-glucopyranosyl)-N-octadecyl-dodecanoyl-amide hydroacetate); CALCITRIOL™ (1-alpha,25-dihydroxy-vitamin D3); calcium phosphate gel; CAP™ (calcium phosphate nanoparticles); cholera holotoxin, cholera-toxin-A1-protein-A-D-fragment fusion protein, sub-unit B of the cholera toxin; CRL 1005 (block copolymer P1205); cytokine-containing liposomes; DDA (dimethyldioctadecylammonium bromide); DHEA (dehydroepiandrosterone); DMPC (dimyristoylphosphatidylcholine); DMPG (dimyristoylphosphatidylglycerol); DOC/alum complex (deoxycholic acid sodium salt); Freund”s complete adjuvant; Freund's incomplete adjuvant; gamma inulin; Gerbu adjuvant (mixture of: i) N-acetylglucosaminyl-(P1-4)-N-acetylmuramyl-L-alanyl-D-glutamine (GMDP), ii) dimethyldioctadecylammonium chloride (DDA), iii) zinc-L-proline salt complex (ZnPro-8); GM-CSF); GMDP (N-acetylglucosaminyl-(b1-4)-N-acetylmuramyl-L-alanyl-D-isoglutamine); imiquimod (1-(2-methypropyl)-1H-imidazo[4,5-c]quinoline-4-amine); ImmTher™ (N-acetylglucosaminyl-N-acetylmuramyl-L-Ala-D-isoGlu-L-Ala-glycerol dipalmitate); DRVs (immunoliposomes prepared from dehydration-rehydration vesicles); interferon-gamma; interleukin-1beta; interleukin-2; interleukin-7; interleukin-12; ISCOMS™; ISCOPREP 7.0.3™; liposomes; LOXORIBINE™ (7-allyl-8-oxoguanosine); LT oral adjuvant (E. coli labile enterotoxin-protoxin); microspheres and microparticles of any composition; MF59™; (squalene-water emulsion); MONTANIDE ISA 51™ (purified incomplete Freund's adjuvant); MONTANIDE ISA 720™ (metabolisable oil adjuvant); MPL™ (3-Q-desacyl-4″-monophosphoryl lipid A); MTP-PE and MTP-PE liposomes ((N-acetyl-L-alanyl-D-isoglutaminyl-L-alanine-2-(1,2-dipalmitoyl-sn-glycero-3-(hydroxyphosphoryloxy))-ethylamide, monosodium salt); MURAMETIDE™ (Nac-Mur-L-Ala-D-Gln-OCH3); MURAPALMITINE™ and D-MURAPALMITINE™ (Nac-Mur-L-Thr-D-isoGln-sn-glyceroldipalmitoyl); NAGO (neuraminidase-galactose oxidase); nanospheres or nanoparticles of any composition; NISVs (non-ionic surfactant vesicles); PLEURAN™ (β-glucan); PLGA, PGA and PLA (homo- and co-polymers of lactic acid and glycolic acid; microspheres/nanospheres); PLURONIC L121™; PMMA (polymethyl methacrylate); PODDS™ (proteinoid microspheres); polyethylene carbamate derivatives; poly-rA: poly-rU (polyadenylic acid-polyuridylic acid complex); polysorbate 80 (Tween 80); protein cochleates (Avanti Polar Lipids, Inc., Alabaster, Ala.); STIMULON™ (QS-21); Quil-A (Quil-A saponin); S-28463 (4-amino-otec-dimethyl-2-ethoxymethyl-1H-imidazo[4,5 c]quinoline-1-ethanol); SAF-1™ (“Syntex adjuvant formulation”); Sendai proteoliposomes and Sendai-containing lipid matrices; Span-85 (sorbitan trioleate); Specol (emulsion of Marcol 52, Span 85 and Tween 85); squalene or Robane® (2,6,10,15,19,23-hexamethyltetracosan and 2,6,10,15,19,23-hexamethyl-2,6,10,14,18,22-tetracosahexane); stearyltyrosine (octadecyltyrosine hydrochloride); Theramid® (N-acetylglucosaminyl-N-acetylmuramyl-L-Ala-D-isoGlu-L-Ala-dipalmitoxypropylamide); Theronyl-MDP (Termurtide™ or [thr 1]-MDP; N-acetylmuramyl-L-threonyl-D-isoglutamine); Ty particles (Ty-VLPs or virus-like particles); Walter-Reed liposomes (liposomes containing lipid A adsorbed on aluminium hydroxide), and lipopeptides, including Pam3Cys, in particular aluminium salts, such as Adju-phos, Alhydrogel, Rehydragel; emulsions, including CFA, SAF, IFA, MF59, Provax, TiterMax, Montanide, Vaxfectin; copolymers, including Optivax (CRL1005), L121, Poloaxmer4010), etc.; liposomes, including Stealth, cochleates, including BIORAL; plant derived adjuvants, including QS21, Quil A, Iscomatrix, ISCOM; adjuvants suitable for costimulation including Tomatine, biopolymers, including PLG, PMM, Inulin; microbe derived adjuvants, including Romurtide, DETOX, MPL, CWS, Mannose, CpG nucleic acid sequences, CpG7909, ligands of human TLR 1-10, ligands of murine TLR 1-13, ISS-1018, IC31, Imidazoquinolines, Ampligen, Ribi529, IMOxine, IRIVs, VLPs, cholera toxin, heat-labile toxin, Pam3Cys, Flagellin, GPI anchor, LNFPIII/Lewis X, antimicrobial peptides, UC-1V150, RSV fusion protein, cdiGMP; and adjuvants suitable as antagonists including CGRP neuropeptide.

Suitable adjuvants may also be selected from (poly-)cationic compounds as described herein as complexation agents (cf. section headed “(poly-)cationic compounds and carriers”), in particular the (poly-)cationic peptides or proteins, (poly-)cationic polysaccharides, (poly-)cationic lipids, or polymeric carriers described herein. Associating or complexing the artificial nucleic acid (RNA) molecule of the (pharmaceutical) composition or vaccine with these (poly-)cationic compounds or carriers may preferably provide adjuvant properties and confer a stabilizing effect.

The ratio of the artificial nucleic acid (RNA) molecule to the (poly-)cationic compound in the adjuvant component may be calculated on the basis of the nitrogen/phosphate ratio (N/P-ratio) of the entire complex, i.e. the ratio of positively charged (nitrogen) atoms of the (poly-)cationic compound to the negatively charged phosphate atoms of the artificial nucleic acid (RNA) molecule.

In the following, when referring to “RNA”, it will be understood that the respective disclosure is applicable to other artificial nucleic acid molecules as well, mutatis mutandis.

For example, 1 μg of RNA may contain about 3 nmol phosphate residues, provided said RNA exhibits a statistical distribution of bases. Additionally, 1 μg of peptide typically contains about x nmol nitrogen residues, dependent on the molecular weight and the number of basic amino acids. When exemplarily calculated for (Arg)9 (molecular weight 1424 g/mol, 9 nitrogen atoms), 1 μg (Arg)9 contains about 700 pmol (Arg)9 and thus 700×9=6300 pmol basic amino acids=6.3 nmol nitrogen atoms. For a mass ratio of about 1:1 RNA/(Arg)9 an N/P ratio of about 2 can be calculated. When exemplarily calculated for protamine (molecular weight about 4250 g/mol, 21 nitrogen atoms, when protamine from salmon is used) with a mass ratio of about 2:1 with 2 μg of RNA, 6 nmol phosphate are to be calculated for the RNA; 1 μg protamine contains about 235 pmol protamine molecules and thus 235×21=4935 pmol basic nitrogen atoms=4.9 nmol nitrogen atoms. For a mass ratio of about 2:1 RNA/protamine an N/P ratio of about 0.81 can be calculated. For a mass ratio of about 8:1 RNA/protamine an N/P ratio of about 0.2 can be calculated. In the context of the present invention, an N/P-ratio is preferably in the range of about 0.1-10, preferably in a range of about 0.3-4 and most preferably in a range of about 0.5-2 or 0.7-2 regarding the ratio of RNA:peptide in the complex, and most preferably in the range of about 0.7-1.5.

The (pharmaceutical) composition or vaccine of the present invention may be obtained in two separate steps in order to obtain both, an efficient immunostimulatory effect and efficient translation of the artificial nucleic acid (RNA) molecule comprised by said (pharmaceutical) composition or vaccine.

In a first step, an RNA is complexed with a (poly-)cationic compound in a specific ratio to form a stable complex (“complexed (RNA”). In this context, it is important, that no free (poly-)cationic compound or only a negligible small amount remains in the fraction of the complexed RNA. Accordingly, the ratio of the RNA and the (poly-)cationic compound is typically selected in a range that the RNA is entirely complexed and no free (poly-)cationic compound or only a neglectably small amount remains in the composition. Preferably the ratio of the RNA to the (poly-)cationic compound is selected from a range of about 6:1 (w/w) to about 0,25:1 (w/w), more preferably from about 5:1 (w/w) to about 0,5:1 (w/w), even more preferably of about 4:1 (w/w) to about 1:1 (w/w) or of about 3:1 (w/w) to about 1:1 (w/w), and most preferably a ratio of about 3:1 (w/w) to about 2:1 (w/w).

In a second step, an RNA is added to the complexed RNA in order to obtain the (pharmaceutical) composition or vaccine of the invention. Therein, said added RNA is present as free RNA, preferably as free mRNA, which is not complexed by other compounds. Prior to addition, the free RNA is not complexed and will preferably not undergo any detectable or significant complexation reaction upon the addition to the complexed RNA. This is due to the strong binding of the (poly-)cationic compound to the complexed RNA. In other words, when the free RNA is added to the complexed RNA, preferably no free or substantially no free (poly-)cationic compound is present, which could form a complex with said free RNA. Accordingly, the free RNA of the inventive (pharmaceutical) composition or vaccine can efficiently be transcribed in vivo.

It may be preferred that the free RNA may be identical or different to the complexed RNA, depending on the specific requirements of therapy. Even more preferably, the free RNA, which is comprised in the (pharmaceutical) composition or vaccine, is identical to the complexed epitope-encoding RNA, in other words, the combination, (pharmaceutical) composition or vaccine comprises an otherwise identical RNA in both free and complexed form.

In particularly preferred embodiments, the inventive (pharmaceutical) composition or vaccine thus comprises the RNA as defined herein, wherein said RNA is present in said (pharmaceutical) composition or vaccine partially as free RNA and partially as complexed RNA. Preferably, the RNA as defined herein, preferably an mRNA, is complexed as described above and the same (m)RNA is then added in the form of free RNA, wherein preferably the compound, which is used for complexing the RNA is not present in free form in the composition at the moment of addition of the free RNA.

The ratio of the complexed RNA and the free RNA may be selected depending on the specific requirements of a particular therapy. Typically, the ratio of the complexed RNA and the free RNA is selected such that a significant stimulation of the innate immune system is elicited due to the presence of the complexed RNA. In parallel, the ratio is selected such that a significant amount of the free epitope-encoding RNA can be provided in vivo leading to an efficient translation and concentration of the expressed antigenic fusion protein in vivo. Preferably the ratio of the complexed RNA to free RNA in the inventive (pharmaceutical) composition or vaccine is selected from a range of about 5:1 (w/w) to about 1:10 (w/w), more preferably from a range of about 4:1 (w/w) to about 1:8 (w/w), even more preferably from a range of about 3:1 (w/w) to about 1:5 (w/w) or 1:3 (w/w), and most preferably about 1:1 (w/w).

Additionally or alternatively, the ratio of the complexed RNA and the free RNA may be calculated on the basis of the nitrogen/phosphate ratio (N/P-ratio) of the entire RNA complex. In the context of the present invention, an N/P-ratio is preferably in the range of about 0.1-10, preferably in a range of about 0.3-4 and most preferably in a range of about 0.5-2 or 0.7-2 regarding the ratio of RNA:peptide in the complex, and most preferably in the range of about 0.7-1.5.

Additionally or alternatively, the ratio of the complexed RNA and the free RNA may also be selected on the basis of the molar ratio of both RNAs to each other. Typically, the molar ratio of the complexed RNA to the free RNA may be selected such, that the molar ratio suffices the above (w/w) and/or N/P-definitions. More preferably, the molar ratio of the complexed RNA to the free RNA may be selected e.g. from a molar ratio of about 0.001:1, 0.01:1, 0.1:1, 0.2:1, 0.3:1, 0.4:1, 0.5:1, 0.6:1, 0.7:1, 0.8:1, 0.9:1, 1:1, 1:0.9, 1:0.8, 1:0.7, 1:0.6, 1:0.5, 1:0.4, 1:0.3, 1:0.2, 1:0.1, 1:0.01, 1:0.001, etc. or from any range formed by any two of the above values, e.g. a range selected from about 0.001:1 to 1:0.001, including a range of about 0.01:1 to 1:0.001, 0.1:1 to 1:0.001, 0.2:1 to 1:0.001, 0.3:1 to 1:0.001, 0.4:1 to 1:0.001, 0.5:1 to 1:0.001, 0.6:1 to 1:0.001, 0.7:1 to 1:0.001, 0.8:1 to 1:0.001, 0.9:1 to 1:0.001, 1:1 to 1:0.001, 1:0.9 to 1:0.001, 1:0.8 to 1:0.001, 1:0.7 to 1:0.001, 1:0.6 to 1:0.001, 1:0.5 to 1:0.001, 1:0.4 to 1:0.001, 1:0.3 to 1:0.001, 1:0.2 to 1:0.001, 1:0.1 to 1:0.001, 1:0.01 to 1:0.001, or a range of about 0.01:1 to 1:0.01, 0.1:1 to 1:0.01, 0.2:1 to 1:0.01, 0.3:1 to 1:0.01, 0.4:1 to 1:0.01, 0.5:1 to 1:0.01, 0.6:1 to 1:0.01, 0.7:1 to 1:0.01, 0.8:1 to 1:0.01, 0.9:1 to 1:0.01, 1:1 to 1:0.01, 1:0.9 to 1:0.01, 1:0.8 to 1:0.01, 1:0.7 to 1:0.01, 1:0.6 to 1:0.01, 1:0.5 to 1:0.01, 1:0.4 to 1:0.01, 1:0.3 to 1:0.01, 1:0.2 to 1:0.01, 1:0.1 to 1:0.01, 1:0.01 to 1:0.01, or including a range of about 0.001:1 to 1:0.01, 0.001:1 to 1:0.1, 0.001:1 to 1:0.2, 0.001:1 to 1:0.3, 0.001:1 to 1:0.4, 0.001:1 to 1:0.5, 0.001:1 to 1:0.6, 0.001:1 to 1:0.7, 0.001:1 to 1:0.8, 0.001:1 to 1:0.9, 0.001:1 to 1:1, 0.001 to 0.9:1, 0.001 to 0.8:1, 0.001 to 0.7:1, 0.001 to 0.6:1, 0.001 to 0.5:1, 0.001 to 0.4:1, 0.001 to 0.3:1, 0.001 to 0.2:1, 0.001 to 0.1:1, or a range of about 0.01:1 to 1:0.01, 0.01:1 to 1:0.1, 0.01:1 to 1:0.2, 0.01:1 to 1:0.3, 0.01:1 to 1:0.4, 0.01:1 to 1:0.5, 0.01:1 to 1:0.6, 0.01:1 to 1:0.7, 0.01:1 to 1:0.8, 0.01:1 to 1:0.9, 0.01:1 to 1:1, 0.001 to 0.9:1, 0.001 to 0.8:1, 0.001 to 0.7:1, 0.001 to 0.6:1, 0.001 to 0.5:1, 0.001 to 0.4:1, 0.001 to 0.3:1, 0.001 to 0.2:1, 0.001 to 0.1:1, etc.

Even more preferably, the molar ratio of the complexed RNA to the free RNA may be selected e.g. from a range of about 0.01:1 to 1:0.01. Most preferably, the molar ratio of the complexed RNA to the free RNA may be selected e.g. from a molar ratio of about 1:1. Any of the above definitions with regard to (w/w) and/or N/P ratio may also apply.

According to preferred embodiments, the (pharmaceutical) composition or vaccine comprises another nucleic acid, preferably as an adjuvant.

Accordingly, the (pharmaceutical) composition or vaccine of the invention further comprises a non-coding nucleic acid, preferably RNA, selected from the group consisting of small interfering RNA (siRNA), antisense RNA (asRNA), circular RNA (circRNA), ribozymes, aptamers, riboswitches, immunostimulating RNA (isRNA), transfer RNA (tRNA), ribosomal RNA (rRNA), small nuclear RNA (snRNA), small nucleolar RNA (snoRNA), microRNA (miRNA), and Piwi-interacting RNA (piRNA).

In the context of the present invention, non-coding nucleic acids, preferably RNAs, of particular interest include “immune-stimulatory” or “is” nucleic acids, preferably RNAs. “Immune-stimulatory” or “is” nucleic acids or RNAs are typically employed as adjuvants in the (pharmaceutical) composition or vaccine according to the invention.

According to a particularly preferred embodiment, the adjuvant nucleic acid comprises a nucleic acid of the following formula (VI) or (VII):

GₗXₘGₙ (formula (VI))

wherein:
G is a nucleotide comprising guanine, uracil or an analogue of guanine or uracil;
X is a nucleotide comprising guanine, uracil, adenine, thymine, cytosine or an analogue thereof;
l is an integer from 1 to 40,
wherein
when l=1 G is a nucleotide comprising guanine or an analogue thereof,
when l>1 at least 50% of the nucleotides comprise guanine or an analogue thereof;
m is an integer and is at least 3;
wherein
when m=3, X is a nucleotide comprising uracil or an analogue thereof,
when m>3, at least 3 successive nucleotides comprising uracils or analogues of uracil occur;
n is an integer from 1 to 40,
wherein
when n=1, G is a nucleotide comprising guanine or an analogue thereof,
when n>1, at least 50% of the nucleotides comprise guanine or an analogue thereof;

CₗXₘCₙ (formula (VII))

wherein:
C is a nucleotide comprising cytosine, uracil or an analogue of cytosine or uracil;
X is a nucleotide comprising guanine, uracil, adenine, thymine, cytosine or an analogue thereof;
l is an integer from 1 to 40,
wherein
when l=1, C is a nucleotide comprising cytosine or an analogue thereof,
when l>1, at least 50% of the nucleotides comprise cytosine or an analogue thereof;
m is an integer and is at least 3;
wherein
when m=3, X comprises uracil or an analogue thereof,
when m>3, at least 3 successive nucleotides comprise uracils or analogues of uracil occur;
n is an integer from 1 to 40,
wherein
when n=1, C is a nucleotide comprising cytosine or an analogue thereof,
when n>1, at least 50% of the nucleotides comprise cytosine or an analogue thereof.

The nucleic acids of formula (VI) or (VII), which may be used as isRNA may be relatively short nucleic acid molecules with a typical length of approximately from 5 to 100 (but may also be longer than 100 nucleotides for specific embodiments, e.g. up to 200 nucleotides), from 5 to 90 or from 5 to 80 nucleotides, preferably a length of approximately from 5 to 70, more preferably a length of approximately from 8 to 60 and, more preferably a length of approximately from 15 to 60 nucleotides, more preferably from 20 to 60, most preferably from 30 to 60 nucleotides. If the epitope-encoding RNA (or any other nucleic acid, in particular RNA, as disclosed herein) has a maximum length of, for example, 100 nucleotides, m will typically be ≤98.

The number of nucleotides “G” in the nucleic acid of formula (VI) is determined by l or n. l and n, independently of one another, are each an integer from 1 to 40, wherein when l or n=1 G is a nucleotide comprising guanine or an analogue thereof, and when l or n>1 at least 50% of the nucleotides comprise guanine, or an analogue thereof.

For example, without implying any limitation, when l or n=4 Gl or Gn can be, for example, a GUGU, GGUU, UGUG, UUGG, GUUG, GGGU, GGUG, GUGG, UGGG or GGGG, etc.; when l or n=5 Gl or Gn can be, for example, a GGGUU, GGUGU, GUGGU, UGGGU, UGGUG, UGUGG, UUGGG, GUGUG, GGGGU, GGGUG, GGUGG, GUGGG, UGGGG, or GGGGG, etc.

A nucleotide adjacent to Xm in the nucleic acid of formula (VI) preferably does not comprise uracil.

Similarly, the number of nucleotides “C” in the nucleic acid of formula (VII) is determined by l or n. l and n, independently of one another, are each an integer from 1 to 40, wherein when l or n=1 C is a nucleotide comprising cytosine or an analogue thereof, and when l or n>1 at least 50% of the nucleotides comprise cytosine or an analogue thereof.

For example, without implying any limitation, when l or n=4, Cl or Cn can be, for example, a CUCU, CCUU, UCUC, UUCC, CUUC, CCCU, CCUC, CUCC, UCCC or CCCC, etc.; when l or n=5 Cl or Cn can be, for example, a CCCUU, CCUCU, CUCCU, UCCCU, UCCUC, UCUCC, UUCCC, CUCUC, CCCCU, CCCUC, CCUCC, CUCCC, UCCCC, or CCCCC, etc.

A nucleotide adjacent to Xm in the nucleic acid of formula (VII) preferably does not comprise uracil. Preferably, for formula (VI), when l or n>1, at least 60%, 70%, 80%, 90% or even 100% of the nucleotides comprise guanine or an analogue thereof, as defined above.

The remaining nucleotides to 100% (when nucleotides comprising guanine constitutes less than 100% of the nucleotides) in the flanking sequences G1 and/or Gn are uridine or an analogue thereof, as defined hereinbefore. Also preferably, l and n, independently of one another, are each an integer from 2 to 30, more preferably an integer from 2 to 20 and yet more preferably an integer from 2 to 15. The lower limit of l or n can be varied if necessary and is at least 1, preferably at least 2, more preferably at least 3, 4, 5, 6, 7, 8, 9 or 10. This definition applies correspondingly to formula (VII).

According to a further preferred embodiment, the isRNA as described herein consists of or comprises a nucleic acid of formula (VIII) or (IX):

(NᵤGₗXₘGₙNᵥ)ₐ (formula (VIII))

wherein:
G is a nucleotide comprising guanine, uracil or an analogue of guanine or uracil, preferably comprising guanine or an analogue thereof;
X is a nucleotide comprising guanine, uracil, adenine, thymine, cytosine, or an analogue thereof, preferably comprising uracil or an analogue thereof;
N is a nucleic acid sequence having a length of about 4 to 50, preferably of about 4 to 40, more preferably of about 4 to 30 or 4 to 20 nucleic acids, each N independently being selected from a nucleotide comprising guanine, uracil, adenine, thymine, cytosine or an analogue thereof;
a is an integer from 1 to 20, preferably from 1 to 15, most preferably from 1 to 10;
l is an integer from 1 to 40,
wherein when l=1, G is a nucleotide comprising guanine or an analogue thereof,
when l>1, at least 50% of these nucleotides comprise guanine or an analogue thereof;
m is an integer and is at least 3;
wherein when m=3, X is a nucleotide comprising uracil or an analogue thereof, and
when m>3, at least 3 successive nucleotides comprising uracils or analogues of uracils occur;
n is an integer from 1 to 40,
wherein when n=1, G is a nucleotide comprising guanine or an analogue thereof,
when n>1, at least 50% of these nucleotides comprise guanine or an analogue thereof;
u,v may be independently from each other an integer from 0 to 50,
preferably wherein when u=0, v≥1, or when v=0, u≥1;
wherein the nucleic acid molecule of formula (VIII) has a length of at least 50 nucleotides, preferably of at least 100 nucleotides, more preferably of at least 150 nucleotides, even more preferably of at least 200 nucleotides and most preferably of at least 250 nucleotides.

(NᵤCₗXₘCₙNᵥ)ₐ (formula (IX))

wherein:
C is a nucleotide comprising cytosine, uracil or an analogue of cytosine or uracil, preferably cytosine or an analogue thereof;
X is a nucleotide comprising guanine, uracil, adenine, thymine, cytosine or an analogue thereof, preferably comprising uracil or an analogue thereof;
N is each a nucleic acid sequence having independent from each other a length of about 4 to 50, preferably of about 4 to 40, more preferably of about 4 to 30 or 4 to 20 nucleic acids, each N independently being selected from a nucleotide comprising guanine, uracil, adenine, thymine, cytosine or an analogue thereof;
a is an integer from 1 to 20, preferably from 1 to 15, most preferably from 1 to 10;
l is an integer from 1 to 40,
wherein when l=1, C is a nucleotide comprising cytosine or an analogue thereof,
when l>1, at least 50% of these nucleotides comprise cytosine or an analogue thereof;
m is an integer and is at least 3;
wherein when m=3, X is a nucleotide comprising uracil or an analogue thereof,
when m>3, at least 3 successive nucleotides comprising uracils or analogues of uracil occur;
n is an integer from 1 to 40,
wherein when n=1, C is a nucleotide comprising cytosine or an analogue thereof,
when n>1, at least 50% of these nucleotides comprise cytosine or an analogue thereof.
u, v may be independently from each other an integer from 0 to 50,
preferably wherein when u=0, v≥1, or when v=0, u≥1;
wherein the nucleic acid molecule of formula (IX) according to the invention has a length of at least 50 nucleotides, preferably of at least 100 nucleotides, more preferably of at least 150 nucleotides, even more preferably of at least 200 nucleotides and most preferably of at least 250 nucleotides.

For formula (IX), any of the definitions given above for elements N (i.e. Nu and Nv) and X (Xm), particularly the core structure as defined above, as well as for integers a, l, m, n, u and v, similarly apply to elements of formula (V) correspondingly, wherein in formula (IX) the core structure is defined by CₗXₘCₙ. The definition of bordering elements Nu and Nv is identical to the definitions given above for Nu and Nv.

In particular in the context of formulas (VI)-(IX) above, a “nucleotide” is understood as a molecule comprising or preferably consisting of a nitrogenous base (preferably selected from adenine (A), cytosine (C), guanine (G), thymine (T), or uracil (U), a pentose sugar (ribose or deoxyribose), and at least one phosphate group. “Nucleosides” consist of a nucleobase and a pentose sugar (i.e. could be referred to as “nucleotides without phosphate groups”). Thus, a “nucleotide” comprising a specific base (A, C, G, T or U) preferably also comprises the respective nucleoside (adenosine, cytidine, guanosine, thymidine or uridine, respectively) in addition to one (two, three or more) phosphate groups

That is, the term “nucleotides” includes nucleoside monophosphates (AMP, CMP, GMP, TMP and UMP), nucleoside diphosphates (ADP, CDP, GDP, TDP and UDP), nucleoside triphosphates (ATP, CTP, GTP, TTP and UTP). In the context of formulas (VI)-(IX) above, nucleoside monophosphates are particularly preferred. The expression “a nucleotide comprising ( . . . ) or an analogue thereof” refers to modified nucleotides comprising a modified (phosphate) backbone, pentose sugar(s), or nucleobases. In this context, modifications of the nucleobases are particularly preferred. By way of example, when referring “to a nucleotide comprising guanine, uracil, adenine, thymine, cytosine or an analogue thereof”, the term “analogue thereof” refers to both the nucleotide and the recited nucleobases, preferably to the recited nucleobases.

In preferred embodiments, the (pharmaceutical) composition or vaccine of the invention comprises at least one immunostimulating RNA comprising or consisting of a nucleic acid sequence according to formula (VI) (GₗXₘGₙ), formula (VII) (CₗXₘCₙ), formula (VIII) (NᵤGₗXₘGₙNᵥ)ₐ, and/or formula (IX) (NᵤCₗXₘCₙNᵥ)ₐ). In particularly preferred embodiments, the (pharmaceutical) composition or vaccine of the invention comprises at least one immunostimulating RNA comprising or consisting of a nucleic acid sequence according to any SEQ ID NO as shown in WO2008014979, WO2009030481, WO2009095226, or WO2015149944.

In particularly preferred embodiments, the (pharmaceutical) composition or vaccine of the invention comprises a polymeric carrier cargo complex, formed by a polymeric carrier, preferably comprising disulfide-crosslinked cationic peptides, preferably Cys-Arg₁₂, and/or Cys-Arg₁₂-Cys, and at least one isRNA, preferably comprising or consisting of a nucleic acid sequence according to any SEQ ID NO as shown in WO2008014979, WO2009030481, WO2009095226, or WO2015149944.

The (pharmaceutical) composition or vaccine of the invention may additionally contain one or more auxiliary substances in order to increase its immunogenicity or immunostimulatory capacity, if desired. A synergistic action of the inventive polymeric carrier cargo complex as defined herein and of an auxiliary substance, which may be optionally contained in the (pharmaceutical) composition or vaccine of the invention as defined herein, is preferably achieved thereby. Depending on the various types of auxiliary substances, various mechanisms can come into consideration in this respect. For example, compounds that permit the maturation of dendritic cells (DCs), for example lipopolysaccharides, TNF-alpha or CD40 ligand, form a first class of suitable auxiliary substances. In general, it is possible to use as auxiliary substance any agent that influences the immune system in the manner of a “danger signal” (LPS, GP96, etc.) or cytokines, such as GM-CFS, which allow an immune response to be enhanced and/or influenced in a targeted manner. Particularly preferred auxiliary substances are cytokines, such as monokines, lymphokines, interleukins or chemokines, that further promote the innate immune response, such as IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, INF-alpha, IFN-beta, INF-gamma, GM-CSF, G-CSF, M-CSF, LT-beta or TNF-alpha, growth factors, such as hGH.

The (pharmaceutical) composition or vaccine of the invention may additionally contain any further compound, which is known to be immunostimulating due to its binding affinity (as ligands) to human Toll-like receptors TLR1, TLR2, TLR3, TLR4, TLR5, TLR6, TLR7, TLR8, TLR9, TLR10, or due to its binding affinity (as ligands) to murine Toll-like receptors TLR1, TLR2, TLR3, TLR4, TLR5, TLR6, TLR7, TLR8, TLR9, TLR10, TLR11, TLR12 or TLR13.

The (pharmaceutical) composition or vaccine of the invention may additionally contain CpG nucleic acids, in particular CpG-RNA or CpG-DNA. A CpG-RNA or CpG-DNA can be a single-stranded CpG-DNA (ss CpG-DNA), a double-stranded CpG-DNA (dsDNA), a single-stranded CpG-RNA (ss CpG-RNA) or a double-stranded CpG-RNA (ds CpG-RNA). The CpG nucleic acid is preferably in the form of CpG-RNA, more preferably in the form of single-stranded CpG-RNA (ss CpG-RNA). The CpG nucleic acid preferably contains at least one or more (mitogenic) cytosine/guanine dinucleotide sequence(s) (CpG motif(s)). According to a first preferred alternative, at least one CpG motif contained in these sequences, that is to say the C (cytosine) and the G (guanine) of the CpG motif, is unmethylated. All further cytosines or guanines optionally contained in these sequences can be either methylated or unmethylated. According to a further preferred alternative, however, the C (cytosine) and the G (guanine) of the CpG motif can also be present in methylated form.

### Kit

In a further aspect, the present invention relates to a kit or kit-of-parts comprising the artificial nucleic acid (RNA) molecule, and/or the (pharmaceutical) composition or vaccine of the invention.

In the inventive kit or kit-of-parts, the at least one artificial nucleic acid (RNA) molecule in lyophilized or liquid form, optionally together with one or more pharmaceutically acceptable carrier(s), excipients or further agents as described above in the context of the pharmaceutical composition.

Optionally, the kit or kit-of-parts of the invention may comprise at least one further agent as defined herein in the context of the pharmaceutical composition, antimicrobial agents, RNAse inhibitors, solubilizing agents or the like.

The kit-of-parts may be a kit of two or more parts and typically comprises its components in suitable containers. For example, each container may be in the form of vials, bottles, squeeze bottles, jars, sealed sleeves, envelopes or pouches, tubes or blister packages or any other suitable form provided the container is configured so as to prevent premature mixing of components. Each of the different components may be provided separately, or some of the different components may be provided together (i.e. in the same container).

A container may also be a compartment or a chamber within a vial, a tube, a jar, or an envelope, or a sleeve, or a blister package or a bottle, provided that the contents of one compartment are not able to associate physically with the contents of another compartment prior to their deliberate mixing by a pharmacist or physician.

The kit-of-parts may furthermore contain technical instructions with information on the administration and dosage of any of its components.

### Medical Use and Treatment

The artificial nucleic acid (RNA) molecule, or the (pharmaceutical) composition or vaccine or kit of the invention may be used for human and also for veterinary medical purposes, preferably for human medical purposes.

According to a further aspect, the invention thus relates to the artificial nucleic acid (RNA) molecule, (pharmaceutical) composition or vaccine or kit of the invention for use as a medicament.

The artificial nucleic acid (RNA) molecule, (pharmaceutical) composition or vaccine or kit of the invention may be used for treatment of genetic diseases, cancer, autoimmune diseases, inflammatory diseases, and infectious diseases, or other diseases or conditions.

According to a further aspect, the invention thus relates to the artificial nucleic acid (RNA) molecule, (pharmaceutical) composition or vaccine or kit of the invention for use in a method of treatment of genetic diseases, cancer, autoimmune diseases, inflammatory diseases, and infectious diseases, or other diseases or conditions.

“Gene therapy” preferably involves modulating (i.e. restoring, enhancing, decreasing or inhibiting) gene expression in a subject in order to achieve a therapeutic effect. To this end, gene therapy typically encompasses the introduction of nucleic acids into cells. The term generally refers to the manipulation of a genome for therapeutic purposes and includes the use of genome-editing technologies for correction of mutations that cause disease, the addition of therapeutic genes to the genome, the removal of deleterious genes or genome sequences, and the modulation of gene expression. Gene therapy may involve in vivo or ex vivo transformation of the host cells.

The term “treatment” or “treating” of a disease includes preventing or protecting against the disease (that is, causing the clinical symptoms not to develop); inhibiting the disease (i.e., arresting or suppressing the development of clinical symptoms; and/or relieving the disease (i.e., causing the regression of clinical symptoms). As will be appreciated, it is not always possible to distinguish between “preventing” and “suppressing” a disease or disorder since the ultimate inductive event or events may be unknown or latent. Accordingly, the term “prophylaxis” will be understood to constitute a type of “treatment” that encompasses both “preventing” and “suppressing.” The term “treatment” thus includes “prophylaxis”.

The term “subject”, “patient” or “individual” as used herein generally includes humans and non-human animals and preferably mammals (e.g., non-human primates, including marmosets, tamarins, spider monkeys, owl monkeys, vervet monkeys, squirrel monkeys, and baboons, macaques, chimpanzees, orangutans, gorillas; cows; horses; sheep; pigs; chicken; cats; dogs; mice; rat; rabbits; guinea pigs; etc.), including chimeric and transgenic animals and disease models. In the context of the present invention, the term “subject” preferably refers a non-human primate or a human, most preferably a human.

Accordingly, the present invention further provides methods of treating a disease as disclosed herein, by administering to a subject in need thereof a pharmaceutically effective amount of the artificial nucleic acid (RNA) molecule, (pharmaceutical) composition or vaccine or kit. Such methods may comprise an optional first step of preparing the inventive artificial nucleic acid (RNA) molecule, (pharmaceutical) composition or vaccine or kit, and a second step, comprising administering (a pharmaceutically effective amount of) said artificial nucleic acid (RNA) molecule, (pharmaceutical) composition or vaccine or kit to a patient/subject in need thereof.

### Administration Routes

The inventive artificial nucleic acid (RNA) molecule, the (pharmaceutical) composition or vaccine or kit may be administered, for example, systemically or locally.

Routes for systemic administration in general include, for example, transdermal, oral, parenteral routes, including subcutaneous, intravenous, intramuscular, intraarterial, intradermal and intraperitoneal injections and/or intranasal administration routes.

Routes for local administration in general include, for example, topical administration routes but also intradermal, transdermal, subcutaneous, or intramuscular injections or intralesional, intratumoral, intracranial, intrapulmonal, intracardial, and sublingual injections.

In case more than one different artificial nucleic acid (RNA) molecule is to be administered, different administration routes can be used for each of said different artificial nucleic acid (RNA) molecules.

According to preferred embodiments, the artificial nucleic acid (RNA) molecule, (pharmaceutical) composition or vaccine or kit is administered by a parenteral route, preferably via intradermal, subcutaneous, or intramuscular routes. Preferably, said artificial nucleic acid (RNA) molecule, (pharmaceutical) composition or vaccine or kit may be administered by injection, e.g. subcutaneous, intramuscular or intradermal injection, which may be needle-free and/or needle injection. Accordingly, in preferred embodiments, the medical use and/or method of treatment according to the present invention involves administration of said artificial nucleic acid (RNA) molecule, (pharmaceutical) composition or vaccine or kit by subcutaneous, intramuscular or intradermal injection, preferably by intramuscular or intradermal injection, more preferably by intradermal injection. Such injection may be carried out by using conventional needle injection or (needle-free) jet injection, preferably by using (needle-free) jet injection.

### Administration Regimen

The artificial nucleic acid (RNA) molecule, (pharmaceutical) composition or vaccine or kit of the invention may be administered to a subject in need thereof several times a day, daily, every other day, weekly, or monthly; and may be administered sequentially or simultaneously.

In case different artificial nucleic acid (RNA) molecules are administered, or the (pharmaceutical) composition or vaccine or kit comprises several components, e.g. different artificial nucleic acid (RNA) molecules and optionally additional active agents as described herein, each component may be administered simultaneously (at the same time via the same or different administration routes) or separately (at different times via the same or different administration routes). Such a sequential administration scheme is also referred to as “time-staggered” administration. Time-staggered administration may mean that an artificial nucleic acid (RNA) molecule of the invention is administrated e.g. prior, concurrent or subsequent to a different artificial nucleic acid (RNA) molecule of the invention, or any other additional active agent.

### Dose

The inventive artificial nucleic acid (RNA) molecule, (pharmaceutical) composition or vaccine or kit may preferably be administered in a safe and therapeutically effective amount.

As used herein, “safe and (therapeutically) effective amount” means an amount of the active agent(s) that is sufficient to elicit a desired biological or medicinal response in a tissue, system, animal or human that is being sought. A safe and therapeutically effective amount is preferably sufficient for the inducing a positive modification of the disease to be treated, i.e. for alleviation of the symptoms of the disease being treated, reduction of disease progression, or prophylaxis of the symptoms of the disease being prevented. At the same time, however, a “safe and therapeutically effective amount” is preferably small enough to avoid serious side-effects, that is to say to permit a sensible relationship between advantage and risk.

A “safe and (therapeutically) effective amount” will furthermore vary in connection with the particular condition to be treated and also with the age, physical condition, body weight, sex and diet of the patient to be treated, the severity of the condition, the duration of the treatment, the nature of the accompanying therapy, of the particular pharmaceutically acceptable carrier or excipient used, the treatment regimen and similar factors.

A “safe and (therapeutically) effective amount” of the artificial nucleic acid (RNA) molecule, may furthermore be selected depending on the type of artificial nucleic acid (RNA) molecule, e.g. monocistronic, bi- or even multicistronic RNA, since a bi- or even multicistronic RNA may lead to a significantly higher expression of the encoded (poly-)peptide or protein of interest an equal amount of a monocistronic RNA.

Therapeutic efficacy and toxicity of the inventive artificial nucleic acid (RNA) molecule, (pharmaceutical) composition or vaccine or kit may be determined by standard pharmaceutical procedures in cell cultures or experimental animals, e.g., for determining the LD50 (the dose lethal to 50% of the population) and the ED50 (the dose therapeutically effective in 50% of the population). Exemplary animal models suitable for determining a “safe and (therapeutically) effective amount of artificial nucleic acid (RNA) molecules, (pharmaceutical) compositions or kits disclosed herein include, without implying any limitation, rabbit, sheep, mouse, rat, dog and non-human primate models. The dose ratio between toxic and therapeutic effects is the therapeutic index and can be expressed as the ratio LD50/ED50. Artificial nucleic acid (RNA) molecules, (pharmaceutical) compositions or kits which exhibit large therapeutic indices are generally preferred. The data obtained from the cell culture assays and animal studies can be used in formulating a range of dosage for use in humans. The dosage of such compounds lies preferably within a range of circulating concentrations that include the ED50 with little or no toxicity.

For instance, therapeutically effective doses of the inventive artificial nucleic acid (RNA) molecule, (pharmaceutical) composition or vaccine or kit described herein may range from about 0.001 mg to 10 mg, preferably from about 0.01 mg to 5 mg, more preferably from about 0.1 mg to 2 mg per dosage unit or from about 0.01 nmol to 1 mmol per dosage unit, in particular from 1 nmol to 1 mmol per dosage unit, preferably from 1 pmol to 1 mmol per dosage unit. It is also envisaged that the therapeutically effective dose of the inventive artificial nucleic acid (RNA) molecule, (pharmaceutical) composition or vaccine or kit may range (per kg body weight) from about 0.01 mg/kg to 10 g/kg, preferably from about 0.05 mg/kg to 5 g/kg, more preferably from about 0.1 mg/kg to 2.5 g/kg.

### Genetic Diseases

In preferred embodiments, artificial nucleic acid (RNA) molecules, (pharmaceutical) composition or vaccine or kit is used for treatment or prophylaxis of genetic diseases.

As used herein, the term “genetic disease” includes any disease, disorder or conditions caused by, characterized by or related to abnormalities (i.e. deviations from the wild-type, healthy and non-symptomatic state) in the genome. Such abnormalities may include a change in chromosomal copy number (e.g., aneuploidy), or a portion thereof (e.g., deletions, duplications, amplifications); or a change in chromosomal structure (e.g., translocations, point mutations). Genomes abnormality may be hereditary (either recessive or dominant) or non-hereditary. Genome abnormalities may be present in some cells of an organism or in all cells of that organism and include autosomal, X-linked, Y-linked and mitochondrial abnormalities.

Further, the present invention allows treating all diseases, hereditary diseases or genetic diseases as mentioned in WO 2012/013326 A1, which is incorporated by reference in its entirety herein.

### Cancer

In preferred embodiments, artificial nucleic acid (RNA) molecules, (pharmaceutical) composition or vaccine or kit is used for treatment or prophylaxis of cancer.

As used herein, the term “cancer” refers to a neoplasm characterized by the uncontrolled and usually rapid proliferation of cells that tend to invade surrounding tissue and to metastasize to distant body sites. The term encompasses benign and malignant neoplasms. Malignancy in cancers is typically characterized by anaplasia, invasiveness, and metastasis; whereas benign malignancies typically have none of those properties. The terms includes neoplasms characterized by tumor growth as well as cancers of blood and lymphatic system.

In some embodiments, artificial nucleic acid (RNA) molecules, (pharmaceutical) composition or vaccine or kit according to the invention may be used as a medicament, in particular for treatment of tumor or cancer diseases. In this context, treatment preferably involves intratumoral application, especially by intratumoral injection. Accordingly, the artificial nucleic acid (RNA) molecules, (pharmaceutical) composition or vaccine or kit according to the invention may be used for preparation of a medicament for treatment of tumor or cancer diseases, said medicament being particularly suitable for intratumoral application (administration) for treatment of tumor or cancer diseases.

Preferably, tumor and cancer diseases as mentioned herein are selected from tumor or cancer diseases which preferably include e.g. Acute lymphoblastic leukemia, Acute myeloid leukemia, Adrenocortical carcinoma, AIDS-related cancers, AIDS-related lymphoma, Anal cancer, Appendix cancer, Astrocytoma, Basal cell carcinoma, Bile duct cancer, Bladder cancer, Bone cancer, Osteosarcoma/Malignant fibrous histiocytoma, Brainstem glioma, Brain tumor, cerebellar astrocytoma, cerebral astrocytoma/malignant glioma, ependymoma, medulloblastoma, supratentorial primitive neuroectodermal tumors, visual pathway and hypothalamic glioma, Breast cancer, Bronchial adenomas/carcinoids, Burkitt lymphoma, childhood Carcinoid tumor, gastrointestinal Carcinoid tumor, Carcinoma of unknown primary, primary Central nervous system lymphoma, childhood Cerebellar astrocytoma, childhood Cerebral astrocytoma/Malignant glioma, Cervical cancer, Childhood cancers, Chronic lymphocytic leukemia, Chronic myelogenous leukemia, Chronic myeloproliferative disorders, Colon Cancer, Cutaneous T-cell lymphoma, Desmoplastic small round cell tumor, Endometrial cancer, Ependymoma, Esophageal cancer, Ewing's sarcoma in the Ewing family of tumors, Childhood Extracranial germ cell tumor, Extragonadal Germ cell tumor, Extrahepatic bile duct cancer, Intraocular melanoma, Retinoblastoma, Gallbladder cancer, Gastric (Stomach) cancer, Gastrointestinal Carcinoid Tumor, Gastrointestinal stromal tumor (GIST), extracranial, extragonadal, or ovarian Germ cell tumor, Gestational trophoblastic tumor, Glioma of the brain stem, Childhood Cerebral Astrocytoma, Childhood Visual Pathway and Hypothalamic Glioma, Gastric carcinoid, Hairy cell leukemia, Head and neck cancer, Heart cancer, Hepatocellular (liver) cancer, Hodgkin lymphoma, Hypopharyngeal cancer, childhood Hypothalamic and visual pathway glioma, Intraocular Melanoma, Islet Cell Carcinoma (Endocrine Pancreas), Kaposi sarcoma, Kidney cancer (renal cell cancer), Laryngeal Cancer, Leukemias, acute lymphoblastic Leukemia, acute myeloid Leukemia, chronic lymphocytic Leukemia, chronic myelogenous Leukemia, hairy cell Leukemia, Lip and Oral Cavity Cancer, Liposarcoma, Liver Cancer, Non-Small Cell Lung Cancer, Small Cell Lung Cancer, Lymphomas, AIDS-related Lymphoma, Burkitt Lymphoma, cutaneous T-Cell Lymphoma, Hodgkin Lymphoma, Non-Hodgkin Lymphomas, Primary Central Nervous System Lymphoma, Waldenström Macroglobulinemia, Malignant Fibrous Histiocytoma of Bone/Osteosarcoma, Childhood Medulloblastoma, Melanoma, Intraocular (Eye) Melanoma, Merkel Cell Carcinoma, Adult Malignant Mesothelioma, Childhood Mesothelioma, Metastatic Squamous Neck Cancer with Occult Primary, Mouth Cancer, Childhood Multiple Endocrine Neoplasia Syndrome, Multiple Myeloma/Plasma Cell Neoplasm, Mycosis Fungoides, Myelodysplastic Syndromes, Myelodysplastic/Myeloproliferative Diseases, Chronic Myelogenous Leukemia, Adult Acute Myeloid Leukemia, Childhood Acute Myeloid Leukemia, Multiple Myeloma (Cancer of the Bone-Marrow), Chronic Myeloproliferative Disorders, Nasal cavity and paranasal sinus cancer, Nasopharyngeal carcinoma, Neuroblastoma, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, Pancreatic cancer, islet cell Pancreatic cancer, Paranasal sinus and nasal cavity cancer, Parathyroid cancer, Penile cancer, Pharyngeal cancer, Pheochromocytoma, Pineal astrocytoma, Pineal germinoma, childhood Pineoblastoma and supratentorial primitive neuroectodermal tumors, Pituitary adenoma, Plasma cell neoplasia/Multiple myeloma, Pleuropulmonary blastoma, Primary central nervous system lymphoma, Prostate cancer, Rectal cancer, Renal cell carcinoma (kidney cancer), Cancer of the Renal pelvis and ureter, Retinoblastoma, childhood Rhabdomyosarcoma, Salivary gland cancer, Sarcoma of the Ewing family of tumors, Kaposi Sarcoma, soft tissue Sarcoma, uterine Sarcoma, Sezary syndrome, Skin cancer (nonmelanoma), Skin cancer (melanoma), Merkel cell Skin carcinoma, Small intestine cancer, Squamous cell carcinoma, metastatic Squamous neck cancer with occult primary, childhood Supratentorial primitive neuroectodermal tumor, Testicular cancer, Throat cancer, childhood Thymoma, Thymoma and Thymic carcinoma, Thyroid cancer, childhood Thyroid cancer, Transitional cell cancer of the renal pelvis and ureter, gestational Trophoblastic tumor, Urethral cancer, endometrial Uterine cancer, Uterine sarcoma, Vaginal cancer, childhood Visual pathway and hypothalamic glioma, Vulvar cancer, Waldenström macroglobulinemia, and childhood Wilms tumor (kidney cancer).

Further, the present invention allows treating all diseases or cancer diseases as mentioned in WO 2012/013326 A1 or WO 2017/109134 A1, which is incorporated by reference in its entirety herein.

### Infectious Diseases

In preferred embodiments, artificial nucleic acid (RNA) molecules, (pharmaceutical) composition or vaccine or kit is used for treatment or prophylaxis of infectious diseases.

The term “infection” or “infectious disease” relates to the invasion and multiplication of microorganisms such as bacteria, viruses, and parasites that are not normally present within the body. An infection may cause no symptoms and be subclinical, or it may cause symptoms and be clinically apparent. An infection may remain localized, or it may spread through the blood or lymphatic system to become systemic. Infectious diseases in this context, preferably include viral, bacterial, fungal or protozoological infectious diseases.

In particular, infectious diseases may be selected from, Acinetobacter infections, African sleeping sickness (African trypanosomiasis), AIDS (Acquired immunodeficiency syndrome), Amoebiasis, Anaplasmosis, Anthrax, Appendicitis, Arcanobacterium haemolyticum infections, Argentine hemorrhagic fever, Ascariasis, Aspergillosis, Astrovirus infections, Athlete's foot, Babesiosis, Bacillus cereus infections, Bacterial meningitis, Bacterial pneumonia, Bacterial vaginosis (BV), Bacteroides infections, Balantidiasis, Baylisascaris infections, Bilharziosis, BK virus infections, Black piedra, Blastocystis hominis infections, Blastomycosis, Bolivian hemorrhagic fever, Borrelia infections (Borreliosis), Botulism (and Infant botulism), Bovine tapeworm, Brazilian hemorrhagic fever, Brucellosis, Burkholderia infections, Buruli ulcer, Calicivirus infections (Norovirus and Sapovirus), Campylobacteriosis, Candidiasis (Candidosis), Canine tapeworm infections, Cat-scratch disease, Chagas Disease (American trypanosomiasis), Chancroid, Chickenpox, Chlamydia infections, Chlamydia trachomatis infections, Chlamydophila pneumoniae infections, Cholera, Chromoblastomycosis, Climatic bubo, Clonorchiasis, Clostridium difficile infections, Coccidioidomycosis, Cold, Colorado tick fever (CTF), Common cold (Acute viral rhinopharyngitis; Acute coryza), Condyloma acuminata, Conjunctivitis, Creutzfeldt-Jakob disease (CJD), Crimean-Congo hemorrhagic fever (CCHF), Cryptococcosis, Cryptosporidiosis, Cutaneous larva migrans (CLM), Cutaneous Leishmaniosis, Cyclosporiasis, Cysti-cercosis, Cytomegalovirus infections, Dengue fever, Dermatophytosis, Dienta-moebiasis, Diphtheria, Diphyllobothriasis, Donavanosis, Dracunculiasis, Early summer meningoencephalitis (FSME), Ebola hemorrhagic fever, Echinococcosis, Ehrlichiosis, Enterobiasis (Pinworm infections), Enterococcus infections, Enterovirus infections, Epidemic typhus, Epiglottitis, Epstein-Barr Virus Infectious Mononucleosis, Erythema infectiosum (Fifth disease), Exanthem subitum, Fasciolopsiasis, Fasciolosis, Fatal familial insomnia (FFI), Fifth disease, Filariasis, Fish poisoning (Ciguatera), Fish tapeworm, Flu, Food poisoning by Clostridium perfringens, Fox tapeworm, Free-living amebic infections, Fusobacterium infections, Gas gangrene, Geotrichosis, Gerstmann-Straussler-Scheinker syndrome (GSS), Giardiasis, Glanders, Gnathostomiasis, Gonorrhea, Granuloma inguinale (Donovanosis), Group A streptococcal infections, Group B streptococcal infections, Haemophilus influenzae infections, Hand foot and mouth disease (HFMD), Hantavirus Pulmonary Syndrome (HPS), Helicobacter pylori infections, Hemolytic-uremic syndrome (HUS), Hemorrhagic fever with renal syndrome (HFRS), Henipavirus infections, Hepatitis A, Hepatitis B, Hepatitis C, Hepatitis D, Hepatitis E, Herpes simplex, Herpes simplex type I, Herpes simplex type II, Herpes zoster, Histoplasmosis, Hollow warts, Hookworm infections, Human bocavirus infections, Human ewingii ehrlichiosis, Human granulocytic anaplasmosis (HGA), Human metapneumovirus infections, Human monocytic ehrlichiosis, Human papillomavirus (HPV) infections, Human parainfluenza virus infections, Hymenolepiasis, Influenza, Isosporiasis, Japanese encephalitis, Kawasaki disease, Keratitis, Kingella kingae infections, Kuru, Lambliasis (Giardiasis), Lassa fever, Legionellosis (Legionnaires' disease, Pontiac fever), Leishmaniasis, Leprosy, Leptospirosis, Lice, Listeriosis, Lyme borreliosis, Lyme disease, Lymphatic filariasis (Elephantiasis), Lymphocytic choriomeningitis, Malaria, Marburg hemorrhagic fever (MHF), Marburg virus, Measles, Melioidosis (Whitmore's disease), Meningitis, Meningococcal disease, Metagonimiasis, Microsporidiosis, Miniature tapeworm, Miscarriage (prostate inflammation), Molluscum contagiosum (MC), Mononucleosis, Mumps, Murine typhus (Endemic typhus), Mycetoma, Mycoplasma hominis, Mycoplasma pneumonia, Myiasis, Nappy/diaper dermatitis, Neonatal conjunctivitis (Ophthalmia neonatorum), Neonatal sepsis (Chorioamnionitis), Nocardiosis, Noma, Norwalk virus infections, Onchocerciasis (River blindness), Osteomyelitis, Otitis media, Paracoccidioidomycosis (South American blastomycosis), Paragonimiasis, Paratyphus, Pasteurellosis, Pediculosis capitis (Head lice), Pediculosis corporis (Body lice), Pediculosis pubis (Pubic lice, Crab lice), Pelvic inflammatory disease (PID), Pertussis (Whooping cough), Pfeiffer's glandular fever, Plague, Pneumococcal infections, Pneumocystis pneumonia (PCP), Pneumonia, Polio (childhood lameness), Poliomyelitis, Porcine tapeworm, Prevotella infections, Primary amoebic meningoencephalitis (PAM), Progressive multifocal leukoencephalopathy, Pseudo-croup, Psittacosis, Q fever, Rabbit fever, Rabies, Rat-bite fever, Reiter's syndrome, Respiratory syncytial virus infections (RSV), Rhinosporidiosis, Rhinovirus infections, Rickettsial infections, Rickettsialpox, Rift Valley fever (RVF), Rocky mountain spotted fever (RMSF), Rotavirus infections, Rubella, Salmonella paratyphus, Salmonella typhus, Salmonellosis, SARS (Severe Acute Respiratory Syndrome), Scabies, Scarlet fever, Schistosomiasis (Bilharziosis), Scrub typhus, Sepsis, Shigellosis (Bacillary dysentery), Shingles, Smallpox (Variola), Soft chancre, Sporotrichosis, Staphylococcal food poisoning, Staphylococcal infections, Strongyloidiasis, Syphilis, Taeniasis, Tetanus, Three-day fever, Tick-borne encephalitis, Tinea barbae (Barber's itch), Tinea capitis (Ringworm of the Scalp), Tinea corporis (Ringworm of the Body), Tinea cruris (Jock itch), Tinea manuum (Ringworm of the Hand), Tinea nigra, Tinea pedis (Athlete's foot), Tinea unguium (Onychomycosis), Tinea versicolor (Pityriasis versicolor), Toxocariasis (Ocular Larva Migrans (OLM) and Visceral Larva Migrans (VLM)), Toxoplasmosis, Trichinellosis, Trichomoniasis, Trichuriasis (Whipworm infections), Tripper, Trypanosomiasis (sleeping sickness), Tsutsugamushi disease, Tuberculosis, Tularemia, Typhus, Typhus fever, Ureaplasma urealyticum infections, Vaginitis (Colpitis), Variant Creutzfeldt-Jakob disease (vCJD, nvCJD), Venezuelan equine encephalitis, Venezuelan hemorrhagic fever, Viral pneumonia, Visceral Leishmaniosis, Warts, West Nile Fever, Western equine encephalitis, White piedra (Tinea blanca), Whooping cough, Yeast fungus spots, Yellow fever, Yersinia pseudotuberculosis infections, Yersiniosis, and Zygomycosis.

Further infectious diseases include infections caused by Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus, Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O157:H7, O111 and O104:H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus, Hepatitis C Virus, Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family, Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis. In this context, an infectious disease, preferably a viral, bacterial or protozoan infectious diseases, is typically selected from influenza, malaria, SARS, yellow fever, AIDS, Lyme borreliosis, Leishmaniasis, anthrax, meningitis, viral infectious diseases such as AIDS, Condyloma acuminata, hollow warts, Dengue fever, three-day fever, Ebola virus, cold, early summer meningoencephalitis (FSME), flu, shingles, hepatitis, herpes simplex type I, herpes simplex type II, Herpes zoster, influenza, Japanese encephalitis, Lassa fever, Marburg virus, measles, foot-and-mouth disease, mononucleosis, mumps, Norwalk virus infection, Pfeiffer's glandular fever, smallpox, polio (childhood lameness), pseudo-croup, fifth disease, rabies, warts, West Nile fever, chickenpox, cytomegalic virus (CMV), bacterial infectious diseases such as miscarriage (prostate inflammation), anthrax, appendicitis, borreliosis, botulism, Camphylobacter, Chlamydia trachomatis (inflammation of the urethra, conjunctivitis), cholera, diphtheria, donavanosis, epiglottitis, typhus fever, gas gangrene, gonorrhoea, rabbit fever, Helicobacter pylori, whooping cough, climatic bubo, osteomyelitis, Legionnaire's disease, leprosy, listeriosis, pneumonia, meningitis, bacterial meningitis, anthrax, otitis media, Mycoplasma hominis, neonatal sepsis (Chorioamnionitis), noma, paratyphus, plague, Reiter's syndrome, Rocky Mountain spotted fever, Salmonella paratyphus, Salmonella typhus, scarlet fever, syphilis, tetanus, tripper, tsutsugamushi disease, tuberculosis, typhus, vaginitis (colpitis), soft chancre, and infectious diseases caused by parasites, protozoa or fungi, such as amoebiasis, bilharziosis, Chagas disease, Echinococcus, fish tapeworm, fish poisoning (Ciguatera), fox tapeworm, athlete's foot, canine tapeworm, candidosis, yeast fungus spots, scabies, cutaneous Leishmaniosis, lambliasis (giardiasis), lice, malaria, microscopy, onchocercosis (river blindness), fungal diseases, bovine tapeworm, schistosomiasis, porcine tapeworm, toxoplasmosis, trichomoniasis, trypanosomiasis (sleeping sickness), visceral Leishmaniosis, nappy/diaper dermatitis or miniature tapeworm.

### Autoimmune Diseases

In preferred embodiments, artificial nucleic acid (RNA) molecules, (pharmaceutical) composition or vaccine or kit is used for treatment or prophylaxis of autoimmune diseases.

The term “autoimmune disease” refers to any disease, disorder or condition in a subject characterized by cellular, tissue and/or organ injury caused by an immunologic reaction of the subject to its own cells, tissues and/or organs. Typically, “autoimmune diseases” result from, or are aggravated by, the production of antibodies that are reactive with autoantigens, i.e. antigens expressed by healthy body cells.

Autoimmune diseases can be broadly divided into systemic and organ-specific or localised autoimmune disorders, depending on the principal clinico-pathologic features of each disease. Autoimmune diseases may be divided into the categories of systemic syndromes, including, but not limited to, systemic lupus erythematosus (SLE), Sjögren's syndrome, Scleroderma, Rheumatoid Arthritis and polymyositis or local syndromes which may be endocrinologic (type I diabetes (Diabetes mellitus Type 1), Hashimoto's thyroiditis, Addison's disease etc.), dermatologic (pemphigus vulgaris), haematologic (autoimmune haemolytic anaemia), neural (multiple sclerosis) or can involve virtually any circumscribed mass of body tissue. Autoimmune diseases in the context of the present invention may be selected from the group consisting of type I autoimmune diseases or type II autoimmune diseases or type III autoimmune diseases or type IV autoimmune diseases, such as, for example, multiple sclerosis (MS), rheumatoid arthritis, diabetes, type I diabetes (Diabetes mellitus Type 1), chronic polyarthritis, Basedow's disease, autoimmune forms of chronic hepatitis, colitis ulcerosa, type I allergy diseases, type II allergy diseases, type III allergy diseases, type IV allergy diseases, fibromyalgia, hair loss, Bechterew's disease, Crohn's disease, Myasthenia gravis, neurodermitis, Polymyalgia rheumatica, progressive systemic sclerosis (PSS), Reiter's syndrome, rheumatic arthritis, psoriasis, vasculitis, and type II diabetes.

### Inflammatory Diseases

In preferred embodiments, artificial nucleic acid (RNA) molecules, (pharmaceutical) composition or vaccine or kit is used for treatment or prophylaxis of inflammatory diseases.

The term “inflammatory disease” refers to any disease, disorder or condition in a subject characterized by, caused by, resulting from, or accompanied by inflammation, preferably chronic inflammation. Autoimmune disorders may or may not be associated with inflammation. Moreover, inflammation may or may not be caused by an autoimmune disorder. Thus, certain disorders may be characterized as both autoimmune and inflammatory disorders.

Exemplary inflammatory diseases in the context of the present invention include, without limitation, rheumatoid arthritis, Crohn's disease, diabetic retinopathy, psoriasis, endometriosis, Alzheimer's, ankylosing spondylitis, arthritis (osteoarthritis, rheumatoid arthritis (RA), psoriatic arthritis), asthma, atherosclerosis, colitis, dermatitis, diverticulitis, fibromyalgia, hepatitis, irritable bowel syndrome (IBS), systemic lupus erythematous (SLE), nephritis, Parkinson's disease, and ulcerative colitis.

### Allergies

In preferred embodiments, artificial nucleic acid (RNA) molecules, (pharmaceutical) composition or vaccine or kit is used for treatment or prophylaxis of allergies.

The term “allergy” or “allergic hypersensitivity” refers to any disease, disorder or condition caused by or characterized by a hypersensitivity reaction initiated by immunologic mechanisms in response to a substance (allergen), often in a genetically predisposed individual (atopy). Allergy can be antibody- or cell-mediated. In most patients, the antibody typically responsible for an allergic reaction belongs to the IgE isotype (IgE-mediated allergy, type-I allergy). In non IgE-mediated allergy, the antibody may belong to the IgG isotype. Allergies may be classified according to the source of the antigen evoking the hypersensitive reaction. In the context of the present invention, allergies may be selected from (a) food allergy, (b) drug allergy, (c) house dust allergy, (d) insect venom or bite allergy, and (e) pollen allergy. Alternatively, allergies may be classified based on the major symptoms of the hypersensitive reaction. In the context of the present invention, allergies may be selected from the group of (a) asthma, (b) rhinitis, (c) conjunctivitis, (d) rhinoconjuctivitis, (e) dermatitis, (f) urticaria and (g) anaphylaxis.

### Combination Therapy

The inventive artificial nucleic acid (RNA) molecule, (pharmaceutical) composition or vaccine or kit may also be used in combination therapy. Any other therapy useful for treating or preventing the diseases and disorders defined herein may be combined with the uses and methods disclosed herein.

For instance, the subject receiving the inventive artificial nucleic acid (RNA) molecule, (pharmaceutical) composition or vaccine or kit may be a patient with cancer, preferably as defined herein, or a related condition, receiving chemotherapy (e.g. first-line or second-line chemotherapy), radiotherapy, chemoradiation (combination of chemotherapy and radiotherapy), tyrosine kinase inhibitors (e.g. EGFR tyrosine kinase inhibitors), antibody therapy and/or inhibitory and/or stimulatory checkpoint molecules (e.g. CTLA4 inhibitors), or a patient, who has achieved partial response or stable disease after having received one or more of the treatments specified above. Or, the subject receiving the inventive artificial nucleic acid (RNA) molecule, (pharmaceutical) composition or vaccine or kit may be a patient with an infectious disease, preferably as defined herein, receiving antibiotic, antifungal or antiviral therapy.

In a further aspect, the present invention thus also relates to the use of the inventive artificial nucleic acid (RNA) molecule, (pharmaceutical) composition or vaccine or kit-of-parts for supporting another therapy of cancer, an infectious disease, or any other disease amenable by treatment with said artificial nucleic acid molecule, (pharmaceutical) composition or vaccine or kit.

Administration of the inventive artificial nucleic acid (RNA) molecule, (pharmaceutical) composition or vaccine or kit-of-parts may be accomplished prior to, simultaneously and/or subsequently to administering another therapeutic or subjecting the patient to another therapy that is useful for treatment of the particular disease or condition to be treated.

### In Vitro Methods

In further aspects, the present invention provides useful in vitro methods that allow to determine and prepare suitable UTR combinations artificial nucleic acid molecules comprising the same, preferably capable of increasing the expression efficiency of an operably linked coding sequence.

Thus, the present invention provides a method for increasing the expression efficacy of an artificial nucleic acid (RNA) molecule comprising at least one coding region encoding a (poly-)peptide or protein preferably as disclosed herein, said method comprising (a) associating said coding region with a at least one 5′ UTR element derived from a 5′ UTR of a gene selected from the group consisting of HSD17B4, ASAH1, ATP5A1, MP68, NDUFA4, NOSIP, RPL31, SLC7A3, TUBB4B and UBQLN2, or from a corresponding RNA sequence, homolog, a fragment or a variant thereof; (b) associating said coding region with at least one 3′ UTR element derived from a 3′ UTR of a gene selected from the group consisting of PSMB3, CASP1, COX6B1, GNAS, NDUFA1 and RPS9, or from a corresponding RNA sequence, homolog, a fragment or a variant thereof; and (c) obtaining an artificial nucleic acid (RNA) molecule.

In a further aspect, the present invention provides a method of identifying a combination of 5′ UTR and 3′ UTR capable of increasing the expression efficiency in a desired tissue or a cell derived from the desired tissue, comprising: a) generating a library of artificial nucleic acid molecules (“test constructs”), each comprising a “reporter ORF” encoding a detectable reporter polynucleotide, preferably selected luciferase or eGFP, operably linked to one of the 5′ UTRs and/or one of the 3′ UTRs as defined in claim 3; b) providing an artificial nucleic acid molecule comprising said “reporter ORF” operably linked to reference 5′ and 3′ UTRs, preferably RPL32 and ALB7 as a “reference construct”; c) introducing said test constructs and said reference constructs into the desired tissue or cell under suitable conditions allowing their expression; d) detecting and quantifying the expression of said polypeptide from the “reporter ORF” from the test constructs and the reference construct; e) comparing the polypeptide expression from the test constructs and reference constructs; wherein test constructs characterized by an increased polypeptide expression as compared to the reference construct are identified as being capable of increasing the expression efficiency in the desired tissue or cell.

### DESCRIPTION OF THE FIGURES

FIG. 1: Mean expression profiles of selected (poly-)peptides and proteins of interest from RNA constructs comprising inventive UTR combinations.

FIG. 2: Mean expression profiles from RNA constructs comprising inventive UTR combinations operably linked to coding regions encoding different (poly-)peptides or proteins of interest and an A64 poly(A) sequence followed by N5 as 3′ UTR.

FIG. 3: Mean expression profiles of RNA constructs comprising polyC and histone stem loop in addition to inventive UTR combinations operably linked to coding region encoding different (poly-)peptides or proteins of interest in different cell lines.

FIG. 4: Mean expression profiles of RNA constructs comprising inventive UTR combinations operably linked to coding region encoding erythropoietin (EPO) in different cell lines.

FIG. 5: Mean expression profiles of RNA constructs comprising inventive UTR combinations operably linked to coding region encoding different (poly-)peptides or proteins of interest in human diploid fibroblasts (HDF).

FIG. 6: Mean expression profiles of RNA constructs comprising inventive UTR combinations operably linked to coding region encoding antigen construct of interest protein in different cell lines.

FIG. 7: Mean expression profiles of RNA constructs comprising inventive UTR combinations operably linked to coding region encoding different (poly-)peptides or proteins of interest in HeLa cells.

FIG. 8: Mean expression profiles of RNA constructs comprising inventive UTR combinations operably linked to coding region encoding different (poly-)peptides or proteins of interest in HepG2 cells.

FIG. 9: Mean expression profiles of RNA constructs comprising inventive UTR combinations operably linked to coding region encoding different (poly-)peptides or proteins of interest in HSkMC cells.

FIG. 10: Mean expression profiles of RNA constructs comprising inventive UTR combinations operably linked to coding region encoding Rabies Virus Glycoprotein (RAVG) in different cell lines.

FIG. 11: Mean expression profiles of RNA constructs comprising inventive UTR combinations operably linked to coding region encoding different (poly-)peptides or proteins of interest in HEK293T cells.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Increase of RAV-G Expression by Using Specific UTR-Combinations

Cells were seeded on 96 well plates with black rim & clear optical bottom (Nunc Microplate; Thermo Fisher). HeLa cells or HDF were seeded 24 hours before transfection in a compatible complete cell medium (10,000 cells in 200 μl/well). HSkMC were seeded 48 hours before transfection in Differentiation Medium containing 2% horse serum (Gibco) to induce differentiation (48,000 cells in 200 μl/well). Cells were maintained at 37° C., 5% CO₂.

The day of transfection, the complete medium on HeLa or HDF was replaced with serum-free Opti-MEM medium (Thermo Fisher). Medium on HSkMC was exchanged for fresh complete Differentiation Medium.

Each RNA was complexed with either Lipofectamine2000 at a ratio of 1/1.5 (w/v) (HeLa & HDF) or Lipofectamine3000 at a ratio of 1/2.5 (w/v) (HSkMC) for 20 minutes in Opti-MEM.

Lipocomplexed mRNAs were then added to cells for transfection with either 100 ng of RNA (HeLa & HDF) or 70 ng of RNA (HSkMC) per well in a total volume of 200 μl.

90 minutes post start of transfection, 150 μl/well of transfection solution on HeLa or HDF was exchanged for 150 μl/well of complete medium. Cells were further maintained at 37° C., 5% CO2 before performing In-cell-Western. 24, 48 or 72 hours post start of transfection, RAV-G expression was quantified by In-Cell-Western using a primary antibody directed against an E-tag (rabbit polyclonal IgG; Bethyl), followed by an IRDye-coupled secondary antibody (IRDye 800CW goat anti-rabbit IgG; LI-COR). All steps of the In-Cell-Western were performed at room temperature.

First, cells were washed once with PBS and fixed with 3.7% formaldehyde in PBS for 20 minutes. After washing once in PBS, cells were permeabilized with 0.1% Triton X-100 in PBS for 10 minutes. After washing 3 times with 0.1% Tween 20 in PBS, cells were blocked for 30 minutes with Odyssey blocking buffer (PBS) (LI-COR).

Next, cells were incubated for 90 minutes with primary antibody (diluted 1:1000 in Odyssey blocking buffer (PBS)). Cells were then washed 3 times (Tween/PBS).

Subsequently, cells were incubated with a mixture of secondary antibody and Cell-Tag 700 Stain (LI-COR) (diluted 1:200 and 1:1000, respectively, in Odyssey blocking buffer (PBS)) for one hour in the dark.

After washing 4 times (Tween/PBS), PBS was added to cells and plates scanned using an Odyssey® CLx Imaging system (LI-COR).

Fluorescence (800 nm) was quantified using Image Studio Lite Software and the results compared to expression from a reference construct containing the RPL32/ALB7-UTR-combination set to 100%. The sequences of RPL32-derived 5′-UTRs are shown in SEQ ID NO: 21 (DNA) and 22 (RNA). The sequences of ALB7-derived 3′-UTRs are shown in SEQ ID NO: 35 (DNA) and 36 (RNA).

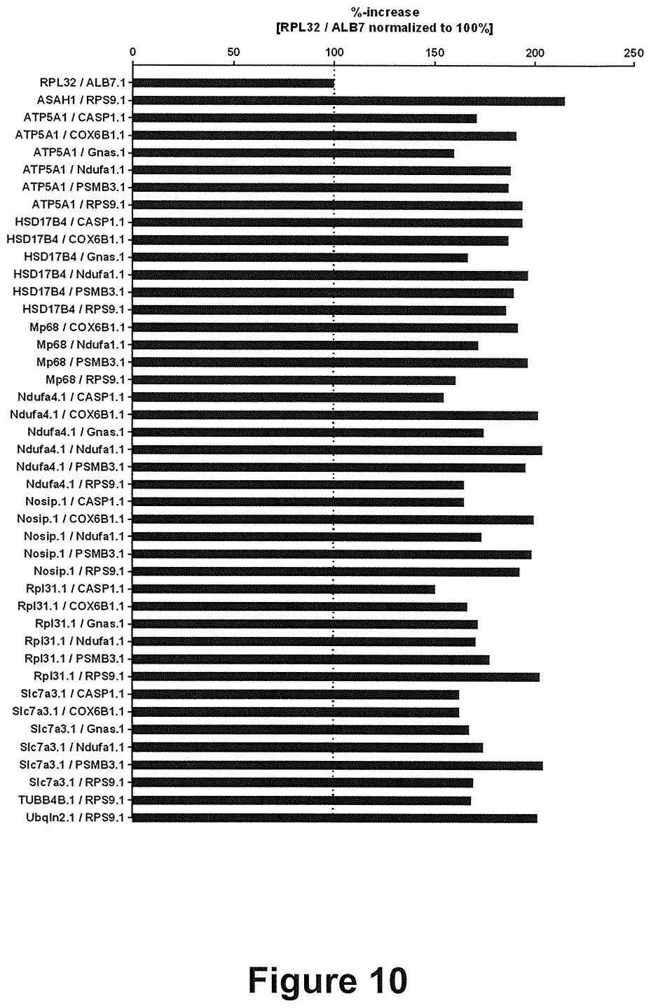

Mean expression profiles of RNA constructs comprising inventive UTR combinations operably linked to coding region encoding Rabies Virus Glycoprotein (RAVG) in different cell lines are shown in FIG. 10.

As apparent, it was possible to significantly increase expression by using the inventive UTR combinations operably linked to the coding region.

Further detailed results regarding the use of different mRNA 3′ sequences, i.e. A64N5 (i.e. a poly(A) sequence with 64A followed by N5) and C30-HSL as a 3′ sequence (i.e. a poly(C) sequence having 30C followed by a Histone stem-loop; histone SL or HSL as described above) are shown in Table 4A-I herein below. The left side of Table 4A-I shows results for A64N5, the right side shows results for C30-HSL. FIG. 10 as described above is the average value of both experiments. As in all examples, the UTR-combination RPL32/ALB7.1 was normalized to 100%.

TABLE 4A-I
detailed results for RAV-G carrying
A64N5 or C30-HSL 3′-end sequences

| | |
|---|---|
| % | UTRs |

target: RAV-G, A64N5

| | |
|---|---|
| 100 | RPL32/ALB7.1 |
| 149 | Rpl31.1/CASP1.1 |
| 153 | Ndufa4.1/CASP1.1 |
| 158 | ATP5A1/CASP1.1 |
| 160 | Slc7a3.1/COX6B1.1 |
| 161 | Slc7a3.1/CASP1.1 |
| 173 | Rpl31.1/Ndufa1.1 |
| 177 | Mp68/RPS9.1 |
| 181 | Nosip.1/CASP1.1 |
| 182 | ATP5A1/Gnas.1 |
| 183 | Rpl31.1/COX6B1.1 |
| 184 | Slc7a3.1/Gnas.1 |
| 184 | Rpl31.1/PSMB3.1 |
| 185 | TUBB4B.1/RPS9.1 |
| 187 | Nosip.1/Ndufa1.1 |
| 187 | HSD17B4/CASP1.1 |
| 188 | Slc7a3.1/Ndufa1.1 |
| 190 | Mp68/Ndufa1.1 |
| 190 | HSD17B4/Gnas.1 |
| 192 | Nosip.1/RPS9.1 |
| 192 | HSD17B4/COX6B1.1 |
| 194 | Slc7a3.1/RPS9.1 |
| 195 | Rpl31.1/Gnas.1 |
| 196 | HSD17B4/RPS9.1 |
| 196 | ATP5A1/COX6B1.1 |
| 197 | Mp68/COX6B1.1 |
| 199 | Ndufa4.1/COX6B1.1 |
| 200 | Ndufa4.1/Gnas.1 |
| 202 | ATP5A1/RPS9.1 |
| 203 | Rpl31.1/RPS9.1 |
| 203 | ATP5A1/Ndufa1.1 |
| 206 | HSD17B4/PSMB3.1 |
| 206 | ATP5A1/PSMB3.1 |
| 206 | Ndufa4.1/RPS9.1 |
| 209 | HSD17B4/Ndufa1.1 |
| 216 | Ndufa4.1/PSMB3.1 |
| 219 | Slc7a3.1/PSMB3.1 |
| 220 | Nosip.1/COX6B1.1 |
| 223 | Mp68/PSMB3.1 |
| 224 | Ndufa4.1/Ndufa1.1 |
| 226 | ASAH1/RPS9.1 |
| 229 | Nosip.1/PSMB3.1 |

target: RAV-G, C30-HSL

| | |
|---|---|
| 100 | RPL32/ALB7.1 |
| 116 | ATP5A1/Gnas.1 |
| 119 | HSD17B4/Gnas.1 |
| 123 | Slc7a3.1/RPS9.1 |
| 125 | Rpl31.1/Gnas.1 |
| 126 | Ndufa4.1/Gnas.1 |
| 128 | Mp68/RPS9.1 |
| 133 | Nosip.1/CASP1.1 |
| 135 | Rpl31.1/COX6B1.1 |
| 136 | Slc7a3.1/Gnas.1 |
| 136 | Mp68/Ndufa1.1 |
| 137 | TUBB4B.1/RPS9.1 |
| 138 | Nosip.1/PSMB3.1 |
| 146 | Mp68/PSMB3.1 |
| 149 | Nosip.1/Ndufa1.1 |
| 149 | ATP5A1/PSMB3.1 |
| 150 | Slc7a3.1/Ndufa1.1 |
| 155 | Rpl31.1/CASP1.1 |
| 155 | Ndufa4.1/PSMB3.1 |
| 157 | ATP5A1/Ndufa1.1 |
| 159 | HSD17B4/PSMB3.1 |
| 159 | Ndufa4.1/CASP1.1 |
| 160 | Nosip.1/COX6B1.1 |
| 164 | Ndufa4.1/Ndufa1.1 |
| 165 | Slc7a3.1/CASP1.1 |
| 167 | HSD17B4/RPS9.1 |
| 167 | Rpl31.1/PSMB3.1 |
| 168 | Rpl31.1/Ndufa1.1 |
| 169 | Slc7a3.1/COX6B1.1 |
| 174 | HSD17B4/Ndufa1.1 |
| 177 | HSD17B4/COX6B1.1 |
| 179 | Slc7a3.1/PSMB3.1 |
| 180 | ATP5A1/RPS9.1 |
| 181 | ATP5A1/COX6B1.1 |
| 183 | Mp68/COX6B1.1 |
| 195 | ASAH1/RPS9.1 |
| 195 | Nosip.1/RPS9.1 |
| 197 | ATP5A1/CASP1.1 |
| 202 | Rpl31.1/RPS9.1 |
| 207 | HSD17B4/CASP1.1 |
| 208 | Ndufa4.1/COX6B1.1 |

The sequences which were used in this example are shown in Table 4A-II.

TABLE 4A-II
sequences used in example 1

| SEQ | sequence | |
|---|---|---|
| ID NO | type | UTR-combination and ORF |
| 42 | protein | protein sequence (wt) from RAV_M13215.1_glycoprotein_RAV-G |
| 46 | RNA | CDS sequence (wt) from RAV_M13215.1_glycoprotein_RAV-G |
| 50 | RNA | CDS sequence (GC) from RAV_M13215.1_glycoprotein_RAV-G(GC) |
| 54 | RNA | HSD17B4_RAV-G(GC)_PSMB3_A64-C30-histoneSL |
| 55 | RNA | HSD17B4_RAV-G(GC)_PSMB3_A64 |
| 61 | RNA | HSD17B4_RAV-G(GC)_CASP1_A64-C30-histoneSL |
| 62 | RNA | HSD17B4_RAV-G(GC)_CASP1_A64 |
| 68 | RNA | HSD17B4_RAV-G(GC)_COX6B1_A64-C30-histoneSL |
| 69 | RNA | HSD17B4_RAV-G(GC)_COX6B1_A64 |
| 75 | RNA | HSD17B4_RAV-G(GC)_Gnas_A64-C30-histoneSL |
| 76 | RNA | HSD17B4_RAV-G(GC)_Gnas_A64 |
| 82 | RNA | HSD17B4_RAV-G(GC)_Ndufa1_A64-C30-histoneSL |
| 83 | RNA | HSD17B4_RAV-G(GC)_Ndufa1_A64 |
| 89 | RNA | HSD17B4_RAV-G(GC)_RPS9_A64-C30-histoneSL |
| 90 | RNA | HSD17B4_RAV-G(GC)_RPS9_A64 |
| 96 | RNA | ASAH1_RAV-G(GC)_RPS9_A64-C30-histoneSL |
| 97 | RNA | ASAH1_RAV-G(GC)_RPS9_A64 |
| 103 | RNA | ATP5A1_RAV-G(GC)_PSMB3_A64-C30-histoneSL |
| 104 | RNA | ATP5A1_RAV-G(GC)_PSMB3_A64 |
| 110 | RNA | ATP5A1_RAV-G(GC)_CASP1_A64-C30-histoneSL |
| 111 | RNA | ATP5A1_RAV-G(GC)_CASP1_A64 |
| 117 | RNA | ATP5A1_RAV-G(GC)_COX6B1_A64-C30-histoneSL |
| 118 | RNA | ATP5A1_RAV-G(GC)_COX6B1_A64 |
| 124 | RNA | ATP5A1_RAV-G(GC)_Gnas_A64-C30-histoneSL |
| 125 | RNA | ATP5A1_RAV-G(GC)_Gnas_A64 |
| 131 | RNA | ATP5A1_RAV-G(GC)_Ndufa1_A64-C30-histoneSL |
| 132 | RNA | ATP5A1_RAV-G(GC)_Ndufa1_A64 |
| 138 | RNA | ATP5A1_RAV-G(GC)_RPS9_A64-C30-histoneSL |
| 139 | RNA | ATP5A1_RAV-G(GC)_RPS9_A64 |
| 145 | RNA | Mp68_RAV-G(GC)_PSMB3_A64-C30-histoneSL |
| 146 | RNA | Mp68_RAV-G(GC)_PSMB3_A64 |
| 152 | RNA | Mp68_RAV-G(GC)_CASP1_A64-C30-histoneSL |
| 153 | RNA | Mp68_RAV-G(GC)_CASP1_A64 |
| 159 | RNA | Mp68_RAV-G(GC)_COX6B1_A64-C30-histoneSL |
| 160 | RNA | Mp68_RAV-G(GC)_COX6B1_A64 |
| 166 | RNA | Mp68_RAV-G(GC)_Gnas_A64-C30-histoneSL |
| 167 | RNA | Mp68_RAV-G(GC)_Gnas_A64 |
| 173 | RNA | Mp68_RAV-G(GC)_Ndufa1_A64-C30-histoneSL |
| 174 | RNA | Mp68_RAV-G(GC)_Ndufa1_A64 |
| 180 | RNA | Mp68_RAV-G(GC)_RPS9_A64-C30-histoneSL |
| 181 | RNA | Mp68_RAV-G(GC)_RPS9_A64 |
| 187 | RNA | Ndufa4_RAV-G(GC)_PSMB3_A64-C30-histoneSL |
| 188 | RNA | Ndufa4_RAV-G(GC)_PSMB3_A64 |
| 194 | RNA | Ndufa4_RAV-G(GC)_CASP1_A64-C30-histoneSL |
| 195 | RNA | Ndufa4_RAV-G(GC)_CASP1_A64 |
| 201 | RNA | Ndufa4_RAV-G(GC)_COX6B1_A64-C30-histoneSL |
| 202 | RNA | Ndufa4_RAV-G(GC)_COX6B1_A64 |
| 208 | RNA | Ndufa4_RAV-G(GC)_Gnas_A64-C30-histoneSL |
| 209 | RNA | Ndufa4_RAV-G(GC)_Gnas_A64 |
| 215 | RNA | Ndufa4_RAV-G(GC)_Ndufa1_A64-C30-histoneSL |
| 216 | RNA | Ndufa4_RAV-G(GC)_Ndufa1_A64 |
| 222 | RNA | Ndufa4_RAV-G(GC)_RPS9_A64-C30-histoneSL |
| 223 | RNA | Ndufa4_RAV-G(GC)_RPS9_A64 |
| 229 | RNA | Nosip_RAV-G(GC)_PSMB3_A64-C30-histoneSL |
| 230 | RNA | Nosip_RAV-G(GC)_PSMB3_A64 |
| 236 | RNA | Nosip_RAV-G(GC)_CASP1_A64-C30-histoneSL |
| 237 | RNA | Nosip_RAV-G(GC)_CASP1_A64 |
| 243 | RNA | Nosip_RAV-G(GC)_COX6B1_A64-C30-histoneSL |
| 244 | RNA | Nosip_RAV-G(GC)_COX6B1_A64 |
| 250 | RNA | Nosip_RAV-G(GC)_Gnas_A64-C30-histoneSL |

### Example 2: Increase of HsEpo and Ppluc Expression by Using Specific UTR-Combinations

Cells were seeded on 96 well plates. HDF and HepG2 (10,000 cells in 200 μl/well) were seeded 24 hours before transfection in a compatible complete cell medium. HSkMC (48,000 cells in 200 μl/well) were seeded 48 hours before transfection in Differentiation Medium containing 2% horse serum (Gibco) to induce differentiation. Cells were maintained at 37° C., 5% CO₂.

The day of transfection, the complete medium (HDF and HepG2) was replaced with serum-free Opti-MEM medium (Thermo Fisher). Medium on HSkMC was exchanged for fresh complete Differentiation Medium.

Each RNA was complexed with either Lipofectamine2000 at a ratio of 1/1.5 (w/v) (HDF and HepG2) or Lipofectamine3000 at a ratio of 1/2.5 (w/v) (HSkMC) for 20 minutes in Opti-MEM.

Lipocomplexed mRNAs were then added to cells for transfection with 100 ng per well in a total volume of 200 μl.

90 minutes post start of transfection, 150 μl/well of transfection solution on HDF and HepG2 was exchanged for 150 μl/well of complete medium. Cells were further maintained at 37° C., 5% CO2 before performing In-cell-Western.

### HsEPO:

24 hours post start of transfection, HsEpo expression was measured in cell supernatants using a commercially available ELISA kit (RNDsystems, Cat. DEP00) and a Hidex Chameleon plate reader.

### PPluc:

24 hours post start of transfection, Ppluc expression was measured in cell lysates. Cells were lysed by adding 100 μl of 1× passive lysis buffer (Promega, Cat. E1941) for at least 15 minutes. Lysed cells were incubated at −80° C. for at least 1 hour. Lysed cells were thawed and 20 μl were added to white LIA assay plates (Greiner Cat. 655075). Plates were introduced into a Hidex Chameleon plate reader with injection device for Beetle-juice containing substrate for firefly luciferase. Per well, 100 μl of beetle-juice were added. Ppluc luminescence was measured by Hidex Chameleon plate reader.

Results were compared to expression from a reference construct containing the RPL32/ALB7-UTR-combination set to 100%. The sequences of RPL32-derived 5′-UTRs are shown in SEQ ID NO: 21 (DNA) and 22 (RNA). The sequences of ALB7-derived 3′-UTRs are shown in SEQ ID NO: 35 (DNA) and 36 (RNA).

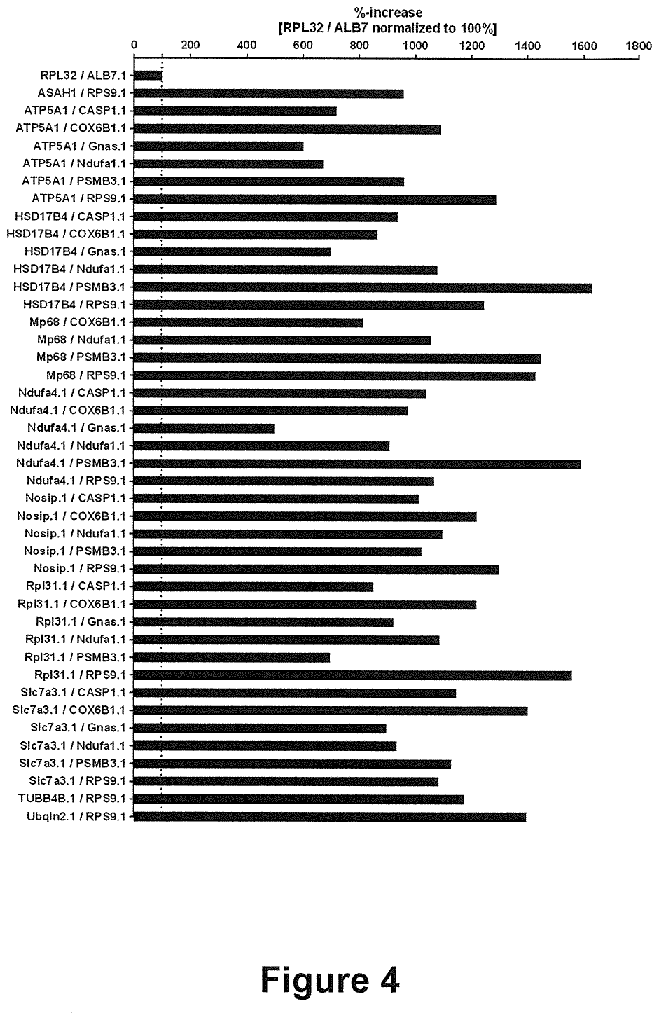

Mean expression profiles of RNA constructs comprising inventive UTR combinations operably linked to coding region encoding EPO in different cell lines are shown in FIG. 4.

As apparent, it was possible to significantly increase expression by using the inventive UTR combinations operably linked to the coding region.

Further detailed results for EPO regarding the use of different mRNA 3′ sequences, i.e. A64N5 (i.e. a poly(A) sequence with 64A followed by N5) and C30-HSL as a 3′ sequence (i.e. a poly(C) sequence having 30C followed by a Histone stem-loop; histone SL or HSL as described above) are shown in Table 4B-I herein below. The left side of Table 4B-I shows results for A64N5, the right side shows results for C30-HSL. FIG. 4 as described above is the average value of both experiments. As in all examples, the UTR-combination RPL32/ALB7.1 was normalized to 100%.

TABLE 4B-I
detailed results for EPO carrying
A64N5 or C30-HSL 3′-end sequences

| | |
|---|---|
| % | UTRs |

target: EPO; A64N5

| | |
|---|---|
| 100 | RPL32/ALB7.1 |
| 414 | HSD17B4/CASP1.1 |
| 440 | ATP5A1/CASP1.1 |
| 494 | HSD17B4/COX6B1.1 |
| 574 | Ndufa4.1/CASP1.1 |
| 575 | ATP5A1/Gnas.1 |
| 637 | Mp68/COX6B1.1 |
| 645 | Ndufa4.1/Gnas.1 |
| 711 | ATP5A1/RPS9.1 |
| 718 | Ndufa4.1/RPS9.1 |
| 720 | Rpl31.1/COX6B1.1 |
| 736 | ASAH1/RPS9.1 |
| 759 | Ndufa4.1/COX6B1.1 |
| 766 | Ndufa4.1/Ndufa1.l |
| 800 | Rpl31.1/CASP1.1 |
| 822 | ATP5A1/COX6B1.1 |
| 840 | Mp68/Ndufa1.1 |
| 852 | ATP5A1/Ndufa1.1 |
| 858 | Nosip.1/PSMB3.1 |
| 898 | Rpl31.1/Gnas.1 |
| 902 | Nosip.1/COX6B1.1 |
| 911 | Mp68/PSMB3.1 |
| 931 | Rpl31.1/PSMB3.1 |
| 945 | ATP5A1/PSMB3.1 |
| 965 | HSD17B4/Gnas.1 |
| 984 | Nosip.1/RPS9.1 |
| 987 | Rpl31.1/Ndufa1.1 |
| 997 | Nosip.1/CASP1.1 |
| 1003 | TUBB4B.1/RPS9.1 |
| 1014 | Slc7a3.1/RPS9.1 |
| 1064 | Slc7a3.1/Gnas.1 |
| 1078 | Rpl31.1/RPS9.1 |
| 1088 | Mp68/RPS9.1 |
| 1102 | Nosip.1/Ndufa1.1 |
| 1247 | HSD17B4/RPS9.1 |
| 1250 | Slc7a3.1/Ndufa1.1 |
| 1259 | Ndufa4.1/PSMB3.1 |
| 1278 | Slc7a3.1/PSMB3.1 |
| 1304 | HSD17B4/Ndufa1.1 |
| 1319 | Slc7a3.1/CASP1.1 |
| 1334 | Slc7a3.1/COX6B1.1 |
| 1507 | HSD17B4/PSMB3.1 |

target: EPO; C30-HSL

| | |
|---|---|
| 100 | RPL32/ALB7.1 |
| 358 | Ndufa4.1/Gnas.1 |
| 438 | HSD17B4/Gnas.1 |
| 471 | Rpl31.1/PSMB3.1 |
| 494 | ATP5A1/Ndufa1.1 |
| 628 | ATP5A1/Gnas.1 |
| 630 | Slc7a3.1/Ndufa1.1 |
| 740 | Slc7a3.1/Gnas.1 |
| 857 | HSD17B4/Ndufa1.1 |
| 905 | Rpl31.1/CASP1.1 |
| 955 | Rpl31.1/Gnas.1 |
| 979 | ATP5A1/PSMB3.1 |
| 987 | Slc7a3.1/PSMB3.1 |
| 998 | Mp68/COX6B1.1 |
| 999 | ATP5A1/CASP1.1 |
| 1024 | Slc7a3.1/CASP1.1 |
| 1035 | Nosip.1/CASP1.1 |
| 1055 | Ndufa4.1/Ndufa1.1 |
| 1099 | Nosip.1/Ndufa1.1 |
| 1164 | Slc7a3.1/RPS9.1 |
| 1182 | ASAH1/RPS9.1 |
| 1192 | Nosip.1/PSMB3.1 |
| 1195 | Rpl31.1/Ndufa1.1 |
| 1195 | Ndufa4.1/COX6B1.1 |
| 1239 | HSD17B4/COX6B1.1 |
| 1274 | Mp68/Ndufa1.1 |
| 1358 | ATP5A1/COX6B1.1 |
| 1359 | TUBB4B.1/RPS9.1 |
| 1423 | Ndufa4.1/RPS9.1 |
| 1467 | HSD17B4/CASP1.1 |
| 1479 | Slc7a3.1/COX6B1.1 |
| 1506 | Ndufa4.1/CASP1.1 |
| 1542 | Nosip.1/COX6B1.1 |
| 1618 | Nosip.1/RPS9.1 |
| 1726 | Rpl31.1/COX6B1.1 |
| 1757 | HSD17B4/PSMB3.1 |
| 1773 | Mp68/RPS9.1 |
| 1868 | ATP5A1/RPS9.1 |
| 1924 | Ndufa4.1/PSMB3.1 |
| 1992 | Mp68/PSMB3.1 |
| 2051 | Rpl31.1/RPS9.1 |

The sequences which were used in this example are shown in Table 4B-II.

TABLE 4B-II
sequences used in example 2

| SEQ | sequence | |
|---|---|---|
| ID NO | type | UTR-combination and ORF |
| 43 | protein | protein sequence (wt) from Homo sapiens_NM_000799.2_erythropoietin_HsEPO |
| 47 | RNA | CDS sequence (wt) from Homo sapiens_NM000799.2_erythropoietin_HsEPO |
| 51 | RNA | CDS sequence (GC)from Homo sapiens_NM000799.2_erythropoietin_HsEPO(GC) |
| 56 | RNA | HSD17B4_HsEPO(GC)_PSMB3_A64-C30-histoneSL |
| 57 | RNA | HSD17B4_HsEPO(GC)_PSMB3_A64 |
| 63 | RNA | HSD17B4_HsEPO(GC)_CASP1_A64-C30-histoneSL |
| 64 | RNA | HSD17B4_HsEPO(GC)_CASP1_A64 |
| 70 | RNA | HSD17B4_HsEPO(GC)_COX6B1_A64-C30-histoneSL |
| 71 | RNA | HSD17B4_HsEPO(GC)_COX6B1_A64 |
| 77 | RNA | HSD17B4_HsEPO(GC)_Gnas_A64-C30-histoneSL |
| 78 | RNA | HSD17B4_HsEPO(GC)_Gnas_A64 |
| 84 | RNA | HSD17B4_HsEPO(GC)_Ndufa1_A64-C30-histoneSL |
| 85 | RNA | HSD17B4_HsEPO(GC)_Ndufa1_A64 |
| 91 | RNA | HSD17B4_HsEPO(GC)_RPS9_A64-C30-histoneSL |
| 92 | RNA | HSD17B4_HsEPO(GC)_RPS9_A64 |
| 98 | RNA | ASAH1_HsEPO(GC)_RPS9_A64-C30-histoneSL |
| 99 | RNA | ASAH1_HsEPO(GC)_RPS9_A64 |
| 105 | RNA | ATP5A1_HsEPO(GC)_PSMB3_A64-C30-histoneSL |
| 106 | RNA | ATP5A1_HsEPO(GC)_PSMB3_A64 |
| 112 | RNA | ATP5A1_HsEPO(GC)_CASP1_A64-C30-histoneSL |
| 113 | RNA | ATP5A1_HsEPO(GC)_CASP1_A64 |
| 119 | RNA | ATP5A1_HsEPO(GC)_COX6B1_A64-C30-histoneSL |
| 120 | RNA | ATP5A1_HsEPO(GC)_COX6B1_A64 |
| 126 | RNA | ATP5A1_HsEPO(GC)_Gnas_A64-C30-histoneSL |
| 127 | RNA | ATP5A1_HsEPO(GC)_Gnas_A64 |
| 133 | RNA | ATP5A1_HsEPO(GC)_Ndufa1_A64-C30-histoneSL |
| 134 | RNA | ATP5A1_HsEPO(GC)_Ndufa1_A64 |
| 140 | RNA | ATP5A1_HsEPO(GC)_RPS9_A64-C30-histoneSL |
| 141 | RNA | ATP5A1_HsEPO(GC)_RPS9_A64 |
| 147 | RNA | Mp68_HsEPO(GC)_PSMB3_A64-C30-histoneSL |
| 148 | RNA | Mp68_HsEPO(GC)_PSMB3_A64 |
| 154 | RNA | Mp68_HsEPO(GC)_CASP1_A64-C30-histoneSL |
| 155 | RNA | Mp68_HsEPO(GC)_CASP1_A64 |
| 161 | RNA | Mp68_HsEPO(GC)_COX6B1_A64-C30-histoneSL |
| 162 | RNA | Mp68_HsEPO(GC)_COX6B1_A64 |
| 168 | RNA | Mp68_HsEPO(GC)_Gnas_A64-C30-histoneSL |
| 169 | RNA | Mp68_HsEPO(GC)_Gnas_A64 |
| 175 | RNA | Mp68_HsEPO(GC)_Ndufa1_A64-C30-histoneSL |
| 176 | RNA | Mp68_HsEPO(GC)_Ndufa1_A64 |
| 182 | RNA | Mp68_HsEPO(GC)_RPS9_A64-C30-histoneSL |
| 183 | RNA | Mp68_HsEPO(GC)_RPS9_A64 |
| 189 | RNA | Ndufa4_HsEPO(GC)_PSMB3_A64-C30-histoneSL |
| 190 | RNA | Ndufa4_HsEPO(GC)_PSMB3_A64 |
| 196 | RNA | Ndufa4_HsEPO(GC)_CASP1_A64-C30-histoneSL |
| 197 | RNA | Ndufa4_HsEPO(GC)_CASP1_A64 |
| 203 | RNA | Ndufa4_HsEPO(GC)_COX6B1_A64-C30-histoneSL |
| 204 | RNA | Ndufa4_HsEPO(GC)_COX6B1_A64 |
| 210 | RNA | Ndufa4_HsEPO(GC)_Gnas_A64-C30-histoneSL |
| 211 | RNA | Ndufa4_HsEPO(GC)_Gnas_A64 |
| 217 | RNA | Ndufa4_HsEPO(GC)_Ndufa1_A64-C30-histoneSL |
| 218 | RNA | Ndufa4_HsEPO(GC)_Ndufa1_A64 |
| 224 | RNA | Ndufa4_HsEPO(GC)_RPS9_A64-C30-histoneSL |
| 225 | RNA | Ndufa4_HsEPO(GC)_RPS9_A64 |
| 231 | RNA | Nosip_HsEPO(GC)_PSMB3_A64-C30-histoneSL |
| 232 | RNA | Nosip_HsEPO(GC)_PSMB3_A64 |
| 238 | RNA | Nosip_HsEPO(GC)_CASP1_A64-C30-histoneSL |
| 239 | RNA | Nosip_HsEPO(GC)_CASP1_A64 |
| 245 | RNA | Nosip_HsEPO(GC)_COX6B1_A64-C30-histoneSL |
| 246 | RNA | Nosip_HsEPO(GC)_COX6B1_A64 |
| 252 | RNA | Nosip_HsEPO(GC)_Gnas_A64-C30-histoneSL |
| 253 | RNA | Nosip_HsEPO(GC)_Gnas_A64 |
| 259 | RNA | Nosip_HsEPO(GC)_Ndufa1_A64-C30-histoneSL |
| 260 | RNA | Nosip_HsEPO(GC)_Ndufa1_A64 |
| 266 | RNA | Nosip_HsEPO(GC)_RPS9_A64-C30-histoneSL |
| 267 | RNA | Nosip_HsEPO(GC)_RPS9_A64 |
| 273 | RNA | Rpl31_HsEPO(GC)_PSMB3_A64-C30-histoneSL |
| 274 | RNA | Rpl31_HsEPO(GC)_PSMB3_A64 |
| 280 | RNA | Rpl31_HsEPO(GC)_CASP1_A64-C30-histoneSL |
| 281 | RNA | Rpl31_HsEPO(GC)_CASP1_A64 |
| 287 | RNA | Rpl31_HsEPO(GC)_COX6B1_A64-C30-histoneSL |
| 288 | RNA | Rpl31_HsEPO(GC)_COX6B1_A64 |
| 294 | RNA | Rpl31_HsEPO(GC)_Gnas_A64-C30-histoneSL |
| 295 | RNA | Rpl31_HsEPO(GC)_Gnas_A64 |
| 301 | RNA | Rpl31_HsEPO(GC)_Ndufa1_A64-C30-histoneSL |
| 302 | RNA | Rpl31_HsEPO(GC)_Ndufa1_A64 |
| 308 | RNA | Rpl31_HsEPO(GC)_RPS9_A64-C30-histoneSL |
| 309 | RNA | Rpl31_HsEPO(GC)_RPS9_A64 |
| 315 | RNA | Slc7a3_HsEPO(GC)_PSMB3_A64-C30-histoneSL |
| 316 | RNA | Slc7a3_HsEPO(GC)_PSMB3_A64 |
| 322 | RNA | Slc7a3_HsEPO(GC)_CASP1_A64-C30-histoneSL |
| 323 | RNA | Slc7a3_HsEPO(GC)_CASP1_A64 |
| 329 | RNA | Slc7a3_HsEPO(GC)_COX6B1_A64-C30-histoneSL |
| 330 | RNA | Slc7a3_HsEPO(GC)_COX6B1_A64 |
| 336 | RNA | Slc7a3_HsEPO(GC)_Gnas_A64-C30-histoneSL |
| 337 | RNA | Slc7a3_HsEPO(GC)_Gnas_A64 |
| 343 | RNA | Slc7a3_HsEPO(GC)_Ndufa1_A64-C30-histoneSL |
| 344 | RNA | Slc7a3_HsEPO(GC)_Ndufa1_A64 |
| 350 | RNA | Slc7a3_HsEPO(GC)_RPS9_A64-C30-histoneSL |
| 351 | RNA | Slc7a3_HsEPO(GC)_RPS9_A64 |
| 357 | RNA | TUBB4B_HsEPO(GC)_RPS9_A64-C30-histoneSL |
| 358 | RNA | TUBB4B_HsEPO(GC)_RPS9_A64 |
| 364 | RNA | Ubqln2_HsEPO(GC)_RPS9_A64-C30-histoneSL |
| 365 | RNA | Ubqln2_HsEPO(GC)_RPS9_A64 |
| 373 | RNA | RPL32_HsEPO(GC)_ALB7_A64-C30-histoneSL |
| 374 | RNA | RPL32_HsEPO(GC)_ALB7_A64 |
| 44 | protein | protein sequence (wt) from Photinus pyralis_U47122.2_luciferase_PpLuc |
| 48 | RNA | CDS sequence (wt) from Photinus pyralis_U47122.2_luciferase_PpLuc |
| 52 | RNA | CDS sequence (GC)from Photinus pyralis_U47122.2_luciferase_PpLuc(GC) |
| 58 | RNA | HSD17B4_PpLuc(GC)_PSMB3_A64-C30-histoneSL |
| 59 | RNA | HSD17B4_PpLuc(GC)_PSMB3_A64 |
| 65 | RNA | HSD17B4_PpLuc(GC)_CASP1_A64-C30-histoneSL |
| 66 | RNA | HSD17B4_PpLuc(GC)_CASP1_A64 |
| 72 | RNA | HSD17B4_PpLuc(GC)_COX6B1_A64-C30-histoneSL |
| 73 | RNA | HSD17B4_PpLuc(GC)_COX6B1_A64 |
| 79 | RNA | HSD17B4_PpLuc(GC)_Gnas_A64-C30-histoneSL |
| 80 | RNA | HSD17B4_PpLuc(GC)_Gnas_A64 |
| 86 | RNA | HSD17B4_PpLuc(GC)_Ndufa1_A64-C30-histoneSL |
| 87 | RNA | HSD17B4_PpLuc(GC)_Ndufa1_A64 |
| 93 | RNA | HSD17B4_PpLuc(GC)_RPS9_A64-C30-histoneSL |
| 94 | RNA | HSD17B4_PpLuc(GC)_RPS9_A64 |
| 100 | RNA | ASAH1_PpLuc(GC)_RPS9_A64-C30-histoneSL |
| 101 | RNA | ASAH1_PpLuc(GC)_RPS9_A64 |
| 107 | RNA | ATP5A1_PpLuc(GC)_PSMB3_A64-C30-histoneSL |
| 108 | RNA | ATP5A1_PpLuc(GC)_PSMB3_A64 |
| 114 | RNA | ATP5A1_PpLuc(GC)_CASP1_A64-C30-histoneSL |
| 115 | RNA | ATP5A1_PpLuc(GC)_CASP1_A64 |
| 121 | RNA | ATP5A1_PpLuc(GC)_COX6B1_A64-C30-histoneSL |
| 122 | RNA | ATP5A1_PpLuc(GC)_COX6B1_A64 |
| 128 | RNA | ATP5A1_PpLuc(GC)_Gnas_A64-C30-histoneSL |
| 129 | RNA | ATP5A1_PpLuc(GC)_Gnas_A64 |
| 135 | RNA | ATP5A1_PpLuc(GC)_Ndufa1_A64-C30-histoneSL |
| 136 | RNA | ATP5A1_PpLuc(GC)_Ndufa1_A64 |
| 142 | RNA | ATP5A1_PpLuc(GC)_RPS9_A64-C30-histoneSL |
| 143 | RNA | ATP5A1_PpLuc(GC)_RPS9_A64 |
| 149 | RNA | Mp68_PpLuc(GC)_PSMB3_A64-C30-histoneSL |
| 150 | RNA | Mp68_PpLuc(GC)_PSMB3_A64 |
| 156 | RNA | Mp68_PpLuc(GC)_CASP1_A64-C30-histoneSL |
| 157 | RNA | Mp68_PpLuc(GC)_CASP1_A64 |
| 163 | RNA | Mp68_PpLuc(GC)_COX6B1_A64-C30-histoneSL |
| 164 | RNA | Mp68_PpLuc(GC)_COX6B1_A64 |
| 170 | RNA | Mp68_PpLuc(GC)_Gnas_A64-C30-histoneSL |
| 171 | RNA | Mp68_PpLuc(GC)_Gnas_A64 |
| 177 | RNA | Mp68_PpLuc(GC)_Ndufa1_A64-C30-histoneSL |
| 178 | RNA | Mp68_PpLuc(GC)_Ndufa1_A64 |
| 184 | RNA | Mp68_PpLuc(GC)_RPS9_A64-C30-histoneSL |
| 185 | RNA | Mp68_PpLuc(GC)_RPS9_A64 |
| 191 | RNA | Ndufa4_PpLuc(GC)_PSMB3_A64-C30-histoneSL |
| 192 | RNA | Ndufa4_PpLuc(GC)_PSMB3_A64 |
| 198 | RNA | Ndufa4_PpLuc(GC)_CASP1_A64-C30-histoneSL |
| 199 | RNA | Ndufa4_PpLuc(GC)_CASP1_A64 |
| 205 | RNA | Ndufa4_PpLuc(GC)_COX6B1_A64-C30-histoneSL |
| 206 | RNA | Ndufa4_PpLuc(GC)_COX6B1_A64 |
| 212 | RNA | Ndufa4_PpLuc(GC)_Gnas_A64-C30-histoneSL |
| 213 | RNA | Ndufa4_PpLuc(GC)_Gnas_A64 |
| 219 | RNA | Ndufa4_PpLuc(GC)_Ndufa1_A64-C30-histoneSL |
| 220 | RNA | Ndufa4_PpLuc(GC)_Ndufa1_A64 |
| 226 | RNA | Ndufa4_PpLuc(GC)_RPS9_A64-C30-histoneSL |
| 227 | RNA | Ndufa4_PpLuc(GC)_RPS9_A64 |
| 233 | RNA | Nosip_PpLuc(GC)_PSMB3_A64-C30-histoneSL |
| 234 | RNA | Nosip_PpLuc(GC)_PSMB3_A64 |
| 240 | RNA | Nosip_PpLuc(GC)_CASP1_A64-C30-histoneSL |
| 241 | RNA | Nosip_PpLuc(GC)_CASP1_A64 |
| 247 | RNA | Nosip_PpLuc(GC)_COX6B1_A64-C30-histoneSL |
| 248 | RNA | Nosip_PpLuc(GC)_COX6B1_A64 |
| 254 | RNA | Nosip_PpLuc(GC)_Gnas_A64-C30-histoneSL |
| 255 | RNA | Nosip_PpLuc(GC)_Gnas_A64 |
| 261 | RNA | Nosip_PpLuc(GC)_Ndufa1_A64-C30-histoneSL |
| 262 | RNA | Nosip_PpLuc(GC)_Ndufa1_A64 |
| 268 | RNA | Nosip_PpLuc(GC)_RPS9_A64-C30-histoneSL |
| 269 | RNA | Nosip_PpLuc(GC)_RPS9_A64 |
| 275 | RNA | Rpl31_PpLuc(GC)_PSMB3_A64-C30-histoneSL |
| 276 | RNA | Rpl31_PpLuc(GC)_PSMB3_A64 |
| 282 | RNA | Rpl31_PpLuc(GC)_CASP1_A64-C30-histoneSL |
| 283 | RNA | Rpl31_PpLuc(GC)_CASP1_A64 |
| 289 | RNA | Rpl31_PpLuc(GC)_COX6B1_A64-C30-histoneSL |
| 290 | RNA | Rpl31_PpLuc(GC)_COX6B1_A64 |
| 296 | RNA | Rpl31_PpLuc(GC)_Gnas_A64-C30-histoneSL |
| 297 | RNA | Rpl31_PpLuc(GC)_Gnas_A64 |
| 303 | RNA | Rpl31_PpLuc(GC)_Ndufa1_A64-C30-histoneSL |
| 304 | RNA | Rpl31_PpLuc(GC)_Ndufa1_A64 |
| 310 | RNA | Rpl31_PpLuc(GC)_RPS9_A64-C30-histoneSL |
| 311 | RNA | Rpl31_PpLuc(GC)_RPS9_A64 |
| 317 | RNA | Slc7a3_PpLuc(GC)_PSMB3_A64-C30-histoneSL |
| 318 | RNA | Slc7a3_PpLuc(GC)_PSMB3_A64 |
| 324 | RNA | Slc7a3_PpLuc(GC)_CASP1_A64-C30-histoneSL |
| 325 | RNA | Slc7a3_PpLuc(GC)_CASP1_A64 |
| 331 | RNA | Slc7a3_PpLuc(GC)_COX6B1_A64-C30-histoneSL |
| 332 | RNA | Slc7a3_PpLuc(GC)_COX6B1_A64 |
| 338 | RNA | Slc7a3_PpLuc(GC)_Gnas_A64-C30-histoneSL |
| 339 | RNA | Slc7a3_PpLuc(GC)_Gnas_A64 |
| 345 | RNA | Slc7a3_PpLuc(GC)_Ndufa1_A64-C30-histoneSL |
| 346 | RNA | Slc7a3_PpLuc(GC)_Ndufa1_A64 |
| 352 | RNA | Slc7a3_PpLuc(GC)_RPS9_A64-C30-histoneSL |
| 353 | RNA | Slc7a3_PpLuc(GC)_RPS9_A64 |
| 359 | RNA | TUBB4B_PpLuc(GC)_RPS9_A64-C30-histoneSL |
| 360 | RNA | TUBB4B_PpLuc(GC)_RPS9_A64 |
| 366 | RNA | Ubqln2_PpLuc(GC)_RPS9_A64-C30-histoneSL |
| 367 | RNA | Ubqln2_PpLuc(GC)_RPS9_A64 |
| 375 | RNA | RPL32_PpLuc(GC)_ALB7_A64-C30-histoneSL |
| 376 | RNA | RPL32_PpLuc(GC)_ALB7_A64 |

### Example 3: Increase of Protein of Interest (POI) Expression by Using Specific UTR-Combinations

HeLa, HDF and HSkM cells were analyzed via in-cell-western blotting:

Cells were seeded on 96 well plates with black rim & clear optical bottom (Nunc Microplate; Thermo Fisher). HeLa cells or HDF (10,000 cells in 200 μl/well) were seeded 24 hours before transfection in a compatible complete cell medium. HSkMC (48,000 cells in 200 μl/well) were seeded 48 hours before transfection in Differentiation Medium containing 2% horse serum (Gibco) to induce differentiation. Cells were maintained at 37° C., 5% CO2.

The day of transfection, the complete medium on HeLa or HDF was replaced with serum-free Opti-MEM medium (Thermo Fisher). Medium on HSkMC was exchanged for fresh complete Differentiation Medium.

Each RNA was complexed with either Lipofectamine2000 at a ratio of 1/1.5 (w/v) (HeLa & HDF) or Lipofectamine3000 at a ratio of 1/2.5 (w/v) (HSkMC) for 20 minutes in Opti-MEM.

Lipocomplexed mRNAs were then added to cells for transfection with either 200 ng of RNA (HeLa & HDF) or 100 ng of RNA (HSkMC) per well in a total volume of 150 μl.

90 minutes post start of transfection, 100 μl/well of transfection solution on HeLa or HDF was exchanged for 100 μl/well of complete medium. Cells were further maintained at 37° C., 5% CO2 before performing In-cell-Western.

36 hours post start of transfection, POI expression was quantified by In-Cell-Western using a primary antibody directed against POI (mouse monoclonal anti-POI; Santa Cruz), followed by an IRDye-coupled secondary antibody (IRDye 800CW goat anti-rabbit IgG; LI-COR). All steps of the In-Cell-Western were performed at room temperature.

First, cells were washed once with PBS and fixed with 3.7% formaldehyde in PBS for 10 minutes. After washing once in PBS, cells were permeabilized with Perm/Wash buffer (BD) for 30 minutes. Cells were blocked for 30 minutes with a mix of Odyssey blocking buffer (PBS) (LI-COR) and Perm/Wash buffer (BD) (1:1).

Next, cells were incubated for 150 minutes with primary antibody (diluted 1:200 in Perm/Wash buffer (BD)). Cells were then washed 3 times (Perm/Wash buffer (BD)).

Subsequently, cells were incubated with a mixture of secondary antibody and Cell-Tag 700 Stain (LI-COR) (diluted 1:200 and 1:1000, respectively, in Perm/Wash buffer (BD)) for one hour in the dark.

After washing 4 times (Perm/Wash buffer (BD)), PBS was added to cells and plates scanned using an Odyssey® CLx Imaging system (LI-COR).

Fluorescence (800 nm) was quantified using Image Studio Lite Software and the results compared to expression from a reference construct containing the RPL32/ALB7-UTR-combination set to 100%. The sequences of RPL32-derived 5′-UTRs are shown in SEQ ID NO: 21 (DNA) and 22 (RNA). The sequences of ALB7-derived 3′-UTRs are shown in SEQ ID NO: 35 (DNA) and 36 (RNA).

Sol8 cells were analyzed via routine FACS analysis.

Cells were seeded on TC Plate 24 well standard F plates (Sarstedt). Sol8 cells (40,000 cells in 1000 μl/well) were seeded 24 hours before transfection in a compatible complete cell medium. Cells were maintained at 37° C., 5% CO₂.

The day of transfection, the complete medium was replaced with serum-free Opti-MEM medium (Thermo Fisher).

Each RNA was complexed with either Lipofectamine2000 at a ratio of 1/1.5 (w/v) for 20 minutes in Opti-MEM.

Lipocomplexed mRNAs were then added to cells for transfection with 500 ng of RNA (per well in a total volume of 1500 μl).

190 minutes post start of transfection; the total transfection solution (1500 μl) on Sol8 cells was exchanged for 2000 μl/well of complete medium. Cells were further maintained at 37° C., 5% CO₂ before performing FACS analysis.

36 hours post start of transfection, pri expression was quantified by FACS analysis using a primary antibody directed against POI (mouse monoclonal anti-POI; Santa Cruz), followed by an APC-coupled secondary antibody (Goat anti-mouse IgG APC; Biolegend). All steps of the FACS analysis were performed at room temperature or 4° C.

First, cells were detached (40 mM Tris HCl pH 7.5 150 mM NaCl, 1 mM EDTA in H20; 5 min at RT) washed once with PBS.

After washing in PBS, intracellular staining was performed by using antibody directed against POI. Therefore the cells were incubated first with Cytofix/Cytoperm (BD) for 30 minutes at 4° C. Next, cells were washed in Perm/Wash buffer (0.5% BSA and 0.1% Saponin in PBS) for 3 minutes. Following, the cells were incubated with primary antibody (diluted 1:200 in Perm/Wash buffer) for 30 min at 4° C.

After washing on the cells in Perm/Wash buffer (BD), the cells were incubated with the secondary antibody (diluted 1:500 in Perm/Wash buffer) for 30 min at 4° C.

Cells were then washed (Perm/Wash buffer (BD)), resuspended in 100 μl PFEA buffer (PBS+2% FCS+2 mM EDTA+0.01% NaN3) and analyzed by using BD FACS Canto II.

Live/Dead staining was performed with Aqua fluorescent reactive dye (Invitrogen).

Mean fluorescence intensity was measured and the results compared to expression from a reference construct containing the RPL32/ALB7-UTR-combination set to 100%. The sequences of RPL32-derived 5′-UTRs are shown in SEQ ID NO: 21 (DNA) and 22 (RNA). The sequences of ALB7-derived 3′-UTRs are shown in SEQ ID NO: 35 (DNA) and 36 (RNA).

As apparent, it was possible to significantly increase expression by using the inventive UTR combinations operably linked to the coding region.

### Example 4: Increase of Single Chain Antibody Construct of Interest Expression by Using Specific UTR-Combinations

Cells were seeded on 96 well plates with black rim & clear optical bottom (Nunc Microplate; Thermo Fisher). HeLa cells (10,000 cells in 200 μl/well) were seeded 24 hours before transfection in a compatible complete cell medium. Cells were maintained at 37° C., 5% CO2.

The day of transfection, the complete medium on HeLa or HDF was replaced with serum-free Opti-MEM medium (Thermo Fisher). Medium on HSkMC was exchanged for fresh complete differentiation medium.

1 μg of single chain antibody construct of interest mRNA [c=0.1 g/l] was complexed with Lipofectamine2000. Part of the transfection complexes was then diluted 5-fold and part was diluted 10-fold (medium dose). 500 ng of single chain antibody construct of interest was then transfected into the cells. 24 hours after transfection, cells were inspected through the microscope. Supernatant was taken and quantified in an Antibody-ELISA/anti Fc-ELISA assay using the coating antibody Goat Anti-Human IgG (SouthernBiotech) and the detection antibody Goat Anti-Human IgG Biotin (Dianova).

As apparent, it was possible to significantly increase expression by using the inventive UTR combinations operably linked to the coding region.

An overview of the sequences which were used in this example is shown in Table 4D below, wherein the sequence of the undisclosed antibody construct of interest from Example 4 consists of 496 amino acids and the CDS consists of 1491 nucleic acids, while the antigen construct of interest from Example 5 (Table 4D) consists of 553 amino acids and the CDS consists of 1662 nucleic acids. A person skilled in the art is able to derive a corresponding sequence from the disclosure of Table 4D for Example 4.

### Example 5: Increase of Antigen Construct of Interest Expression by Using Specific UTR-Combinations

HEK293T cells were analyzed by FACS. 293T cells were seeded at a density of 200 000 cells/well (200 000 cells/2 ml) in a 6-well plate. Each RNA was complexed with Lipofectamine2000 at a ratio of 1/1.5 (w/v) for 20 minutes in Opti-MEM. Lipocomplexed mRNAs were then added to cells for transfection with 2 μg of RNA per well in a total volume of 500 μl. 4h post start of transfection the transfection solution was exchanged for 2000 μl/well of complete medium. Cells were further maintained at 37° C., 5% CO₂ before performing FACS analysis. The sequences which were used in this example are shown in Table 4D.

TABLE 4D
sequences used for protein of interest from example 5

| SEQ | sequence | |
|---|---|---|
| ID NO | type | UTR-combination |
| 45 | protein | protein sequence (wt) from protein of |
| | | interest example 5 |
| 49 | RNA | CDS sequence (wt) POI |
| 53 | RNA | CDS sequence (GC) POI |
| 60 | RNA | HSD17B4_POI_PSMB3_A64-C30-histoneSL |
| 67 | RNA | HSD17B4_POI_CASP1_A64-C30-histoneSL |
| 74 | RNA | HSD17B4_POI_COX6B1_A64-C30-histoneSL |
| 81 | RNA | HSD17B4_POI_Gnas_A64-C30-histoneSL |
| 88 | RNA | HSD17B4_POI_Ndufa1_A64-C30-histoneSL |
| 95 | RNA | HSD17B4_POI_RPS9_A64-C30-histoneSL |
| 102 | RNA | ASAH1_POI_RPS9_A64-C30-histoneSL |
| 109 | RNA | ATP5A1_POI_PSMB3_A64-C30-histoneSL |
| 116 | RNA | ATP5A1_POI_CASP1_A64-C30-histoneSL |
| 123 | RNA | ATP5A1_POI_COX6B1_A64-C30-histoneSL |
| 130 | RNA | ATP5A1_POI_Gnas_A64-C30-histoneSL |
| 137 | RNA | ATP5A1_POI_Ndufa1_A64-C30-histoneSL |
| 144 | RNA | ATP5A1_POI_RPS9_A64-C30-histoneSL |
| 151 | RNA | Mp68_POI_PSMB3_A64-C30-histoneSL |
| 158 | RNA | Mp68_POI_CASP1_A64-C30-histoneSL |
| 165 | RNA | Mp68_POI_COX6B1_A64-C30-histoneSL |
| 172 | RNA | Mp68_POI_Gnas_A64-C30-histoneSL |
| 179 | RNA | Mp68_POI_Ndufa1_A64-C30-histoneSL |
| 186 | RNA | Mp68_POI_RPS9_A64-C30-histoneSL |
| 193 | RNA | Ndufa4_POI_PSMB3_A64-C30-histoneSL |
| 200 | RNA | Ndufa4_POI_CASP1_A64-C30-histoneSL |
| 207 | RNA | Ndufa4_POI_COX6B1_A64-C30-histoneSL |
| 214 | RNA | Ndufa4_POI_Gnas_A64-C30-histoneSL |
| 221 | RNA | Ndufa4_POI_Ndufa1_A64-C30-histoneSL |
| 228 | RNA | Ndufa4_POI_RPS9_A64-C30-histoneSL |
| 235 | RNA | Nosip_POI_PSMB3_A64-C30-histoneSL |
| 242 | RNA | Nosip_POI_CASP1_A64-C30-histoneSL |
| 249 | RNA | Nosip_POI_COX6B1_A64-C30-histoneSL |
| 256 | RNA | Nosip_POI_Gnas_A64-C30-histoneSL |
| 263 | RNA | Nosip_POI_Ndufa1_A64-C30-histoneSL |
| 270 | RNA | Nosip_POI_RPS9_A64-C30-histoneSL |
| 277 | RNA | Rpl31_POI_PSMB3_A64-C30-histoneSL |
| 284 | RNA | Rpl31_POI_CASP1_A64-C30-histoneSL |
| 291 | RNA | Rpl31_POI_COX6B1_A64-C30-histoneSL |
| 298 | RNA | Rpl31_POI_Gnas_A64-C30-histoneSL |
| 305 | RNA | Rpl31_POI_Ndufa1_A64-C30-histoneSL |
| 312 | RNA | Rpl31_POI_RPS9_A64-C30-histoneSL |
| 319 | RNA | Slc7a3_POI_PSMB3_A64-C30-histoneSL |
| 326 | RNA | Slc7a3_POI_CASP1_A64-C30-histoneSL |
| 333 | RNA | Slc7a3_POI_COX6B1_A64-C30-histoneSL |
| 340 | RNA | Slc7a3_POI_Gnas_A64-C30-histoneSL |
| 347 | RNA | Slc7a3_POI_Ndufa1_A64-C30-histoneSL |
| 354 | RNA | Slc7a3_POI_RPS9_A64-C30-histoneSL |
| 361 | RNA | TUBB4B_POI_RPS9_A64-C30-histoneSL |
| 368 | RNA | Ubqln2_POI_RPS9_A64-C30-histoneSL |
| 377 | RNA | RPL32_POI_ALB7_A64-C30-histoneSL |

24 hours after transfection, expression of antigen of interest was quantified by FACS analysis using standard procedures. Briefly, cells were detached (40 mM Tris HCl pH 7,5 150 mM NaCl, 1 mM EDTA in H20; 5 min at RT), washed with PBS, and stained on the surface with a mouse antibody against the antigen. Cells were resuspended in 100 μl PFEA buffer (PBS+2% FCS+2 mM EDTA+0.01% NaN₃) and analyzed using a BD FACS Canto II. Live/Dead staining was performed with Aqua fluorescent reactive dye (Invitrogen).

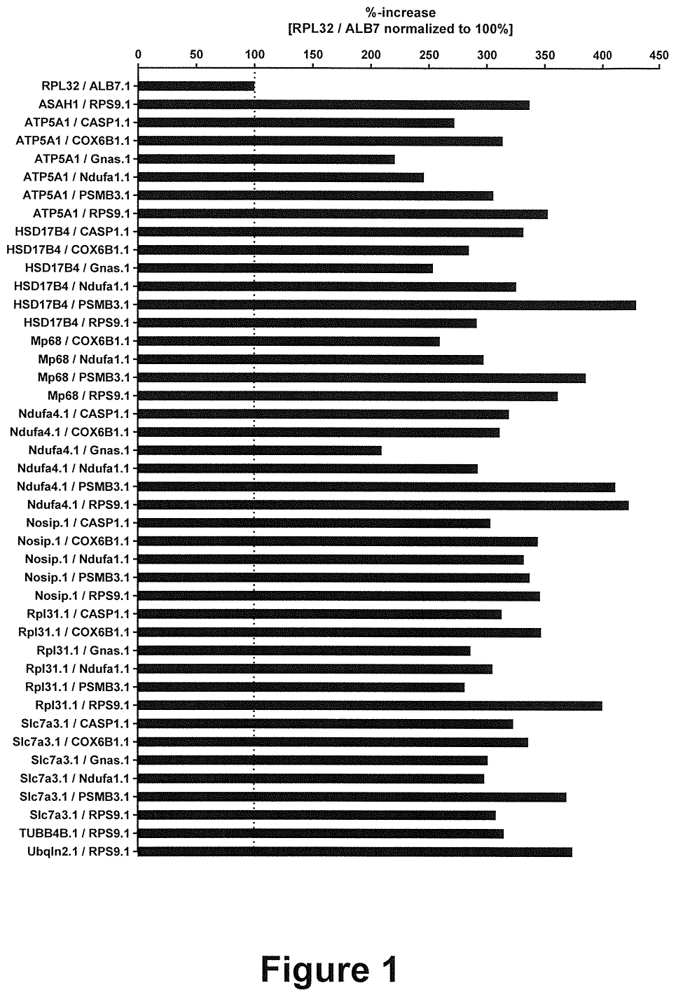

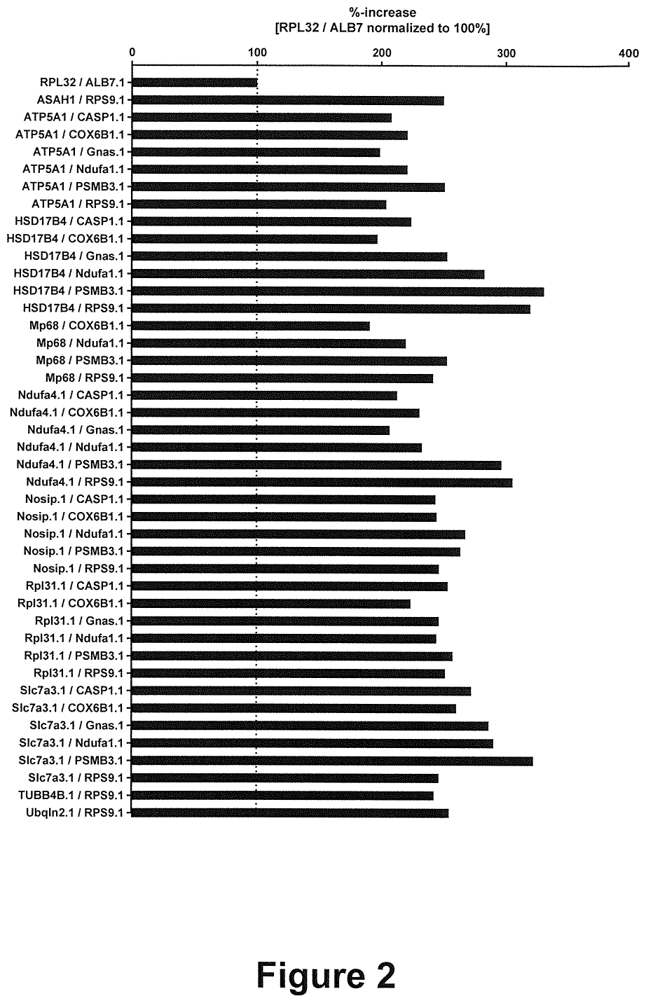

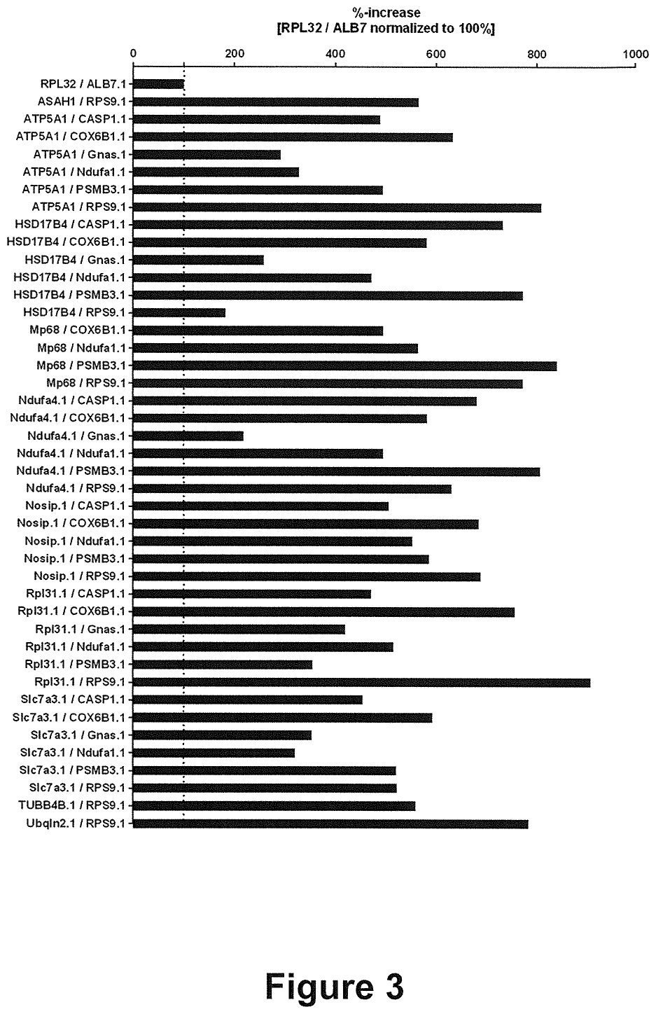

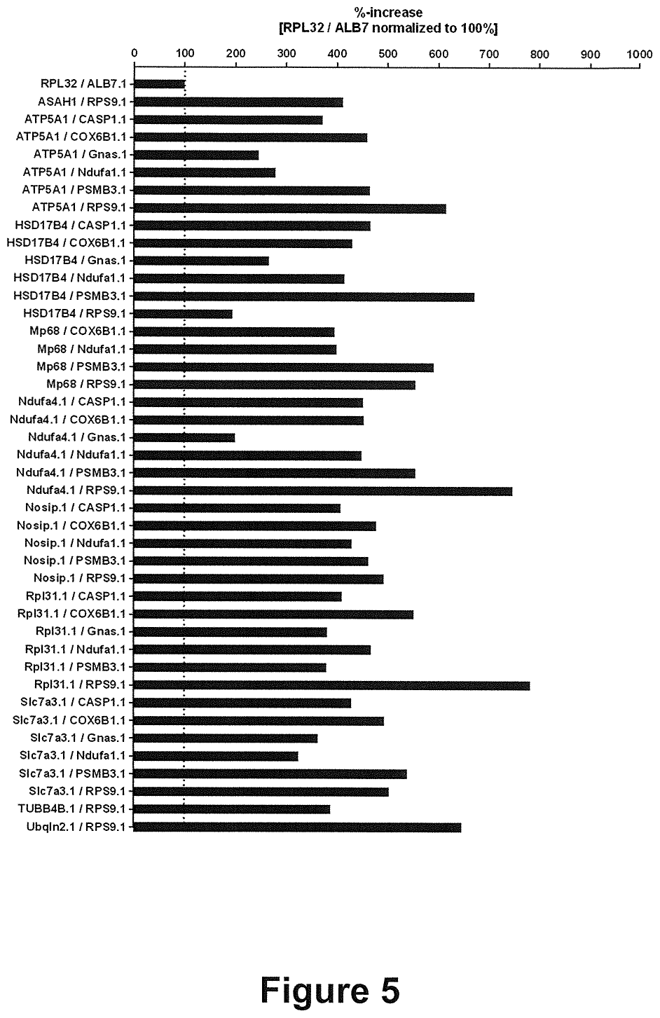

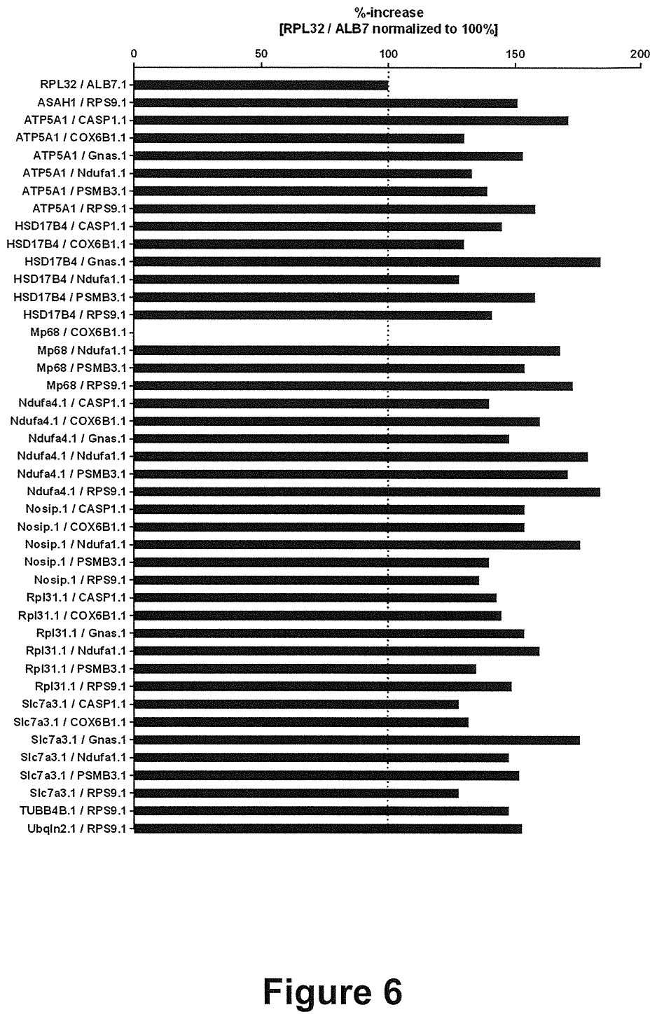

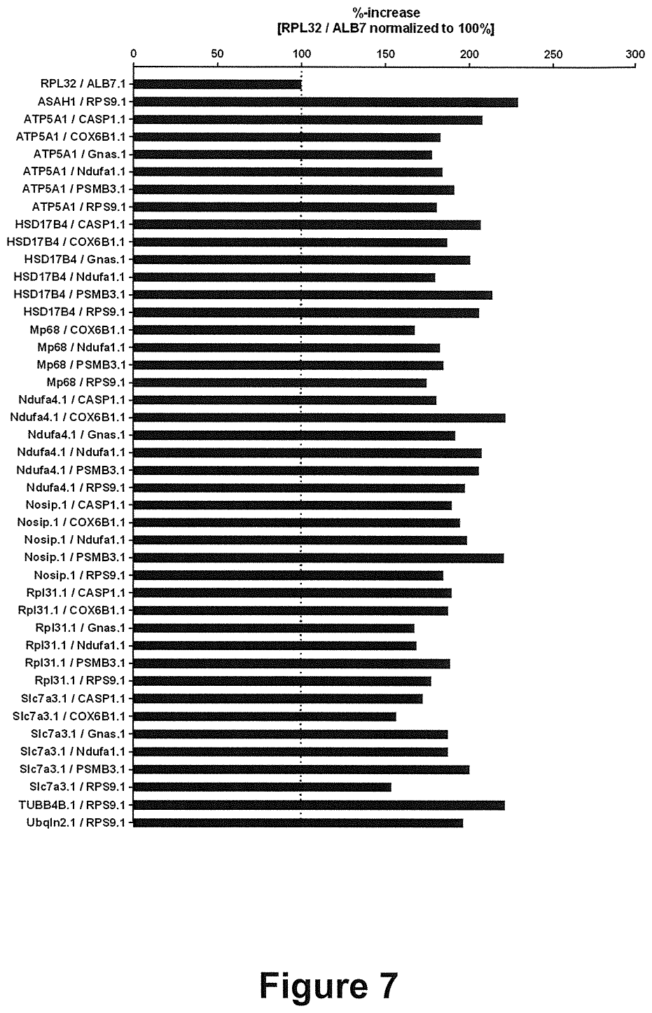

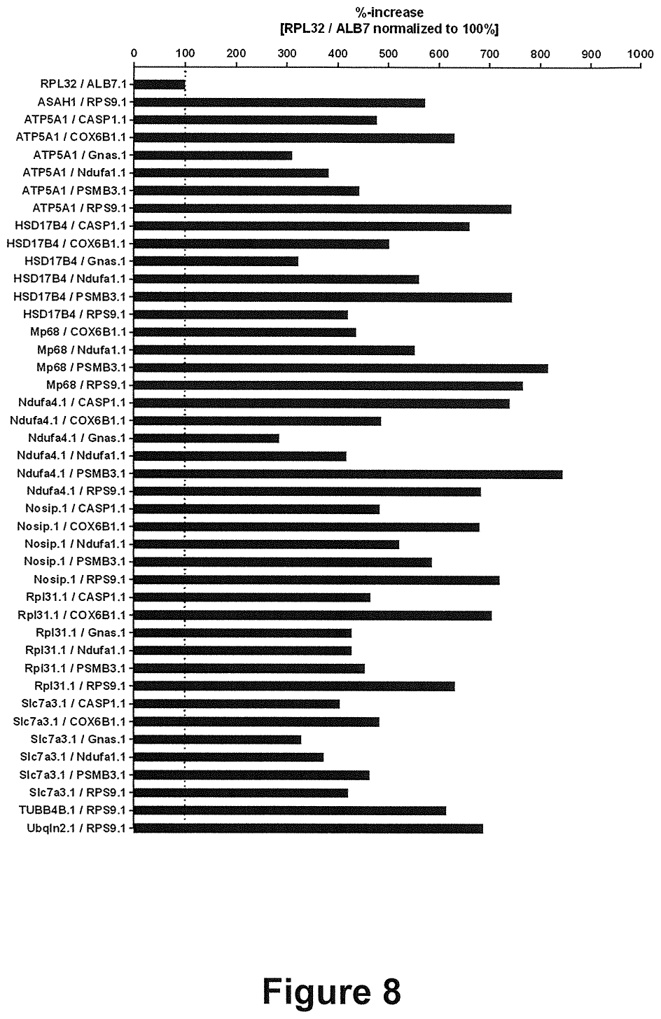

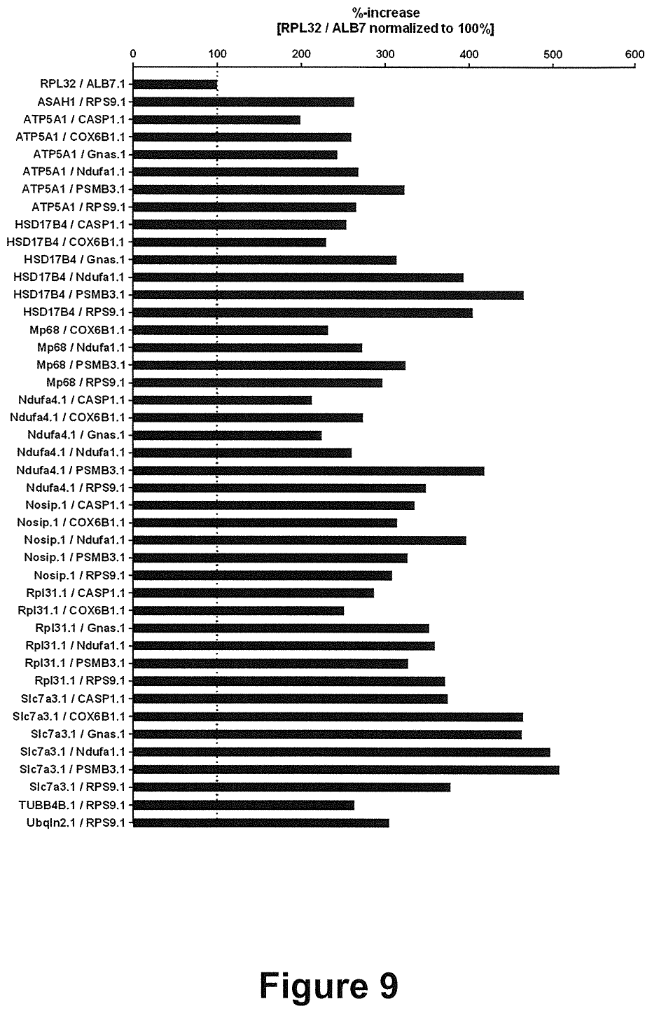

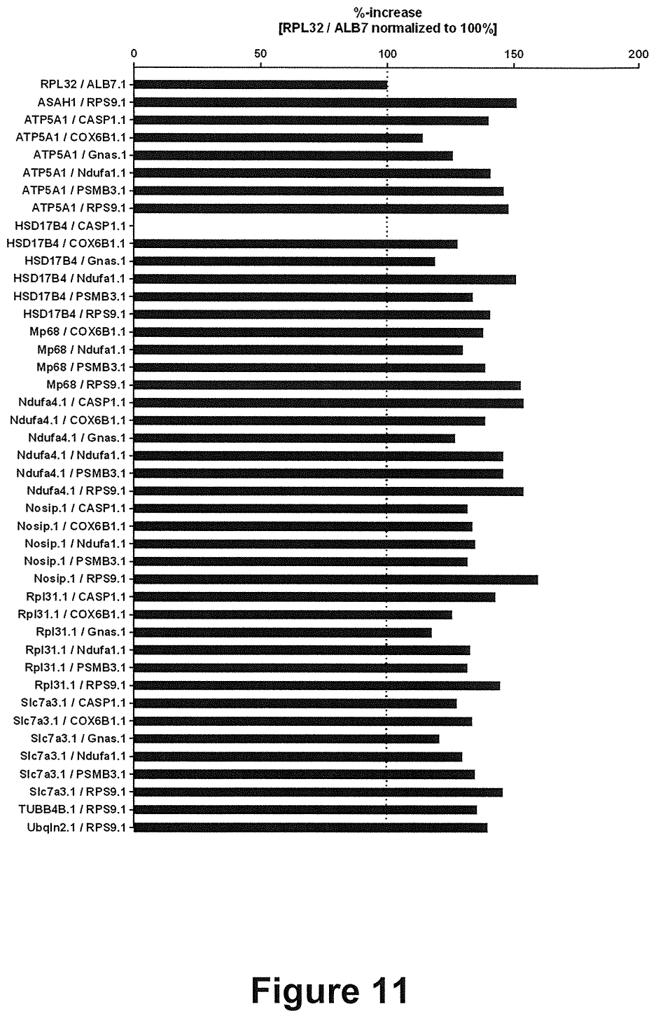

The results of protein expression from RNAs comprising the inventive UTR combinations operably linked to coding sequences encoding various proteins of interest are shown in FIG. 1-11.

As apparent, it was possible to significantly and synergically increase expression by using the inventive UTR combinations operably linked to the coding region.

### Example 6: Test for Synergy of UTR Combinations by Luciferase Expression after mRNA Transfection

Human dermal fibroblasts (HDF) were seeded 24 hours before transfection in a compatible complete cell medium on 96 well plates (10,000 cells in 200 μl/well). The day of transfection, the complete medium was replaced with serum-free Opti-MEM medium (Thermo Fisher).

Each RNA was complexed with Lipofectamine2000 at a ratio of 1/1.5 (w/v). Lipocomplexed mRNAs were then added to cells for transfection with 25 ng per well in a total volume of 200 μl. 90 minutes post start of transfection, 150 μl/well of transfection solution on HDF was exchanged for 150 μl/well of complete medium. Cells were further maintained at 37° C., 5% CO2. The sequences which were used in this Example correspond to the sequences as shown in Example 2, either with or without 5′ or respectively 3′ UTR or with both 5′ and 3′ UTRs.

In a first set of experiments, Ppluc expression was measured in cell lysates after 6 hours post start of transfection. Further sets of experiments followed after 24, 48, or 72 hours post start of transfection.

Cells were lysed by adding 100 μl of 1× passive lysis buffer (Promega, Cat. E1941) for at least 15 minutes. Lysed cells were incubated at −80° C. for at least 1 hour. Lysed cells were thawed and 20 μl were added to white LIA assay plates (Greiner Cat. 655075).

Luciferase activity was measured as relative light units (RLU) in a Plate Reader (Berthold Technologies TriStar2 LB 942). Plates were introduced into the plate reader with injection device for Beetle-juice (PJK GmbH) containing substrate for firefly luciferase. Per well, 50 μl of beetle-juice were added.

At the various time points the effect of the various UTR combinations was then determined:
the increase in expression by the 5′-UTR;
the increase in expression by the 3′-UTR;
the increase in expression by the combination of 5′-UTR and 3′-UTR in one mRNA molecule.

Next, the actual increase by combinations of 5′-UTR and 3′-UTR was divided by the predicted increase if 5′- and 3′-UTRs were acting additively to calculate the Synergy-level. Values of >1 indicates synergy i.e. more than additive effect.

The results of these experiments are shown in tables 4, 5, 6, and 7, i.e. Ppluc expression after 6, 24, 48, or 72 hours from the start of transfection.

TABLE 4
Ppluc expression in cell lysates after 6 hours post start of transfection.
Plus and minus signs in columns 2 to 5 show the result in presence
or in absence of the respective 5′-UTR or 3′-UTR

| UTR- | 5′/3′- | 5′/3′- | 5′/3′- | 5′/3′- | Synergy- |
|---|---|---|---|---|---|
| combination | UTR −/− | UTR +/− | UTR −/+ | UTR +/+ | level |
| Nosip/CASP1 | 135575 | 381228.5 | 146589 | 527953 | 1.53 |
| Nosip/COX6B1 | 135575 | 381228.5 | 91638 | 368075 | 1.15 |
| Ubqln2/CASP1 | 63374 | 149985 | 73066 | 163658 | 1.04 |

TABLE 5
Ppluc expression in cell lysates after 24 hours post start of transfection.
Plus and minus signs in columns 2 to 5 show the result in presence
or in absence of the respective 5′-UTR or 3′-UTR

| UTR- | 5′/3′- | 5′/3′- | 5′/3′- | 5′/3′- | Synergy- |
|---|---|---|---|---|---|
| combination | UTR −/− | UTR +/− | UTR −/+ | UTR +/+ | level |
| ASAH1/CASP1 | 149542 | 484451 | 212570 | 600505 | 1.13 |
| ASAH1/COX6B1 | 149542 | 484451 | 106921 | 532920 | 1.31 |
| HSD17B4/CASP1 | 149542 | 406947 | 212570 | 674820 | 1.64 |
| HSD17B4/COX6B1 | 149542 | 406947 | 106921 | 407179 | 1.20 |
| HSD17B4/RPS9 | 149542 | 406947 | 138388.5 | 490861 | 1.39 |
| Ndufa4/COX6B1 | 149542 | 274520 | 106921 | 365047 | 2.62 |
| Ndufa4/Ndufa1 | 149542 | 274520 | 90965 | 484980 | 5.05 |
| Nosip/RPS9 | 149542 | 406780.5 | 138388.5 | 632710 | 1.96 |
| Slc7a3/COX6B1 | 149542 | 329237 | 106921 | 472606 | 2.36 |
| Slc7a3/Ndufa1 | 149542 | 329237 | 90965 | 414179 | 2.18 |
| Slc7a3/PSMB3 | 149542 | 329237 | 167562.5 | 404766 | 1.29 |
| Slc7a3/RPS9 | 149542 | 329237 | 138388.5 | 399851 | 1.49 |

TABLE 6
Ppluc expression in cell lysates after 48 hours post start of transfection.
Plus and minus signs in columns 2 to 5 show the result in presence
or in absence of the respective 5′-UTR or 3′-UTR

| UTR- | 5′/3′- | 5′/3′- | 5′/3′- | 5′/3′- | Synergy- |
|---|---|---|---|---|---|
| combination | UTR −/− | UTR +/− | UTR −/+ | UTR +/+ | level |
| ASAH1/Ndufa1 | 54975 | 170288.5 | 35595 | 213333 | 1.65 |
| ASAH1/RPS9 | 54975 | 170288.5 | 60014.5 | 209830 | 1.29 |
| ATP5A1/Ndufa1 | 54975 | 217186 | 35595 | 289947 | 1.65 |
| HSD17B4/Ndufa1 | 54975 | 165220.5 | 35595 | 299354 | 2.69 |
| Mp68/Ndufa1 | 54975 | 160203 | 35595 | 257522 | 2.36 |
| Ndufa4/CASP1 | 54975 | 120018 | 98627 | 198338 | 1.32 |
| Ndufa4/PSMB3 | 54975 | 120018 | 82027 | 252222 | 2.14 |
| Ndufa4/RPS9 | 54975 | 120018 | 60014.5 | 186654 | 1.88 |
| Nosip/PSMB3 | 54975 | 149163 | 82027 | 240696 | 1.53 |
| Rpl31/CASP1 | 54975 | 121611 | 98627 | 258646 | 1.85 |
| Rpl31/COX6B1 | 54975 | 121611 | 60551 | 198202 | 1.98 |
| Rpl31/Ndufa1 | 54975 | 121611 | 35595 | 184597 | 2.74 |
| Rpl31/RPS9 | 54975 | 121611 | 60014.5 | 200615 | 2.03 |
| Slc7a3/CASP1 | 54975 | 162686 | 98627 | 267008 | 1.40 |

TABLE 7
Ppluc expression in cell lysates after 72 hours post start of transfection.
Plus and minus signs in columns 2 to 5 show the result in presence
or in absence of the respective 5′-UTR or 3′-UTR

| UTR- | 5′/3′- | 5′/3′- | 5′/3′- | 5′/3′- | Synergy- |
|---|---|---|---|---|---|
| combination | UTR −/− | UTR +/− | UTR −/+ | UTR +/+ | level |
| ASAH1/PSMB3 | 20629 | 71091.5 | 34584 | 87632 | 1.04 |
| ATP5A1/RPS9 | 20629 | 70800 | 31140 | 90507 | 1.15 |
| Mp68/COX6B1 | 20629 | 55916 | 25431 | 75541 | 1.37 |
| Mp68/PSMB3 | 20629 | 55916 | 34584 | 90988 | 1.43 |
| Rpl31/PSMB3 | 20629 | 48122 | 34584 | 143358 | 2.96 |
| TUBB4B/Ndufa1 | 20629 | 60919 | 19792 | 109249 | 2.25 |
| TUBB4B/RPS9 | 20629 | 60919 | 31140 | 79304 | 1.15 |
| Ubqln2/COX6B1 | 20629 | 63721 | 25431 | 76275 | 1.16 |
| Ubqln2/Ndufa1 | 20629 | 63721 | 19792 | 100361 | 1.89 |
| Ubqln2/PSMB3 | 20629 | 63721 | 34584 | 117729 | 1.70 |
| Ubqln2/RPS9 | 20629 | 63721 | 31140 | 89655 | 1.29 |

As apparent, it was clearly possible to prove synergy effects of UTR combinations by Luciferase expression.

1. An artificial nucleic acid molecule comprising
a) at least one 5′ untranslated region (5′ UTR) element derived from a 5′ UTR of a gene selected from the group consisting of HSD17B4, ASAH1, ATP5A1, MP68, NDUFA4, NOSIP, RPL31, SLC7A3, TUBB4B and UBQLN2;
b) at least one 3′ untranslated region (3′ UTR) element derived from a 3′ UTR of a gene selected from the group consisting of PSMB3, CASP1, COX6B1, GNAS, NDUFA1 and RPS9; and optionally
c) at least one coding region operably linked to said 5′ UTR and said 3′ UTR.

2. The artificial nucleic acid molecule according to claim 1, wherein said 5′ UTR and/or said 3′ UTR is heterologous to said coding region.

3. (canceled)

4. The artificial nucleic acid molecule according to claim 1, comprising
a-1) at least one 5′ UTR element derived from a 5′UTR of a HSD17B4 gene, or from a corresponding RNA sequence; or
a-2) at least one 5′ UTR element derived from a 5′UTR of a NDUFA4 gene, or from a corresponding RNA sequence; or
a-3) at least one 5′ UTR element derived from a 5′UTR of a SLC7A3 gene, or from a corresponding RNA sequence; or
a-4) at least one 5′ UTR element derived from a 5′UTR of a NOSIP gene, or from a corresponding RNA sequence; or
a-5) at least one 5′ UTR element derived from a 5′UTR of a MP68 gene, or from a corresponding RNA sequence; or
b-1) at least one 5′ UTR element derived from a 5′UTR of a UBQLN2 gene, or from a corresponding RNA sequence; or
b-2) at least one 5′ UTR element derived from a 5′UTR of a ASAH1 gene, or from a corresponding RNA sequence; or
b-3) at least one 5′ UTR element derived from a 5′UTR of a HSD17B4 gene, or from a corresponding RNA sequence; or
b-4) at least one 5′ UTR element derived from a 5′UTR of a HSD17B4 gene, or from a corresponding RNA sequence; or
b-5) at least one 5′ UTR element derived from a 5′UTR of a NOSIP gene, or from a corresponding RNA sequence; or
c-1) at least one 5′ UTR element derived from a 5′UTR of a NDUFA4 gene, or from a corresponding RNA sequence; or
c-2) at least one 5′ UTR element derived from a 5′UTR of a NOSIP gene, or from a corresponding RNA sequence; or
c-3) at least one 5′ UTR element derived from a 5′UTR of a NDUFA4 gene, or from a corresponding RNA sequence; or
c-4) at least one 5′ UTR element derived from a 5′UTR of a NDUFA4 gene, or from a corresponding RNA sequence; or
c-5) at least one 5′ UTR element derived from a 5′UTR of a ATP5A1 gene, or from a corresponding RNA sequence; or
d-1) at least one 5′ UTR element derived from a 5′UTR of a RPL31 gene, or from a corresponding RNA sequence; or
d-2) at least one 5′ UTR element derived from a 5′UTR of a ATP5A1 gene, or from a corresponding RNA sequence; or
d-3) at least one 5′ UTR element derived from a 5′UTR of a SLC7A3 gene, or from a corresponding RNA sequence; or
d-4) at least one 5′ UTR element derived from a 5′UTR of a HSD17B4 gene, or from a corresponding RNA sequence; or
d-5) at least one 5′ UTR element derived from a 5′UTR of a SLC7A3 gene, or from a corresponding RNA sequence; or
e-1) at least one 5′ UTR element derived from a 5′UTR of a TUBB4B gene, or from a corresponding RNA sequence; or
e-2) at least one 5′ UTR element derived from a 5′UTR of a RPL31 gene, or from a corresponding RNA sequence, or
e-3) at least one 5′ UTR element derived from a 5′UTR of a MP68 gene, or from a corresponding RNA sequence; or
e-4) at least one 5′ UTR element derived from a 5′UTR of a NOSIP gene, or from a corresponding RNA sequence; or
e-5) at least one 5′ UTR element derived from a 5′UTR of a ATP5A1 gene, or from a corresponding RNA sequence; or
e-6) at least one 5′ UTR element derived from a 5′UTR of a ATP5A1 gene, or from a corresponding RNA sequence; or
f-1) at least one 5′ UTR element derived from a 5′UTR of a ATP5A1 gene, or from a corresponding RNA sequence; or
f-2) at least one 5′ UTR element derived from a 5′UTR of a ATP5A1 gene, or from a corresponding RNA sequence; or
f.3) at least one 5′ UTR element derived from a 5′UTR of a HSD17B4 gene, or from a corresponding RNA sequence; or
f-4) at least one 5′ UTR element derived from a 5′UTR of a HSD17B4 gene, or from a corresponding RNA sequence; or
f-5) at least one 5′ UTR element derived from a 5′UTR of a MP68 gene, or from a corresponding RNA sequence; or
g-1) at least one 5′ UTR element derived from a 5′UTR of a MP68 gene, or from a corresponding RNA sequence; or
g-2) at least one 5′ UTR element derived from a 5′UTR of a NDUFA4 gene or from a corresponding RNA sequence; or
g-3) at least one 5′ UTR element derived from a 5′UTR of a NDUFA4 gene, or from a corresponding RNA sequence; or
g-4) at least one 5′ UTR element derived from a 5′UTR of a NOSIP gene, or from a corresponding RNA sequence; or
g-5) at least one 5′ UTR element derived from a 5′UTR of a RPL31 gene, or from a corresponding RNA sequence; or
h-1) at least one 5′ UTR element derived from a 5′UTR of a RPL31 gene, or from a corresponding RNA sequence; or
h-2) at least one 5′ UTR element derived from a 5′UTR of a RPL31 gene, or from a corresponding RNA sequence; or
h-3) at least one 5′ UTR element derived from a 5′UTR of a RPL31 gene, or from a corresponding RNA sequence; or
h-4) at least one 5′ UTR element derived from a 5′UTR of a SLC7A3 gene, or from a corresponding RNA sequence; or
h-5) at least one 5′ UTR element derived from a 5′UTR of a SLC7A3 gene, or from a corresponding RNA sequence; or
i-1) at least one 5′ UTR element derived from a 5′UTR of a SLC7A3 gene, or from a corresponding RNA sequence; or
i-2) at least one 5′ UTR element derived from a 5′UTR of a Ndufa4.1 gene, or from a corresponding RNA sequence.

5. The artificial nucleic acid molecule according to claim 4, comprising UTR elements according to a-1, a-2, a-3, a-4 or a-5.

6-10. (canceled)

11. The artificial nucleic acid molecule according to claim 1, wherein the at least one coding region encodes at least one (poly-)peptide or protein of interest.

12. The artificial nucleic acid molecule according to claim 11, wherein said at least one antigenic (poly-)peptide or protein is selected from a tumor antigen, a pathogenic antigen, an autoantigen, an alloantigen, or an allergenic antigen.

13. The artificial nucleic acid molecule according to claim 12, wherein said at least one pathogenic antigen is selected from a bacterial, viral, fungal or protozoal antigen.

14-18. (canceled)

19. The artificial nucleic acid molecule according to claim 1, wherein said artificial nucleic acid molecule is an RNA.

20. (canceled)

21. The artificial nucleic acid molecule according to claim 19, wherein the RNA is an mRNA, a viral RNA, self-replicating RNA or a replicon RNA.

22. (canceled)

23. The artificial nucleic acid molecule according to claim 1, wherein
the G/C content of the at least one coding region of the artificial nucleic acid is increased compared to the G/C content of the corresponding coding sequence of the corresponding wild-type artificial nucleic acid, and/or wherein
the C content of the at least one coding region of the artificial nucleic acid is increased compared to the C content of the corresponding coding sequence of the corresponding wild-type artificial nucleic acid, and/or wherein
the codons in the at least one coding region of the artificial nucleic acid are adapted to human codon usage, wherein the codon adaptation index (CAI) is preferably increased or maximised in the at least one coding sequence of the artificial nucleic acid,
wherein the amino acid sequence encoded by the artificial nucleic acid is preferably not being modified compared to the amino acid sequence encoded by the corresponding wild-type artificial nucleic acid.

24. The artificial nucleic acid molecule according to claim 1, which comprises a 5′-CAP structure.

25. The artificial nucleic acid molecule according to claim 19, which comprises at least one histone stem-loop.

26-27. (canceled)

28. The artificial nucleic acid molecule according to claim 19, comprising a poly(A) sequence of 10 to 200 adenosine nucleotides.

29-30. (canceled)

31. A composition comprising at least one or a plurality of artificial nucleic acid molecule(s) according to claim 1 and a pharmaceutically acceptable carrier and/or excipient.

32-43. (canceled)

44. A kit comprising the artificial nucleic acid molecule according to claim 1, and optionally a liquid vehicle and/or optionally technical instructions with information on the administration and dosage of the artificial nucleic acid molecule.

45-51. (canceled)

52. A method of treating or preventing a disorder optionally selected from genetic diseases, cancer, infectious diseases, inflammatory diseases, (auto)immune diseases, allergies, and/or for use in gene therapy and/or immunomodulation, wherein said method comprises administering to a subject in need thereof an effective amount of the artificial nucleic acid molecule according to claim 1.

53-54. (canceled)

55. The artificial nucleic acid molecule according to claim 4, comprising UTR elements according to a-1.

56. The artificial nucleic acid molecule according to claim 55, wherein said 5′UTR element derived from a HSD17B4 gene comprises a sequence at least 95% identical to the sequence of SEQ ID NO: 1.

57. The artificial nucleic acid molecule according to claim 1, wherein said 3′UTR element derived from a PSMB3 gene comprises a sequence at least 95% identical to the sequence of SEQ ID NO: 23.

58. The artificial nucleic acid molecule according to claim 1, wherein the artificial nucleic acid sequence is a RNA and wherein the RNA comprises:
a 5′ UTR element derived from a HSD17B4 gene comprises a sequence at least 95% identical to the sequence of SEQ ID NO: 1; and
3′ UTR element derived from a PSMB3 gene comprises a sequence at least 95% identical to the sequence of SEQ ID NO: 23.
